(19) United States
(12) Patent Application Publication
(10) Pub. No.: US 2006/0205697 A1
(43) Pub. Date: Sep. 14, 2006

(54) Novel betulin derivatives, preparation thereof and use thereof

(76) Inventors: Gary N. Robinson, Gaithersburg, MD (US); Carl T. Wild, Gaithersburg, MD (US); Mark Ashton, Oxford (GB); Russell Thomas, Siena (IT); Christian Montalbetti, Wallingford (GB); Thomas Stephen Coulter, Wantage (GB); Filippo Magaraci, Didcot (GB); Robert James Townsend, Abingdon (GB); Theodore John Nitz, Pottstown, PA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/272,019

(22) Filed: Nov. 14, 2005

Related U.S. Application Data
(60) Provisional application No. 60/626,886, filed on Nov. 12, 2004.
(60) Provisional application No. 60/653,080, filed on Feb. 16, 2005.

(51) Int. Cl. A61K 31/66; C07J 51/00; C07J 53/00; A61K 31/58

(52) U.S. Cl. 514/129; 514/169; 514/176; 552/506; 552/514

(57) ABSTRACT

The present invention relates to novel synthetic derivatives of betulin and the use of such derivatives as pharmaceuticals. The present invention is directed to novel compounds of Formula I: 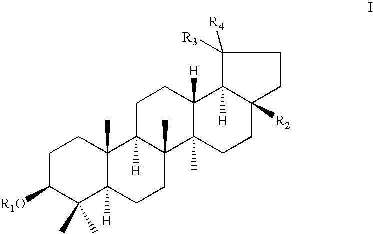 or a pharmaceutically acceptable salt or prodrug thereof.

This application claims the benefit of the filing date of U.S. Appl. No. 60/626,886, filed Nov. 12, 2004 and U.S. Appl. No. 60/653,080, filed Feb. 16, 2005, both of which are incorporated by reference herein in their entirety.

### BACKGROUND OF THE INVENTION

1. Field of the Invention

The present invention relates to novel synthetic derivatives of betulin and the use of such derivatives as pharmaceuticals.

2. Related Art

Retroviruses are small, single-stranded positive-sense RNA viruses. A retroviral particle comprises two identical single-stranded positive sense RNA molecules. Their genome contains, among other things, the sequence of the RNA-dependent DNA polymerase, also known as reverse transcriptase. Many molecules of reverse transcriptase are found in close association with the genomic RNA in the mature viral particles. Upon entering a cell, this reverse transcriptase produces a double-stranded DNA copy of the viral genome, which is then inserted into the chromatin of a host cell. Once inserted, the viral sequence is called a provirus. Retroviral integration is directly dependent upon viral proteins. Linear viral DNA termini (the LTRs) are the immediate precursors to the integrated proviral DNA. There is a characteristic duplication of short stretches of the host's DNA at the site of integration.

Progeny viral genomes and mRNAs are transcribed from the inserted proviral sequence by host cell RNA polymerase in response to transcriptional, regulatory signals in the terminal regions of the proviral sequence, the long terminal repeats, or LTRs. The host cell's protein production machinery is used to produce viral proteins, many of which are inactive until processed by virally encoded proteases. Typically, progeny viral particles bud from the cell surface in a non-lytic manner. Retroviral infection does not necessarily interfere with the normal life cycle of an infected cell or organism. However, neither is it always benign with respect to the host organism. While most classes of DNA viruses can be implicated in tumorigenesis, retroviruses are the only taxonomic group of RNA viruses that are oncogenic. Various retroviruses, such as Human Immunodeficiency Virus (HIV), which is the etiological agent responsible for acquired immune deficiency syndrome (AIDS) in humans, are also responsible for several very unusual diseases of the immune system of higher animals.

Human Immunodeficiency Virus (HIV) is a member of the lentiviruses, a subfamily of retroviruses. HIV infects and invades cells of the immune system; it breaks down the body's immune system and renders the patient susceptible to opportunistic infections and neoplasms. The immune defect appears to be progressive and irreversible, with a high mortality rate that approaches 100% over several years.

HIV-1 is trophic and cytopathic for T4 lymphocytes, cells of the immune system which express the cell surface differentiation antigen CD4, also known as OKT4, T4 and leu3. The viral tropism is due to the interactions between the viral envelope glycoprotein, gp120, and the cell-surface CD4 molecules (Dalgleish et al., Nature 312:763-767 (1984)). These interactions not only mediate the infection of susceptible cells by HIV, but are also responsible for the virus-induced fusion of infected and uninfected T cells. This cell fusion results in the formation of giant multinucleated syncytia, cell death, and progressive depletion of CD4 cells in HIV-infected patients. These events result in HIV-induced immunosuppression and its subsequent sequelae, opportunistic infections and neoplasms.

In addition to CD4+ T cells, the host range of HIV includes cells of the mononuclear phagocytic lineage (Dalgleish et al., supra), including blood monocytes, tissue macrophages, Langerhans cells of the skin and dendritic reticulum cells within lymph nodes. HIV is also neurotropic, capable of infecting monocytes and macrophages in the central nervous system causing severe neurologic damage. Macrophage/monocytes are a major reservoir of HIV. They can interact and fuse with CD4-bearing T cells, causing T cell depletion and thus contributing to the pathogenesis of AIDS.

Considerable progress has been made in the development of drugs for HIV-1 therapy during the past few years. Therapeutic agents for HIV include, but are not limited to, AZT, 3TC, ddC, d4T, ddI, tenofovir, abacavir, nevirapine, delavirdine, emtricitabine, efavirenz, saquinavir, ritonavir, indinavir, nelfinavir, lopinavir, amprenavir, fosamprenavir, tipranavir, and atazanavir or any other antiretroviral drugs or antibodies in combination with each other, or associated with a biologically based therapeutic, such as, for example, gp41-derived peptides enfuvirtide (Fuzeon; Trimeris-Roche) and T-1249 (Trimeris), or soluble CD4, antibodies to CD4, and conjugates of CD4 or anti-CD4. Combinations of these drugs are particularly effective and can reduce levels of viral RNA to undetectable levels in the plasma and slow the development of viral resistance, with resulting improvements in patient health and life span.

Despite these advances, there are still problems with the currently available drug regimens. Many of the drugs exhibit severe toxicities, have other side-effects (e.g., fat redistribution) or require complicated dosing schedules that reduce compliance and thereby limit efficacy. Resistant strains of HIV often appear over extended periods of time even on combination therapy. The high cost of these drugs is also a limitation to their widespread use, especially outside of developed countries.

There is still a major need for the development of additional drugs to circumvent these issues. Ideally these would target different stages in the viral life cycle, adding to the armamentarium for combination therapy, and exhibit minimal toxicity, yet have lower manufacturing costs.

Previously, betulinic acid and platanic acid were isolated as anti-HIV principles from Syzigium claviflorum. Betulinic acid and platanic acid exhibited inhibitory activity against HIV-1 replication in H9 lymphocyte cells with EC₅₀ values of 1.4 μM and 6.5 μM, respectively, and T.I. values of 9.3 and 14, respectively. Hydrogenation of betulinic acid yielded dihydrobetulinic acid, which showed slightly more potent anti-HIV activity with an EC₅₀ value of 0.9 and a T.I. value of 14 (Fujioka, T., et al., J. Nat. Prod. 57:243-247 (1994)).

Esterification of betulinic acid with certain substituted acyl groups, such as 3′,3′-dimethylglutaryl and 3′,3′-dimethylsuccinyl groups produced derivatives having enhanced activity (Kashiwada, Y., et al., J. Med. Chem. 39:1016-1017 (1996)). Acylated betulinic acid and dihydrobetulinic acid derivatives that are potent anti-HIV agents are also described in U.S. Pat. No. 5,679,828.

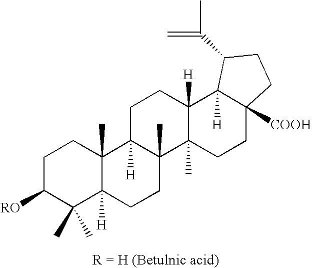

U.S. Pat. No. 5,468,888 discloses 28-amido derivatives of lupanes that are described as having a cytoprotecting effect for HIV-infected cells.

Japanese Patent Application No. JP 01 143,832 discloses that betulin and 3,28-diesters thereof are useful in the anti-cancer field.

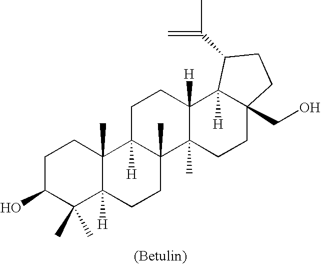

Esterification of the 3 carbon of betulin with succinic acid produced a compound capable of inhibiting HIV-1 activity (Pokrovskii, A. G. et al., Gos. Nauchnyi Tsentr Virusol. Biotekhnol. “Vector” 9:485-491 (2001)).

A need continues to exist for compounds which possess potent antiretroviral activity, especially anti-HIV activity, with improved biodistribution properties and different modes of action. Such compounds are urgently needed to add to existing anti-HIV therapies. There is also a need for safe and effective compounds that can be topically applied to vaginal or other mucosa to prevent HIV infections between individuals.

### SUMMARY OF THE INVENTION

The present invention is related to novel betulin derivative compounds having Formula I, 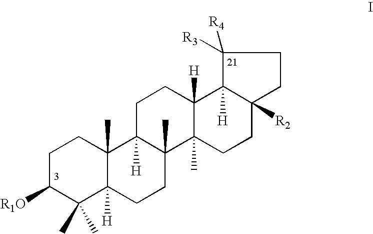
or a pharmaceutically acceptable salt or prodrug thereof, wherein the substituents are as defined herein.

Another aspect of the present invention is directed to pharmaceutical compositions, comprising one or more compounds of Formula I, and a pharmaceutically acceptable carrier or diluent. One or more additional pharmaceutically active compounds can also be included in these compositions.

The compounds of Formula I are useful as anti-retroviral agents. Therefore, the present invention provides methods for inhibiting a retroviral infection in cells or tissue of an animal, comprising administering an effective retroviral inhibiting amount of a compound of Formula I. Some embodiments are directed to a method for treating a patient suffering from a retroviral-related pathology, comprising administering to the subject a retroviral inhibiting effective amount of a pharmaceutical composition that includes a compound of Formula I. Also included is a method of treating HIV-infected cells, wherein the HIV infecting said cells does not respond to other HIV therapies.

The betulin derivatives of Formula I can be used in a combination therapy with one or more antiviral agents. Thus, the present invention provides a method of treating a patient suffering from a retroviral-related pathology, comprising administering to the patient a retroviral inhibiting effective amount of at least one compound of Formula I in combination with one or more antiviral agents. The present invention is also directed to a method for treating a subject infected with HIV-1 by administering at least one of the above-noted betulin derivatives, optionally in combination with any one or more of the known anti-AIDS therapeutics or an immunostimulant.

The present invention also provides a method of preventing transmission of HIV infection between individuals. In particular, the present invention provides a method of preventing transmission of HIV infection from an HIV infected pregnant woman to a fetus, comprising administering to the woman and/or the fetus a retroviral inhibiting effective amount of one or more compounds of Formula I during pregnancy or immediately prior to, at, or subsequent to birth.

Further, the present invention provides a method of preventing transmission of HIV infection during sexual intercourse, comprising applying a retroviral inhibiting effective amount of a topical composition including one or more compounds of Formula I to vaginal or other mucosa prior to sexual intercourse.

Furthermore, the present invention is directed to a method for making compounds of Formula I.

Additional embodiments and advantages of the invention will be set forth in part in the description as follows, and in part will be obvious from the description, or can be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DETAILED DESCRIPTION

The present invention is directed to compounds having Formula I: 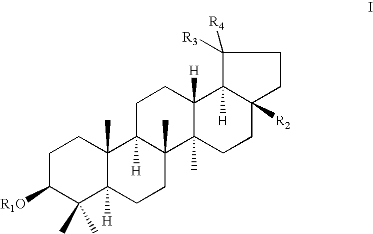
or a pharmaceutically acceptable salt or prodrug thereof, wherein:

R₁ is C₃-C₂₀ alkanoyl, carboxyalkanoyl, carboxyalkenoyl, alkoxycarbonylalkanoyl, alkenyloxycarbonylalkanoyl, cyanoalkanoyl, hydroxyalkanoyl, aminocarbonylalkanoyl, hydroxyaminocarbonylalkanoyl, monoalkylaminocarbonylalkanoyl, dialkylaminocarbonylalkanoyl, heteroarylalkanoyl, heterocyclylalkanoyl, heterocyclylcarbonylalkanoyl, heteroarylaminocarbonylalkanoyl, heterocyclylaminocarbonylalkanoyl, cyanoaminocarbonylalkanoyl, alkylsulfonylaminocarbonylalkanoyl, arylsulfonylaminocarbonylalkanoyl, sulfoaminocarbonylalkanoyl, phosphonoaminocarbonylalkanoyl, phosphono, sulfo, phosphonoalkanoyl, sulfoalkanoyl, alkylsulfonylalkanoyl, or alkylphosphonoalkanoyl;

R₂ is formyl, carboxyalkenyl, heterocyclyl, heteroaryl, —CH₂SR₁₄, CH₂SOR₁₄, CH₂SO₂R₁₄,

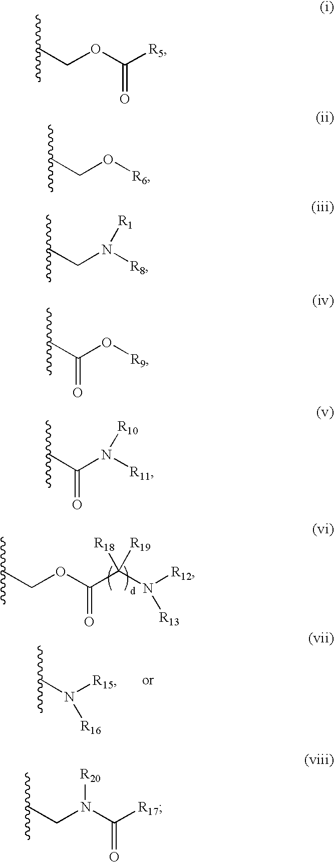

R₃ is hydrogen, hydroxyl, isopropenyl, isopropyl, 1′-hydroxyisopropyl, 1′-haloisopropyl, 1′-thioisopropyl, 1′-trifluoromethylisopropyl, 2′-hydroxyisopropyl, 2′-haloisopropyl, 2′-thioisopropyl, 2′-trifluoromethylisopropyl, 1′-hydroxyethyl, 1′-(alkoxy)ethyl, 1′-(alkoxyalkoxy)ethyl, 1′-(arylalkoxy)ethyl; 1′-(arylcarbonyloxy)ethyl, acetyl, 1′-(hydroxyl)-1′-(hydroxyalkyl)ethyl, (2′-oxo)tetrahydrooxazolyl, 1′,2′-epoxyisopropyl, 2′-haloisopropenyl, 2′-hydroxyisopropenyl, 2′-aminoisopropenyl, 2′-thioisopropenyl, 3′-haloisopropenyl, 3′-hydroxyisopropenyl, 3′-aminoisopropenyl, 3′-thioisopropenyl, 1′-alkoxyethyl, 1′-hydroxyiminoethyl, 1′-alkoxyiminoethyl, or

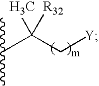

wherein Y is —SR₃₃ or —NR₃₃R₃₄;

R₃₂ is hydrogen or hydroxy;

R₃₃ and R₃₄ are independently hydrogen, alkyl, alkanoyl, arylalkyl, heteroarylalkyl, arylsulfonyl or arylaminocarbonyl; or

R₃₃ and R₃₄ can be taken together with the nitrogen to which they are attached to form a heterocycle, wherein the heterocycle can optionally include one or more additional nitrogen, sulfur or oxygen atoms;

m is zero to three;

R₄ is hydrogen; or

R₃ and R₄ can be taken together to form oxo, alkylimino, alkoxyimino or benzyloxyimino;

R₅ is C₂-C₂₀ alkyl, alkenyl, alkynyl, carboxy(C₂-C₂₀)alkyl, amino, aminoalkyl, dialkylamino, monoalkylaminoalkyl, dialkylaminoalkyl, alkoxyalkyl, cyanoalkyl, alkylthioalkyl, cycloalkyl, cycloalkylalkyl, aryl, arylalkyl, heterocyclyl, heteroaryl, heterocyclylalkyl, heteroarylalkyl, sulfo, phosphono, sulfoalkyl, phosphonoalkyl, alkylsulfonyl, alkylphosphono, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, arylphosphonoaminocarbonylalkyl, alkylphosphonoaminocarbonylalkyl, or hydroxyimino(amino)alkyl;

R₆ is hydrogen, phosphono, sulfo, alkyl, sulfoalkyl, phosphonoalkyl, alkylsulfonyl, alkylphosphono, cycloalkyl, cycloalkenyl, heterocyclyl, aryl, heteroaryl, carboxyalkyl, alkoxycarbonylalkyl, cyanoalkyl; CH₂CONR₇R₈, trialkylsilyl, ethoxyethyl (OEE), or tetrahydropyranyl ether (OTHP);

R₇ and R₈ are independently hydrogen, alkyl, aminoalkyl, monoalkylaminoalkyl, dialkylaminoalkyl, carboxyalkyl, alkoxyalkyl, alkoxyalkoxyalkyl, alkoxycarbonylaminoalkoxyalkyl, alkoxycarbonylaminoalkyl, aminoalkoxyalkyl, alkylcarbonylaminoalkyl, heterocyclyl, heterocyclylalkyl, aryl, arylalkyl, arylcarbonylaminoalkyl, cycloalkyl, alkylsulfonyl, arylsulfonyl, or heteroarylsulfonyl, heterocyclylsulfonyl, or R₇ and R₈ can together with the nitrogen atom to which they are attached form a heterocyclyl or heteroaryl group, wherein the heterocyclyl or heteroaryl can optionally include one or more additional nitrogen, sulfur or oxygen atoms;

R₉ is hydrogen, phosphono, sulfo, alkyl, alkenyl, trialkylsilyl, cycloalkyl, carboxyalkyl, alkoxycarbonyloxyalkyl, aminoalkyl, monoalkylaminoalkyl, dialkylaminoalkyl, alkoxyalkyl, cyanoalkyl, phosphonoalkyl, sulfoalkyl, alkylsulfonyl, alkylphosphono, aryl, heteroaryl, heterocyclyl, arylalkyl, heteroarylalkyl, heterocyclylalkyl, or dialkoxyalkyl;

R₁₀ and R₁₁ are independently hydrogen, alkyl, amino, aminoalkyl, monoalkylaminoalkyl, dialkylaminoalkyl, carboxyalkyl, alkanoyloxyalkyl, alkoxyalkyl, hydroxyalkyl, cyanoalkyl, alkoxyalkoxyalkyl, alkoxycarbonylaminoalkoxyalkyl, alkoxycarbonylalkyl, hydroxycarbonylalkyl, alkoxycarbonylaminoalkyl, aminoalkoxyalkyl, alkylcarbonylaminoalkyl, heterocyclyl, heterocyclylalkyl, aryl, heteroarylalkyl, arylalkyl, arylcarbonylaminoalkyl, heterocyclylheterocyclylalkyl, heterocyclylarylalkyl, arylaminoalkyl, aminocycloalkyl, alkylsulfonyl, arylsulfonyl, alkylsulfonylaminoalkyl, arylsulfonylaminoalkyl, or cycloalkyl, or alkyl interrupted by one or more oxygen atoms, or R₁₀ and R₁₁ can together with the nitrogen atom to which they are attached form a heterocyclyl group, wherein the heterocyclyl can optionally include one or more additional nitrogen, sulfur or oxygen atoms;

R₁₂ and R₁₃ are independently hydrogen, alkyl, alkenyl, alkylamino, alkynyl, alkoxy, alkoxycarbonyl, alkoxyaminoalkyl, cycloalkyloxo, heterocyclylaminoalkyl, cycloalkyl, cyanoalkyl, cyano, sulfo, phosphono, sulfoalkyl, phosphonoalkyl, alkylsulfonyl, alkylphosphono, alkoxyalkyl, heteroaryl, heteroarylalkyl, dialkylaminoalkyl, heterocyclylalkyl, or R₁₂ and R₁₃ can together with the nitrogen atom to which they are attached form a heterocyclyl group or a heteroaryl group, wherein the heterocyclyl or heteroaryl can optionally include one or more additional nitrogen, sulfur or oxygen atoms, or R₁₂ and R₁₃ can together with the nitrogen atom to which they are attached form an alkylazo group, and d is one to six;

R₁₄ is hydrogen, alkyl, alkenyl, arylalkyl, carboxyalkyl, carboxyalkenyl, alkoxycarbonylalkyl, alkenyloxycarbonylalkyl, cyanoalkyl, hydroxyalkyl, carboxybenzyl, aminocarbonylalkyl;
R₁₅ and R₁₆ are independently hydrogen, alkyl, alkoxycarbonyl, alkoxyaminoalkyl, cyclo(oxo)alkyl, cycloalkylcarbonyl, heterocyclylaminoalkyl, cycloalkyl, cyanoalkyl, cyano, sulfo, phosphono, sulfoalkyl, phosphonoalkyl, alkylsulfonyl, alkylphosphono, alkoxyalkyl, heterocyclylalkyl, or R₁₅ and R₁₆ can together with the nitrogen atom to which they are attached form a heterocyclyl group, wherein the heterocyclyl can optionally include one or more additional nitrogen, sulfur or oxygen atoms, or R₁₅ and R₁₆ can together with the nitrogen atom to which they are attached form an alkylazo;

R₁₇ is hydrogen, alkyl, perhaloalkyl, alkoxy, alkenyl, carboxyalkyl, amino, aminoalkyl, monoalkylaminoalkyl, dialkylaminoalkyl, alkoxyalkyl, alkoxycarbonyl, cyanoalkyl, alkylthioalkyl, alkylsulfinylalkyl, alkylsulfonylalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, or hydroxyimino(amino)alkyl;

R₁₈ and R₁₉ are independently hydrogen, methyl or ethyl, preferably hydrogen or methyl; and d is from one to six; and

R₂₀ is hydrogen, C₁-C₆ alkyl, or aryl;

wherein any alkyl, aryl, cycloalkyl, heterocyclyl, or heteroaryl group, or any substitutent which includes any of these groups, is optionally substituted.

Preferred compounds of Formula I are defined as above, with the provisos that:

when R₁ is C₃-C₂₀ alkanoyl, carboxyalkanoyl or alkoxycarbonyl, and R₃ is isopropenyl, isopropyl, 2′-hydroxyisopropyl, 2′-haloisopropyl, or 2′-thioisopropyl, and R₂ is formula (i), formula (ii) or formula (Iv), then R₅ cannot be C₂-C₂₀ alkyl or carboxy(C₂-C₂₀)alkyl, or R₆ cannot be hydrogen or carboxyalkyl, or R₉ cannot be hydrogen;

when R₁ is carboxyalkanoyl, and R₃ is isopropenyl, isopropyl, isobutyl, isobutenyl, or 2′-hydroxyisopropyl, and R₂ is formula (ii), formula (Iv) or formula (v), then R₆ cannot be alkyl, R₉ cannot be alkyl or carboxyalkyl, and R₁₀ and R₁, cannot be carboxyalkyl;

when R₁ is carboxyalkenoyl, R₂ is formula (ii), and R₃ is isopropenyl, then R₆ cannot be hydrogen; and

when R₁ is 3′,3′-dimethylsuccinyl, R₂ is formula (Iv), and R₉ is hydrogen, then R₃ cannot be 1′-hydroxyethyl, 1′-(oxo)ethyl or 1′-(alkoxy)ethyl.

In certain embodiments, R₁ is C₃-C₂₀ alkanoyl, carboxyalkanoyl, carboxyalkenoyl, alkoxycarbonylalkanoyl, alkenyloxycarbonylalkanoyl, cyanoalkanoyl, hydroxyalkanoyl, aminocarbonylalkanoyl, monoalkylaminocarbonylalkanoyl, dialkylaminocarbonylalkanoyl, heteroarylalkanoyl, heteroarylaminocarbonylalkanoyl, cyanoaminocarbonylalkanoyl, alkylsulfonylaminocarbonylalkanoyl, arylsulfonylaminocarbonylalkanoyl, tetrazolylalkanoyl, phosphonoalkyl, or sulfoalkyl. In certain other embodiments, R₁ is C₃-C₂₀ alkanoyl, carboxyalkanoyl, carboxyalkenoyl, alkoxycarbonylalkanoyl, alkenyloxycarbonylalkanoyl, cyanoalkanoyl, hydroxyalkanoyl, aminocarbonylalkanoyl, alkylaminocarbonylalkanoyl, alkylsulfonylaminocarbonylalkanoyl, arylsulfonylaminocarbonylalkanoyl, or tetrazolylalkanoyl.

In other embodiments, R₁ can be carboxyalkanoyl, wherein the carboxyalkanoyl is succinyl, glutaryl, 3′-methylglutaryl, 3′-methylsuccinyl, 3′,3′-dimethylsuccinyl or 3′,3′-dimethylglutaryl. Additional suitable carboxyalkanoyl include 2′,2′-dimethylmalonyl, 2′,3′-dihydroxysuccinyl, 2′,2′,3′,3′-tetramethylsuccinyl, 3′-methylsuccinyl, or 2′,2′-dimethylsuccinyl. In certain preferred embodiments, R₁ is a carboxyalkanoyl selected from the group consisting of:

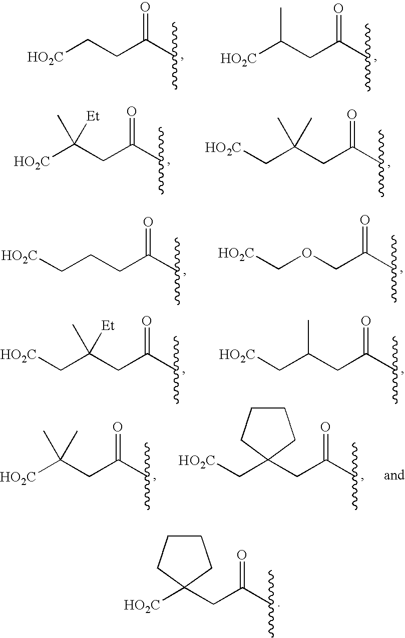

In some embodiments, R₁ is alkenyloxycarbonylalkanoyl, wherein the alkenyloxycarbonylalkanoyl is C₁-C₄ alkene ester of 3′,3′-dimethylsuccinyl or 3′,3′-dimethylglutaryl. In some embodiments, a suitable C₁-C₄ alkene ester is an allyl ester of succinyl, glutaryl, 3′-methylglutaryl, 3′-methylsuccinyl, 3′,3′-dimethylsuccinyl or 3′,3′-dimethylglutaryl.

In some embodiments, R₁ is alkoxycarbonylalkanoyl. Suitable alkoxycarbonylalkanoyl can include C₁-C₄ alkyl esters of succinyl, glutaryl, 3′-methylglutaryl, 3′-methylsuccinyl, 3′,3′-dimethylsuccinyl or 3′,3′-dimethylglutaryl. Preferably, the C₁-C₄ alkyl ester is a methyl, ethyl or propyl ester of succinyl, glutaryl, 3′-methylglutaryl, 3′-methylsuccinyl, 3′,3′-dimethylsuccinyl or 3′,3′-dimethylglutaryl.

Suitable R₁ substituents include alkanoyl. Preferably, the alkanoyl is tert-butylcarbonyl or isopropylcarbonyl. Suitable R₁ substituents include carboxyalkenoyl. Preferably, the carboxyalkenoyl is alk-2-enyloyl. Suitable R₁ substituents include cyanoalkanoyl. Preferably the cyanoalkanoyl is 4′-cyanopropanoyl or 4′-cyanobutanoyl. Suitable R₁ substituents include hydroxyalkanoyl. Preferably, the hydroxyalkanoyl is 3′,3′-dimethyl-4′-hydroxybutanoyl. Suitable R₁ substituents include aminocarbonylalkanoyl. Preferably, the aminocarbonylalkanoyl is 4′-amino-3′,3′-dimethylsuccinyl or 4′-aminosuccinyl. Suitable R₁ substituents include alkylsulfonylaminocarbonylalkanoyl. Preferably, the alkylsulfonylaminocarbonylalkanoyl is 4′-methylsulfonylamino-3′,3′-dimethylsuccinyl. Suitable R₁ substituents include arylsulfonylaminocarbonylalkanoyl. Preferably, the arylsulfonylaminocarbonylalkanoyl is 4′-phenylsulfonylamino-3′,3′-dimethylsuccinyl. Suitable R₁ substituents include tetrazolylalkanoyl. Preferably, the tetrazolylalkanoyl is C₂-C₆ tetrazolylalkanoyl. Suitable R₁ substituents include phosphonoalkyl. Preferably, the phosphonoalkyl is C₁-C₆ phosphonoalkyl. Suitable R₁ substituents include sulfoalkyl. Preferably, the sulfoalkyl is C₁-C₆ sulfoalkyl. Suitable R₁ substituents include heterocyclylcarbonylalkanoyl. Preferably, the heterocyclylcarbonylalkanoyl is 5′-morpholino-3′,3′-dimethylglutaryl. Suitable R₁ substituents include hydroxyaminocarbonylalkanoyl.

In some other embodiments, R₁ can be C₃-C₂₀ alkanoyl, carboxyalkanoyl, carboxyalkenoyl, alkoxycarbonylalkanoyl, alkenyloxycarbonylalkanoyl, cyanoalkanoyl, hydroxyalkanoyl, aminocarbonylalkanoyl, hydroxyaminocarbonylalkanoyl, monoalkylaminocarbonylalkanoyl, dialkylaminocarbonylalkanoyl, heteroarylalkanoyl, heterocyclylalkanoyl, heterocycylcarbonylalkanoyl, heteroarylaminocarbonylalkanoyl, heterocyclylaminocarbonylalkanoyl, cyanoaminocarbonylalkanoyl, alkylsulfonylaminocarbonylalkanoyl, arylsulfonylaminocarbonylalkanoyl, sulfoaminocarbonylalkanoyl, phosphonoaminocarbonylalkanoyl, tetrazolylalkanoyl, phosphono, sulfo, phosphonoalkanoyl, sulfoalkanoyl, alkylsulfonylalkanoyl, or alkylphosphonoalkanoyl.

In some embodiments, R₂ is formyl, carboxyalkenyl, heterocyclyl, heteroaryl, —CH₂SR₁₄, CH₂SOR₁₄, or CH₂SO₂R₁₄.

In some other embodiments, R₁₄ is hydrogen, alkyl, alkenyl, arylalkyl, carboxyalkyl, carboxyalkenyl, alkoxycarbonylalkyl, alkenyloxycarbonylalkyl, cyanoalkyl, hydroxyalkyl, carboxybenzyl, aminocarbonylalkyl.

In some embodiments, R₂ is heterocyclyl. Suitable heterocyclyl groups include, but are not limited to, oxazolyl, morpholinyl, piperidinyl, piperazinyl, pyranyl, azetidinyl, dihydropyrrolyl, dihydrofuranyl, 1,3-oxazinyl, isoxazinyl, and oxathiazinyl, 1,2-dithiolyl, 1,3-dithiolyl, 1,2-oxathiolyl, 1,3-oxathiolyl, 1,3-dioxolanyl, 1,3-dithiolanyl, 1,3-dioxanyl, 1,3-dioxathianyl, and 1,3-dithianyl any of which can be optionally substituted.

In some embodiments, R₂ is heteroaryl. Suitable heteroaryl groups include, but are not limited to, tetrazolyl, pyridinyl, imidazolyl, isoxazolyl, furanyl, oxazolyl, thiazolyl, pyrrolyl, thienyl, pyrazolyl, triazolyl, oxazolyl, isothiazolyl, oxadiazolyl, oxatriazolyl, pyridazinyl, pyrimidinyl, pyrazinyl, and triazinyl, any of which can be optionally substituted.

A group of compounds useful in the present invention are those wherein R₂ is

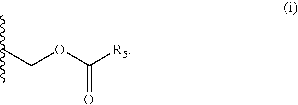

In some embodiments, R₅ is C₂-C₂₀ alkyl, alkenyl, alkynyl, carboxy(C₂-C₂₀)alkyl, amino, aminoalkyl, dialkylamino, monoalkylaminoalkyl, dialkylaminoalkyl, alkoxyalkyl, cyanoalkyl, alkylthioalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, or hydroxyimino(amino)alkyl. In some embodiments, R₅ is alkyl, preferably C₁-C₆ alkyl. In some embodiments, R₅ is alkenyl, preferably propen-2-yl, buten-2-yl, or penten-2-yl. In some embodiments, R₅ is C₂-C₁₀ carboxyalkyl, preferably 2′-carboxy-2′,2′-dimethylethyl or 3′-carboxy-3′,3′-dimethylpropyl. R₅ can also be heterocyclyl, heterocyclylalkyl, heterocycloalkanoyl, or heteroarylalkyl. Preferable heterocyclyls include tetrazolyl, pyridinyl, imidazolyl, isoxazolyl, morpholinyl, or furanyl. Preferable heterocycloalkyls include heterocyclyl(C₁-C₆)alkyl, wherein the heterocyclyls are as previously defined.

In some embodiments, R₅ is C₂-C₂₀ alkyl, alkenyl, C₂-C₂₀ carboxyalkyl, amino, aminoalkyl, monoalkylaminoalkyl, dialkylaminoalkyl, alkoxyalkyl, cyano, cyanoalkyl, alkylthioalkyl, cycloalkyl, cycloalkylalkyl, aryl, arylalkyl, heterocyclyl, heteroaryl, heterocyclylalkyl, heteroarylalkyl, sulfo, phosphono, sulfoalkyl, phosphonoalkyl, alkylsulfonyl, alkylphosphono, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, arylphosphonoaminocarbonylalkyl, alkylphosphonoaminocarbonylalkyl, or hydroxyimino(amino)alkyl.

A group of compounds useful in the present invention are those wherein R₂ is

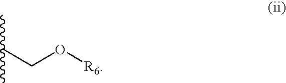

Suitable R₆ substituents include hydrogen, phosphono, sulfo. Suitable R₆ substituents also include alkyl, sulfoalkyl, phosphonoalkyl, alkylsulfonyl, alkylphosphono, cycloalkyl, cycloalkenyl, aryl, heterocyclyl, heteroaryl, carboxyalkyl, alkoxycarbonylalkyl, cyanoalkyl; CH₂CONR₇R₈, trialkylsilyl, ethoxyethyl (OEE), or tetrahydropyranyl ether (OTHP). In some embodiments, R₆ can be one of the protecting groups listed above, or any other suitable protecting group known in the art, e.g., a suitable protecting group as described in Protective Groups in Organic Synthesis, 3^{rd} ed. (eds. T. W. Greene and P. G. M. Wuts, John Wiley and Sons, Inc. (1999)), incorporated herein by reference. More preferred substituents include hydrogen, cycloalkyl, heterocyclyl, heteroaryl, carboxyalkyl, alkoxycarbonylalkyl, or cyanoalkyl; more preferably cycloalkyl, heterocyclyl, heteroaryl, carboxyalkyl, alkoxycarbonylalkyl, or cyanoalkyl. In certain embodiments, R₆ is cycloalkyl or heterocycloalkyl. In other embodiments, R₆ is cyclopropyl, cyclopentyl, cyclohexyl, pyridinylmethyl or octacyclen-2-yl, preferably, pyridinylmethyl or octacyclen-2-yl. In other embodiments, R₆ is carboxyalkyl or R₆ is alkoxycarbonylalkyl or R₆ is cyanoalkyl.

In some embodiments, R₆ is hydrogen, phosphono, sulfo, alkyl, sulfoalkyl, phosphonoalkyl, alkylsulfonyl, alkylphosphono, cycloalkyl, heterocyclyl, aryl, heteroaryl, carboxyalkyl, alkoxycarbonylalkyl, or cyanoalkyl.

A group of compounds useful in the present invention are compounds wherein R₂ is

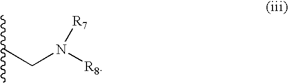

In some other embodiments, R₇ and R₈ are independently alkoxyalkylamine or hydrogen. In some embodiments, R₇ and R₈ are independently alkyl. Preferably, R₇ is methoxyethyl and R₈ is hydrogen, or R₇ is methoxyethyl and R₈ is methyl. In some other embodiments, R₇ and R₈ are alkylsulfonyl, arylsulfonyl, or heteroarylsulfonyl, heterocyclylsulfonyl. Alternatively, R₇ and R₈ together with the nitrogen atom to which they are attached can form a heterocyclyl group, wherein the heterocyclyl group can optionally include one or more additional nitrogen, sulfur or oxygen groups. Preferable heterocyclyl groups include, but are not limited to, pyrrolidinyl, morpholinyl, piperazinyl, piperidinyl, and thiomorpholinyl. In some embodiments, the heterocyclyl group is optionally substituted.

In some other embodiments, R₇ or R₈ are independently hydrogen, alkyl, aminoalkyl, monoalkylaminoalkyl, dialkylaminoalkyl, carboxyalkyl, alkoxyalkyl, alkoxyalkoxyalkyl, alkoxycarbonylaminoalkoxyalkyl, alkoxycarbonylaminoalkyl, aminoalkoxyalkyl, alkylcarbonylaminoalkyl, heterocyclyl, heterocyclylalkyl, aryl, arylalkyl, arylcarbonylaminoalkyl, or cycloalkyl, or R₇ and R₉ can together with the nitrogen atom to which they are attached form a heterocyclyl or heteroaryl group, wherein the heterocyclyl or heteroaryl can optionally include one or more additional nitrogen, sulfur or oxygen atoms.

A group of compounds useful in the present invention are compounds wherein R₂ is

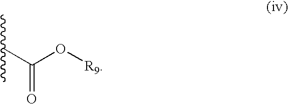

Suitable R₉ substituents include hydrogen, phosphono, sulfo, alkyl, alkenyl, trialkylsilyl, carboxyalkyl, alkoxycarbonyloxyalkyl, aminoalkyl, monoalkylaminoalkyl, dialkylaminoalkyl, alkoxyalkyl, cyanoalkyl, phosphonoalkyl, sulfoalkyl, alkylsulfonyl, alkylphosphono, aryl, heteroaryl, heterocyclyl, or dialkoxyalkyl, preferably hydrogen, phosphono, sulfo, alkoxycarbonyloxyalkyl, cyanoalkyl, phosphonoalkyl, sulfoalkyl, alkylsulfonyl, aryl, heteroaryl, heterocyclyl, or dialkoxyalkyl, more preferably hydrogen, alkoxycarbonyloxyalkyl, cyanoalkyl, alkoxyalkyl, or dialkoxyalkyl. In some embodiments, R₉ is alkoxycarbonyloxyalkyl. Suitable alkoxycarbonyloxyalkyl include tert-butoxycarbonyloxymethyl and tert-butoxycarbonyloxymethyl(methyl). In some embodiments, R₉ is dialkylaminoalkyl, preferably dimethylaminoalkyl, more preferably dimethylaminoethyl. In some embodiments, R₉ is heterocyclyl, preferably tetrahydrofuranyl or tetrahydropyranyl, more preferably tetrahydrofuran-3-yl or tetrahydropyran-4-yl. In some embodiments, R₉ is phosphono or sulfo. In some embodiments, R₉ is dialkoxyalkyl, for example

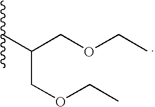

In some other embodiments, R₉ is hydrogen, phosphono, sulfo, alkyl, alkylsilyl, cycloalkyl, carboxyalkyl, alkoxycarbonyloxyalkyl, aminoalkyl, monoalkylaminoalkyl, dialkylaminoalkyl, cyanoalkyl, phosphonoalkyl, sulfoalkyl, alkylsulfonyl, alkylphosphono, aryl, heteroaryl, heterocyclyl, arylalkyl, heteroarylalkyl, heterocyclylalkyl, or dialkoxyalkyl.

A group of compounds useful in the present invention can be wherein R₂ is

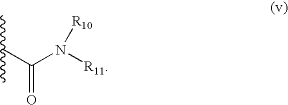

R₁₀ and R₁₁ can both be hydrogen. In some embodiments, R₁₀ and R₁₁ can be independently alkyl, amino, aminoalkyl, monoalkylaminoalkyl, dialkylaminoalkyl, carboxyalkyl, alkanoyloxyalkyl, alkoxyalkyl, hydroxyalkyl, cyanoalkyl, alkoxyalkoxyalkyl, alkoxycarbonylaminoalkoxyalkyl, alkoxycarbonylalkyl, hydroxyalkoxyalkyl, alkoxycarbonylaminoalkyl, aminoalkoxyalkyl, alkoxycarbonylamino, alkoxycarbonylalkyl, heterocyclylheterocyclylalkyl, heterocyclylarylalkyl, arylaminoalkyl, aminocycloalkyl, alkylcarbonylaminoalkyl, heterocyclyl, heterocyclylalkyl, aryl, heteroarylalkyl, arylalkyl, arylcarbonylaminoalkyl, alkysulfonyl, arylsulfonyl, alklysulfonylaminoalkyl, arlysulfonylaminoalkyl, or cycloalkyl. In some embodiments, R₁₀ and R₁₁ can be independently alkyl interrupted by one or more oxygen atoms. Alternatively, R₁₀ and R₁₁ can be independently alkyl, aminoalkyl, aminoalkoxyalkyl, alkoxyalkyl, cycloalkyl, alkoxycarbonylaminoalkoxyalkyl, alkoxycarbonylaminoalkyl, alkylcarbonylaminoalkyl, alkoxyalkoxyalkyl, or dialkylaminoalkyl. Preferably, R₁₀ and R₁₁ are alkyl or aminoalkyl. In other embodiments, one of R₁₀ and R₁₁ is hydrogen, and one of R₁₀ and R₁₁ is heterocyclyl, aryl, arylalkyl, arylcarbonylaminoalkyl, or heterocycloalkyl. In other embodiments, one of R₁₀ and R₁₁ is hydrogen, and one of R₁₀ and R₁₁ is alkoxycarbonylamino, alkoxycarbonylalkyl, cyanoalkyl, alkylsulfonyl. In some embodiments, R₁₀ and R₁₁ are taken together to form a heterocyclyl group, wherein the heterocyclyl group can optionally include one or more additional nitrogen, sulfur or oxygen atoms. Preferred heterocyclyl groups include, but are not limited to, morpholinyl, piperidinyl, pyrrolidinyl, thiomorpholinyl, and piperazinyl. In some embodiments, R₁₀ is phenylsulfonyl and R₁₁ is hydrogen. In some embodiments, both R₁₀ and R₁₁ are alkoxyalkyl, preferably both R₁₀ and R₁₁ are methoxyethyl.

In some embodiments, one of R₁₀ and R₁₁ is hydrogen, and one of R₁₀ and R₁₁ is alkyl, wherein the alkyl group is selected from methyl, 2-hydroxyethyl, 2-hydroxy-2-methylpropyl, propyl, ethyl, isopropyl, (R)-2-[2,3-dihydroxypropyl], (S)-2-[2,3-dihydroxypropyl], (S)-2-[1-hydroxy-4-methylpentyl)], (R)-2-[1-hydroxy-4-methylpentyl)], or (S)-1-carboxy-3-methylbutyl. In some embodiments, one of R₁₀ and R₁₁ is hydrogen, and one of R₁₀ and R₁₁ is aminoalkyl, wherein the aminoalkyl is 2-(1-amino-2-methylpropyl). In some embodiments, one of R₁₀ and R₁₁ is hydrogen, and one of R₁₀ and R₁₁ is alkoxyalkyl, wherein the alkoxyalkyl group is 2-methoxyethyl or 2-hydroxyethoxyethyl. In some embodiments, one of R₁₀ and R₁₁ is hydrogen, and one of R₁₀ and R₁₁ is alkoxycarbonylaminoalkyl, wherein the alkoxycarbonylaminoalkyl group is 2-(tert-butoxycarbonylamino)ethyl. In some embodiments, one of R₁₀ and R₁₁ is hydrogen, and one of R₁₀ and R₁₁ is dialkylaminoalkyl, wherein the dialkylaminoalkyl group is 2-N,N-dimethylaminoethyl, 2-N,N-dimethylaminopropyl, (1R,3R)-3-N,N-dimethylaminocyclopentyl, or (1S,3S)-3-N,N-dimethylaminocyclopentyl.

In some embodiments, one of R₁₀ and R₁₁ is hydrogen, and one of R₁₀ and R₁₁ is cycloalkyl, heterocyclyl, aryl, arylalkyl, arylcarbonylaminoalkyl, arylsulfonyl, heterocyclylheterocyclylalkyl, heterocyclylarylalkyl, arylaminoalkyl, aminocycloalkyl, or heterocycloalkyl. In some embodiments, one of R₁₀ and R₁₁ is hydrogen, and one of R₁₀ and R₁₁ is cycloalkyl, wherein the cycloalkyl group is cyclopropyl. In some embodiments, one of R₁₀ and R₁₁ is hydrogen, and one of R₁₀ and R₁₁ is heterocyclyl, wherein the heterocyclyl group is selected from (S)-1-[(tert-butoxycarbonyl)pyrrolidinyl], (R)-1-[(tert-butoxycarbonyl)pyrrolidinyl], (S)-3-pyrrolidinyl, (R)-3-pyrrolidinyl. (S)-3-(1-methylpyrrolidinyl), (R)-3-(1-methylpyrrolidinyl), (S)-3-(1-acetylpyrrolidinyl), (R)-3-(1-acetylpyrrolidinyl), (S)-3-(1-methylsulfonylpyrrolidinyl), (R)-3-(1-methylsulfonylpyrrolidinyl), 4-(1-(tert-butoxycarbonyl)piperdinyl), 4-piperidinyl, 4-(1-methylpiperidinyl), or 4-[1-(1-hydroxyethyl)piperidinyl)].

In some embodiments, one of R₁₀ and R₁₁ is hydrogen, and one of R₁₀ and R₁₁ is aryl, wherein the aryl group is 4-fluorophenyl, 2-(1,3,4-thiadiazolyl)methyl, or 2,3-dichlorobenzyl, 4-azido-2,3,5,6-tetrafluorobenzyl. In some embodiments, one of R₁₀ and R₁₁ is hydrogen, and one of R₁₀ and R₁₁ is arylalkyl, wherein the arylalkyl group is selected from 4-fluorobenzyl, 3-fluorobenzyl, 2-fluorobenzyl, 4-chlorobenzyl, 3-chlorobenzyl, 2-chlorobenzyl, 4-methylbenzyl, 3-methylbenzyl, 2-methylbenzyl, 4-methyoxybenzyl, 3-methoxybenzyl, 2-methoxybenzyl, 4-N,N-dimethylaminobenzyl, 4-trifluoromethylbenzyl, 4-carboxybenzyl, 3,4-dichlorobenzyl, 2,4-dichlorobenzyl, 2-pyridinylmethyl, 3-pyridinylmethyl, 4-pyridinylmethyl, 2-benzyl, 3-trifluoromethylbenzyl, 4-tert-butylbenzyl, 4-aminobenzyl, 4-acetamidobenzyl, (R)-1-phenylethyl, (S)-1-phenylethyl, (R)-2-hydroxy-1-phenylethyl, (S)-2-hydroxy-1-phenylethyl, or 2-phenylethyl.

In some embodiments, one of R₁₀ and R₁₁ is hydrogen, and one of R₁₀ and R₁₁ is heterocycloalkyl, wherein the heterocycloalkyl group is selected from 4-(1-methylimidazolyl)methyl, 3-(5-methylisoxazolyl)methyl, 3-(4-morpholinyl)propyl, 3-(1-imidazolyl)propyl, 2-(4-methylmorpholinyl)methyl, 2-morpholinylmethyl, or 2-(4-tert-butoxycarbonyl morpholinyl)methyl. In some embodiments, one of R₁₀ and R₁₁ is hydrogen, and one of R₁₀ and R₁₁ heterocyclylarylalkyl, wherein the heterocyclylarylalkyl group is selected from 4-(4-morpholinyl)benzyl or 4-(4-methylpiperazinyl)benzyl. In some embodiments, one of R₁₀ and R₁₁ is hydrogen, and one of R₁₀ and R₁₁ heterocyclylheterocyclylalkyl, wherein the heterocyclylheterocyclylalkyl group is 3-[6-(4-morpholinyl)pyridinyl]methyl. In some embodiments, one of R₁₀ and R₁₁ is hydrogen, and one of R₁₀ and R₁₁ is arylaminoalkyl, wherein the arylaminoalkyl is 2-[(4-azido-2,3,5,6-tetrafluorobenzoyl)amino]ethyl. In some embodiments, R₁₀ and R₁₁ is hydrogen, and one of R₁₀ and R₁₁ is aminocycloalkyl, wherein the aminocycloalkyl is (1R,3R)-3-aminocyclopentyl, (1S,3S)-3-aminocyclopentyl, (1r,4r)-4-aminocyclohexyl, or (1s,4s)-4-aminocyclohexyl.

In some embodiments, one of R₁₀ and R₁₁ is hydrogen, and one of R₁₀ and R₁₁ is dialkylaminocycloalkyl, wherein the dialkylaminocycloalkyl is (1r,4r)-4-N,N-dimethylaminocyclohexyl or (1s,4s)-4-N,N-dimethylaminocyclohexyl.

In some embodiments, R₁₀ and R₁₁ are taken together to form one of 4-(tert-butoxycarbonyl)piperazinyl, morpholinyl, piperidinyl, piperazinyl, 4-(4-morpholinylcarbonyl)piperazinyl, 4-methylpiperazinyl, 4-ethylpiperazinyl, 4-isopropylpiperazinyl, 4-(cyclopropylmethyl)piperazinyl, 4-benzylpiperazinyl, 4-[3-(5-methylisoxazolyl)methyl]piperazinyl, 4-(4-pyridinylmethyl)piperazinyl, 4-acetylpiperazinyl, 4-(isopropylaminocarbonyl)piperazinyl, 4-(methylsulfonyl)piperazinyl, 4-cyclopropylpiperazinyl, 4-(2-methoxyethylaminocarbonyl)piperazinyl, 4-(2-hydroxyethyl)piperazinyl, 4-(2-methoxyethyl)piperazinyl, 4-(3-dimethylaminopropyl)piperazinyl, 4-(aminocarbonyl)piperazinyl, 4-(aminosulfonyl)piperazinyl, 3-oxopiperazinyl, 4-methyl-3-oxopiperazinyl, 4-(hydroxyethyl)-3-oxopiperazinyl, 4-(2-hydroxybenzoyl)piperazinyl, 4-[3-(1,2,4-oxadiazolyl)methyl]piperazinyl, 4-[4-(dimethylaminosulfonyl)benzyl]piperazinyl, 4-[1-(1,2,3,4-tetrahydronaphthyl)]piperazinyl, 4-[4-(acetamidobenzyl)]piperazinyl, (1S,4S)-5-methyl-2,5-diazabicyclo[2.2.1]heptanyl, (1R,4R)-5-methyl-2,5-diazabicyclo[2.2.1]heptanyl, (1S,4S)-2,5-diazabicyclo[2.2.1]heptanyl, (1R,4R)-2,5-diazabicyclo[2.2.1]heptanyl, (1S,4S)-5-(tert-butoxycarbonyl)-2,5-diazabicyclo[2.2.1]heptanyl, (1R,4R)-5-(tert-butoxycarbonyl)-2,5-diazabicyclo[2.2.1]heptanyl, 4-(4-azido-2,3,5,6-tetrafluorobenzyl)piperazinyl, pyrrolidinyl, (R,S)-3-hydroxypyrrolidinyl, (R)-3-hydroxypyrrolidinyl, (S)-3-hydroxypyrrolidinyl, (R)-3-(tert-butoxycarbonylamino)pyrrolidinyl, (S)-3-(tert-butoxycarbonylamino)pyrrolidinyl, (R)-3-aminopyrrolidinyl, (S)-3-aminopyrrolidinyl, (R)-2-(hydroxymethyl)pyrrolidinyl, (S)-2-(hydroxymethyl)pyrrolidinyl, (S)-2-(hydroxymethyl)pyrrolidinyl, (S)-2-(hydroxymethyl)pyrrolidinyl, (S)-2-(hydroxymethyl)pyrrolidinyl, (R)-3-N-methylaminopyrrolidinyl, (S)-3-N-methylaminopyrrolidinyl, (R)-3-N,N-dimethylaminopyrrolidinyl, (S)-3-N,N-dimethylaminopyrrolidinyl, (R)-3-N,N-diethylaminopyrrolidinyl, (S)-3-N,N-diethylaminopyrrolidinyl, (R)-3-N-ethylaminopyrrolidinyl, (S)-3-N-ethylaminopyrrolidinyl, (R)-3-(4-morpholinyl)pyrrolidinyl, (S)-3-(4-morpholinyl)pyrrolidinyl, (R)-3-(1-pyrrolidinyl)pyrrolidinyl, (S)-3-(1-pyrrolidinyl)pyrrolidinyl, 4-aminopiperidinyl, 4-oxopiperidinyl, 4-hydroxypiperidinyl, 4-N,N-diaminopiperidinyl, 4-(4-morpholinyl)piperidinyl, 4-acetamidopiperidinyl, 4-(methylsulfonamide)piperidinyl, (R)-3-acetamidopyrrolidinyl, (S)-3-acetamidopyrrolidinyl, (R)-3-(cyclopropanecarboxamido)pyrrolidinyl, (S)-3-(cyclopropanecarboxamido)pyrrolidinyl, (R)-3-(2-hydroxyacetamido)pyrrolidinyl, (S)-3-(2-hydroxyacetamido)pyrrolidinyl, (R)-3-(methylsulfonamido)pyrrolidinyl, (S)-3-(methylsulfonamido)pyrrolidinyl, (R)-2-(aminomethyl)pyrrolidinyl, (S)-2-(aminomethyl)pyrrolidinyl, (R)-2-(N,N-dimethylaminomethyl)pyrrolidinyl, (S)-2-(N,N-dimethylaminomethyl)pyrrolidinyl, (R)-2-(acetamidomethyl)pyrrolidinyl, (S)-2-(acetamidomethyl)pyrrolidinyl, (R)-2-(methylsulfonamidomethyl)pyrrolidinyl, (S)-2-(methylsulfonamidomethyl)pyrrolidinyl, (R)-2-(N,N-diethylaminomethyl)pyrrolidinyl, (S)-2-(N,N-diethylaminomethyl)pyrrolidinyl, (R)-2-(4-morpholinylmethyl)pyrrolidinyl, (S)-2-(4-morpholinylmethyl)pyrrolidinyl, 2,6-dimethylmorpholinyl, 1,4-oxazepanyl, thiomorpholinyl, thiomorpholinyl 1-oxide, or thiomorpholinyl 1,1-dioxide.

In some other embodiments, R₁₀ and R₁₁ are independently hydrogen, hydroxy, cyano, alkyl, amino, aminoalkyl, monoalkylaminoalkyl, dialkylaminoalkyl, carboxyl, carboxyalkyl, alkanoyloxyalkyl, alkoxyalkyl, alkoxyalkoxyalkyl, alkoxycarbonylaminoalkoxyalkyl, alkoxycarbonylaminoalkyl, aminoalkoxyalkyl, alkylcarbonylaminoalkyl, heterocyclyl, heterocyclylalkyl, aryl, arylalkyl, arylcarbonylaminoalkyl, arylsulfonyl, or cycloalkyl, or alkyl interrupted by one or more oxygen atoms, or R₁₀ and R₁₁ can together with the nitrogen atom to which they are attached form a heterocyclyl group, wherein the heterocyclyl can optionally include one or more additional nitrogen, sulfur or oxygen atoms.

A group of compounds useful in the present invention are those compounds wherein R₂ is

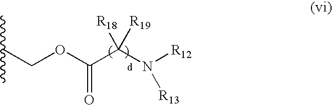

In some embodiments, one of R₁₂ and R₁₃ are hydrogen and one of R₁₂ and R₁₃ is alkylamino, alkenyl, alkynyl, alkoxy, alkoxyalkyl, alkoxycarbonyl, cycloalkyl, cycloalkyloxo, heteroaryl, heteroarylalkyl, dialkylaminoalkyl, or cyanoalkyl. R₁₂ and R₁₃ can be hydrogen. In some embodiments, one or both of R₁₂ and R₁₃ can be cyano, sulfo, phosphono, sulfoalkyl, phosphonoalkyl, or alkylsulfonyl. Alternatively, R₁₂ and R₁₃ can together with the nitrogen atom to which they are attached form a heterocyclyl or heteroaryl, wherein the heterocyclyl or heteroaryl group can optionally include one or more additional nitrogen, sulfur or oxygen atoms. In some embodiments R₁₈ and R₁₉ can be independently hydrogen or C₁-C₆ alkyl. In some embodiments, R₁₈ and R₁₉ can both be hydrogen. In some embodiments, R₁₈ and R₁₉ can both be methyl. In some embodiments, d can be one to six, preferably one to four, most preferably one to two. In some embodiments, d is one.

In some other embodiments, R₁₂ and R₁₃ are independently hydrogen, alkyl, alkoxycarbonyl, alkoxyaminoalkyl, cycloalkyloxy, heterocyclylaminoalkyl, cycloalkyl, cyanoalkyl, cyano, sulfo, phosphono, sulfoalkyl, phosphonoalkyl, alkylsulfonyl, alkylphosphono, alkoxyalkyl, heterocyclylalkyl, or R₁₂ and R₁₃ can together with the nitrogen atom to which they are attached form a heterocyclyl group, wherein the heterocyclyl can optionally include one or more additional nitrogen, sulfur or oxygen atoms, or R₁₂ and R₁₃ can together with the nitrogen atom to which they are attached form an alkylazo group, and b is one to six.

A group of compounds useful in the present invention are those compounds wherein R₂ is

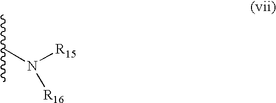

R₁₅ and R₁₆ are independently hydrogen, alkyl, alkoxycarbonyl, alkoxyaminoalkyl, cyclo(oxo)alkyl, cycloalkylcarbonyl, heterocyclylaminoalkyl, cycloalkyl, cyanoalkyl, alkoxyalkyl, or heterocyclylalkyl. In some embodiments, R₁₅ and R₁₆ are independently cyano, sulfo, phosphono, sulfoalkyl, phosphonoalkyl, or alkylsulfonyl. In some embodiments, R₁₅ and R₁₆ can together with the nitrogen atom to which they are attached form a heterocyclyl group, wherein the heterocyclyl can optionally include one or more additional nitrogen, sulfur or oxygen atoms. In some embodiments, R₁₅ and R₁₆ together with the nitrogen atom to which they are attached form an alkylazo group.

In some embodiments, R₁₅ and R₁₆ are independently hydrogen, alkyl, alkoxycarbonyl, alkoxyaminoalkyl, cycloalkyloxy, heterocyclylaminoalkyl, cycloalkyl, cyanoalkyl, cyano, sulfo, phosphono, sulfoalkyl, phosphonoalkyl, alkylsulfonyl, alkylphosphono, alkoxyalkyl, heterocyclylalkyl, or R₁₅ and R₁₆ can together with the nitrogen atom to which they are attached form a heterocyclyl group, wherein the heterocyclyl can optionally include one or more additional nitrogen, sulfur or oxygen atoms, or R₁₅ and R₁₆ can together with the nitrogen atom to which they are attached form an alkylazo group.

A group of compounds useful in the present invention are compounds wherein R₂

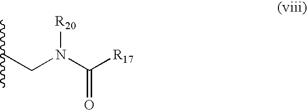

R₁₇ is hydrogen, alkyl, perhaloalkyl, alkoxy, alkenyl, carboxyalkyl, amino, aminoalkyl, monoalkylaminoalkyl, dialkylaminoalkyl, alkoxyalkyl, alkoxycarbonyl, cyanoalkyl, alkylthioalkyl, alkylsulfinylalkyl, alkylsulfonylalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, or hydroxyimino(amino)alkyl, preferably alkenyl, carboxyalkyl, amino, aminoalkyl, monoalkylaminoalkyl, dialkylaminoalkyl, alkoxyalkyl, cyanoalkyl, alkylthioalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, alkylaminocarbonylalkyl, dialkylaminocarbonylalkyl, heterocyclylcarbonylalkyl, or cycloalkylcarbonylalkyl. In some embodiments, R₁₇ is hydrogen. In some embodiments, R₁₇ is alkenyl, carboxyalkyl, amino, aminoalkyl, monoalkylaminoalkyl, dialkylaminoalkyl, alkoxyalkyl, cyanoalkyl, alkylthioalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, or alkylaminocarbonylalkyl.

In some embodiments, R₁₇ is hydrogen, alkyl, alkenyl, carboxyalkyl, amino, aminoalkyl, monoalkylaminoalkyl, dialkylaminoalkyl, alkoxyalkyl, alkoxycarbonyl, cyanoalkyl, alkylthioalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, or hydroxyimino(amino)alkyl.

In some embodiments, R₂₀ is hydrogen, C₁-C₆ alkyl, or aryl. In some embodiments, R₂₀ is methyl or ethyl. In some embodiments, R₂₀ is phenyl.

In some embodiments, R₃ can include hydroxyl, isopropenyl, isopropyl, 1′-hydroxyisopropyl, 1′-haloisopropyl, 1′-thioisopropyl, 1′-trifluoromethylisopropyl, 2′-hydroxyisopropyl, 2′-haloisopropyl, 2′-thioisopropyl, 2′-trifluoromethylisopropyl, 1′-hydroxyethyl, 1′-(alkoxy) ethyl, 1′-(alkoxyalkoxy) ethyl, 1′-(arylalkoxy) ethyl; 1′-(arylcarbonyloxy)ethyl, 1′-(oxo)ethyl, 1′-(hydroxyl)-1′-(hydroxyalkyl)ethyl, 1′-(oxo)oxazolidinyl, 1′,2′-epoxyisopropyl, 2′-haloisopropenyl, 2′-hydroxyisopropenyl, 2′-aminoisopropenyl, 2′-thioisopropenyl, 3′-haloisopropenyl, 3′-hydroxyisopropenyl, 3′-aminoisopropenyl, 3′-thioisopropenyl, 1′-alkoxyethyl, 1′-hydroximoylethyl, 1′-alkoxyimoyl, or

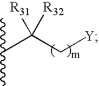

wherein Y is —SR₃₃ or —NR₃₃R₃₄;

R₃₁ is methyl;

R₃₂ is hydrogen or hydroxyl;

R₃₃ and R₃₄ are independently hydrogen, alkyl, alkanoyl, arylalkyl, heteroarylalkyl, arylsulfonyl or arylaminocarbonyl; or

R₃₃ and R₃₄ can be taken together with the nitrogen to which they are attached to form a heterocycle, wherein the heterocycle can optionally include one or more additional nitrogen, sulfur or oxygen atoms;

m is zero to three;

R₄ is hydrogen; or

R₃ and R₄ can be taken together to form oxo, alkylimino, alkoxyimino or benzyloxyimino.

R₃ useful groups include, but are not limited to, hydrogen, hydroxyl, isopropenyl, 1′-hydroxyethyl, 1′-(alkoxy)ethyl, 1′-(alkoxyalkoxy)ethyl, 1′-(arylalkoxy)ethyl; 1′-(arylcarbonyloxy)ethyl, acetyl, 1′-(hydroxyl)-1′-(hydroxyalkyl)ethyl, (2′-oxo)tetrahydrooxazolyl, 2′-haloisopropenyl, 2′-hydroxyisopropenyl, 2′-aminoisopropenyl, 2′-thioisopropenyl, 3′-haloisopropenyl, 3′-hydroxyisopropenyl, 3′-aminoisopropenyl, 3′-thioisopropenyl, 1′-alkoxyethyl, 1′-hydroxyiminoethyl, or 1′-alkoxyiminoethyl. In some embodiments, R₃ can include, but is not limited to hydroxyl, isopropenyl, 1′-hydroxyethyl, 1′-(alkoxy)ethyl, 1′-(alkoxyalkoxy)ethyl, 1′-(arylalkoxy)ethyl; 1′-(arylcarbonyloxy)ethyl, acetyl, 1′-(hydroxyl)-1′-(hydroxyalkyl)ethyl, or (2′-oxo)tetrahydrooxazolyl. In some embodiments, R₃ includes, but is not limited to, 1′-alkoxyethyl, 1′-hydroxyiminoethyl, or 1′-alkoxyiminoethyl. In some embodiments, R₃ includes, but is not limited to 3′-haloisopropenyl, 3′-hydroxyisopropenyl, 3′-aminoisopropenyl, or 3′-thioisopropenyl. In some embodiments, R₃ is 1′-methoxyiminoethyl. In some embodiments, R₄ is hydrogen, and R₃ is 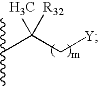
wherein Y is —SR₃₃ or —NR₃₃R₃₄, R₃₁ is hydrogen, R₃₂ is methyl, R₃₃ and R₃₄ are independently hydrogen, alkyl, alkanoyl, arylalkyl, heteroarylalkyl, arylsulfonyl or arylaminocarbonyl. In some embodiments, R₃₁ is hydrogen, R₃₂ is methyl, and R₃₃ and R₃₄ are taken together with the nitrogen to which they are attached to form heterocyclyl, wherein the heterocyclyl can optionally include one or more additional nitrogen, sulfur or oxygen atoms. The value of m can be zero to three.

In some embodiments, R₄ is hydrogen, and R₃ is 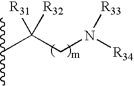
wherein R₃₁ is hydrogen, R₃₂ is methyl, R₃₃ and R₃₄ are independently hydrogen, alkyl, alkanoyl, arylalkyl, heteroarylalkyl, arylsulfonyl or arylaminocarbonyl. In some embodiments, R₃₁ is hydrogen, R₃₂ is methyl, and R₃₃ and R₃₄ can be taken together with the nitrogen to which they are attached to form heterocyclyl, wherein the heterocyclyl can optionally include one or more additional nitrogen, sulfur or oxygen atoms. The value of m can be zero to three.

Preferred compounds include those in which R₂ is (i), and R₃ is isopropenyl; wherein R₂ is (ii), and R₃ is isopropenyl; wherein R₂ is (iii), and R₃ is isopropenyl; wherein R₂ is (iv), and R₃ is isopropenyl; or wherein R₂ is (v), and R₃ is isopropenyl. Most preferred compounds include those in which R₂ is (v) and R₃ is isopropenyl. Additional preferred compounds include those in which R₂ is (i), and R₃ is isopropyl; wherein R₂ is (ii), and R₃ is isopropyl; wherein R₂ is (iii), and R₃ is isopropyl; wherein R₂ is (iv), and R₃ is isopropyl; or wherein R₂ is (v), and R₃ is isopropyl. Most preferred compounds include those in which R₂ is (v) and R₃ is isopropyl.

Preferred compounds include compounds wherein R₁ is succinyl, glutaryl, 3′-methylglutaryl, 3′-methylsuccinyl, 3′,3′-dimethylsuccinyl or 3′,3′-dimethylglutaryl or an allyl or alkyl ester of succinyl, glutaryl, 3′-methylglutaryl, 3′-methylsuccinyl, 3′,3′-dimethylsuccinyl or 3′,3′-dimethylglutaryl; R₂ is heteroaryl; and R₃ is isopropenyl. More preferred compounds can include compounds wherein R₁ is succinyl, glutaryl, 3′-methylglutaryl, 3′-methylsuccinyl, 3′,3′-dimethylsuccinyl, 3′,3′-dimethylglutaryl, or an allyl or alkyl ester of succinyl, glutaryl, 3′-methylglutaryl, 3′-methylsuccinyl, 3′,3′-dimethylsuccinyl, or 3′,3′-dimethylglutaryl; R₂ is dihydrooxazolyl; and R₃ is isopropenyl.

Preferred compounds include compounds wherein R₁ is succinyl, glutaryl, 3′-methylglutaryl, 3′-methylsuccinyl, 3′,3′-dimethylsuccinyl, 3′,3′-dimethylglutaryl, or an allyl or alkyl ester or arylalkyl ester of succinyl, glutaryl, 3′-methylglutaryl, 3′-methylsuccinyl, 3′,3′-dimethylsuccinyl, or 3′,3′-dimethylglutaryl; R₂ is (i), (ii) or (iv); and R₃ is isopropenyl. Preferred compounds include compounds wherein R₁ is succinyl, glutaryl, 3′-methylglutaryl, 3′-methylsuccinyl, 3′,3′-dimethylsuccinyl, 3′,3′-dimethylglutaryl, or an allyl or alkyl ester of succinyl, glutaryl, 3′-methylglutaryl, 3′-methylsuccinyl, 3′,3′-dimethylsuccinyl, or 3′,3′-dimethylglutaryl; R₂ is (iii), (v) or (vi); and R₃ is isopropenyl. Preferred compounds include compounds wherein R₁ is succinyl, glutaryl, 3′-methylglutaryl, 3′-methylsuccinyl, 3′,3′-dimethylsuccinyl, 3′,3′-dimethylglutaryl, or an allyl or alkyl ester of succinyl, glutaryl, 3′-methylglutaryl, 3′-methylsuccinyl, 3′,3′-dimethylsuccinyl, or 3′,3′-dimethylglutaryl; R₂ is (v) and R₃ is isopropenyl.

Additional preferred compounds include those wherein R₂ is (i), and R₅ is a heteroarylalkyl; wherein R₂ is (ii), and R₆ is a heteroaryl; wherein R₂ is (iv), and R₉ is cyanoalkyl; wherein R₂ is (iii), and R₇ and R₈ taken together with the nitrogen to which they are attached to form a heterocycloalkyl or heteroaryl; wherein R₂ is (v), and R₁₀ and R₁₁ taken together with the nitrogen to which they are attached to form a heterocycloalkyl or heteroaryl; wherein R₂ is (vi), and R₁₂ and R₁₃ taken together with the nitrogen to which they are attached to form a heterocycloalkyl or heteroaryl.

One preferred subgenus of compounds are those having Formula I, or a pharmaceutically acceptable salt thereof, wherein R₁ is 3′,3′-dimethylglutaryl or 3′,3′-dimethylsuccinyl; R₂ is formula (v); R₃ isopropenyl, or isopropyl; R₁₀ is hydrogen, C₁₋₄alkyl, preferably methyl, or C₁₋₄alkoxy(C₁₋₄)alkyl, preferably methoxyethyl; and R₁₁ is hydrogen, C₁₋₆ alkyl, amino, C₃₋₇ cycloalkyl, C₆₋₁₀aryl, C₆₋₁₀aryl(C₁₋₄)alkyl, C₁₋₄ alkylsulfonyl, phenylsulfonyl, piperidinyl, or pyrrolidinyl, any of which is optionally substituted by 1-5, preferably 1-3 groups independently selected from halo, trifluoromethyl, hydroxy, carboxy, amino, azido, C₁₋₄ alkoxy, monoalkylamino, dialkylamino, morpholinyl, cyano, acetyl, acetamido, pyridinyl, furanyl, thienyl, methylimidazolyl, methylisoxazolyl, methylpiperazinyl, methylmorpholinyl, tert-butoxycarbonyl, tert-butoxy-2-oxoethyl, and 4-tert-butoxycarbonylmorpholinyl, and wherein the C₆₋₁₀aryl, C₆₋₁₀aryl(C₁₋₄)alkyl, phenylsulfonyl, piperidinyl, and pyrrolidinyl can be also substituted by C₁₋₄alkyl, C₁₋₄hydroxyalkyl or C₁₋₄alkoxy(C₁₋₄)alkyl.

Preferred compounds wherein R₂ is (i) include, but are not limited to, those found in Table 1:

TABLE 1

| # | R₁ | R₃ | R₅ |
|---|---|---|---|
| 1 | 3′,3′-dimethylsuccinyl | isopropenyl | dimethylamino |
| 2 | 3′,3′-dimethylglutaryl | isopropenyl | dimethylamino |
| 3 | 3′,3′-dimethylsuccinyl | isopropenyl | 1-piperidinylmethyl |
| 4 | 3′,3′-dimethylglutaryl | isopropenyl | 1-piperidinylmethyl |
| 5 | 3′,3′-dimethylsuccinyl | isopropenyl | 5-tetrazolylmethyl |
| 6 | 3′,3′-dimethylglutaryl | isopropenyl | 5-tetrazolylmethyl |
| 7 | 3′,3′-dimethylsuccinyl | isopropenyl | 3-(5-methylisoxazolyl)methyl |
| 8 | 3′,3′-dimethylglutaryl | isopropenyl | 3-(5-methylisoxazolyl)methyl |
| 9 | 3′,3′-dimethylsuccinyl | isopropenyl | 2-(acetamido)ethyl |
| 10 | 3′,3′-dimethylglutaryl | isopropenyl | 2-(acetamido)ethyl |
| 11 | 3′,3′-dimethylsuccinyl | isopropenyl | 2- |
| | | | (dimethylaminocarbonyl)ethyl |
| 12 | 3′,3′-dimethylglutaryl | isopropenyl | 2- |
| | | | (dimethylaminocarbonyl)ethyl |
| 13 | 3′,3′-dimethylsuccinyl | isopropyl | dimethylamino |
| 14 | 3′,3′-dimethylglutaryl | isopropyl | dimethylamino |
| 15 | 3′,3′-dimethylsuccinyl | isopropyl | 1-piperidinylmethyl |
| 16 | 3′,3′-dimethylglutaryl | isopropyl | 1-piperidinylmethyl |
| 17 | 3′,3′-dimethylsuccinyl | isopropyl | 5-tetrazolylmethyl |
| 18 | 3′,3′-dimethylglutaryl | isopropyl | 5-tetrazolylmethyl |
| 19 | 3′,3′-dimethylsuccinyl | isopropyl | 3-(5-methylisoxazolyl)methyl |
| 20 | 3′,3′-dimethylglutaryl | isopropyl | 3-(5-methylisoxazolyl)methyl |
| 21 | 3′,3′-dimethylsuccinyl | isopropyl | 2-(acetamido)ethyl |
| 22 | 3′,3′-dimethylglutaryl | isopropyl | 2-(acetamido)ethyl |
| 23 | 3′,3′-dimethylsuccinyl | isopropyl | 2- |
| | | | (dimethylaminocarbonyl)ethyl |
| 24 | 3′,3′-dimethylglutaryl | isopropyl | 2- |
| | | | (dimethylaminocarbonyl)ethyl |

Preferred compounds wherein R₂ is (ii) include, but are not limited to, those found in Table 2:

TABLE 2

| # | R₁ | R₃ | R₆ |
|---|---|---|---|
| 25 | 3′,3′-dimethylsuccinyl | isopropenyl | 2-pyridinylmethyl |
| 26 | 3′,3′-dimethylglutaryl | isopropenyl | 2-pyridinylmethyl |
| 27 | 3′,3′-dimethylsuccinyl | isopropenyl | tert-butoxycarbonylmethyl |
| 28 | 3′,3′-dimethylglutaryl | isopropenyl | tert-butoxycarbonylmethyl |
| 29 | 3′,3′-dimethylsuccinyl | isopropenyl | 2-cyanoethyl |
| 30 | 3′,3′-dimethylglutaryl | isopropenyl | 2-cyanoethyl |
| 31 | 3′,3′-dimethylsuccinyl | isopropenyl | cycloocten-1-yl |
| 32 | 3′,3′-dimethylglutaryl | isopropenyl | cycloocten-1-yl |
| 33 | 3′,3′-dimethylsuccinyl | isopropyl | 2-pyridinylmethyl |
| 34 | 3′,3′-dimethylglutaryl | isopropyl | 2-pyridinylmethyl |
| 35 | 3′,3′-dimethylsuccinyl | isopropyl | tert-butoxycarbonylmethyl |
| 36 | 3′,3′-dimethylglutaryl | isopropyl | tert-butoxycarbonylmethyl |
| 37 | 3′,3′-dimethylsuccinyl | isopropyl | 2-cyanoethyl |
| 38 | 3′,3′-dimethylglutaryl | isopropyl | 2-cyanoethyl |
| 39 | 3′,3′-dimethylsuccinyl | isopropyl | cycloocten-1-yl |
| 40 | 3′,3′-dimethylglutaryl | isopropyl | cycloocten-1-yl |

Preferred compounds wherein R₂ is (iii) include, but are not limited to, those found in Table 3:

TABLE 3

| # | R₁ | R₃ | R₇ | R₈ |
|---|---|---|---|---|
| 41 | 3′,3′-dimethylsuccinyl | isopropenyl | hydrogen | 2-methoxyethyl |
| 42 | 3′,3′-dimethylglutaryl | isopropenyl | hydrogen | 2-methoxyethyl |
| 43 | 3′,3′-dimethylsuccinyl | isopropenyl | methyl | methoxymethyl |
| 44 | 3′,3′-dimethylglutaryl | isopropenyl | methyl | methoxymethyl |
| 45 | 3′,3′-dimethylsuccinyl | isopropyl | hydrogen | 2-methoxyethyl |
| 46 | 3′,3′-dimethylglutaryl | isopropyl | hydrogen | 2-methoxyethyl |
| 47 | 3′,3′-dimethylsuccinyl | isopropyl | methyl | methoxymethyl |
| 48 | 3′,3′-dimethylglutaryl | isopropyl | methyl | methoxymethyl |
| 49 | 3′,3′-dimethylglutaryl | isopropenyl | hydrogen | hydrogen |
| 50 | 3′,3′-dimethylglutaryl | isopropyl | hydrogen | hydrogen |
| 51 | 3′,3′-dimethylsuccinyl | isopropenyl | hydrogen | hydrogen |
| 52 | 3′,3′-dimethylsuccinyl | isopropyl | hydrogen | hydrogen |
| 53 | 3′,3′-dimethylglutaryl | isopropenyl | methyl | hydrogen |
| 54 | 3′,3′-dimethylglutaryl | isopropyl | methyl | hydrogen |
| 55 | 3′,3′-dimethylsuccinyl | isopropenyl | methyl | hydrogen |
| 56 | 3′,3′-dimethylsuccinyl | isopropyl | methyl | hydrogen |
| 57 | 3′,3′-dimethylglutaryl | isopropenyl | methyl | methyl |
| 58 | 3′,3′-dimethylglutaryl | isopropyl | methyl | methyl |
| 59 | 3′,3′-dimethylsuccinyl | isopropenyl | methyl | methyl |
| 60 | 3′,3′-dimethysuccinyl | isopropyl | methyl | methyl |
| 61 | 3′,3′-dimethylglutaryl | isopropenyl | ethyl | hydrogen |
| 62 | 3′,3′-dimethylglutaryl | isopropyl | ethyl | hydrogen |
| 63 | 3′,3′-dimethylsuccinyl | isopropenyl | ethyl | hydrogen |
| 64 | 3′,3′-dimethylsuccinyl | isopropyl | ethyl | hydrogen |
| 65 | 3′,3′-dimethylglutaryl | isopropenyl | ethyl | ethyl |
| 66 | 3′,3′-dimethylglutaryl | isopropyl | ethyl | ethyl |
| 67 | 3′,3′-dimethylsuccinyl | isopropenyl | ethyl | ethyl |
| 68 | 3′,3′-dimethylsuccinyl | isopropyl | ethyl | ethyl |
| 69 | 3′,3′-dimethylglutaryl | isopropenyl | ethyl | methyl |
| 70 | 3′,3′-dimethylglutaryl | isopropyl | ethyl | methyl |
| 71 | 3′,3′-dimethylsuccinyl | isopropenyl | ethyl | methyl |
| 72 | 3′,3′-dimethylsuccinyl | isopropyl | ethyl | methyl |
| 73 | 3′,3′-dimethylglutaryl | isopropenyl | propyl | methyl |
| 74 | 3′,3′-dimethylglutaryl | isopropyl | propyl | methyl |
| 75 | 3′,3′-dimethylsuccinyl | isopropenyl | propyl | methyl |
| 76 | 3′,3′-dimethylsuccinyl | isopropyl | propyl | methyl |
| 77 | 3′,3′-dimethylglutaryl | isopropenyl | propyl | propyl |
| 78 | 3′,3′-dimethylglutaryl | isopropyl | propyl | propyl |
| 79 | 3′,3′-dimethylsuccinyl | isopropenyl | propyl | propyl |
| 80 | 3′,3′-dimethylsuccinyl | isopropyl | propyl | propyl |
| 81 | 3′,3′-dimethylglutaryl | isopropenyl | cyclopropyl | methyl |
| 82 | 3′,3′-dimethylglutaryl | isopropyl | cyclopropyl | methyl |
| 83 | 3′,3′-dimethylsuccinyl | isopropenyl | cyclopropyl | methyl |
| 84 | 3′,3′-dimethylsuccinyl | isopropyl | cyclopropyl | methyl |
| 85 | 3′,3′-dimethylglutaryl | isopropenyl | cyclopropylmethyl | methyl |
| 86 | 3′,3′-dimethylglutaryl | isopropyl | cyclopropylmethyl | methyl |
| 87 | 3′,3′-dimethylsuccinyl | isopropenyl | cyclopropylmethyl | methyl |
| 88 | 3′,3′-dimethylsuccinyl | isopropyl | cyclopropylmethyl | methyl |
| 89 | 3′,3′-dimethylglutaryl | isopropenyl | hydroxyethyl | methyl |
| 90 | 3′,3′-dimethylglutaryl | isopropyl | hydroxyethyl | methyl |
| 91 | 3′,3′-dimethylsuccinyl | isopropenyl | hydroxyethyl | methyl |
| 92 | 3′,3′-dimethylsuccinyl | isopropyl | hydroxyethyl | methyl |
| 93 | 3′,3′-dimethylglutaryl | isopropenyl | methylsulfonyl | hydrogen |
| 94 | 3′,3′-dimethylglutaryl | isopropyl | methylsulfonyl | hydrogen |
| 95 | 3′,3′-dimethylsuccinyl | isopropenyl | methylsulfonyl | hydrogen |
| 96 | 3′,3′-dimethylsuccinyl | isopropyl | methylsulfonyl | hydrogen |
| 97 | 3′,3′-dimethylglutaryl | isopropenyl | methylsulfonyl | methyl |
| 98 | 3′,3′-dimethylglutaryl | isopropyl | methylsulfonyl | methyl |
| 99 | 3′,3′-dimethylsuccinyl | isopropenyl | methylsulfonyl | methyl |
| 100 | 3′,3′-dimethylsuccinyl | isopropyl | methylsulfonyl | methyl |

Preferred compounds wherein R₂ is (iii) and R₇ and R₈ are taken together to form a heterocycle include, but are not limited to, those found in Table 4:

TABLE 4

| | | | R₇ and R₈ taken with the nitrogen to which |
|---|---|---|---|
| # | R₁ | R₃ | they are attached |
| 101 | 3′,3′-dimethylsuccinyl | isopropenyl | pyrrolidinyl |
| 102 | 3′,3′-dimethylglutaryl | isopropenyl | pyrrolidinyl |
| 103 | 3′,3′-dimethylsuccinyl | isopropenyl | morpholinyl |
| 104 | 3′,3′-dimethylglutaryl | isopropenyl | morpholinyl |
| 105 | 3′,3′-dimethylsuccinyl | isopropenyl | piperazinyl |
| 106 | 3′,3′-dimethylglutaryl | isopropenyl | piperazinyl |
| 107 | 3′,3′-dimethylsuccinyl | isopropyl | pyrrolidinyl |
| 108 | 3′,3′-dimethylglutaryl | isopropyl | pyrrolidinyl |
| 109 | 3′,3′-dimethylsuccinyl | isopropyl | morpholinyl |
| 110 | 3′,3′-dimethylglutaryl | isopropyl | morpholinyl |
| 111 | 3′,3′-dimethylsuccinyl | isopropyl | piperazinyl |
| 112 | 3′,3′-dimethylglutaryl | isopropyl | piperazinyl |
| 113 | 3′,3′-dimethylglutaryl | isopropenyl | 4-methylpiperazinyl |
| 114 | 3′,3′-dimethylglutaryl | isopropyl | 4-methylpiperazinyl |
| 115 | 3′,3′-dimethylsuccinyl | isopropenyl | 4-methylpiperazinyl |
| 116 | 3′,3′-dimethylsuccinyl | isopropyl | 4-methylpiperazinyl |
| 117 | 3′,3′-dimethylglutaryl | isopropenyl | 4-ethylpiperazinyl |
| 118 | 3′,3′-dimethylglutaryl | isopropyl | 4-ethylpiperazinyl |
| 119 | 3′,3′-dimethylsuccinyl | isopropenyl | 4-ethylpiperazinyl |
| 120 | 3′,3′-dimethylsuccinyl | isopropyl | 4-ethylpiperazinyl |
| 121 | 3′,3′-dimethylglutaryl | isopropenyl | 4-cyclopropylpiperazinyl |
| 122 | 3′,3′-dimethylglutaryl | isopropyl | 4-cyclopropylpiperazinyl |
| 123 | 3′,3′-dimethylsuccinyl | isopropenyl | 4-cyclopropylpiperazinyl |
| 124 | 3′,3′-dimethylsuccinyl | isopropyl | 4-cyclopropylpiperazinyl |
| 125 | 3′,3′-dimethylglutaryl | isopropenyl | 4-(cyclopropylmethyl)piperazinyl |
| 126 | 3′,3′-dimethylglutaryl | isopropyl | 4-(cyclopropylmethyl)piperazinyl |
| 127 | 3′,3′-dimethylsuccinyl | isopropenyl | 4-(cyclopropylmethyl)piperazinyl |
| 128 | 3′,3′-dimethylsuccinyl | isopropyl | 4-(cyclopropylmethyl)piperazinyl |
| 129 | 3′,3′-dimethylglutaryl | isopropenyl | 4-acetylpiperazinyl |
| 130 | 3′,3′-dimethylglutaryl | isopropyl | 4-acetylpiperazinyl |
| 131 | 3′,3′-dimethylsuccinyl | isopropenyl | 4-acetylpiperazinyl |
| 132 | 3′,3′-dimethylsuccinyl | isopropyl | 4-acetylpiperazinyl |
| 133 | 3′,3′-dimethylglutaryl | isopropenyl | 4-(methylsulfonyl)piperazinyl |
| 134 | 3′,3′-dimethylglutaryl | isopropyl | 4-(methylsulfonyl)piperazinyl |
| 135 | 3′,3′-dimethylsuccinyl | isopropenyl | 4-(methylsulfonyl)piperazinyl |
| 136 | 3′,3′-dimethylsuccinyl | isopropyl | 4-(methylsulfonyl)piperazinyl |
| 137 | 3′,3′-dimethylglutaryl | isopropenyl | 4-(hydroxyethyl)piperazinyl |
| 138 | 3′,3′-dimethylglutaryl | isopropyl | 4-(hydroxyethyl)piperazinyl |
| 139 | 3′,3′-dimethylsuccinyl | isopropenyl | 4-(hydroxyethyl)piperazinyl |
| 140 | 3′,3′-dimethylsuccinyl | isopropyl | 4-(hydroxyethyl)piperazinyl |
| 141 | 3′,3′-dimethylglutaryl | isopropenyl | 4-(methoxyethyl)piperazinyl |
| 142 | 3′,3′-dimethylglutaryl | isopropyl | 4-(methoxyethyl)piperazinyl |
| 143 | 3′,3′-dimethylsuccinyl | isopropenyl | 4-(methoxyethyl)piperazinyl |
| 144 | 3′,3′-dimethylsuccinyl | isopropyl | 4-(methoxyethyl)piperazinyl |
| 145 | 3′,3′-dimethylglutaryl | isopropenyl | 4-isopropylpiperazinyl |
| 146 | 3′,3′-dimethylglutaryl | isopropyl | 4-isopropylpiperazinyl |
| 147 | 3′,3′-dimethylsuccinyl | isopropenyl | 4-isopropylpiperazinyl |
| 148 | 3′,3′-dimethylsuccinyl | isopropyl | 4-isopropylpiperazinyl |
| 149 | 3′,3′-dimethylglutaryl | isopropenyl | 3-aminopyrrolidinyl |
| 150 | 3′,3′-dimethylglutaryl | isopropyl | 3-aminopyrrolidinyl |
| 151 | 3′,3′-dimethylsuccinyl | isopropenyl | 3-aminopyrrolidinyl |
| 152 | 3′,3′-dimethylsuccinyl | isopropyl | 3-aminopyrrolidinyl |
| 153 | 3′,3′-dimethylglutaryl | isopropenyl | 3-N,N-dimethylaminopyrrolidinyl |
| 154 | 3′,3′-dimethylglutaryl | isopropyl | 3-N,N-dimethylaminopyrrolidinyl |
| 155 | 3′,3′-dimethylsuccinyl | isopropenyl | 3-N,N-dimethylaminopyrrolidinyl |
| 156 | 3′,3′-dimethylsuccinyl | isopropyl | 3-N,N-dimethylaminopyrrolidinyl |
| 157 | 3′,3′-dimethylglutaryl | isopropenyl | 3-hydroxypyrrolidinyl |
| 158 | 3′,3′-dimethylglutaryl | isopropyl | 3-hydroxypyrrolidinyl |
| 159 | 3′,3′-dimethylsuccinyl | isopropenyl | 3-hydroxypyrrolidinyl |
| 160 | 3′,3′-dimethylsuccinyl | isopropyl | 3-hydroxypyrrolidinyl |
| 161 | 3′,3′-dimethylglutaryl | isopropenyl | 3-acetamidopyrrolidinyl |
| 162 | 3′,3′-dimethylglutaryl | isopropyl | 3-acetamidopyrrolidinyl |
| 163 | 3′,3′-dimethylsuccinyl | isopropenyl | 3-acetamidopyrrolidinyl |
| 164 | 3′,3′-dimethylsuccinyl | isopropyl | 3-acetamidopyrrolidinyl |
| 165 | 3′,3′-dimethylglutaryl | isopropenyl | 3-(methylsulfonamido)pyrrolidinyl |
| 166 | 3′,3′-dimethylglutaryl | isopropyl | 3-(methylsulfonamido)pyrrolidinyl |
| 167 | 3′,3′-dimethylsuccinyl | isopropenyl | 3-(methylsulfonamido)pyrrolidinyl |
| 168 | 3′,3′-dimethylsuccinyl | isopropyl | 3-(methylsulfonamido)pyrrolidinyl |
| 169 | 3′,3′-dimethylglutaryl | isopropenyl | 4-benzylpiperazinyl |
| 170 | 3′,3′-dimethylglutaryl | isopropyl | 4-benzylpiperazinyl |
| 171 | 3′,3′-dimethylsuccinyl | isopropenyl | 4-benzylpiperazinyl |
| 172 | 3′,3′-dimethylsuccinyl | isopropyl | 4-benzylpiperazinyl |
| 173 | 3′,3′-dimethylglutaryl | isopropenyl | thiomorpholinyl |
| 174 | 3′,3′-dimethylglutaryl | isopropyl | thiomorpholinyl |
| 175 | 3′,3′-dimethylsuccinyl | isopropenyl | thiomorpholinyl |
| 176 | 3′,3′-dimethylsuccinyl | isopropyl | thiomorpholinyl |
| 177 | 3′,3′-dimethylglutaryl | isopropenyl | thiomorpholinyl 1-oxide |
| 178 | 3′,3′-dimethylglutaryl | isopropyl | thiomorpholinyl 1-oxide |
| 179 | 3′,3′-dimethylsuccinyl | isopropenyl | thiomorpholinyl 1-oxide |
| 180 | 3′,3′-dimethylsuccinyl | isopropyl | thiomorpholinyl 1-oxide |
| 181 | 3′,3′-dimethylglutaryl | isopropenyl | thiomorpholinyl 1,1-dioxide |
| 182 | 3′,3′-dimethylglutaryl | isopropyl | thiomorpholinyl 1,1-dioxide |
| 183 | 3′,3′-dimethylsuccinyl | isopropenyl | thiomorpholinyl 1,1-dioxide |
| 184 | 3′,3′-dimethylsuccinyl | isopropyl | thiomorpholinyl 1,1-dioxide |
| 185 | 3′,3′-dimethylglutaryl | isopropenyl | 4-aminopiperidinyl |
| 186 | 3′,3′-dimethylglutaryl | isopropyl | 4-aminopiperidinyl |
| 187 | 3′,3′-dimethylsuccinyl | isopropenyl | 4-aminopiperidinyl |
| 188 | 3′,3′-dimethylsuccinyl | isopropyl | 4-aminopiperidinyl |
| 189 | 3′,3′-dimethylglutaryl | isopropenyl | 4-N,N-dimethylaminopiperidinyl |
| 190 | 3′,3′-dimethylglutaryl | isopropyl | 4-N,N-dimethylaminopiperidinyl |
| 191 | 3′,3′-dimethylsuccinyl | isopropenyl | 4-N,N-dimethylaminopiperidinyl |
| 192 | 3′,3′-dimethylsuccinyl | isopropyl | 4-N,N-dimethylaminopiperidinyl |
| 193 | 3′,3′-dimethylglutaryl | isopropenyl | 4-acetamidopiperidinyl |
| 194 | 3′,3′-dimethylglutaryl | isopropyl | 4-acetamidopiperidinyl |
| 195 | 3′,3′-dimethylsuccinyl | isopropenyl | 4-acetamidopiperidinyl |
| 196 | 3′,3′-dimethylsuccinyl | isopropyl | 4-acetamidopiperidinyl |
| 197 | 3′,3′-dimethylglutaryl | isopropenyl | 4-(methylsulfonamido)piperidinyl |
| 198 | 3′,3′-dimethylglutaryl | isopropyl | 4-(methylsulfonamido)piperidinyl |
| 199 | 3′,3′-dimethylsuccinyl | isopropenyl | 4-(methylsulfonamido)piperidinyl |
| 200 | 3′,3′-dimethylsuccinyl | isopropyl | 4-(methylsulfonamido)piperidinyl |

Preferred compounds wherein R₂ is (iv) include, but are not limited to, those found in Table 5:

TABLE 5

| # | R₁ | R₃ | R₉ |
|---|---|---|---|
| 201 | 3′,3′-dimethylsuccinyl | isopropenyl | tert-butoxycarbonyloxymethyl |
| 202 | 3′,3′-dimethylglutaryl | isopropenyl | tert-butoxycarbonyloxymethyl |
| 203 | 3′,3′-dimethylsuccinyl | isopropenyl | (1′-ethoxycarbonyloxy) |
| | | | (1′-methyl)methyl |
| 204 | 3′,3′-dimethylglutaryl | isopropenyl | (1′-ethoxycarbonyloxy) |
| | | | (1′-methyl)methyl |
| 205 | 3′,3′-dimethylsuccinyl | isopropenyl | 2-cyanoethyl |
| 206 | 3′,3′-dimethylglutaryl | isopropenyl | 2-cyanoethyl |
| 207 | 3′,3′-dimethylsuccinyl | isopropenyl | (1′-ethoxymethyl)ethoxyethyl |
| 208 | 3′,3′-dimethylglutaryl | isopropenyl | (1′-ethoxymethyl)ethoxyethyl |
| 209 | 3′,3′-dimethylsuccinyl | isopropyl | tert-butoxycarbonyloxymethyl |
| 210 | 3′,3′-dimethylglutaryl | isopropyl | tert-butoxycarbonyloxymethyl |
| 211 | 3′,3′-dimethylsuccinyl | isopropyl | ethoxycarbonyloxy(1′methyl)methyl |
| 212 | 3′,3′-dimethylglutaryl | isopropyl | ethoxycarbonyloxy(1′methyl)methyl |
| 213 | 3′,3′-dimethylsuccinyl | isopropyl | 2-cyanoethyl |
| 214 | 3′,3′-dimethylglutaryl | isopropyl | 2-cyanoethyl |
| 215 | 3′,3′-dimethylsuccinyl | isopropyl | (1′-ethoxymethyl)ethoxyethyl |
| 216 | 3′,3′-dimethylglutaryl | isopropyl | (1′-ethoxymethyl)ethoxyethyl |
| 217 | 3′,3′-dimethylsuccinyl | isopropenyl | 2-dimethylaminoethyl |
| 218 | 3′,3′-dimethylglutaryl | isopropenyl | 2-dimethylaminoethyl |
| 219 | 3′,3′-dimethylsuccinyl | isopropyl | 2-dimethylaminoethyl |
| 220 | 3′,3′-dimethylglutaryl | isopropyl | 2-dimethylaminoethyl |
| 221 | 3′,3′-dimethylsuccinyl | isopropenyl | 2-methoxyethyl |
| 222 | 3′,3′-dimethylsuccinyl | isopropyl | 2-methoxyethyl |
| 223 | 3′,3′-dimethylglutaryl | isopropenyl | 2-methoxyethyl |
| 224 | 3′,3′-dimethylglutaryl | isopropyl | 2-methoxyethyl |
| 225 | 3′,3′-dimethylsuccinyl | isopropenyl | 3-[1-(tert- |
| | | | butoxycarbonyl)pyrrolidinyl] |
| 226 | 3′,3′-dimethylsuccinyl | isopropyl | 3-[1-(tert- |
| | | | butoxycarbonyl)pyrrolidinyl] |
| 227 | 3′,3′-dimethylglutaryl | isopropenyl | 3-[1-(tert- |
| | | | butoxycarbonyl)pyrrolidinyl] |
| 228 | 3′,3′-dimethylglutaryl | isopropyl | 3-[1-(tert- |
| | | | butoxycarbonyl)pyrrolidinyl] |
| 229 | 3′,3′-dimethylsuccinyl | isopropenyl | 3-tetrahydrofuranyl |
| 230 | 3′,3′-dimethylsuccinyl | isopropyl | 3-tetrahydrofuranyl |
| 231 | 3′,3′-dimethylglutaryl | isopropenyl | 3-tetrahydrofuranyl |
| 232 | 3′,3′-dimethylglutaryl | isopropyl | 3-tetrahydrofuranyl |
| 233 | 3′,3′-dimethylsuccinyl | isopropenyl | ethyl |
| 234 | 3′,3′-dimethylsuccinyl | isopropyl | ethyl |
| 235 | 3′,3′-dimethylglutaryl | isopropenyl | ethyl |
| 236 | 3′,3′-dimethylglutaryl | isopropyl | ethyl |
| 237 | 3′,3′-dimethylsuccinyl | isopropenyl | isopropyl |
| 238 | 3′,3′-dimethylsuccinyl | isopropyl | isopropyl |
| 239 | 3′,3′-dimethylglutaryl | isopropenyl | isopropyl |
| 240 | 3′,3′-dimethylglutaryl | isopropyl | isopropyl |
| 241 | 3′,3′-dimethylsuccinyl | isopropenyl | tert-butyl |
| 242 | 3′,3′-dimethylsuccinyl | isopropyl | tert-butyl |
| 243 | 3′,3′-dimethylglutaryl | isopropenyl | tert-butyl |
| 244 | 3′,3′-dimethylglutaryl | isopropyl | tert-butyl |

Preferred compounds wherein R₂ is (v) can include, but are not limited to, those found in Table 6:

TABLE 6

| # | R₁ | R₃ | R₁₁ | R₁₀ |
|---|---|---|---|---|
| 245 | 3′,3′-dimethylsuccinyl | isopropenyl | propyl | hydrogen |
| 246 | 3′,3′-dimethylglutaryl | isopropenyl | propyl | hydrogen |
| 247 | 3′,3′-dimethylsuccinyl | isopropenyl | 2-methoxyethyl | hydrogen |
| 248 | 3′,3′-dimethylglutaryl | isopropenyl | 2-methoxyethyl | hydrogen |
| 249 | 3′,3′-dimethylsuccinyl | isopropenyl | 2-(tert- | hydrogen |
| | | | butoxycarbonylamino) | |
| | | | ethyl | |
| 250 | 3′,3′-dimethylglutaryl | isopropenyl | 2-(tert- | hydrogen |
| | | | butoxycarbonylamino) | |
| | | | ethyl | |
| 251 | 3′,3′-dimethylsuccinyl | isopropenyl | hydrogen | hydrogen |
| 252 | 3′,3′-dimethylglutaryl | isopropenyl | hydrogen | hydrogen |
| 253 | 3′,3′-dimethylsuccinyl | isopropenyl | ethyl | hydrogen |
| 254 | 3′,3′-dimethylglutaryl | isopropenyl | ethyl | hydrogen |
| 255 | 3′,3′-dimethylsuccinyl | isopropenyl | cyclopropyl | hydrogen |
| 256 | 3′,3′-dimethylglutaryl | isopropenyl | cyclopropyl | hydrogen |
| 257 | 3′,3′-dimethylsuccinyl | isopropenyl | isopropyl | hydrogen |
| 258 | 3′,3′-dimethylglutaryl | isopropenyl | isopropyl | hydrogen |
| 259 | 3′,3′-dimethylsuccinyl | isopropenyl | 2-(4- | hydrogen |
| | | | morpholinyl)ethyl | |
| 260 | 3′,3′-dimethylglutaryl | isopropenyl | 2-(4- | hydrogen |
| | | | morpholinyl)ethyl | |
| 261 | 3′,3′-dimethylsuccinyl | isopropenyl | 4-fluorophenyl | hydrogen |
| 262 | 3′,3′-dimethylglutaryl | isopropenyl | 4-fluorophenyl | hydrogen |
| 263 | 3′,3′-dimethylsuccinyl | isopropenyl | 4-fluorobenzyl | hydrogen |
| 264 | 3′,3′-dimethylglutaryl | isopropenyl | 4-fluorobenzyl | hydrogen |
| 265 | 3′,3′-dimethylsuccinyl | isopropenyl | 2-aminoethyl | hydrogen |
| 266 | 3′,3′-dimethylglutaryl | isopropenyl | 2-aminoethyl | hydrogen |
| 267 | 3′,3′-dimethylsuccinyl | isopropyl | propyl | hydrogen |
| 268 | 3′,3′-dimethylglutaryl | isopropyl | propyl | hydrogen |
| 269 | 3′,3′-dimethylsuccinyl | isopropyl | 2-methoxyethyl | hydrogen |
| 270 | 3′,3′-dimethylglutaryl | isopropyl | 2-methoxyethyl | hydrogen |
| 271 | 3′,3′-dimethylsuccinyl | isopropyl | 2-(tert- | hydrogen |
| | | | butoxycarbonylamino) | |
| | | | ethyl | |
| 272 | 3′,3′-dimethylglutaryl | isopropyl | 2-(tert- | hydrogen |
| | | | butoxycarbonylamino) | |
| | | | ethyl | |
| 273 | 3′,3′-dimethylsuccinyl | isopropyl | hydrogen | hydrogen |
| 274 | 3′,3′-dimethylglutaryl | isopropyl | hydrogen | hydrogen |
| 275 | 3′,3′-dimethylsuccinyl | isopropyl | ethyl | hydrogen |
| 276 | 3′,3′-dimethylglutaryl | isopropyl | ethyl | hydrogen |
| 277 | 3′,3′-dimethylsuccinyl | isopropyl | cyclopropyl | hydrogen |
| 278 | 3′,3′-dimethylglutaryl | isopropyl | cyclopropyl | hydrogen |
| 279 | 3′,3′-dimethylsuccinyl | isopropyl | isopropyl | hydrogen |
| 280 | 3′,3′-dimethylglutaryl | isopropyl | isopropyl | hydrogen |
| 281 | 3′,3′-dimethylsuccinyl | isopropyl | 2-(4- | hydrogen |
| | | | morpholinyl)ethyl | |
| 282 | 3′,3′-dimethylglutaryl | isopropyl | 2-(4- | hydrogen |
| | | | morpholinyl)ethyl | |
| 283 | 3′,3′-dimethylsuccinyl | isopropyl | 4-fluorophenyl | hydrogen |
| 284 | 3′,3′-dimethylglutaryl | isopropyl | 4-fluorophenyl | hydrogen |
| 285 | 3′,3′-dimethylsuccinyl | isopropyl | 4-fluorobenzyl | hydrogen |
| 286 | 3′,3′-dimethylglutaryl | isopropyl | 4-fluorobenzyl | hydrogen |
| 287 | 3′,3′-dimethylsuccinyl | isopropyl | 2-aminoethyl | hydrogen |
| 288 | 3′,3′-dimethylglutaryl | isopropyl | 2-aminoethyl | hydrogen |
| 289 | 3′,3′-dimethylsuccinyl | isopropenyl | tert- | hydrogen |
| | | | butoxycarbonylamino | |
| 290 | 3′,3′-dimethylsuccinyl | isopropyl | tert- | hydrogen |
| | | | butoxycarbonylamino | |
| 291 | 3′,3′-dimethylglutaryl | isopropenyl | tert- | hydrogen |
| | | | butoxycarbonylamino | |
| 292 | 3′,3′-dimethylglutaryl | isopropyl | tert- | hydrogen |
| | | | butoxycarbonylamino | |
| 293 | 3′,3′-dimethylglutaryl | isopropenyl | methyl | hydrogen |
| 294 | 3′,3′-dimethylglutaryl | isopropyl | methyl | hydrogen |
| 295 | 3′,3′-dimethylsuccinyl | isopropenyl | methyl | hydrogen |
| 296 | 3′,3′-dimethylsuccinyl | isopropyl | methyl | hydrogen |
| 297 | 3′,3′-dimethylglutaryl | isopropenyl | 2-hydroxyethyl | hydrogen |
| 298 | 3′,3′-dimethylglutaryl | isopropyl | 2-hydroxyethyl | hydrogen |
| 299 | 3′,3′-dimethylsuccinyl | isopropenyl | 2-hydroxyethyl | hydrogen |
| 300 | 3′,3′-dimethylsuccinyl | isopropyl | 2-hydroxyethyl | hydrogen |
| 301 | 3′,3′-dimethylglutaryl | isopropenyl | 2-hydroxy-2- | hydrogen |
| | | | methylpropyl | |
| 302 | 3′,3′-dimethylglutaryl | isopropyl | 2-hydroxy-2- | hydrogen |
| | | | methylpropyl | |
| 303 | 3′,3′-dimethylsuccinyl | isopropenyl | 2-hydroxy-2- | hydrogen |
| | | | methylpropyl | |
| 304 | 3′,3′-dimethylsuccinyl | isopropyl | 2-hydroxy-2- | hydrogen |
| | | | methylpropyl | |
| 305 | 4′- | isopropenyl | phenylsulfonyl | hydrogen |
| | (methylsulfonylamino)- | | | |
| | 4′oxo-3′,3′- | | | |
| | dimethylbutanoyl | | | |
| 306 | 4′- | isopropyl | phenylsulfonyl | hydrogen |
| | (methylsulfonylamino)- | | | |
| | 4′oxo-3′,3′- | | | |
| | dimethylbutanoyl | | | |
| 307 | 5′- | isopropenyl | phenylsulfonyl | hydrogen |
| | (methylsulfonylamino)- | | | |
| | 5′oxo-3′,3′- | | | |
| | dimethylpentanoyl | | | |
| 308 | 5′- | isopropyl | phenylsulfonyl | hydrogen |
| | (methylsulfonylamino)- | | | |
| | 5′oxo-3′,3′- | | | |
| | dimethylpentanoyl | | | |
| 309 | 4′- | isopropenyl | phenylsulfonyl | hydrogen |
| | (phenylsulfonylamino)- | | | |
| | 4′oxo-3′,3′- | | | |
| | dimethylbutanoyl | | | |
| 310 | 4′- | isopropyl | phenylsulfonyl | hydrogen |
| | (phenylsulfonylamino)- | | | |
| | 4′oxo-3′,3′- | | | |
| | dimethylbutanoyl | | | |
| 311 | 4′- | isopropenyl | methylsulfonyl | hydrogen |
| | (methylsulfonylamino)- | | | |
| | 4′oxo-3′,3′- | | | |
| | dimethylbutanoyl | | | |
| 312 | 4′- | isopropyl | methylsulfonyl | hydrogen |
| | (methylsulfonylamino)- | | | |
| | 4′oxo-3′,3′- | | | |
| | dimethylbutanoyl | | | |
| 313 | 3′,3′-dimethylsuccinyl | isopropenyl | phenylsulfonyl | hydrogen |
| 314 | 3′,3′-dimethylsuccinyl | isopropyl | phenylsulfonyl | hydrogen |
| 315 | 3′,3′-dimethylglutaryl | isopropenyl | phenylsulfonyl | hydrogen |
| 316 | 3′,3′-dimethylglutaryl | isopropyl | phenylsulfonyl | hydrogen |
| 317 | 3′,3′-dimethylsuccinyl | isopropenyl | methylsulfonyl | hydrogen |
| 318 | 3′,3′-dimethylsuccinyl | isopropyl | methylsulfonyl | hydrogen |
| 319 | 3′,3′-dimethylglutaryl | isopropenyl | methylsulfonyl | hydrogen |
| 320 | 3′,3′-dimethylglutaryl | isopropyl | methylsulfonyl | hydrogen |
| 321 | 3′,3′-dimethylglutaryl | isopropenyl | 2-hydroxyethoxyethyl | hydrogen |
| 322 | 3′,3′-dimethylglutaryl | isopropyl | 2-hydroxyethoxyethyl | hydrogen |
| 323 | 3′,3′-dimethylsuccinyl | isopropenyl | 2-hydroxyethoxyethyl | hydrogen |
| 324 | 3′,3′-dimethylsuccinyl | isopropyl | 2-hydroxyethoxyethyl | hydrogen |
| 325 | 3′,3′-dimethylglutaryl | isopropenyl | (R,S)-2-[2,3- | hydrogen |
| | | | dihydroxypropyl] | |
| 326 | 3′,3′-dimethylglutaryl | isopropyl | (R,S)-2-[2,3- | hydrogen |
| | | | dihydroxypropyl] | |
| 327 | 3′,3′-dimethylsuccinyl | isopropenyl | (R,S)-2-[2,3- | hydrogen |
| | | | dihydroxypropyl] | |
| 328 | 3′,3′-dimethylsuccinyl | isopropyl | (R,S)-2-[2,3- | hydrogen |
| | | | dihydroxypropyl] | |
| 329 | 3′,3′-dimethylglutaryl | isopropenyl | (S)-2-[2,3- | hydrogen |
| | | | dihydroxypropyl] | |
| 330 | 3′,3′-dimethylglutaryl | isopropyl | (S)-2-[2,3- | hydrogen |
| | | | dihydroxypropyl] | |
| 331 | 3′,3′-dimethylsuccinyl | isopropenyl | (S)-2-[2,3- | hydrogen |
| | | | dihydroxypropyl] | |
| 332 | 3′,3′-dimethylsuccinyl | isopropyl | (S)-2-[2,3- | hydrogen |
| | | | dihydroxypropyl] | |
| 333 | 3′,3′-dimethylglutaryl | isopropenyl | (R)-[2,3- | hydrogen |
| | | | dihydroxypropyl] | |
| 334 | 3′,3′-dimethylglutaryl | isopropyl | (R)-[2,3- | hydrogen |
| | | | dihydroxypropyl] | |
| 335 | 3′,3′-dimethylsuccinyl | isopropenyl | (R)-[2,3- | hydrogen |
| | | | dihydroxypropyl] | |
| 336 | 3′,3′-dimethylsuccinyl | isopropyl | (R)-[2,3- | hydrogen |
| | | | dihydroxypropyl] | |
| 337 | 3′3′-dimethylglutaryl | isopropenyl | (S)-2-[(1-hydroxy-4- | hydrogen |
| | | | methylpentyl)] | |
| 338 | 3′,3′-dimethylglutaryl | isopropyl | (S)-2-[(1-hydroxy-4- | hydrogen |
| | | | methylpentyl)] | |
| 339 | 3′,3′-dimethylsuccinyl | isopropenyl | (S)-2-[(1-hydroxy-4- | hydrogen |
| | | | methylpentyl)] | |
| 340 | 3′,3′-dimethylsuccinyl | isopropyl | (S)-2-[(1-hydroxy-4- | hydrogen |
| | | | methylpentyl)] | |
| 341 | 3′,3′-dimethylglutaryl | isopropenyl | (R)-2-[(1-hydroxy-4- | hydrogen |
| | | | methylpentyl)] | |
| 342 | 3′,3′-dimethylglutaryl | isopropyl | (R)-2-[(1-hydroxy-4- | hydrogen |
| | | | methylpentyl)] | |
| 343 | 3′,3′-dimethylsuccinyl | isopropenyl | (R)-2-[(1-hydroxy-4- | hydrogen |
| | | | methylpentyl)] | |
| 344 | 3′,3′-dimethylsuccinyl | isopropyl | (R)-2-[(1-hydroxy-4- | hydrogen |
| | | | methylpentyl)] | |
| 345 | 3′,3′-dimethylglutaryl | isopropenyl | 2-methoxyethyl | methyl |
| 346 | 3′,3′-dimethylglutaryl | isopropyl | 2-methoxyethyl | methyl |
| 347 | 3′,3′-dimethylsuccinyl | isopropenyl | 2-methoxyethyl | methyl |
| 348 | 3′,3′-dimethylsuccinyl | isopropyl | 2-methoxyethyl | methyl |
| 349 | 3′,3′-dimethylglutaryl | isopropenyl | 2-methoxyethyl | 2- |
| | | | | methoxyethyl |
| 350 | 3′,3′-dimethylglutaryl | isopropyl | 2-methoxyethyl | 2- |
| | | | | methoxyethyl |
| 351 | 3′,3′-dimethylsuccinyl | isopropenyl | 2-methoxyethyl | 2- |
| | | | | methoxyethyl |
| 352 | 3′,3′-dimethylsuccinyl | isopropyl | 2-methoxyethyl | 2- |
| | | | | methoxyethyl |
| 353 | 3′,3′-dimethylglutaryl | isopropenyl | 2-tert-butoxy-2- | hydrogen |
| | | | oxoethyl | |
| 354 | 3′,3′-dimethylglutaryl | isopropyl | 2-tert-butoxy-2- | hydrogen |
| | | | oxoethyl | |
| 355 | 3′,3′-dimethylsuccinyl | isopropenyl | 2-tert-butoxy-2- | hydrogen |
| | | | oxoethyl | |
| 356 | 3′,3′-dimethylsuccinyl | isopropyl | 2-tert-butoxy-2- | hydrogen |
| | | | oxoethyl | |
| 357 | 3′,3′-dimethylglutaryl | isopropenyl | (S)-1-carboxy-3- | hydrogen |
| | | | methylbutyl | |
| 358 | 3′,3′-dimethylglutaryl | isopropyl | (S)-1-carboxy-3- | hydrogen |
| | | | methylbutyl | |
| 359 | 3′,3′-dimethylsuccinyl | isopropenyl | (S)-1-carboxy-3- | hydrogen |
| | | | methylbutyl | |
| 360 | 3′,3′-dimethylsuccinyl | isopropyl | (S)-1-carboxy-3- | hydrogen |
| | | | methylbutyl | |
| 361 | 3′,3′-dimethylglutaryl | isopropenyl | 2-cyanoethyl | hydrogen |
| 362 | 3′,3′-dimethylglutaryl | isopropyl | 2-cyanoethyl | hydrogen |
| 363 | 3′,3′-dimethylsuccinyl | isopropenyl | 2-cyanoethyl | hydrogen |
| 364 | 3′,3′-dimethylsuccinyl | isopropyl | 2-cyanoethyl | hydrogen |
| 365 | 3′,3′-dimethylglutaryl | isopropenyl | 2-acetamidoethyl | hydrogen |
| 366 | 3′,3′-dimethylglutaryl | isopropyl | 2-acetamidoethyl | hydrogen |
| 367 | 3′,3′-dimethylsuccinyl | isopropenyl | 2-acetamidoethyl | hydrogen |
| 368 | 3′,3′-dimethylsuccinyl | isopropyl | 2-acetamidoethyl | hydrogen |
| 369 | 3′,3′-dimethylglutaryl | isopropenyl | (S)-1-[(tert- | hydrogen |
| | | | butoxycarbonyl)pyrrolidinyl] | |
| 370 | 3′,3′-dimethylglutaryl | isopropyl | (S)-1-[(tert- | hydrogen |
| | | | butoxycarbonyl)pyrrolidinyl] | |
| 371 | 3′,3′-dimethylsuccinyl | isopropenyl | (S)-1-[(tert- | hydrogen |
| | | | butoxycarbonyl)pyrrolidinyl] | |
| 372 | 3′,3′-dimethylsuccinyl | isopropyl | (S)-1-[(tert- | hydrogen |
| | | | butoxycarbonyl)pyrrolidinyl] | |
| 373 | 3′,3′-dimethylglutaryl | isopropenyl | (R)-1-[(tert- | hydrogen |
| | | | butoxycarbonyl)pyrrolidinyl] | |
| 374 | 3′,3′-dimethylglutaryl | isopropyl | (R)-1-[(tert- | hydrogen |
| | | | butoxycarbonyl)pyrrolidinyl] | |
| 375 | 3′,3′-dimethylsuccinyl | isopropenyl | (R)-1-[(tert- | hydrogen |
| | | | butoxycarbonyl)pyrrolidinyl] | |
| 376 | 3′,3′-dimethylsuccinyl | isopropyl | (R)-1-[(tert- | hydrogen |
| | | | butoxycarbonyl)pyrrolidinyl] | |
| 377 | 3′,3′-dimethylglutaryl | isopropenyl | (S)-3-pyrrolidinyl | hydrogen |
| 378 | 3′,3′-dimethylglutaryl | isopropyl | (S)-3-pyrrolidinyl | hydrogen |
| 379 | 3′,3′-dimethylsuccinyl | isopropenyl | (S)-3-pyrrolidinyl | hydrogen |
| 380 | 3′,3′-dimethylsuccinyl | isopropyl | (S)-3-pyrrolidinyl | hydrogen |
| 381 | 3′,3′-dimethylglutaryl | isopropenyl | (R)-3-pyrrolidinyl | hydrogen |
| 382 | 3′,3′-dimethylglutaryl | isopropyl | (R)-3-pyrrolidinyl | hydrogen |
| 383 | 3′,3′-dimethylsuccinyl | isopropenyl | (R)-3-pyrrolidinyl | hydrogen |
| 384 | 3′,3′-dimethylsuccinyl | isopropyl | (R)-3-pyrrolidinyl | hydrogen |
| 385 | 3′,3′-dimethylglutaryl | isopropenyl | (S)-3-(1- | hydrogen |
| | | | methylpyrrolidinyl) | |
| 386 | 3′,3′-dimethylglutaryl | isopropyl | (S)-3-(1- | hydrogen |
| | | | methylpyrrolidinyl) | |
| 387 | 3′,3′-dimethylsuccinyl | isopropenyl | (S)-3-(1- | hydrogen |
| | | | methylpyrrolidinyl) | |
| 388 | 3′,3′-dimethylsuccinyl | isopropyl | (S)-3-(1- | hydrogen |
| | | | methylpyrrolidinyl) | |
| 389 | 3′,3′-dimethylglutaryl | isopropenyl | (R)-3-(1- | hydrogen |
| | | | methylpyrrolidinyl) | |
| 390 | 3′,3′-dimethylglutaryl | isopropyl | (R)-3-(1- | hydrogen |
| | | | methylpyrrolidinyl) | |
| 391 | 3′,3′-dimethylsuccinyl | isopropenyl | (R)-3-(1- | hydrogen |
| | | | methylpyrrolidinyl) | |
| 392 | 3′,3′-dimethylsuccinyl | isopropyl | (R)-3-(1- | hydrogen |
| | | | methylpyrrolidinyl) | |
| 393 | 3′,3′-dimethylglutaryl | isopropenyl | (S)-3-(1- | hydrogen |
| | | | acetylpyrrolidinyl) | |
| 394 | 3′,3′-dimethylglutaryl | isopropyl | (S)-3-(1- | hydrogen |
| | | | acetylpyrrolidinyl) | |
| 395 | 3′,3′-dimethylsuccinyl | isopropenyl | (S)-3-(1- | hydrogen |
| | | | acetylpyrrolidinyl) | |
| 396 | 3′,3′-dimethylsuccinyl | isopropyl | (S)-3-(1- | hydrogen |
| | | | acetylpyrrolidinyl) | |
| 397 | 3′,3′-dimethylglutaryl | isopropenyl | (R)-3-(1- | hydrogen |
| | | | acetylpyrrolidinyl) | |
| 398 | 3′,3′-dimethylglutaryl | isopropyl | (R)-3-(1- | hydrogen |
| | | | acetylpyrrolidinyl) | |
| 399 | 3′,3′-dimethylsuccinyl | isopropenyl | (R)-3-(1- | hydrogen |
| | | | acetylpyrrolidinyl) | |
| 400 | 3′,3′-dimethylsuccinyl | isopropyl | (R)-3-(1- | hydrogen |
| | | | acetylpyrrolidinyl) | |
| 401 | 3′,3′-dimethylglutaryl | isopropenyl | (S)-3-(1- | hydrogen |
| | | | methylsulfonylpyrrolidinyl) | |
| 402 | 3′,3′-dimethylglutaryl | isopropyl | (S)-3-(1- | hydrogen |
| | | | methylsulfonylpyrrolidinyl) | |
| 403 | 3′,3′-dimethylsuccinyl | isopropenyl | (S)-3-(1- | hydrogen |
| | | | methylsulfonylpyrrolidinyl) | |
| 404 | 3′,3′-dimethylsuccinyl | isopropyl | (S)-3-(1- | hydrogen |
| | | | methylsulfonylpyrrolidinyl) | |
| 405 | 3′,3′-dimethylglutaryl | isopropenyl | (R)-3-(1- | hydrogen |
| | | | methylsulfonylpyrrolidinyl) | |
| 406 | 3′,3′-dimethylglutaryl | isopropenyl | (R)-3-(1- | hydrogen |
| | | | methylsulfonylpyrrolidinyl) | |
| 407 | 3′,3′-dimethylglutaryl | isopropenyl | (R)-3-(1- | hydrogen |
| | | | methylsulfonylpyrrolidinyl) | |
| 408 | 3′,3′-dimethylglutaryl | isopropenyl | (R)-3-(1- | hydrogen |
| | | | methylsulfonylpyrrolidinyl) | |
| 409 | 3′,3′-dimethylglutaryl | isopropenyl | 4-(1-(tert- | hydrogen |
| | | | butoxycarbonyl)piperidinyl | |
| 410 | 3′,3′-dimethylsuccinyl | isopropenyl | 4-(1-(tert- | hydrogen |
| | | | butoxycarbonyl)piperidinyl | |
| 411 | 3′,3′-dimethylglutaryl | isopropyl | 4-(1-(tert- | hydrogen |
| | | | butoxycarbonyl)piperidinyl | |
| 412 | 3′,3′-dimethylsuccinyl | isopropyl | 4-(1-(tert- | hydrogen |
| | | | butoxycarbonyl)piperidinyl | |
| 413 | 3′,3′-dimethylglutaryl | isopropenyl | 4-piperidinyl | hydrogen |
| 414 | 3′,3′-dimethylsuccinyl | isopropenyl | 4-piperidinyl | hydrogen |
| 415 | 3′,3′-dimethylglutaryl | isopropyl | 4-piperidinyl | hydrogen |
| 416 | 3′,3′-dimethylsuccinyl | isopropyl | 4-piperidinyl | hydrogen |
| 417 | 3′,3′-dimethylglutaryl | isopropenyl | 4-(1- | hydrogen |
| | | | methylpiperidinyl) | |
| 418 | 3′,3′-dimethylglutaryl | isopropyl | 4-(1- | hydrogen |
| | | | methylpiperidinyl) | |
| 419 | 3′,3′-dimethylsuccinyl | isopropenyl | 4-(1- | hydrogen |
| | | | methylpiperidinyl) | |
| 420 | 3′,3′-dimethylsuccinyl | isopropyl | 4-(1- | hydrogen |
| | | | methylpiperidinyl) | |
| 421 | 3′,3′-dimethylglutaryl | isopropenyl | 4-[1-(1- | hydrogen |
| | | | hydroxyethyl)piperidinyl | |
| 422 | 3′,3′-dimethylglutaryl | isopropyl | 4-[1-(1- | hydrogen |
| | | | hydroxyethyl)piperidinyl | |
| 423 | 3′,3′-dimethylsuccinyl | isopropenyl | 4-[1-(1- | hydrogen |
| | | | hydroxyethyl)piperidinyl | |
| 424 | 3′,3′-dimethylsuccinyl | isopropyl | 4-[1-(1- | hydrogen |
| | | | hydroxyethyl)piperidinyl | |
| 425 | 3′,3′-dimethylglutaryl | isopropenyl | 4-[1-(1- | hydrogen |
| | | | methoxyethyl)piperidinyl | |
| 426 | 3′,3′-dimethylglutaryl | isopropyl | 4-[1-(1- | hydrogen |
| | | | methoxyethyl)piperidinyl | |
| 427 | 3′,3′-dimethylsuccinyl | isopropenyl | 4-[1-(1- | hydrogen |
| | | | methoxyethyl)piperidinyl | |
| 428 | 3′,3′-dimethylsuccinyl | isopropyl | 4-[1-(1- | hydrogen |
| | | | methoxyethyl)piperidinyl | |
| 429 | 3′,3′-dimethylglutaryl | isopropenyl | 3-fluorobenzyl | hydrogen |
| 430 | 3′,3′-dimethylglutaryl | isopropyl | 3-fluorobenzyl | hydrogen |
| 431 | 3′,3′-dimethylsuccinyl | isopropenyl | 3-fluorobenzyl | hydrogen |
| 432 | 3′,3′-dimethylsuccinyl | isopropyl | 3-fluorobenzyl | hydrogen |
| 433 | 3′,3′-dimethylglutaryl | isopropenyl | 2-fluorobenzyl | hydrogen |
| 434 | 3′,3′-dimethylglutaryl | isopropyl | 2-fluorobenzyl | hydrogen |
| 435 | 3′,3′-dimethylsuccinyl | isopropenyl | 2-fluorobenzyl | hydrogen |
| 436 | 3′,3′-dimethylsuccinyl | isopropyl | 2-fluorobenzyl | hydrogen |
| 437 | 3′,3′-dimethylglutaryl | isopropenyl | 4-chlorobenzyl | hydrogen |
| 438 | 3′,3′-dimethylglutaryl | isopropyl | 4-chlorobenzyl | hydrogen |
| 439 | 3′,3′-dimethylsuccinyl | isopropenyl | 4-chlorobenzyl | hydrogen |
| 440 | 3′,3′-dimethylsuccinyl | isopropyl | 4-chlorobenzyl | hydrogen |
| 441 | 3′,3′-dimethylglutaryl | isopropenyl | 3-chlorobenzyl | hydrogen |
| 442 | 3′,3′-dimethylglutaryl | isopropyl | 3-chlorobenzyl | hydrogen |
| 443 | 3′,3′-dimethylsuccinyl | isopropenyl | 3-chlorobenzyl | hydrogen |
| 444 | 3′,3′-dimethylsuccinyl | isopropyl | 3-chlorobenzyl | hydrogen |
| 445 | 3′,3′-dimethylglutaryl | isopropenyl | 2-chlorobenzyl | hydrogen |
| 446 | 3′,3′-dimethylglutaryl | isopropyl | 2-chlorobenzyl | hydrogen |
| 447 | 3′,3′-dimethylsuccinyl | isopropenyl | 2-chlorobenzyl | hydrogen |
| 448 | 3′,3′-dimethylsuccinyl | isopropyl | 2-chlorobenzyl | hydrogen |
| 449 | 3′,3′-dimethylglutaryl | isopropenyl | 4-methylbenzyl | hydrogen |
| 450 | 3′,3′-dimethylglutaryl | isopropyl | 4-methylbenzyl | hydrogen |
| 451 | 3′,3′-dimethylsuccinyl | isopropenyl | 4-methylbenzyl | hydrogen |
| 452 | 3′,3′-dimethylsuccinyl | isopropyl | 4-methylbenzyl | hydrogen |
| 453 | 3′,3′-dimethylglutaryl | isopropenyl | 3-methylbenzyl | hydrogen |
| 454 | 3′,3′-dimethylglutaryl | isopropyl | 3-methylbenzyl | hydrogen |
| 455 | 3′,3′-dimethylsuccinyl | isopropenyl | 3-methylbenzyl | hydrogen |
| 456 | 3′,3′-dimethylsuccinyl | isopropyl | 3-methylbenzyl | hydrogen |
| 457 | 3′,3′-dimethylglutaryl | isopropenyl | 2-methylbenzyl | hydrogen |
| 458 | 3′,3′-dimethylglutaryl | isopropyl | 2-methylbenzyl | hydrogen |
| 459 | 3′,3′-dimethylsuccinyl | isopropenyl | 2-methylbenzyl | hydrogen |
| 460 | 3′,3′-dimethylsuccinyl | isopropyl | 2-methylbenzyl | hydrogen |
| 461 | 3′,3′-dimethylglutaryl | isopropenyl | 4-methoxybenzyl | hydrogen |
| 462 | 3′,3′-dimethylglutaryl | isopropyl | 4-methoxybenzyl | hydrogen |
| 463 | 3′,3′-dimethylsuccinyl | isopropenyl | 4-methoxybenzyl | hydrogen |
| 464 | 3′,3′-dimethylsuccinyl | isopropyl | 4-methoxybenzyl | hydrogen |
| 465 | 3′,3′-dimethylglutaryl | isopropenyl | 3-methoxybenzyl | hydrogen |
| 466 | 3′,3′-dimethylglutaryl | isopropyl | 3-methoxybenzyl | hydrogen |
| 467 | 3′,3′-dimethylsuccinyl | isopropenyl | 3-methoxybenzyl | hydrogen |
| 468 | 3′,3′-dimethylsuccinyl | isopropyl | 3-methoxybenzyl | hydrogen |
| 469 | 3′,3′-dimethylglutaryl | isopropenyl | 2-methoxybenzyl | hydrogen |
| 470 | 3′,3′-dimethylglutaryl | isopropyl | 2-methoxybenzyl | hydrogen |
| 471 | 3′,3′-dimethylsuccinyl | isopropenyl | 2-methoxybenzyl | hydrogen |
| 472 | 3′,3′-dimethylsuccinyl | isopropyl | 2-methoxybenzyl | hydrogen |
| 473 | 3′,3′-dimethylglutaryl | isopropenyl | 4-N,N- | hydrogen |
| | | | dimethylaminobenzyl | |
| 474 | 3′,3′-dimethylglutaryl | isopropyl | 4-N,N- | hydrogen |
| | | | dimethylaminobenzyl | |
| 475 | 3′,3′-dimethylsuccinyl | isopropenyl | 4-N,N- | hydrogen |
| | | | dimethylaminobenzyl | |
| 476 | 3′,3′-dimethylsuccinyl | isopropyl | 4-N,N- | hydrogen |
| | | | dimethylaminobenzyl | |
| 477 | 3′,3′-dimethylglutaryl | isopropenyl | 4- | hydrogen |
| | | | trifluoromethylbenzyl | |
| 478 | 3′,3′-dimethylglutaryl | isopropyl | 4- | hydrogen |
| | | | trifluoromethylbenzyl | |
| 479 | 3′,3′-dimethylsuccinyl | isopropenyl | 4- | hydrogen |
| | | | trifluoromethylbenzyl | |
| 480 | 3′,3′-dimethylsuccinyl | isopropyl | 4- | hydrogen |
| | | | trifluoromethylbenzyl | |
| 481 | 3′,3′-dimethylglutaryl | isopropenyl | 4-carboxybenzyl | hydrogen |
| 482 | 3′,3′-dimethylglutaryl | isopropyl | 4-carboxybenzyl | hydrogen |
| 483 | 3′,3′-dimethylsuccinyl | isopropenyl | 4-carboxybenzyl | hydrogen |
| 484 | 3′,3′-dimethylsuccinyl | isopropyl | 4-carboxybenzyl | hydrogen |
| 485 | 3′,3′-dimethylglutaryl | isopropenyl | 3,4-dichlorobenzyl | hydrogen |
| 486 | 3′,3′-dimethylglutaryl | isopropyl | 3,4-dichlorobenzyl | hydrogen |
| 487 | 3′,3′-dimethylsuccinyl | isopropenyl | 3,4-dichlorobenzyl | hydrogen |
| 488 | 3′,3′-dimethylsuccinyl | isopropyl | 3,4-dichlorobenzyl | hydrogen |
| 489 | 3′,3′-dimethylglutaryl | isopropenyl | 2,4-dichlorobenzyl | hydrogen |
| 490 | 3′,3′-dimethylglutaryl | isopropyl | 2,4-dichlorobenzyl | hydrogen |
| 491 | 3′,3′-dimethylsuccinyl | isopropenyl | 2,4-dichlorobenzyl | hydrogen |
| 492 | 3′,3′-dimethylsuccinyl | isopropyl | 2,4-dichlorobenzyl | hydrogen |
| 493 | 3′,3′-dimethylglutaryl | isopropenyl | 2-pyridinylmethyl | hydrogen |
| 494 | 3′,3′-dimethylglutaryl | isopropyl | 2-pyridinylmethyl | hydrogen |
| 495 | 3′,3′-dimethylsuccinyl | isopropenyl | 2-pyridinylmethyl | hydrogen |
| 496 | 3′,3′-dimethylsuccinyl | isopropyl | 2-pyridinylmethyl | hydrogen |
| 497 | 3′,3′-dimethylglutaryl | isopropenyl | 3-pyridinylmethyl | hydrogen |
| 498 | 3′,3′-dimethylglutaryl | isopropyl | 3-pyridinylmethyl | hydrogen |
| 499 | 3′,3′-dimethylsuccinyl | isopropenyl | 3-pyridinylmethyl | hydrogen |
| 500 | 3′,3′-dimethylsuccinyl | isopropyl | 3-pyridinylmethyl | hydrogen |
| 501 | 3′,3′-dimethylglutaryl | isopropenyl | 4-pyridinylmethyl | hydrogen |
| 502 | 3′,3′-dimethylglutaryl | isopropyl | 4-pyridinylmethyl | hydrogen |
| 503 | 3′,3′-dimethylsuccinyl | isopropenyl | 4-pyridinylmethyl | hydrogen |
| 504 | 3′,3′-dimethylsuccinyl | isopropyl | 4-pyridinylmethyl | hydrogen |
| 505 | 3′,3′-dimethylglutaryl | isopropenyl | 2-furanylmethyl | hydrogen |
| 506 | 3′,3′-dimethylglutaryl | isopropyl | 2-furanylmethyl | hydrogen |
| 507 | 3′,3′-dimethylsuccinyl | isopropenyl | 2-furanylmethyl | hydrogen |
| 508 | 3′,3′-dimethylsuccinyl | isopropyl | 2-furanylmethyl | hydrogen |
| 509 | 3′,3′-dimethylglutaryl | isopropenyl | 2-thienylmethyl | hydrogen |
| 510 | 3′,3′-dimethylglutaryl | isopropyl | 2-thienylmethyl | hydrogen |
| 511 | 3′,3′-dimethylsuccinyl | isopropenyl | 2-thienylmethyl | hydrogen |
| 512 | 3′,3′-dimethylsuccinyl | isopropyl | 2-thienylmethyl | hydrogen |
| 513 | 3′,3′-dimethylglutaryl | isopropenyl | 2-benzyl | hydrogen |
| 514 | 3′,3′-dimethylglutaryl | isopropyl | 2-benzyl | hydrogen |
| 515 | 3′,3′-dimethylsuccinyl | isopropenyl | 2-benzyl | hydrogen |
| 516 | 3′,3′-dimethylsuccinyl | isopropyl | 2-benzyl | hydrogen |
| 517 | 3′,3′-dimethylglutaryl | isopropenyl | 3- | hydrogen |
| | | | trifluoromethylbenzyl | |
| 518 | 3′,3′-dimethylglutaryl | isopropyl | 3- | hydrogen |
| | | | trifluoromethylbenzyl | |
| 519 | 3′,3′-dimethylsuccinyl | isopropenyl | 3- | hydrogen |
| | | | trifluoromethylbenzyl | |
| 520 | 3′,3′-dimethylsuccinyl | isopropyl | 3- | hydrogen |
| | | | trifluoromethylbenzyl | |
| 521 | 3′,3′-dimethylglutaryl | isopropenyl | 2-(1,3,4- | hydrogen |
| | | | thiadiazolyl)methyl | |
| 522 | 3′,3′-dimethylglutaryl | isopropyl | 2-(1,3,4- | hydrogen |
| | | | thiadiazolyl)methyl | |
| 523 | 3′,3′-dimethylsuccinyl | isopropenyl | 2-(1,3,4- | hydrogen |
| | | | thiadiazolyl)methyl | |
| 524 | 3′,3′-dimethylsuccinyl | isopropyl | 2-(1,3,4- | hydrogen |
| | | | thiadiazolyl)methyl | |
| 525 | 3′,3′-dimethylglutaryl | isopropenyl | 4-cyanomethyl | hydrogen |
| 526 | 3′,3′-dimethylglutaryl | isopropyl | 4-cyanomethyl | hydrogen |
| 527 | 3′,3′-dimethylsuccinyl | isopropenyl | 4-cyanomethyl | hydrogen |
| 528 | 3′,3′-dimethylsuccinyl | isopropyl | 4-cyanomethyl | hydrogen |
| 529 | 3′,3′-dimethylglutaryl | isopropenyl | 4-tert-butylbenzyl | hydrogen |
| 530 | 3′,3′-dimethylglutaryl | isopropyl | 4-tert-butylbenzyl | hydrogen |
| 531 | 3′,3′-dimethylsuccinyl | isopropenyl | 4-tert-butylbenzyl | hydrogen |
| 532 | 3′,3′-dimethylsuccinyl | isopropyl | 4-tert-butylbenzyl | hydrogen |
| 533 | 3′,3′-dimethylglutaryl | isopropenyl | 4-aminobenzyl | hydrogen |
| 534 | 3′,3′-dimethylglutaryl | isopropyl | 4-aminobenzyl | hydrogen |
| 535 | 3′,3′-dimethylsuccinyl | isopropenyl | 4-aminobenzyl | hydrogen |
| 536 | 3′,3′-dimethylsuccinyl | isopropyl | 4-aminobenzyl | hydrogen |
| 537 | 3′,3′-dimethylglutaryl | isopropenyl | 4-acetamidobenzyl | hydrogen |
| 538 | 3′,3′-dimethylglutaryl | isopropyl | 4-acetamidobenzyl | hydrogen |
| 539 | 3′,3′-dimethylsuccinyl | isopropenyl | 4-acetamidobenzyl | hydrogen |
| 540 | 3′,3′-dimethylsuccinyl | isopropyl | 4-acetamidobenzyl | hydrogen |
| 541 | 3′,3′-dimethylglutaryl | isopropenyl | 1-(1,2,3,4- | hydrogen |
| | | | tetrahydronaphthyl) | |
| 542 | 3′,3′-dimethylglutaryl | isopropyl | 1-(1,2,3,4- | hydrogen |
| | | | tetrahydronaphthyl) | |
| 543 | 3′,3′-dimethylsuccinyl | isopropenyl | 1-(1,2,3,4- | hydrogen |
| | | | tetrahydronaphthyl) | |
| 544 | 3′,3′-dimethylsuccinyl | isopropyl | 1-(1,2,3,4- | hydrogen |
| | | | tetrahydronaphthyl) | |
| 545 | 3′,3′-dimethylglutaryl | isopropenyl | (R)-1-phenylethyl | hydrogen |
| 546 | 3′,3′-dimethylglutaryl | isopropyl | (R)-1-phenylethyl | hydrogen |
| 547 | 3′,3′-dimethylsuccinyl | isopropenyl | (R)-1-phenylethyl | hydrogen |
| 548 | 3′,3′-dimethylsuccinyl | isopropyl | (R)-1-phenylethyl | hydrogen |
| 549 | 3′,3′-dimethylglutaryl | isopropenyl | (S)-1-phenylethyl | hydrogen |
| 560 | 3′,3′-dimethylglutaryl | isopropyl | (S)-1-phenylethyl | hydrogen |
| 561 | 3′,3′-dimethylsuccinyl | isopropenyl | (S)-1-phenylethyl | hydrogen |
| 562 | 3′,3′-dimethylsuccinyl | isopropyl | (S)-1-phenylethyl | hydrogen |
| 563 | 3′,3′-dimethylglutaryl | isopropenyl | 4-(1- | hydrogen |
| | | | methylimidazolyl)methyl | |
| 564 | 3′,3′-dimethylglutaryl | isopropyl | 4-(1- | hydrogen |
| | | | methylimidazolyl)methyl | |
| 565 | 3′,3′-dimethylsuccinyl | isopropenyl | 4-(1- | hydrogen |
| | | | methylimidazolyl)methyl | |
| 566 | 3′,3′-dimethylsuccinyl | isopropyl | 4-(1- | hydrogen |
| | | | methylimidazolyl)methyl | |
| 567 | 3′,3′-dimethylglutaryl | isopropenyl | 3-(5- | hydrogen |
| | | | methylisoxazolyl)methyl | |
| 568 | 3′,3′-dimethylglutaryl | isopropyl | 3-(5- | hydrogen |
| | | | methylisoxazolyl)methyl | |
| 569 | 3′,3′-dimethylsuccinyl | isopropenyl | 3-(5- | hydrogen |
| | | | methylisoxazolyl)methyl | |
| 570 | 3′,3′-dimethylsuccinyl | isopropyl | 3-(5- | hydrogen |
| | | | methylisoxazolyl)methyl | |
| 571 | 3′,3′-dimethylglutaryl | isopropenyl | 2,3-dichlorobenzyl | hydrogen |
| 572 | 3′,3′-dimethylglutaryl | isopropyl | 2,3-dichlorobenzyl | hydrogen |
| 573 | 3′,3′-dimethylsuccinyl | isopropenyl | 2,3-dichlorobenzyl | hydrogen |
| 574 | 3′,3′-dimethylsuccinyl | isopropyl | 2,3-dichlorobenzyl | hydrogen |
| 575 | 3′,3′-dimethylglutaryl | isopropenyl | 4-(4- | hydrogen |
| | | | morpholinyl)benzyl | |
| 576 | 3′,3′-dimethylglutaryl | isopropyl | 4-(4- | hydrogen |
| | | | morpholinyl)benzyl | |
| 577 | 3′,3′-dimethylsuccinyl | isopropenyl | 4-(4- | hydrogen |
| | | | morpholinyl)benzyl | |
| 578 | 3′,3′-dimethylsuccinyl | isopropyl | 4-(4- | hydrogen |
| | | | morpholinyl)benzyl | |
| 579 | 3′,3′-dimethylglutaryl | isopropenyl | 4-(4- | hydrogen |
| | | | methylpiperazinyl)benzyl | |
| 580 | 3′,3′-dimethylglutaryl | isopropyl | 4-(4- | hydrogen |
| | | | methylpiperazinyl)benzyl | |
| 581 | 3′,3′-dimethylsuccinyl | isopropenyl | 4-(4- | hydrogen |
| | | | methylpiperazinyl)benzyl | |
| 582 | 3′,3′-dimethylsuccinyl | isopropyl | 4-(4- | hydrogen |
| | | | methylpiperazinyl)benzyl | |
| 583 | 3′,3′-dimethylglutaryl | isopropenyl | 3-[6-(4- | hydrogen |
| | | | morpholinyl)pyridinyl] | |
| | | | methyl | |
| 584 | 3′,3′-dimethylglutaryl | isopropyl | 3-[6-(4- | hydrogen |
| | | | morpholinyl)pyridinyl] | |
| | | | methyl | |
| 585 | 3′,3′-dimethylsuccinyl | isopropenyl | 3-[6-(4- | hydrogen |
| | | | morpholinyl)pyridinyl] | |
| | | | methyl | |
| 586 | 3′,3′-dimethylsuccinyl | isopropyl | 3-[6-(4- | hydrogen |
| | | | morpholinyl)pyridinyl] | |
| | | | methyl | |
| 587 | 3′,3′-dimethylglutaryl | isopropenyl | 4-azido-2,3,5,6- | hydrogen |
| | | | tetrafluorobenzyl | |
| 588 | 3′,3′-dimethylglutaryl | isopropyl | 4-azido-2,3,5,6- | hydrogen |
| | | | tetrafluorobenzyl | |
| 589 | 3′,3′-dimethylsuccinyl | isopropenyl | 4-azido-2,3,5,6- | hydrogen |
| | | | tetrafluorobenzyl | |
| 590 | 3′,3′-dimethylsuccinyl | isopropyl | 4-azido-2,3,5,6- | hydrogen |
| | | | tetrafluorobenzyl | |
| 591 | 3′,3′-dimethylglutaryl | isopropenyl | 2-[(4-azido-2,3,5,6- | hydrogen |
| | | | tetrafluorobenzoyl)amino] | |
| | | | ethyl | |
| 592 | 3′,3′-dimethylglutaryl | isopropyl | 2-[(4-azido-2,3,5,6- | hydrogen |
| | | | tetrafluorobenzoyl)amino] | |
| | | | ethyl | |
| 593 | 3′,3′-dimethylsuccinyl | isopropenyl | 2-[(4-azido-2,3,5,6- | hydrogen |
| | | | tetrafluorobenzoyl)amino] | |
| | | | ethyl | |
| 594 | 3′,3′-dimethylsuccinyl | isopropyl | 2-[(4-azido-2,3,5,6- | hydrogen |
| | | | tetrafluorobenzoyl)amino] | |
| | | | ethyl | |
| 595 | 3′,3′-dimethylglutaryl | isopropenyl | (R)-2-hydroxy-1- | hydrogen |
| | | | phenylethyl | |
| 596 | 3′,3′-dimethylglutaryl | isopropyl | (R)-2-hydroxy-1- | hydrogen |
| | | | phenylethyl | |
| 597 | 3′,3′-dimethylsuccinyl | isopropenyl | (R)-2-hydroxy-1- | hydrogen |
| | | | phenylethyl | |
| 598 | 3′,3′-dimethylsuccinyl | isopropyl | (R)-2-hydroxy-1- | hydrogen |
| | | | phenylethyl | |
| 599 | 3′,3′-dimethylglutaryl | isopropenyl | (S)-2-hydroxy-1- | hydrogen |
| | | | phenylethyl | |
| 600 | 3′,3′-dimethylglutaryl | isopropyl | (S)-2-hydroxy-1- | hydrogen |
| | | | phenylethyl | |
| 601 | 3′,3′-dimethylsuccinyl | isopropenyl | (S)-2-hydroxy-1- | hydrogen |
| | | | phenylethyl | |
| 602 | 3′,3′-dimethylsuccinyl | isopropyl | (S)-2-hydroxy-1- | hydrogen |
| | | | phenylethyl | |
| 603 | 3′,3′-dimethylglutaryl | isopropenyl | 2-phenylethyl | hydrogen |
| 604 | 3′,3′-dimethylglutaryl | isopropyl | 2-phenylethyl | hydrogen |
| 605 | 3′,3′-dimethylsuccinyl | isopropenyl | 2-phenylethyl | hydrogen |
| 606 | 3′,3′-dimethylsuccinyl | isopropyl | 2-phenylethyl | hydrogen |
| 607 | 3′,3′-dimethylglutaryl | isopropenyl | 2-N,N- | hydrogen |
| | | | dimethylaminoethyl | |
| 608 | 3′,3′-dimethylglutaryl | isopropyl | 2-N,N- | hydrogen |
| | | | dimethylaminoethyl | |
| 609 | 3′,3′-dimethylsuccinyl | isopropenyl | 2-N,N- | hydrogen |
| | | | dimethylaminoethyl | |
| 610 | 3′,3′-dimethylsuccinyl | isopropyl | 2-N,N- | hydrogen |
| | | | dimethylaminoethyl | |
| 611 | 3′,3′-dimethylglutaryl | isopropenyl | 2-(1-amino-2- | hydrogen |
| | | | methylpropyl) | |
| 612 | 3′,3′-dimethylglutaryl | isopropyl | 2-(1-amino-2- | hydrogen |
| | | | methylpropyl) | |
| 613 | 3′,3′-dimethylsuccinyl | isopropenyl | 2-(1-amino-2- | hydrogen |
| | | | methylpropyl) | |
| 614 | 3′,3′-dimethylsuccinyl | isopropyl | 2-(1-amino-2- | hydrogen |
| | | | methylpropyl) | |
| 615 | 3′,3′-dimethylglutaryl | isopropenyl | 2-N,N- | hydrogen |
| | | | dimethylaminopropyl | |
| 616 | 3′,3′-dimethylglutaryl | isopropyl | 2-N,N- | hydrogen |
| | | | dimethylaminopropyl | |
| 617 | 3′,3′-dimethylsuccinyl | isopropenyl | 2-N,N- | hydrogen |
| | | | dimethylaminopropyl | |
| 618 | 3′,3′-dimethylsuccinyl | isopropyl | 2-N,N- | hydrogen |
| | | | dimethylaminopropyl | |
| 619 | 3′,3′-dimethylglutaryl | isopropenyl | 3-(4- | hydrogen |
| | | | morpholinyl)propyl | |
| 620 | 3′,3′-dimethylglutaryl | isopropyl | 3-(4- | hydrogen |
| | | | morpholinyl)propyl | |
| 621 | 3′,3′-dimethylsuccinyl | isopropenyl | 3-(4- | hydrogen |
| | | | morpholinyl)propyl | |
| 622 | 3′,3′-dimethylsuccinyl | isopropyl | 3-(4- | hydrogen |
| | | | morpholinyl)propyl | |
| 623 | 3′,3′-dimethylglutaryl | isopropenyl | 3-(1- | hydrogen |
| | | | imidazolyl)propyl | |
| 624 | 3′,3′-dimethylglutaryl | isopropyl | 3-(1- | hydrogen |
| | | | imidazolyl)propyl | |
| 625 | 3′,3′-dimethylsuccinyl | isopropenyl | 3-(1- | hydrogen |
| | | | imidazolyl)propyl | |
| 626 | 3′,3′-dimethylsuccinyl | isopropyl | 3-(1- | hydrogen |
| | | | imidazolyl)propyl | |
| 627 | 3′,3′-dimethylglutaryl | isopropenyl | 2-(4- | methyl |
| | | | methylmorpholinyl)methyl | |
| 628 | 3′,3′-dimethylglutaryl | isopropyl | 2-(4- | methyl |
| | | | methylmorpholinyl)methyl | |
| 629 | 3′,3′-dimethylsuccinyl | isopropenyl | 2-(4- | methyl |
| | | | methylmorpholinyl)methyl | |
| 630 | 3′,3′-dimethylsuccinyl | isopropyl | 2-(4- | methyl |
| | | | methylmorpholinyl)methyl | |
| 631 | 3′,3′-dimethylglutaryl | isopropenyl | 2-morpholinylmethyl | methyl |
| 632 | 3′,3′-dimethylglutaryl | isopropyl | 2-morpholinylmethyl | methyl |
| 633 | 3′,3′-dimethylsuccinyl | isopropenyl | 2-morpholinylmethyl | methyl |
| 634 | 3′,3′-dimethylsuccinyl | isopropyl | 2-morpholinylmethyl | methyl |
| 635 | 3′,3′-dimethylglutaryl | isopropenyl | 2-(4-tert- | methyl |
| | | | butoxycarbonyl | |
| | | | morpholinyl)methyl | |
| 636 | 3′,3′-dimethylglutaryl | isopropyl | 2-(4-tert- | methyl |
| | | | butoxycarbonyl | |
| | | | morpholinyl)methyl | |
| 637 | 3′,3′-dimethylsuccinyl | isopropenyl | 2-(4-tert- | methyl |
| | | | butoxycarbonyl | |
| | | | morpholinyl)methyl | |
| 638 | 3′,3′-dimethylsuccinyl | isopropyl | 2-(4-tert- | methyl |
| | | | butoxycarbonyl | |
| | | | morpholinyl)methyl | |
| 639 | 3′,3′-dimethylglutaryl | isopropenyl | (1R,3R)-3-N,N- | hydrogen |
| | | | dimethylaminocyclopentyl | |
| 640 | 3′,3′-dimethylglutaryl | isopropyl | (1R,3R)-3-N,N- | hydrogen |
| | | | dimethylaminocyclopentyl | |
| 641 | 3′,3′-dimethylsuccinyl | isopropenyl | (1R,3R)-3-N,N- | hydrogen |
| | | | dimethylaminocyclopentyl | |
| 642 | 3′,3′-dimethylsuccinyl | isopropyl | (1R,3R)-3-N,N- | hydrogen |
| | | | dimethylaminocyclopentyl | |
| 643 | 3′,3′-dimethylglutaryl | isopropenyl | (1S,3S)-3-N,N- | hydrogen |
| | | | dimethylaminocyclopentyl | |
| 644 | 3′,3′-dimethylglutaryl | isopropyl | (1S,3S)-3-N,N- | hydrogen |
| | | | dimethylaminocyclopentyl | |
| 645 | 3′,3′-dimethylsuccinyl | isopropenyl | (1S,3S)-3-N,N- | hydrogen |
| | | | dimethylaminocyclopentyl | |
| 646 | 3′,3′-dimethylsuccinyl | isopropyl | (1S,3S)-3-N,N- | hydrogen |
| | | | dimethylaminocyclopentyl | |
| 647 | 3′,3′-dimethylglutaryl | isopropenyl | (1R,3R)-3- | hydrogen |
| | | | aminocyclopentyl | |
| 648 | 3′,3′-dimethylglutaryl | isopropyl | (1R,3R)-3- | hydrogen |
| | | | aminocyclopentyl | |
| 649 | 3′,3′-dimethylsuccinyl | isopropenyl | (1R,3R)-3- | hydrogen |
| | | | aminocyclopentyl | |
| 650 | 3′,3′-dimethylsuccinyl | isopropyl | (1R,3R)-3- | hydrogen |
| | | | aminocyclopentyl | |
| 651 | 3′,3′-dimethylglutaryl | isopropenyl | (1S,3S)-3- | hydrogen |
| | | | aminocyclopentyl | |
| 652 | 3′,3′-dimethylglutaryl | isopropyl | (1S,3S)-3- | hydrogen |
| | | | aminocyclopentyl | |
| 653 | 3′,3′-dimethylsuccinyl | isopropenyl | (1S,3S)-3- | hydrogen |
| | | | aminocyclopentyl | |
| 654 | 3′,3′-dimethylsuccinyl | isopropyl | (1S,3S)-3- | hydrogen |
| | | | aminocyclopentyl | |
| 655 | 3′,3′-dimethylglutaryl | isopropenyl | (1r,4r)-4-N,N- | hydrogen |
| | | | dimethylaminocyclohexyl | |
| 656 | 3′,3′-dimethylglutaryl | isopropyl | (1r,4r)-4-N,N- | hydrogen |
| | | | dimethylaminocyclohexyl | |
| 657 | 3′,3′-dimethylsuccinyl | isopropenyl | (1r,4r)-4-N,N- | hydrogen |
| | | | dimethylaminocyclohexyl | |
| 658 | 3′,3′-dimethylsuccinyl | isopropyl | (1r,4r)-4-N,N- | hydrogen |
| | | | dimethylaminocyclohexyl | |
| 659 | 3′,3′-dimethylglutaryl | isopropenyl | (1s,4s)-4-N,N- | hydrogen |
| | | | dimethylaminocyclohexyl | |
| 660 | 3′,3′-dimethylglutaryl | isopropyl | (1s,4s)-4-N,N- | hydrogen |
| | | | dimethylaminocyclohexyl | |
| 661 | 3′,3′-dimethylsuccinyl | isopropenyl | (1s,4s)-4-N,N- | hydrogen |
| | | | dimethylaminocyclohexyl | |
| 662 | 3′,3′-dimethylsuccinyl | isopropyl | (1s,4s)-4-N,N- | hydrogen |
| | | | dimethylaminocyclohexyl | |
| 663 | 3′,3′-dimethylglutaryl | isopropenyl | (1r,4r)-4- | hydrogen |
| | | | aminocyclohexyl | |
| 664 | 3′,3′-dimethylglutaryl | isopropyl | (1r,4r)-4- | hydrogen |
| | | | aminocyclohexyl | |
| 665 | 3′,3′-dimethylsuccinyl | isopropenyl | (1r,4r)-4- | hydrogen |
| | | | aminocyclohexyl | |
| 666 | 3′,3′-dimethylsuccinyl | isopropyl | (1r,4r)-4- | hydrogen |
| | | | aminocyclohexyl | |
| 667 | 3′,3′-dimethylglutaryl | isopropenyl | (1s,4s)-4- | hydrogen |
| | | | aminocyclohexyl | |
| 668 | 3′,3′-dimethylglutaryl | isopropyl | (1s,4s)-4- | hydrogen |
| | | | aminocyclohexyl | |
| 669 | 3′,3′-dimethylsuccinyl | isopropenyl | (1s,4s)-4- | hydrogen |
| | | | aminocyclohexyl | |
| 670 | 3′,3′-dimethylsuccinyl | isopropyl | (1s,4s)-4- | hydrogen |
| | | | aminocyclohexyl | |

Preferred compounds wherein R₂ is (v) and R₁₀ and R₁₁ are taken together with the nitrogen to which they are attached to form a heterocycle or heteroaryl include, but are not limited to, those found in Table 7:

TABLE 7

| | | | R₁₀ and R₁₁ taken with the |
|---|---|---|---|
| | | | nitrogen to which they are |
| # | R₁ | R₃ | attached |
| 671 | 3′,3′-dimethylsuccinyl | isopropenyl | 4-(tert-butoxycarbonyl)piperazinyl |
| 672 | 3′,3′-dimethylglutaryl | isopropenyl | 4-(tert-butoxycarbonyl)piperazinyl |
| 673 | 3′,3′-dimethylsuccinyl | isopropenyl | morpholinyl |
| 674 | 3′,3′-dimethylglutaryl | isopropenyl | morpholinyl |
| 675 | 3′,3′-dimethylsuccinyl | isopropenyl | piperidinyl |
| 676 | 3′,3′-dimethylglutaryl | isopropenyl | piperidinyl |
| 677 | 3′,3′-dimethylsuccinyl | isopropenyl | piperazinyl |
| 678 | 3′,3′-dimethylglutaryl | isopropenyl | piperazinyl |
| 679 | 3′,3′-dimethylsuccinyl | isopropyl | 4-(tert-butoxycarbonyl)piperazinyl |
| 680 | 3′,3′-dimethylglutaryl | isopropyl | 4-(tert-butoxycarbonyl)piperazinyl |
| 681 | 3′,3′-dimethylsuccinyl | isopropyl | morpholinyl |
| 682 | 3′,3′-dimethylglutaryl | isopropyl | morpholinyl |
| 683 | 3′,3′-dimethylsuccinyl | isopropyl | piperidinyl |
| 684 | 3′,3′-dimethylglutaryl | isopropyl | piperidinyl |
| 685 | 3′,3′-dimethylsuccinyl | isopropyl | piperazinyl |
| 686 | 3′,3′-dimethylglutaryl | isopropyl | piperazinyl |
| 687 | 3′,3′-dimethyl-4-(4- | isopropenyl | 4-(4- |
| | morpholinyl)-4- | | morpholinylcarbonyl)piperazinyl |
| | oxobutanoyl | | |
| 688 | 3′,3′-dimethyl-4-(4- | isopropyl | 4-(4- |
| | morpholinyl)-4- | | morpholinylcarbonyl)piperazinyl |
| | oxobutanoyl | | |
| 689 | 3′,3′-dimethylglutaryl | isopropenyl | 4-methylpiperazinyl |
| 690 | 3′,3′-dimethylglutaryl | isopropyl | 4-methylpiperazinyl |
| 691 | 3′,3′-dimethylsuccinyl | isopropenyl | 4-methylpiperazinyl |
| 692 | 3′,3′-dimethylsuccinyl | isopropyl | 4-methylpiperazinyl |
| 693 | 3′,3′-dimethylglutaryl | isopropenyl | 4-ethylpiperazinyl |
| 694 | 3′,3′-dimethylglutaryl | isopropyl | 4-ethylpiperazinyl |
| 695 | 3′,3′-dimethylsuccinyl | isopropenyl | 4-ethylpiperazinyl |
| 696 | 3′,3′-dimethylsuccinyl | isopropyl | 4-ethylpiperazinyl |
| 697 | 3′,3′-dimethylglutaryl | isopropenyl | 4-isopropylpiperazinyl |
| 698 | 3′,3′-dimethylglutaryl | isopropyl | 4-isopropylpiperazinyl |
| 699 | 3′,3′-dimethylsuccinyl | isopropenyl | 4-isopropylpiperazinyl |
| 700 | 3′,3′-dimethylsuccinyl | isopropyl | 4-isopropylpiperazinyl |
| 701 | 3′,3′-dimethylglutaryl | isopropenyl | 4-(cyclopropylmethyl)piperazinyl |
| 702 | 3′,3′-dimethylglutaryl | isopropyl | 4-(cyclopropylmethyl)piperazinyl |
| 703 | 3′,3′-dimethylsuccinyl | isopropenyl | 4-(cyclopropylmethyl)piperazinyl |
| 704 | 3′,3′-dimethylsuccinyl | isopropyl | 4-(cyclopropylmethyl)piperazinyl |
| 705 | 3′,3′-dimethylglutaryl | isopropenyl | 4-benzylpiperazinyl |
| 706 | 3′,3′-dimethylglutaryl | isopropyl | 4-benzylpiperazinyl |
| 707 | 3′,3′-dimethylsuccinyl | isopropenyl | 4-benzylpiperazinyl |
| 708 | 3′,3′-dimethylsuccinyl | isopropyl | 4-benzylpiperazinyl |
| 709 | 3′,3′-dimethylglutaryl | isopropenyl | 4-[3-(5- |
| | | | methylisoxazolyl)methyl]piperazinyl |
| 710 | 3′,3′-dimethylglutaryl | isopropyl | 4-[3-(5- |
| | | | methylisoxazolyl)methyl]piperazinyl |
| 711 | 3′,3′-dimethylsuccinyl | isopropenyl | 4-[3-(5- |
| | | | methylisoxazolyl)methyl]piperazinyl |
| 712 | 3′,3′-dimethylsuccinyl | isopropyl | 4-[3-(5- |
| | | | methylisoxazolyl)methyl]piperazinyl |
| 713 | 3′,3′-dimethylglutaryl | isopropenyl | 4-(4-pyridinylmethyl)piperazinyl |
| 714 | 3′,3′-dimethylglutaryl | isopropyl | 4-(4-pyridinylmethyl)piperazinyl |
| 715 | 3′,3′-dimethylsuccinyl | isopropenyl | 4-(4-pyridinylmethyl)piperazinyl |
| 716 | 3′,3′-dimethylsuccinyl | isopropyl | 4-(4-pyridinylmethyl)piperazinyl |
| 717 | 3′,3′-dimethylglutaryl | isopropenyl | 4-acetylpiperazinyl |
| 718 | 3′,3′-dimethylglutaryl | isopropyl | 4-acetylpiperazinyl |
| 719 | 3′,3′-dimethylsuccinyl | isopropenyl | 4-acetylpiperazinyl |
| 720 | 3′,3′-dimethylsuccinyl | isopropyl | 4-acetylpiperazinyl |
| 721 | 3′,3′-dimethylglutaryl | isopropenyl | 4- |
| | | | (isopropylaminocarbonyl)piperazinyl |
| 722 | 3′,3′-dimethylglutaryl | isopropyl | 4- |
| | | | (isopropylaminocarbonyl)piperazinyl |
| 723 | 3′,3′-dimethylsuccinyl | isopropenyl | 4- |
| | | | (isopropylaminocarbonyl)piperazinyl |
| 724 | 3′,3′-dimethylsuccinyl | isopropyl | 4- |
| | | | (isopropylaminocarbonyl)piperazinyl |
| 725 | 3′,3′-dimethylglutaryl | isopropenyl | 4-(methylsulfonyl)piperazinyl |
| 726 | 3′,3′-dimethylglutaryl | isopropyl | 4-(methylsulfonyl)piperazinyl |
| 727 | 3′,3′-dimethylsuccinyl | isopropenyl | 4-(methylsulfonyl)piperazinyl |
| 728 | 3′,3′-dimethylsuccinyl | isopropyl | 4-(methylsulfonyl)piperazinyl |
| 729 | 3′,3′-dimethylglutaryl | isopropenyl | 4-cyclopropylpiperazinyl |
| 730 | 3′,3′-dimethylglutaryl | isopropyl | 4-cyclopropylpiperazinyl |
| 731 | 3′,3′-dimethylsuccinyl | isopropenyl | 4-cyclopropylpiperazinyl |
| 732 | 3′,3′-dimethylsuccinyl | isopropyl | 4-cyclopropylpiperazinyl |
| 733 | 3′,3′-dimethylglutaryl | isopropenyl | 4-(2- |
| | | | methoxyethylaminocarbonyl)piperazinyl |
| 734 | 3′,3′-dimethylglutaryl | isopropyl | 4-(2- |
| | | | methoxyethylaminocarbonyl)piperazinyl |
| 735 | 3′,3′-dimethylsuccinyl | isopropenyl | 4-(2- |
| | | | methoxyethylaminocarbonyl)piperazinyl |
| 736 | 3′,3′-dimethylsuccinyl | isopropyl | 4-(2- |
| | | | methoxyethylaminocarbonyl)piperazinyl |
| 737 | 3′,3′-dimethylglutaryl | isopropenyl | 4-(2-hydroxyethyl)piperazinyl |
| 738 | 3′,3′-dimethylglutaryl | isopropyl | 4-(2-hydroxyethyl)piperazinyl |
| 739 | 3′,3′-dimethylsuccinyl | isopropenyl | 4-(2-hydroxyethyl)piperazinyl |
| 740 | 3′,3′-dimethylsuccinyl | isopropyl | 4-(2-hydroxyethyl)piperazinyl |
| 741 | 3′,3′-dimethylglutaryl | isopropenyl | 4-(2-methoxyethyl)piperazinyl |
| 742 | 3′,3′-dimethylglutaryl | isopropyl | 4-(2-methoxyethyl)piperazinyl |
| 743 | 3′3′-dimethylsuccinyl | isopropenyl | 4-(2-methoxyethyl)piperazinyl |
| 744 | 3′,3′-dimethylsuccinyl | isopropyl | 4-(2-methoxyethyl)piperazinyl |
| 745 | 3′,3′-dimethylglutaryl | isopropenyl | 4-(3- |
| | | | dimethylaminopropyl)piperazinyl |
| 746 | 3′,3′-dimethylglutaryl | isopropyl | 4-(3- |
| | | | dimethylaminopropyl)piperazinyl |
| 747 | 3′,3′-dimethylsuccinyl | isopropenyl | 4-(3- |
| | | | dimethylaminopropyl)piperazinyl |
| 748 | 3′,3′-dimethylsuccinyl | isopropyl | 4-(3- |
| | | | dimethylaminopropyl)piperazinyl |
| 749 | 3′,3′-dimethylglutaryl | isopropenyl | 4-(aminocarbonyl)piperazinyl |
| 750 | 3′,3′-dimethylglutaryl | isopropyl | 4-(aminocarbonyl)piperazinyl |
| 751 | 3′,3′-dimethylsuccinyl | isopropenyl | 4-(aminocarbonyl)piperazinyl |
| 752 | 3′,3′-dimethylsuccinyl | isopropyl | 4-(aminocarbonyl)piperazinyl |
| 753 | 3′,3′-dimethylglutaryl | isopropenyl | 4-(aminosulfonyl)piperazinyl |
| 754 | 3′,3′-dimethylglutaryl | isopropyl | 4-(aminosulfonyl)piperazinyl |
| 755 | 3′,3′-dimethylsuccinyl | isopropenyl | 4-(aminosulfonyl)piperazinyl |
| 756 | 3′,3′-dimethylsuccinyl | isopropyl | 4-(aminosulfonyl)piperazinyl |
| 757 | 3′,3′-dimethylglutaryl | isopropenyl | 3-oxopiperazinyl |
| 758 | 3′,3′-dimethylglutaryl | isopropyl | 3-oxopiperazinyl |
| 759 | 3′,3′-dimethylsuccinyl | isopropenyl | 3-oxopiperazinyl |
| 760 | 3′,3′-dimethylsuccinyl | isopropyl | 3-oxopiperazinyl |
| 761 | 3′,3′-dimethylglutaryl | isopropenyl | 4-methyl-3-oxopiperazinyl |
| 762 | 3′,3′-dimethylglutaryl | isopropyl | 4-methyl-3-oxopiperazinyl |
| 763 | 3′,3′-dimethylsuccinyl | isopropenyl | 4-methyl-3-oxopiperazinyl |
| 764 | 3′,3′-dimethylsuccinyl | isopropyl | 4-methyl-3-oxopiperazinyl |
| 765 | 3′,3′-dimethylglutaryl | isopropenyl | 4-(hydroxyethyl)-3-oxopiperazinyl |
| 766 | 3′,3′-dimethylglutaryl | isopropyl | 4-(hydroxyethyl)-3-oxopiperazinyl |
| 767 | 3′,3′-dimethylsuccinyl | isopropenyl | 4-(hydroxyethyl)-3-oxopiperazinyl |
| 768 | 3′,3′-dimethylsuccinyl | isopropyl | 4-(hydroxyethyl)-3-oxopiperazinyl |
| 769 | 3′,3′-dimethylglutaryl | isopropenyl | 4-(2-hydroxybenzoyl)piperazinyl |
| 770 | 3′,3′-dimethylglutaryl | isopropyl | 4-(2-hydroxybenzoyl)piperazinyl |
| 771 | 3′,3′-dimethylsuccinyl | isopropenyl | 4-(2-hydroxybenzoyl)piperazinyl |
| 772 | 3′,3′-dimethylsuccinyl | isopropyl | 4-(2-hydroxybenzoyl)piperazinyl |
| 773 | 3′,3′-dimethylglutaryl | isopropenyl | 4-[3-(1,2,4- |
| | | | oxadiazolyl)methyl]piperazinyl |
| 774 | 3′,3′-dimethylglutaryl | isopropyl | 4-[3-(1,2,4- |
| | | | oxadiazolyl)methyl]piperazinyl |
| 775 | 3′,3′-dimethylsuccinyl | isopropenyl | 4-[3-(1,2,4- |
| | | | oxadiazolyl)methyl]piperazinyl |
| 776 | 3′,3′-dimethylsuccinyl | isopropyl | 4-[3-(1,2,4- |
| | | | oxadiazolyl)methyl]piperazinyl |
| 777 | 3′,3′-dimethylglutaryl | isopropenyl | 4-[4- |
| | | | (dimethylaminosulfonyl)benzyl]piperazinyl |
| 778 | 3′,3′-dimethylglutaryl | isopropyl | 4-[4- |
| | | | (dimethylaminosulfonyl)benzyl]piperazinyl |
| 779 | 3′,3′-dimethylsuccinyl | isopropenyl | 4-[4- |
| | | | (dimethylaminosulfonyl)benzyl]piperazinyl |
| 780 | 3′,3′-dimethylsuccinyl | isopropyl | 4-[4- |
| | | | (dimethylaminosulfonyl)benzyl]piperazinyl |
| 781 | 3′,3′-dimethylglutaryl | isopropenyl | 4-[1-(1,2,3,4- |
| | | | tetrahydronaphthyl)]piperazinyl |
| 782 | 3′,3′-dimethylglutaryl | isopropyl | 4-[1-(1,2,3,4- |
| | | | tetrahydronaphthyl)]piperazinyl |
| 783 | 3′,3′-dimethylsuccinyl | isopropenyl | 4-[1-(1,2,3,4- |
| | | | tetrahydronaphthyl)]piperazinyl |
| 784 | 3′,3′-dimethylsuccinyl | isopropyl | 4-[1-(1,2,3,4- |
| | | | tetrahydronaphthyl)]piperazinyl |
| 785 | 3′,3′-dimethylglutaryl | isopropenyl | 4-[4- |
| | | | (acetamidobenzyl)]piperazinyl |
| 786 | 3′,3′-dimethylglutaryl | isopropyl | 4-[4- |
| | | | (acetamidobenzyl)]piperazinyl |
| 787 | 3′,3′-dimethylsuccinyl | isopropenyl | 4-[4- |
| | | | (acetamidobenzyl)]piperazinyl |
| 788 | 3′,3′-dimethylsuccinyl | isopropyl | 4-[4- |
| | | | (acetamidobenzyl)]piperazinyl |
| 789 | 3′,3′-dimethylglutaryl | isopropenyl | (1S,4S)-5-methyl-2,5- |
| | | | diazabicyclo[2.2.1]heptanyl |
| 790 | 3′,3′-dimethylglutaryl | isopropyl | (1S,4S)-5-methyl-2,5- |
| | | | diazabicyclo[2.2.1]heptanyl |
| 791 | 3′,3′-dimethylsuccinyl | isopropenyl | (1S,4S)-5-methyl-2,5- |
| | | | diazabicyclo[2.2.1]heptanyl |
| 792 | 3′,3′-dimethylsuccinyl | isopropyl | (1S,4S)-5-methyl-2,5- |
| | | | diazabicyclo[2.2.1]heptanyl |
| 793 | 3′,3′-dimethylglutaryl | isopropenyl | (1R,4R)-5-methyl-2,5- |
| | | | diazabicyclo[2.2.1]heptanyl |
| 794 | 3′,3′-dimethylglutaryl | isopropyl | (1R,4R)-5-methyl-2,5- |
| | | | diazabicyclo[2.2.1]heptanyl |
| 795 | 3′,3′-dimethylsuccinyl | isopropenyl | (1R,4R)-5-methyl-2,5- |
| | | | diazabicyclo[2.2.1]heptanyl |
| 796 | 3′,3′-dimethylsuccinyl | isopropyl | (1R,4R)-5-methyl-2,5- |
| | | | diazabicyclo[2.2.1]heptanyl |
| 797 | 3′,3′-dimethylglutaryl | isopropenyl | (1S,4S)-2,5- |
| | | | diazabicyclo[2.2.1]heptanyl |
| 798 | 3′,3′-dimethylglutaryl | isopropyl | (1S,4S)-2,5- |
| | | | diazabicyclo[2.2.1]heptanyl |
| 799 | 3′,3′-dimethylsuccinyl | isopropenyl | (1S,4S)-2,5- |
| | | | diazabicyclo[2.2.1]heptanyl |
| 800 | 3′,3′-dimethylsuccinyl | isopropyl | (1S,4S)-2,5- |
| | | | diazabicyclo[2.2.1]heptanyl |
| 801 | 3′,3′-dimethylglutaryl | isopropenyl | (1R,4R)-2,5- |
| | | | diazabicyclo[2.2.1]heptanyl |
| 802 | 3′,3′-dimethylglutaryl | isopropyl | (1R,4R)-2,5- |
| | | | diazabicyclo[2.2.1]heptanyl |
| 803 | 3′,3′-dimethylsuccinyl | isopropenyl | (1R,4R)-2,5- |
| | | | diazabicyclo[2.2.1]heptanyl |
| 804 | 3′,3′-dimethylsuccinyl | isopropyl | (1R,4R)-2,5- |
| | | | diazabicyclo[2.2.1]heptanyl |
| 805 | 3′,3′-dimethylglutaryl | isopropenyl | (1S,4S)-5-(tert-butoxycarbonyl)- |
| | | | 2,5-diazabicyclo[2.2.1]heptanyl |
| 806 | 3′,3′-dimethylglutaryl | isopropyl | (1S,4S)-5-(tert-butoxycarbonyl)- |
| | | | 2,5-diazabicyclo[2.2.1]heptanyl |
| 807 | 3′,3′-dimethylsuccinyl | isopropenyl | (1S,4S)-5-(tert-butoxycarbonyl)- |
| | | | 2,5-diazabicyclo[2.2.1]heptanyl |
| 808 | 3′,3′-dimethylsuccinyl | isopropyl | (1S,4S)-5-(tert-butoxycarbonyl)- |
| | | | 2,5-diazabicyclo[2.2.1]heptanyl |
| 809 | 3′,3′-dimethylglutaryl | isopropenyl | (1R,4R)-5-(tert-butoxycarbonyl)- |
| | | | 2,5-diazabicyclo[2.2.1]heptanyl |
| 810 | 3′,3′-dimethylglutaryl | isopropyl | (1R,4R)-5-(tert-butoxycarbonyl)- |
| | | | 2,5-diazabicyclo[2.2.1]heptanyl |
| 811 | 3′,3′-dimethylsuccinyl | isopropenyl | (1R,4R)-5-(tert-butoxycarbonyl)- |
| | | | 2,5-diazabicyclo[2.2.1]heptanyl |
| 812 | 3′,3′-dimethylsuccinyl | isopropyl | (1R,4R)-5-(tert-butoxycarbonyl)- |
| | | | 2,5-diazabicyclo[2.2.1]heptanyl |
| 813 | 3′,3′-dimethylglutaryl | isopropenyl | 4-(4-azido-2,3,5,6- |
| | | | tetrafluorobenzyl)piperazinyl |
| 814 | 3′,3′-dimethylglutaryl | isopropyl | 4-(4-azido-2,3,5,6- |
| | | | tetrafluorobenzyl)piperazinyl |
| 815 | 3′,3′-dimethylsuccinyl | isopropenyl | 4-(4-azido-2,3,5,6- |
| | | | tetrafluorobenzyl)piperazinyl |
| 816 | 3′,3′-dimethylsuccinyl | isopropyl | 4-(4-azido-2,3,5,6- |
| | | | tetrafluorobenzyl)piperazinyl |
| 817 | 3′,3′-dimethylglutaryl | isopropenyl | pyrrolidinyl |
| 818 | 3′,3′-dimethylglutaryl | isopropyl | pyrrolidinyl |
| 819 | 3′,3′-dimethylsuccinyl | isopropenyl | pyrrolidinyl |
| 820 | 3′,3′-dimethylsuccinyl | isopropyl | pyrrolidinyl |
| 821 | 3′,3′-dimethylglutaryl | isopropenyl | (R,S)-3-hydroxypyrrolidinyl |
| 822 | 3′,3′-dimethylglutaryl | isopropyl | (R,S)-3-hydroxypyrrolidinyl |
| 823 | 3′,3′-dimethylsuccinyl | isopropenyl | (R,S)-3-hydroxypyrrolidinyl |
| 824 | 3′,3′-dimethylsuccinyl | isopropyl | (R,S)-3-hydroxypyrrolidinyl |
| 825 | 3′,3′-dimethylglutaryl | isopropenyl | (R)-3-hydroxypyrrolidinyl |
| 826 | 3′,3′-dimethylglutaryl | isopropyl | (R)-3-hydroxypyrrolidinyl |
| 827 | 3′,3′-dimethylsuccinyl | isopropenyl | (R)-3-hydroxypyrrolidinyl |
| 828 | 3′,3′-dimethylsuccinyl | isopropyl | (R)-3-hydroxypyrrolidinyl |
| 829 | 3′,3′-dimethylglutaryl | isopropenyl | (S)-3-hydroxypyrrolidinyl |
| 830 | 3′,3′-dimethylglutaryl | isopropyl | (S)-3-hydroxypyrrolidinyl |
| 831 | 3′,3′-dimethylsuccinyl | isopropenyl | (S)-3-hydroxypyrrolidinyl |
| 832 | 3′,3′-dimethylsuccinyl | isopropyl | (S)-3-hydroxypyrrolidinyl |
| 833 | 3′,3′-dimethylglutaryl | isopropenyl | (R)-3-(tert- |
| | | | butoxycarbonylamino)pyrrolidinyl |
| 834 | 3′,3′-dimethylglutaryl | isopropyl | (R)-3-(tert- |
| | | | butoxycarbonylamino)pyrrolidinyl |
| 835 | 3′,3′-dimethylsuccinyl | isopropenyl | (R)-3-(tert- |
| | | | butoxycarbonylamino)pyrrolidinyl |
| 836 | 3′,3′-dimethylsuccinyl | isopropyl | (R)-3-(tert- |
| | | | butoxycarbonylamino)pyrrolidinyl |
| 837 | 3′,3′-dimethylglutaryl | isopropenyl | (S)-3-(tert- |
| | | | butoxycarbonylamino)pyrrolidinyl |
| 838 | 3′,3′-dimethylglutaryl | isopropyl | (S)-3-(tert- |
| | | | butoxycarbonylamino)pyrrolidinyl |
| 839 | 3′,3′-dimethylsuccinyl | isopropenyl | (S)-3-(tert- |
| | | | butoxycarbonylamino)pyrrolidinyl |
| 840 | 3′,3′-dimethylsuccinyl | isopropyl | (S)-3-(tert- |
| | | | butoxycarbonylamino)pyrrolidinyl |
| 841 | 3′,3′-dimethylglutaryl | isopropenyl | (R)-3-aminopyrrolidinyl |
| 842 | 3′,3′-dimethylglutaryl | isopropyl | (R)-3-aminopyrrolidinyl |
| 843 | 3′,3′-dimethylsuccinyl | isopropenyl | (R)-3-aminopyrrolidinyl |
| 844 | 3′,3′-dimethylsuccinyl | isopropyl | (R)-3-aminopyrrolidinyl |
| 845 | 3′,3′-dimethylglutaryl | isopropenyl | (S)-3-aminopyrrolidinyl |
| 846 | 3′,3′-dimethylglutaryl | isopropyl | (S)-3-aminopyrrolidinyl |
| 847 | 3′,3′-dimethylsuccinyl | isopropenyl | (S)-3-aminopyrrolidinyl |
| 848 | 3′,3′-dimethylsuccinyl | isopropyl | (S)-3-aminopyrrolidinyl |
| 849 | 3′,3′-dimethylglutaryl | isopropenyl | (R)-2- |
| | | | (hydroxymethyl)pyrrolidinyl |
| 850 | 3′,3′-dimethylglutaryl | isopropyl | (R)-2- |
| | | | (hydroxymethyl)pyrrolidinyl |
| 851 | 3′,3′-dimethylsuccinyl | isopropenyl | (R)-2- |
| | | | (hydroxymethyl)pyrrolidinyl |
| 852 | 3′,3′-dimethylsuccinyl | isopropyl | (R)-2- |
| | | | (hydroxymethyl)pyrrolidinyl |
| 853 | 3′,3′-dimethylglutaryl | isopropenyl | (S)-2- |
| | | | (hydroxymethyl)pyrrolidinyl |
| 854 | 3′,3′-dimethylglutaryl | isopropyl | (S)-2- |
| | | | (hydroxymethyl)pyrrolidinyl |
| 855 | 3′,3′-dimethylsuccinyl | isopropenyl | (S)-2- |
| | | | (hydroxymethyl)pyrrolidinyl |
| 856 | 3′,3′-dimethylsuccinyl | isopropyl | (S)-2- |
| | | | (hydroxymethyl)pyrrolidinyl |
| 857 | 3′,3′-dimethylglutaryl | isopropenyl | (R)-3-N-methylaminopyrrolidinyl |
| 858 | 3′,3′-dimethylglutaryl | isopropyl | (R)-3-N-methylaminopyrrolidinyl |
| 859 | 3′,3′-dimethylsuccinyl | isopropenyl | (R)-3-N-methylaminopyrrolidinyl |
| 860 | 3′,3′-dimethylsuccinyl | isopropyl | (R)-3-N-methylaminopyrrolidinyl |
| 861 | 3′,3′-dimethylglutaryl | isopropenyl | (S)-3-N-methylaminopyrrolidinyl |
| 862 | 3′,3′-dimethylglutaryl | isopropyl | (S)-3-N-methylaminopyrrolidinyl |
| 863 | 3′,3′-dimethylsuccinyl | isopropenyl | (S)-3-N-methylaminopyrrolidinyl |
| 864 | 3′,3′-dimethylsuccinyl | isopropyl | (S)-3-N-methylaminopyrrolidinyl |
| 865 | 3′,3′-dimethylglutaryl | isopropenyl | (R)-3-N,N- |
| | | | dimethylaminopyrrolidinyl |
| 866 | 3′,3′-dimethylglutaryl | isopropyl | (R)-3-N,N- |
| | | | dimethylaminopyrrolidinyl |
| 867 | 3′,3′-dimethylsuccinyl | isopropenyl | (R)-3-N,N- |
| | | | dimethylaminopyrrolidinyl |
| 868 | 3′,3′-dimethylsuccinyl | isopropyl | (R)-3-N,N- |
| | | | dimethylaminopyrrolidinyl |
| 869 | 3′,3′-dimethylglutaryl | isopropenyl | (S)-3-N,N- |
| | | | dimethylaminopyrrolidinyl |
| 870 | 3′,3′-dimethylglutaryl | isopropyl | (S)-3-N,N- |
| | | | dimethylaminopyrrolidinyl |
| 871 | 3′,3′-dimethylsuccinyl | isopropenyl | (S)-3-N,N- |
| | | | dimethylaminopyrrolidinyl |
| 872 | 3′,3′-dimethylsuccinyl | isopropyl | (S)-3-N,N- |
| | | | dimethylaminopyrrolidinyl |
| 873 | 3′,3′-dimethylglutaryl | isopropenyl | (R)-3-N,N- |
| | | | diethylaminopyrrolidinyl |
| 874 | 3′,3′-dimethylglutaryl | isopropyl | (R)-3-N,N- |
| | | | diethylaminopyrrolidinyl |
| 875 | 3′,3′-dimethylsuccinyl | isopropenyl | (R)-3-N,N- |
| | | | diethylaminopyrrolidinyl |
| 876 | 3′,3′-dimethylsuccinyl | isopropyl | (R)-3-N,N- |
| | | | diethylaminopyrrolidinyl |
| 877 | 3′,3′-dimethylglutaryl | isopropenyl | (S)-3-N,N- |
| | | | diethylaminopyrrolidinyl |
| 878 | 3′,3′-dimethylglutaryl | isopropyl | (S)-3-N,N- |
| | | | diethylaminopyrrolidinyl |
| 879 | 3′,3′-dimethylsuccinyl | isopropenyl | (S)-3-N,N- |
| | | | diethylaminopyrrolidinyl |
| 880 | 3′,3′-dimethylsuccinyl | isopropyl | (S)-3-N,N- |
| | | | diethylaminopyrrolidinyl |
| 881 | 3′,3′-dimethylglutaryl | isopropenyl | (R)-3-N-ethylaminopyrrolidinyl |
| 882 | 3′,3′-dimethylglutaryl | isopropyl | (R)-3-N-ethylaminopyrrolidinyl |
| 883 | 3′,3′-dimethylsuccinyl | isopropenyl | (R)-3-N-ethylaminopyrrolidinyl |
| 884 | 3′,3′-dimethylsuccinyl | isopropyl | (R)-3-N-ethylaminopyrrolidinyl |
| 885 | 3′,3′-dimethylglutaryl | isopropenyl | (S)-3-N-ethylaminopyrrolidinyl |
| 886 | 3′,3′-dimethylglutaryl | isopropyl | (S)-3-N-ethylaminopyrrolidinyl |
| 887 | 3′,3′-dimethylsuccinyl | isopropenyl | (S)-3-N-ethylaminopyrrolidinyl |
| 888 | 3′,3′-dimethylsuccinyl | isopropyl | (S)-3-N-ethylaminopyrrolidinyl |
| 889 | 3′,3′-dimethylglutaryl | isopropenyl | (R)-3-(4-morpholinyl)pyrrolidinyl |
| 890 | 3′,3′-dimethylglutaryl | isopropyl | (R)-3-(4-morpholinyl)pyrrolidinyl |
| 891 | 3′,3′-dimethylsuccinyl | isopropenyl | (R)-3-(4-morpholinyl)pyrrolidinyl |
| 892 | 3′,3′-dimethylsuccinyl | isopropyl | (R)-3-(4-morpholinyl)pyrrolidinyl |
| 893 | 3′,3′-dimethylglutaryl | isopropenyl | (S)-3-(4-morpholinyl)pyrrolidinyl |
| 894 | 3′,3′-dimethylglutaryl | isopropyl | (S)-3-(4-morpholinyl)pyrrolidinyl |
| 895 | 3′,3′-dimethylsuccinyl | isopropenyl | (S)-3-(4-morpholinyl)pyrrolidinyl |
| 896 | 3′,3′-dimethylsuccinyl | isopropyl | (S)-3-(4-morpholinyl)pyrrolidinyl |
| 897 | 3′,3′-dimethylglutaryl | isopropenyl | (R)-3-(1-pyrrolidinyl)pyrrolidinyl |
| 898 | 3′,3′-dimethylglutaryl | isopropyl | (R)-3-(1-pyrrolidinyl)pyrrolidinyl |
| 899 | 3′,3′-dimethylsuccinyl | isopropenyl | (R)-3-(1-pyrrolidinyl)pyrrolidinyl |
| 900 | 3′,3′-dimethylsuccinyl | isopropyl | (R)-3-(1-pyrrolidinyl)pyrrolidinyl |
| 901 | 3′,3′-dimethylglutaryl | isopropenyl | (S)-3-(1-pyrrolidinyl)pyrrolidinyl |
| 902 | 3′,3′-dimethylglutaryl | isopropyl | (S)-3-(1-pyrrolidinyl)pyrrolidinyl |
| 903 | 3′,3′-dimethylsuccinyl | isopropenyl | (S)-3-(1-pyrrolidinyl)pyrrolidinyl |
| 904 | 3′,3′-dimethylsuccinyl | isopropyl | (S)-3-(1-pyrrolidinyl)pyrrolidinyl |
| 905 | 3′,3′-dimethylglutaryl | isopropenyl | 4-aminopiperidinyl |
| 906 | 3′,3′-dimethylglutaryl | isopropyl | 4-aminopiperidinyl |
| 907 | 3′,3′-dimethylsuccinyl | isopropenyl | 4-aminopiperidinyl |
| 908 | 3′,3′-dimethylsuccinyl | isopropyl | 4-aminopiperidinyl |
| 909 | 3′,3′-dimethylglutaryl | isopropenyl | 4-oxopiperidinyl |
| 910 | 3′,3′-dimethylglutaryl | isopropyl | 4-oxopiperidinyl |
| 911 | 3′,3′-dimethylsuccinyl | isopropenyl | 4-oxopiperidinyl |
| 912 | 3′,3′-dimethylsuccinyl | isopropyl | 4-oxopiperidinyl |
| 913 | 3′,3′-dimethylglutaryl | isopropenyl | 4-hydroxypiperidinyl |
| 914 | 3′,3′-dimethylglutaryl | isopropyl | 4-hydroxypiperidinyl |
| 915 | 3′,3′-dimethylsuccinyl | isopropenyl | 4-hydroxypiperidinyl |
| 916 | 3′,3′-dimethylsuccinyl | isopropyl | 4-hydroxypiperidinyl |
| 917 | 3′,3′-dimethylglutaryl | isopropenyl | 4-N,N-diaminopiperidinyl |
| 918 | 3′,3′-dimethylglutaryl | isopropyl | 4-N,N-diaminopiperidinyl |
| 919 | 3′,3′-dimethylsuccinyl | isopropenyl | 4-N,N-diaminopiperidinyl |
| 920 | 3′,3′-dimethylsuccinyl | isopropyl | 4-N,N-diaminopiperidinyl |
| 921 | 3′,3′-dimethylglutaryl | isopropenyl | 4-(4-morpholinyl)piperidinyl |
| 922 | 3′,3′-dimethylglutaryl | isopropyl | 4-(4-morpholinyl)piperidinyl |
| 923 | 3′,3′-dimethylsuccinyl | isopropenyl | 4-(4-morpholinyl)piperidinyl |
| 924 | 3′,3′-dimethylsuccinyl | isopropyl | 4-(4-morpholinyl)piperidinyl |
| 925 | 3′,3′-dimethylglutaryl | isopropenyl | 4-acetamidopiperidinyl |
| 926 | 3′,3′-dimethylglutaryl | isopropyl | 4-acetamidopiperidinyl |
| 927 | 3′,3′-dimethylsuccinyl | isopropenyl | 4-acetamidopiperidinyl |
| 928 | 3′,3′-dimethylsuccinyl | isopropyl | 4-acetamidopiperidinyl |
| 929 | 3′,3′-dimethylglutaryl | isopropenyl | 4-(methylsulfonamide)piperidinyl |
| 930 | 3′,3′-dimethylglutaryl | isopropyl | 4-(methylsulfonamide)piperidinyl |
| 931 | 3′,3′-dimethylsuccinyl | isopropenyl | 4-(methylsulfonamide)piperidinyl |
| 932 | 3′,3′-dimethylsuccinyl | isopropyl | 4-(methylsulfonamide)piperidinyl |
| 933 | 3′,3′-dimethylglutaryl | isopropenyl | (R)-3-acetamidopyrrolidinyl |
| 934 | 3′,3′-dimethylglutaryl | isopropyl | (R)-3-acetamidopyrrolidinyl |
| 935 | 3′,3′-dimethylsuccinyl | isopropenyl | (R)-3-acetamidopyrrolidinyl |
| 936 | 3′,3′-dimethylsuccinyl | isopropyl | (R)-3-acetamidopyrrolidinyl |
| 937 | 3′,3′-dimethylglutaryl | isopropenyl | (S)-3-acetamidopyrrolidinyl |
| 938 | 3′,3′-dimethylglutaryl | isopropyl | (S)-3-acetamidopyrrolidinyl |
| 939 | 3′,3′-dimethylsuccinyl | isopropenyl | (S)-3-acetamidopyrrolidinyl |
| 940 | 3′,3′-dimethylsuccinyl | isopropyl | (S)-3-acetamidopyrrolidinyl |
| 941 | 3′,3′-dimethylglutaryl | isopropenyl | (R)-3- |
| | | | (cyclopropanecarboxamido)pyrrolidinyl |
| 942 | 3′,3′-dimethylglutaryl | isopropyl | (R)-3- |
| | | | (cyclopropanecarboxamido)pyrrolidinyl |
| 943 | 3′,3′-dimethylsuccinyl | isopropenyl | (R)-3- |
| | | | (cyclopropanecarboxamido)pyrrolidinyl |
| 944 | 3′,3′-dimethylsuccinyl | isopropyl | (R)-3- |
| | | | (cyclopropanecarboxamido)pyrrolidinyl |
| 945 | 3′,3′-dimethylglutaryl | isopropenyl | (S)-3- |
| | | | (cyclopropanecarboxamido)pyrrolidinyl |
| 946 | 3′,3′-dimethylglutaryl | isopropyl | (S)-3- |
| | | | (cyclopropanecarboxamido)pyrrolidinyl |
| 947 | 3′,3′-dimethylsuccinyl | isopropenyl | (S)-3- |
| | | | (cyclopropanecarboxamido)pyrrolidinyl |
| 948 | 3′,3′-dimethylsuccinyl | isopropyl | (S)-3- |
| | | | (cyclopropanecarboxamido)pyrrolidinyl |
| 949 | 3′,3′-dimethylglutaryl | isopropenyl | (R)-3-(2- |
| | | | hydroxyacetamido)pyrrolidinyl |
| 950 | 3′,3′-dimethylglutaryl | isopropyl | (R)-3-(2- |
| | | | hydroxyacetamido)pyrrolidinyl |
| 951 | 3′,3′-dimethylsuccinyl | isopropenyl | (R)-3-(2- |
| | | | hydroxyacetamido)pyrrolidinyl |
| 952 | 3′,3′-dimethylsuccinyl | isopropyl | (R)-3-(2- |
| | | | hydroxyacetamido)pyrrolidinyl |
| 953 | 3′,3′-dimethylglutaryl | isopropenyl | (S)-3-(2- |
| | | | hydroxyacetamido)pyrrolidinyl |
| 954 | 3′,3′-dimethylglutaryl | isopropyl | (S)-3-(2- |
| | | | hydroxyacetamido)pyrrolidinyl |
| 955 | 3′,3′-dimethylsuccinyl | isopropenyl | (S)-3-(2- |
| | | | hydroxyacetamido)pyrrolidinyl |
| 956 | 3′,3′-dimethylsuccinyl | isopropyl | (S)-3-(2- |
| | | | hydroxyacetamido)pyrrolidinyl |
| 957 | 3′,3′-dimethylglutaryl | isopropenyl | (R)-3- |
| | | | (methylsulfonamido)pyrrolidinyl |
| 958 | 3′,3′-dimethylglutaryl | isopropyl | (R)-3- |
| | | | (methylsulfonamido)pyrrolidinyl |
| 959 | 3′,3′-dimethylsuccinyl | isopropenyl | (R)-3- |
| | | | (methylsulfonamido)pyrrolidinyl |
| 960 | 3′,3′-dimethylsuccinyl | isopropyl | (R)-3- |
| | | | (methylsulfonamido)pyrrolidinyl |
| 961 | 3′,3′-dimethylglutaryl | isopropenyl | (S)-3- |
| | | | (methylsulfonamido)pyrrolidinyl |
| 962 | 3′,3′-dimethylglutaryl | isopropyl | (S)-3- |
| | | | (methylsulfonamido)pyrrolidinyl |
| 963 | 3′,3′-dimethylsuccinyl | isopropenyl | (S)-3- |
| | | | (methylsulfonamido)pyrrolidinyl |
| 964 | 3′,3′-dimethylsuccinyl | isopropyl | (S)-3- |
| | | | (methylsulfonamido)pyrrolidinyl |
| 965 | 3′,3′-dimethylglutaryl | isopropenyl | (R)-2-(aminomethyl)pyrrolidinyl |
| 966 | 3′,3′-dimethylglutaryl | isopropyl | (R)-2-(aminomethyl)pyrrolidinyl |
| 967 | 3′,3′-dimethylsuccinyl | isopropenyl | (R)-2-(aminomethyl)pyrrolidinyl |
| 968 | 3′,3′-dimethylsuccinyl | isopropyl | (R)-2-(aminomethyl)pyrrolidinyl |
| 969 | 3′,3′-dimethylglutaryl | isopropenyl | (S)-2-(aminomethyl)pyrrolidinyl |
| 970 | 3′,3′-dimethylglutaryl | isopropyl | (S)-2-(aminomethyl)pyrrolidinyl |
| 971 | 3′,3′-dimethylsuccinyl | isopropenyl | (S)-2-(aminomethyl)pyrrolidinyl |
| 972 | 3′,3′-dimethylsuccinyl | isopropyl | (S)-2-(aminomethyl)pyrrolidinyl |
| 973 | 3′,3′-dimethylglutaryl | isopropenyl | (R)-2-(N,N- |
| | | | dimethylaminomethyl)pyrrolidinyl |
| 974 | 3′,3′-dimethylglutaryl | isopropyl | (R)-2-(N,N- |
| | | | dimethylaminomethyl)pyrrolidinyl |
| 975 | 3′,3′-dimethylsuccinyl | isopropenyl | (R)-2-(N,N- |
| | | | dimethylaminomethyl)pyrrolidinyl |
| 976 | 3′,3′-dimethylsuccinyl | isopropyl | (R)-2-(N,N- |
| | | | dimethylaminomethyl)pyrrolidinyl |
| 977 | 3′,3′-dimethylglutaryl | isopropenyl | (S)-2-(N,N- |
| | | | dimethylaminomethyl)pyrrolidinyl |
| 978 | 3′,3′-dimethylglutaryl | isopropyl | (S)-2-(N,N- |
| | | | dimethylaminomethyl)pyrrolidinyl |
| 979 | 3′,3′-dimethylsuccinyl | isopropenyl | (S)-2-(N,N- |
| | | | dimethylaminomethyl)pyrrolidinyl |
| 980 | 3′,3′-dimethylsuccinyl | isopropyl | (S)-2-(N,N- |
| | | | dimethylaminomethyl)pyrrolidinyl |
| 981 | 3′,3′-dimethylglutaryl | isopropenyl | (R)-2- |
| | | | (acetamidomethyl)pyrrolidinyl |
| 982 | 3′,3′-dimethylglutaryl | isopropyl | (R)-2- |
| | | | (acetamidomethyl)pyrrolidinyl |
| 983 | 3′,3′-dimethylsuccinyl | isopropenyl | (R)-2- |
| | | | (acetamidomethyl)pyrrolidinyl |
| 984 | 3′,3′-dimethylsuccinyl | isopropyl | (R)-2- |
| | | | (acetamidomethyl)pyrrolidinyl |
| 985 | 3′,3′-dimethylglutaryl | isopropenyl | (S)-2- |
| | | | (acetamidomethyl)pyrrolidinyl |
| 986 | 3′,3′-dimethylglutaryl | isopropyl | (S)-2- |
| | | | (acetamidomethyl)pyrrolidinyl |
| 987 | 3′,3′-dimethylsuccinyl | isopropenyl | (S)-2- |
| | | | (acetamidomethyl)pyrrolidinyl |
| 988 | 3′,3′-dimethylsuccinyl | isopropyl | (S)-2- |
| | | | (acetamidomethyl)pyrrolidinyl |
| 989 | 3′,3′-dimethylglutaryl | isopropenyl | (R)-2- |
| | | | (methylsulfonamidomethyl)pyrrolidinyl |
| 990 | 3′,3′-dimethylglutaryl | isopropyl | (R)-2- |
| | | | (methylsulfonamidomethyl)pyrrolidinyl |
| 991 | 3′,3′-dimethylsuccinyl | isopropenyl | (R)-2- |
| | | | (methylsulfonamidomethyl)pyrrolidinyl |
| 992 | 3′,3′-dimethylsuccinyl | isopropyl | (R)-2- |
| | | | (methylsulfonamidomethyl)pyrrolidinyl |
| 993 | 3′,3′-dimethylglutaryl | isopropenyl | (S)-2- |
| | | | (methylsulfonamidomethyl)pyrrolidinyl |
| 994 | 3′,3′-dimethylglutaryl | isopropyl | (S)-2- |
| | | | (methylsulfonamidomethyl)pyrrolidinyl |
| 995 | 3′,3′-dimethylsuccinyl | isopropenyl | (S)-2- |
| | | | (methylsulfonamidomethyl)pyrrolidinyl |
| 996 | 3′,3′-dimethylsuccinyl | isopropyl | (S)-2- |
| | | | (methylsulfonamidomethyl)pyrrolidinyl |
| 997 | 3′,3′-dimethylglutaryl | isopropenyl | (R)-2-(N,N- |
| | | | diethylaminomethyl)pyrrolidinyl |
| 998 | 3′,3′-dimethylglutaryl | isopropyl | (R)-2-(N,N- |
| | | | diethylaminomethyl)pyrrolidinyl |
| 999 | 3′,3′-dimethylsuccinyl | isopropenyl | (R)-2-(N,N- |
| | | | diethylaminomethyl)pyrrolidinyl |
| 1000 | 3′,3′-dimethylsuccinyl | isopropyl | (R)-2-(N,N- |
| | | | diethylaminomethyl)pyrrolidinyl |
| 1001 | 3′,3′-dimethylglutaryl | isopropenyl | (S)-2-(N,N- |
| | | | diethylaminomethyl)pyrrolidinyl |
| 1002 | 3′,3′-dimethylglutaryl | isopropyl | (S)-2-(N,N- |
| | | | diethylaminomethyl)pyrrolidinyl |
| 1003 | 3′,3′-dimethylsuccinyl | isopropenyl | (S)-2-(N,N- |
| | | | diethylaminomethyl)pyrrolidinyl |
| 1004 | 3′,3′-dimethylsuccinyl | isopropyl | (S)-2-(N,N- |
| | | | diethylaminomethyl)pyrrolidinyl |
| 1005 | 3′,3′-dimethylglutaryl | isopropenyl | (R)-2-(4- |
| | | | morpholinylmethyl)pyrrolidinyl |
| 1006 | 3′,3′-dimethylglutaryl | isopropyl | (R)-2-(4- |
| | | | morpholinylmethyl)pyrrolidinyl |
| 1007 | 3′,3′-dimethylsuccinyl | isopropenyl | (R)-2-(4- |
| | | | morpholinylmethyl)pyrrolidinyl |
| 1008 | 3′,3′-dimethylsuccinyl | isopropyl | (R)-2-(4- |
| | | | morpholinylmethyl)pyrrolidinyl |
| 1009 | 3′,3′-dimethylglutaryl | isopropenyl | (S)-2-(4- |
| | | | morpholinylmethyl)pyrrolidinyl |
| 1010 | 3′,3′-dimethylglutaryl | isopropyl | (S)-2-(4- |
| | | | morpholinylmethyl)pyrrolidinyl |
| 1011 | 3′,3′-dimethylsuccinyl | isopropenyl | (S)-2-(4- |
| | | | morpholinylmethyl)pyrrolidinyl |
| 1012 | 3′,3′-dimethylsuccinyl | isopropyl | (S)-2-(4- |
| | | | morpholinylmethyl)pyrrolidinyl |
| 1013 | 3′,3′-dimethylglutaryl | isopropenyl | 2,6-dimethylmorpholinyl |
| 1014 | 3′,3′-dimethylglutaryl | isopropyl | 2,6-dimethylmorpholinyl |
| 1015 | 3′,3′-dimethylsuccinyl | isopropenyl | 2,6-dimethylmorpholinyl |
| 1016 | 3′,3′-dimethylsuccinyl | isopropyl | 2,6-dimethylmorpholinyl |
| 1017 | 3′,3′-dimethylglutaryl | isopropenyl | 1,4-oxazepanyl |
| 1018 | 3′,3′-dimethylglutaryl | isopropyl | 1,4-oxazepanyl |
| 1019 | 3′,3′-dimethylsuccinyl | isopropenyl | 1,4-oxazepanyl |
| 1020 | 3′,3′-dimethylsuccinyl | isopropyl | 1,4-oxazepanyl |
| 1021 | 3′,3′-dimethylglutaryl | isopropenyl | thiomorpholinyl |
| 1022 | 3′,3′-dimethylglutaryl | isopropyl | thiomorpholinyl |
| 1023 | 3′,3′-dimethylsuccinyl | isopropenyl | thiomorpholinyl |
| 1024 | 3′,3′-dimethylsuccinyl | isopropyl | thiomorpholinyl |
| 1025 | 3′,3′-dimethylglutaryl | isopropenyl | thiomorpholinyl 1-oxide |
| 1026 | 3′,3′-dimethylglutaryl | isopropyl | thiomorpholinyl 1-oxide |
| 1027 | 3′,3′-dimethylsuccinyl | isopropenyl | thiomorpholinyl 1-oxide |
| 1028 | 3′,3′-dimethylsuccinyl | isopropyl | thiomorpholinyl 1-oxide |
| 1029 | 3′,3′-dimethylglutaryl | isopropenyl | thiomorpholinyl 1,1-dioxide |
| 1030 | 3′,3′-dimethylglutaryl | isopropyl | thiomorpholinyl 1,1-dioxide |
| 1031 | 3′,3′-dimethylsuccinyl | isopropenyl | thiomorpholinyl 1,1-dioxide |
| 1032 | 3′,3′-dimethylsuccinyl | isopropyl | thiomorpholinyl 1,1-dioxide |

Preferred compounds wherein R₂ is (vi) include, but are not limited to, those found in Table 8, wherein R₁₈ and R₁₉ are hydrogen, and d is 1:

TABLE 8

| # | R₁ | R₃ | R₁₂ | R₁₃ |
|---|---|---|---|---|
| 1033 | 3′,3′-dimethylsuccinyl | isopropenyl | tert-butyl | hydrogen |
| 1034 | 3′,3′-dimethylglutaryl | isopropenyl | tert-butyl | hydrogen |
| 1035 | 3′,3′-dimethylsuccinyl | isopropenyl | tert- | hydrogen |
| | | | butoxycarbonyl | |
| 1036 | 3′,3′-dimethylglutaryl | isopropenyl | tert- | hydrogen |
| | | | butoxycarbonyl | |
| 1037 | 3′,3′-dimethylsuccinyl | isopropenyl | methoxy | hydrogen |
| 1038 | 3′,3′-dimethylglutaryl | isopropenyl | methoxy | hydrogen |
| 1039 | 3′,3′-dimethylsuccinyl | isopropenyl | 5-tetrazolyl | hydrogen |
| 1040 | 3′,3′-dimethylglutaryl | isopropenyl | 5-tetrazolyl | hydrogen |
| 1041 | 3′,3′-dimethylsuccinyl | isopropyl | tert-butyl | hydrogen |
| 1042 | 3′,3′-dimethylglutaryl | isopropyl | tert-butyl | hydrogen |
| 1043 | 3′,3′-dimethylsuccinyl | isopropyl | tert- | hydrogen |
| | | | butoxycarbonyl | |
| 1044 | 3′,3′-dimethylglutaryl | isopropyl | tert- | hydrogen |
| | | | butoxycarbonyl | |
| 1045 | 3′,3′-dimethylsuccinyl | isopropyl | methoxy | hydrogen |
| 1046 | 3′,3′-dimethylglutaryl | isopropyl | methoxy | hydrogen |
| 1047 | 3′,3′-dimethylsuccinyl | isopropyl | 5-tetrazolyl | hydrogen |
| 1048 | 3′,3′-dimethylglutaryl | isopropyl | 5-tetrazolyl | hydrogen |

Preferred compounds wherein R₂ is (vi) and R₁₂ and R₁₃ taken with the nitrogen to which they are attached form a heterocycle or heteroaryl include those found in Table 9:

TABLE 9

| | | | R₁₂ and R₁₃ taken with the |
|---|---|---|---|
| | | | nitrogen to which they are |
| # | R₁ | R₃ | attached |
| 1049 | 3′,3′-dimethylsuccinyl | isopropenyl | 4′-carboxypiperidinyl |
| 1050 | 3′,3′-dimethylglutaryl | isopropenyl | 4′-carboxypiperidinyl |
| 1051 | 3′,3′-dimethylsuccinyl | isopropenyl | 3′-hydroxypyrrolidinyl |
| 1052 | 3′,3′-dimethylglutaryl | isopropenyl | 3′-hydroxypyrrolidinyl |
| 1053 | 3′,3′-dimethylsuccinyl | isopropenyl | 4′,4’-difluoropiperidinyl |
| 1054 | 3′,3′-dimethylglutaryl | isopropenyl | 4′,4’-difluoropiperidinyl |
| 1055 | 3′,3′-dimethylsuccinyl | isopropenyl | 4′-ethylpiperazinyl |
| 1056 | 3′,3′-dimethylglutaryl | isopropenyl | 4′-ethylpiperazinyl |
| 1057 | 3′,3′-dimethylsuccinyl | isopropyl | 4′-carboxypiperidinyl |
| 1058 | 3′,3′-dimethylglutaryl | isopropyl | 4′-carboxypiperidinyl |
| 1059 | 3′,3′-dimethylsuccinyl | isopropyl | 3′-hydroxypyrrolidinyl |
| 1060 | 3′,3′-dimethylglutaryl | isopropyl | 3′-hydroxypyrrolidinyl |
| 1061 | 3′,3′-dimethylsuccinyl | isopropyl | 4′,4′-difluoropiperidinyl |
| 1062 | 3′,3′-dimethylglutaryl | isopropyl | 4′,4′-difluoropiperidinyl |
| 1063 | 3′,3′-dimethylsuccinyl | isopropyl | 4′-ethylpiperazinyl |
| 1064 | 3′,3′-dimethylglutaryl | isopropyl | 4′-ethylpiperazinyl |

Additional preferred compounds wherein R₂ is (viii) include, but are not limited to, those found in Table 10:

TABLE 10

| # | R₁ | R₃ | R₁₇ | R₂₀ |
|---|---|---|---|---|
| 1065 | 3′,3′-dimethylglutaryl | isopropenyl | tert-butoxy | hydrogen |
| 1066 | 3′,3′-dimethylglutaryl | isopropyl | tert-butoxy | hydrogen |
| 1067 | 3′,3′-dimethylsuccinyl | isopropenyl | tert-butoxy | hydrogen |
| 1068 | 3′,3′-dimethylsuccinyl | isopropyl | tert-butoxy | hydrogen |
| 1069 | 3′,3′-dimethylglutaryl | isopropenyl | methyl | hydrogen |
| 1070 | 3′,3′-dimethylglutaryl | isopropyl | methyl | hydrogen |
| 1071 | 3′,3′-dimethylsuccinyl | isopropenyl | methyl | hydrogen |
| 1072 | 3′,3′-dimethylsuccinyl | isopropyl | methyl | hydrogen |
| 1073 | 3′,3′-dimethylglutaryl | isopropenyl | methyl | methyl |
| 1074 | 3′,3′-dimethylglutaryl | isopropyl | methyl | methyl |
| 1075 | 3′,3′-dimethylsuccinyl | isopropenyl | methyl | methyl |
| 1076 | 3′,3′-dimethylsuccinyl | isopropyl | methyl | methyl |
| 1077 | 3′,3′-dimethylglutaryl | isopropenyl | trifluromethyl | hydrogen |
| 1078 | 3′,3′-dimethylglutaryl | isopropyl | trifluromethyl | hydrogen |
| 1079 | 3′,3′-dimethylsuccinyl | isopropenyl | trifluromethyl | hydrogen |
| 1080 | 3′,3′-dimethylsuccinyl | isopropyl | trifluromethyl | hydrogen |
| 1081 | 3′,3′-dimethylglutaryl | isopropenyl | phenyl | hydrogen |
| 1082 | 3′,3′-dimethylglutaryl | isopropyl | phenyl | hydrogen |
| 1083 | 3′,3′-dimethylsuccinyl | isopropenyl | phenyl | hydrogen |
| 1084 | 3′,3′-dimethylsuccinyl | isopropyl | phenyl | hydrogen |
| 1085 | 3′,3′-dimethylglutaryl | isopropenyl | hydrogen | hydrogen |
| 1086 | 3′,3′-dimethylglutaryl | isopropyl | hydrogen | hydrogen |
| 1087 | 3′,3′-dimethylsuccinyl | isopropenyl | hydrogen | hydrogen |
| 1088 | 3′,3′-dimethylsuccinyl | isopropyl | hydrogen | hydrogen |

Additional preferred compounds wherein R₂ is (ii) include the compounds found in Table 11:

TABLE 11

| | | R₂ is (ii) | |
|---|---|---|---|
| # | R₁ | and R₆ is | R₃ |
| 1089 | 3′,3′-dimethylsuccinyl | hydrogen | ethoxymethoxy(methyl)- |
| | | | methyl |
| 1090 | 3′,3′-dimethylglutaryl | hydrogen | ethoxymethoxy(methyl)- |
| | | | methyl |
| 1091 | 3′,3′-dimethylsuccinyl | hydrogen | 1′-oxoethyl |
| 1092 | 3′,3′-dimethylglutaryl | hydrogen | 1′-oxoethyl |
| 1093 | 3′,3′-dimethylsuccinyl | hydrogen | 1′-methoxymethyl |
| 1094 | 3′,3′-dimethylglutaryl | hydrogen | 1′-methoxymethyl |
| 1095 | 3′,3′-dimethylsuccinyl | hydrogen | isobutyl |
| 1096 | 3′,3′-dimethylglutaryl | hydrogen | isobutyl |
| 1097 | 3′,3′-dimethylsuccinyl | hydrogen | 2′-hydroxyisopropyl |
| 1098 | 3′,3′-dimethylglutaryl | hydrogen | 2′-hydroxyisopropyl |

Additional preferred compounds include derivatives of R₃ and R₂ is (iv). Examples can be found in Table 12:

TABLE 12

| | | R₂ is (iv) | |
|---|---|---|---|
| # | R₁ | and R₉ is | R₃ |
| 1099 | 3′,3′-dimethylsuccinyl | hydrogen | ethoxymethoxy(methyl)- |
| | | | methyl |
| 1100 | 3′,3′-dimethylglutaryl | hydrogen | ethoxymethoxy(methyl)- |
| | | | methyl |
| 1101 | 3′,3′-dimethylsuccinyl | hydrogen | 1′-oxoethyl |
| 1102 | 3′,3′-dimethylglutaryl | hydrogen | 1′-oxoethyl |
| 1103 | 3′,3′-dimethylsuccinyl | hydrogen | 1′-methoxymethyl |
| 1104 | 3′,3′-dimethylglutaryl | hydrogen | 1′-methoxymethyl |
| 1105 | 3′,3′-dimethylsuccinyl | hydrogen | isobutyl |
| 1106 | 3′,3′-dimethylglutaryl | hydrogen | isobutyl |
| 1107 | 3′,3′-dimethylsuccinyl | hydrogen | 2′-hydroxyisopropyl |
| 1108 | 3′,3′-dimethylglutaryl | hydrogen | 2′-hydroxyisopropyl |

Additional preferred compounds include allyl or alkyl esters of R₁ for any of the compounds listed in Tables 1-12. Additional preferred compounds include any of the compounds listed in Tables 1-12, wherein the specified R₁ is replaced by succinyl, glutaryl, 3′-methylsuccinyl, or 3′-methylglutaryl.

Additional preferred compounds include derivatives of R₁. Examples can be found in Table 13:

TABLE 13

| | | R₂ is (ii) | |
|---|---|---|---|
| # | R₁ | and R₆ is | R₃ |
| 1109 | 4′-(methylsulfonylamino)-4′oxo-3′,3′- | hydrogen | isopropenyl |
| | dimethylbutanoyl | | |
| 1110 | 4′-(methylsulfonylamino)-4′oxo-3′,3′- | hydrogen | isopropyl |
| | dimethylbutanoyl | | |
| 1111 | 4′-(phenylsulfonylamino)-4′oxo-3′,3′- | hydrogen | isopropenyl |
| | dimethylbutanoyl | | |
| 1112 | 4′-(phenylsulfonylamino)-4′oxo-3′,3′- | hydrogen | isopropyl |
| | dimethylbutanoyl | | |
| 1113 | 5′-(phenylsulfonylamino)-5′-oxo-3′,3′- | hydrogen | isopropenyl |
| | dimethylpentanoyl | | |
| 1114 | 5′-(phenylsulfonylamino)-5′-oxo-3′,3′- | hydrogen | isopropyl |
| | dimethylpentanoyl | | |
| 1115 | 4′-[5-(3-methyl-1,2,4-oxadiazolyl)]-4′- | hydrogen | isopropenyl |
| | oxo-3′,3′-dimethylbutanoyl | | |
| 1116 | 4′-[5-(3-methyl-1,2,4-oxadiazolyl)]-4′- | hydrogen | isopropyl |
| | oxo-3′,3′-dimethylbutanoyl | | |
| 1117 | 4′-(2-thiazolylamino)-4′-oxo-3′,3′- | hydrogen | isopropenyl |
| | dimethylbutanoyl | | |
| 1118 | 4′-(2-thiazolylamino)-4′-oxo-3′,3′- | hydrogen | isopropyl |
| | dimethylbutanoyl | | |
| 1119 | cyanoaminocarbonyl-3′,3′- | hydrogen | isopropenyl |
| | dimethylbutanoyl | | |
| 1120 | cyanoaminocarbonyl-3′,3′- | hydrogen | isopropyl |
| | dimethylbutanoyl | | |
| 1121 | 4′-cyano-3′,3′-dimethylbutanoyl | hydrogen | isopropenyl |
| 1122 | 4′-cyano-3′,3′-dimethylbutanoyl | hydrogen | isopropyl |
| 1123 | 4′-(5-tetrazolyl)-3′,3′-dimethylbutanoyl | hydrogen | isopropenyl |
| 1124 | 4′-(5-tetrazolyl)-3′,3′-dimethylbutanoyl | hydrogen | isopropyl |
| 1125 | methylsulfonylaminocarbonylpropanoyl | hydrogen | isopropenyl |
| 1126 | methylsulfonylaminocarbonylpropanoyl | hydrogen | isopropyl |
| 1127 | phenylsulfonylaminocarbonylpropanoyl | hydrogen | isopropenyl |
| 1128 | phenylsulfonylaminocarbonylpropanoyl | hydrogen | isopropyl |
| 1129 | aminocarbonylpropanoyl | hydrogen | isopropenyl |
| 1130 | aminocarbonylpropanoyl | hydrogen | isopropyl |
| 1131 | tert-butanoyl | hydrogen | isopropenyl |
| 1132 | tert-butanoyl | hydrogen | isopropyl |
| 1133 | isopropanoyl | hydrogen | isopropenyl |
| 1134 | isopropanoyl | hydrogen | isopropyl |

Additional preferred compounds include derivatives of R₁. Examples can be found in Table 14:

TABLE 14

| | | R₂ is (iv) | |
|---|---|---|---|
| # | R₁ | and R₉ is | R₃ |
| 1135 | 4′-(methylsulfonylamino)-4′oxo-3′,3′- | hydrogen | isopropenyl |
| | dimethylbutanoyl | | |
| 1136 | 4′-(methylsulfonylamino)-4′oxo-3′,3′- | hydrogen | isopropyl |
| | dimethylbutanoyl | | |
| 1137 | 4′-(phenylsulfonylamino)-4′oxo-3′,3′- | hydrogen | isopropenyl |
| | dimethylbutanoyl | | |
| 1138 | 4′-(phenylsulfonylamino)-4′oxo-3′,3′- | hydrogen | isopropyl |
| | dimethylbutanoyl | | |
| 1139 | 5′-(phenylsulfonylamino)-5′-oxo- | hydrogen | isopropenyl |
| | 3′,3′-dimethylpentanoyl | | |
| 1140 | 5′-(phenylsulfonylamino)-5′-oxo- | hydrogen | isopropyl |
| | 3′,3′-dimethylpentanoyl | | |
| 1141 | 4′-[5-(3-methyl-1,2,4-oxadiazolyl)]-4′- | hydrogen | isopropenyl |
| | oxo-3′,3′-dimethylbutanoyl | | |
| 1142 | 4′-[5-(3-methyl-1,2,4-oxadiazolyl)]-4′- | hydrogen | isopropyl |
| | oxo-3′,3′-dimethylbutanoyl | | |
| 1143 | 4′-(2-thiazolylamino)-4′-oxo-3′,3′- | hydrogen | isopropenyl |
| | dimethylbutanoyl | | |
| 1144 | 4′-(2-thiazolylamino)-4′-oxo-3′,3′- | hydrogen | isopropyl |
| | dimethylbutanoyl | | |
| 1145 | 4′-cyanoamino-4′-oxo-3′,3′- | hydrogen | isopropenyl |
| | dimethylbutanoyl | | |
| 1146 | 4′-cyanoamino-4′-oxo-3′,3′- | hydrogen | isopropyl |
| | dimethylbutanoyl | | |
| 1147 | 4′-(methylsulfonylamino)-4′-oxo- | hydrogen | isopropenyl |
| | butanoyl | | |
| 1148 | 4′-(methylsulfonylamino)-4′-oxo- | hydrogen | isopropyl |
| | butanoyl | | |
| 1149 | 4′-(phenylsulfonylamino)-4′-oxo- | hydrogen | isopropenyl |
| | butanoyl | | |
| 1150 | 4′-(phenylsulfonylamino)-4′-oxo- | hydrogen | isopropyl |
| | butanoyl | | |
| 1151 | 4′-amino-4′-oxo-butanoyl | hydrogen | isopropenyl |
| 1152 | 4′-amino-4′-oxo-butanoyl | hydrogen | isopropyl |
| 1153 | tert-butanoyl | hydrogen | isopropenyl |
| 1154 | tert-butanoyl | hydrogen | isopropyl |
| 1155 | isopropanoyl | hydrogen | isopropyl |
| 1156 | isopropanoyl | hydrogen | isopropyl |

In some embodiments, 3′,3′-dimethylsuccinyl is at the C-3 position. In some embodiments, the C-3 substituents having dimethyl groups or oxygen at the C-3′ position can be the most active compounds. This observation suggests that these types of substituents might be important to enhanced anti-HIV activity.

Alkyl groups and alkyl containing groups of the compounds of the present invention can be straight chain or branched alkyl groups, preferably having one to ten carbon atoms. Typical C₁₋₁₀ alkyl groups include methyl, ethyl, propyl, isopropyl, butyl, sec-butyl, tert-butyl, pentyl, hexyl, heptyl, and octyl groups. In some embodiments, alkyl groups have one to six carbons. As described herein, any alkyl group, or alkyl containing group, can optionally be substituted with one or more halo, hydroxyl, or thiol.

The term “alkenyl” refers to C₂₋₁₀ alkenyl groups, preferably C₂₋₄ alkenyl. Typical C₂₋₄ alkenyl groups include ethenyl, propenyl, isopropenyl, butenyl, and sec-butenyl. The term alkenyl also refers to all stereoisomers, i.e., cis and trans isomers, as well at the E and Z isomers.

The term “cycloalkyl” refers to cyclized alkyl groups that are saturated or partially unsaturated. Cycloalkyl groups can include C₃₋₈ cycloalkyl. Typical cycloalkyl groups include cyclopropyl, cyclobutyl, cyclopentyl, cyclohexyl and cycloheptyl.

The term “cycloalkylalkyl” refers to any of the above-mentioned C₁₋₁₀ alkyl groups attached to any of the above-listed cycloalkyl groups, such as cyclopropylmethyl or cyclohexylethyl.

The term “heterocyclyl” or “heterocyclic” is used herein to mean saturated or partially unsaturated 3-7 membered monocyclic, or 3-14 membered bicyclic, ring system which consists of carbon atoms and from one to four heteroatoms independently selected from the group consisting of O, N, and S. Examples include, but are not limited to, tetrahydrofuranyl, tetrahydropyranyl, pyrrolidinyl, piperidinyl, piperazinyl, pyrazolidinyl, dihydrofuranyl, morpholinyl, dihydroimidazolyl, dihydropyranyl, dihydrooxazolyl, tetrahydrooxazolyl, 2-azabicyclo[2.2.1]heptanyl, 2,5-diazabicyclo[2.2.1]heptanyl, oxazinyl, isoxazinyl, oxathiazinyl and the like. Heterocyclic groups can be optionally substituted with one or more methyl, ethyl, oxo, halo, hydroxy, amino, alkylamino, dialkylamino, thiol, hydroxymethyl, hydroxyethyl, hydroxypropyl, methoxymethyl, toluenyl, carboxyl, benzyl, C₁-C₄ alkoxycarbonyl, tert-butoxycarbonyl, 4-morpholinylcarbonyl, alkyl, cycloalkyl, cycloalkylalkyl, hydroxyl, alkoxycarbonylamino, aryl, arylalkyl, alkanoyl, alkylthio, alkylsulfinyl, alkylsulfonyl, aminocarbonyl, alkylaminocarbonyl, dialkylaminocarbonyl, aminosulfonyl, alkylaminosulfonyl, dialkylaminosulfonyl, alkoxyalkyl, heteroarylalkyl, heterocyclyl, or dimethoxybenzyl; preferably optionally substituted with one or more methyl, ethyl, oxo, halo, thiol, hydroxymethyl, hydroxyethyl, hydroxypropyl, or methoxymethyl. In some embodiments, the term “heterocyclyl” refers to a cycloalkyl group that contains oxygen in the ring, i.e., a cyclic ether such as tetrahydrofuran or tetrahydropyran.

The term “heterocycloalkyl” refers to any of the above-mentioned C₁₋₁₀ alkyl groups attached to any of the above-mentioned heterocyclic groups.

The term “heterocycloalkylamino” refers to any of the above-mentioned heterocycloalkyl groups attached to an amino nitrogen.

The term “aryl” refers to any aromatic carbon ring structure, or any carbon ring structure with aromatic properties. Preferred aryls include C₆₋₁₄ aryl, especially C₆₋₁₀ aryl, such as phenyl or naphthyl, and most preferably six carbon aryl. Aryl groups are optionally substituted with one or more methyl, ethyl, hydroxyl, alkoxy, amino, alkylamino, dialkylamino, alkanoylamino, alkylsulfonamido, halo, thiol, alkylthio, alkylsulfinyl, alkylsulfonyl, hydroxymethyl, hydroxyethyl, hydroxypropyl, methoxymethyl, toluenyl, carboxyl, benzyl, or dimethoxybenzyl. Preferably aryl groups are optionally substituted with one or more methyl, ethyl, halo, thiol, hydroxymethyl, hydroxyethyl, hydroxypropyl, methoxymethyl, toluenyl, carboxyl, benzyl, or dimethoxybenzyl.

The term “arylalkyl” refers to any of the above-mentioned C₁₋₁₀ alkyl groups attached to any of the above-mentioned C₆₋₁₄ aryl groups. Useful arylalkyl groups include phenyl, phenethyl, and phenpropyl.

The term “arylalkenyl” refers to any of the above-mentioned C₂₋₄ alkenyl groups attached to any of the above-mentioned C₆₋₁₄ aryl groups.

The term “heteroaryl” refers to 5-14 membered heteroaromatic ring systems, especially 5-14 membered heteroaromatic ring systems, and most preferably five or six membered heteroaromatic groups, wherein from one to four atoms in the ring structure are heteroatoms independently selected from the group consisting of O, N, and S. Examples include, but are not limited to, tetrazolyl, pyridinyl, imidazolyl, isoxazolyl, furanyl, oxazolyl, thiazolyl, pyrrolyl, thienyl, pyrazolyl, triazolyl, e.g., 1,2,3-triazolyl and 1,2,4-triazolyl, isothiazolyl, oxadiazolyl, e.g., 1,2,3-oxadiazolyl, 1,2,4-oxadiazolyl, 1,2,5-oxadiazolyl, and 1,3,4-oxadiazolyl, oxatriazolyl, pyridazinyl, pyrimidinyl, pyrazinyl, triazinyl, e.g., 1,2,3-triazinyl and 1,2,4-triazolyl, quinolinyl, isoquinolinyl, indolyl, benzofuranyl, benzothienyl, benzimidazolyl, and indazolyl.

Useful heteroarylalkyl include any of the above-listed heteroaryl groups attached to an alkyl group. Useful heteroarylalkyl groups include: 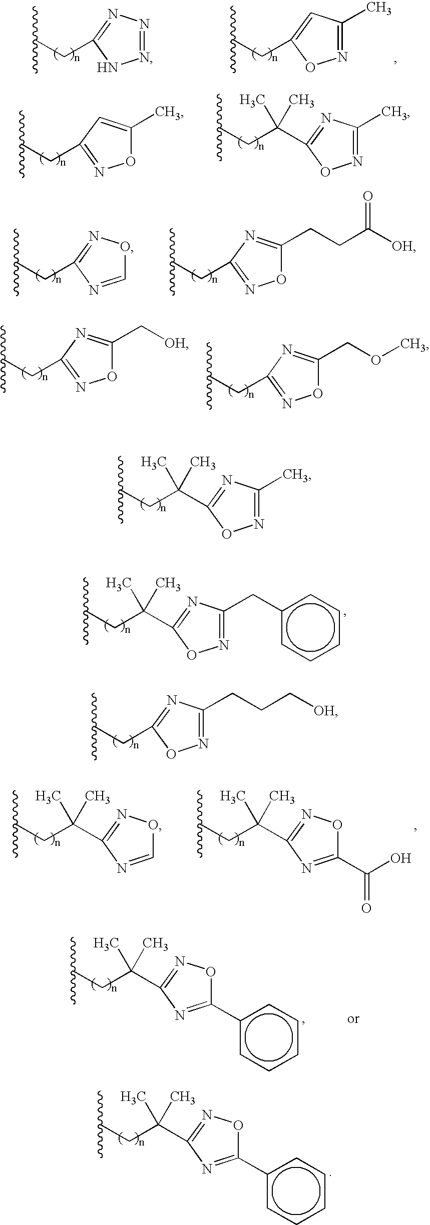
wherein n is one to eight, more preferably one to six.

The term “alkoxy” refers to a C₁₋₁₀ alkyl group as described above, wherein one of the carbon atoms is substituted by an oxygen atom.

The term “alkanoyl” refers to an alkyl group as defined above attached to a carbonyl group.

The term “carboxyalkanoyl” refers to an alkanoyl group as defined above attached to a carboxyl group.

The terms “alkylamino” and “dialkylamino” refer to —NHRₓ and —NRₓR_{y} respectively, wherein Rₓ and R_{y} are C₁₋₁₀ alkyl groups.

The term “dialkylaminoalkyl” refers to any of the above-mentioned C₁₋₁₀ alkyl groups attached to any of the above-mentioned dialkylamino groups.

The term “dialkylaminoalkylamino” refers to any of the above-mentioned dialkylaminoalkyl groups attached to an amino nitrogen, such as dimethylamino ethyl amino.

The term “aminoalkyl” refers to an amino groups (—NH₂) attached to an alkyl chain.

The term “aminocarbonyl” refers to —C(O)NH₂.

The term “alkylaminocarbonyl” and “dialkylaminocarbonyl” refers to carbonyl groups attached to —NHR₁₂ or —NR₁₂R₁₃ respectively, wherein R₁₂ and R₁₃ are C₁₋₁₀ alkyl groups.

The terms “halo” or “halogen” refer to an atom selected from the group consisting of fluorine, chlorine, bromine and iodine.

The terms “carboxyl” and “carboxy” refer to a substituent of formula —COOH.

The term “carboxyacyl” refers to a dicarboxy compound in which a hydroxy has been removed from one of the carboxyl groups, e.g., substituents of formula —C(O)CⱼCO₂H, were j is 0-20.

The term “cyano” refers to a substituent of formula —CN.

The term “alkylazo” refers to a substituent of the general formula —N═N—(CH₂)ₙ—CH₃, wherein n is one to six.

The term “oxo,” refers to ═O.

The term “sulfo” refers to the sulfonic acid group —SO₃H.

The term “sulfonyl” refers to the radical —SO₂—.

The term “sulfinyl” refers to the group —S═O.

The terms “phosphono” refers to the phosphonic acid radical —P(O)(OH)₂.

The term “phosphonoalkyl” refers to a substituent of the general formula —(CH₂)ₙPO₃H₂, wherein n is one to six.

The term “sulfoalkyl” refers to a substituent of the general formula —(CH₂)ₙSO₃H, wherein n is one to six.

The term “formyl” refers to a substituent of the general formula —CH═O. In some embodiments, the formyl group can be substituted with a halogen.

As used herein, the term “isopropenyl” refers to a substituent of formula 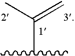
The term “propen-2-yl” is used interchangeably with isopropenyl, with the exception that the numbering of propen-2-yl follows accepted IUPAC rules.

The terms “hydroximino” or “hydroxyimino” refer to a substituent of the general formula ═N—OH. The term “1′-hydroxyiminoethyl” refers to a substituent of the formula —C(═N—OH)CH₃. The term “1″-alkoxyiminoethyl” refers to a substituent of the general formula —C(═N—O—(CH₂)ₚCH₃)CH₃, wherein p is 0 to 6.

The term “optionally substituted” refers to the replacement of a hydrogen in a compound in exchange for an atom or substituent.

Also, included within the scope of the present invention are the non-toxic pharmaceutically acceptable salts of the compounds of the present invention. These salts can be prepared in situ during the final isolation and purification of the compounds or by separately reacting the purified compound in its free acid form with a suitable organic or inorganic base and isolating the salt thus formed. These can include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, and cations of methylamine, dimethylamine, trimethylamine, ethylamine, N-methylglucamine and the like. The salts can also be prepared by reacting the purified betulin compound containing an amine in its base form with a suitable organic or inorganic acid, and isolating the salt thus formed. These base salts can include halides, such as chloride, bromide, and iodide, phosphate, sulfate, and the like; organic acid salts such as citrate, lactate, tartrate, maleate, fumarate, mandelate, acetate, dichloroacetate, trifluoroacetate, oxalate, formate, and the like; and sulfonates such as methanesulfonate, benzenesulfonate, p-toluenesulfonate and the like.

The invention disclosed herein is also meant to encompass prodrugs of the disclosed compounds. The expression “prodrug” refers to compounds that are rapidly transformed in vivo by an enzymatic or chemical process, to yield the parent compound of the above formulas, for example, by hydrolysis in blood. Typical prodrugs are esters of the parent drug. A thorough discussion is provided by Higuchi, T. and V. Stella in Prodrugs as Novel Delivery Systems, Vol. 14, A.C.S. Symposium Series, and in Bioreversible Carriers in Drug Design, Ed. Edward B. Roche, American Pharmaceutical Association, Pergamon Press, 1987. Other examples of prodrugs are drug compounds covalently linked to lipid molecules. Such lipid-linked compounds may have longer half-lives in the body than the drug compounds themselves. They can also be incorporated into liposomes, which may be used to improve the targeting of infected cells, or to enhance the uptake of the drug by infected cells. A thorough discussion of such compositions and methods is provided in U.S. Pat. No. 6,002,029, U.S. Pat. No. 6,448,392 and U.S. Pat. No. 6,599,887. Further examples of prodrugs are drug compounds linked to, or incorporated into, nanometer-sized particles for enhanced absorption by, or improved targeting of, cells within the body. Methods of this sort are described in Weissleder, R. et al., Nature Biotech. 23 Oct. 2005, NBT1159, p. 1-6; Allen, T. and Cullis, P. R., Science 303:1818-1822 (2004); LaVan et al., Nature Rev. Drug Disc. 1:77-84 (2002); and Kralj, M. and Pavelic, K., EMBO Reports 4:1008-1012 (2003).

The invention disclosed herein is also meant to encompass the in vivo metabolic products of the disclosed compounds. Such products may result for example from the oxidation, reduction, hydrolysis, amidation, esterification, glucuronidation and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabeled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein is also meant to encompass the disclosed compounds being isotopically labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ⁸F, and ³⁶Cl, respectively.

Some of the compounds disclosed herein may contain one or more asymmetric centers and may thus give rise to enantiomers, diastereomers, and other stereoisomeric forms. The present invention is also meant to encompass all such possible forms, as well as their racemic and resolved forms and mixtures thereof. In some embodiments, the compounds of the present invention can be separated as a single enantiomer. Alternatively, the individual enantiomers may be separated according to methods that are well known to those of ordinary skill in the art.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule.

The invention is also directed to a method for treating a subject infected with HIV-1 by administering at least one of the above-noted betulin derivatives, optionally in combination with any one or more of the known anti-AIDS therapeutics or an immunostimulant.

Other features, advantages, embodiments, aspects and objects of the present invention will be clear to those skilled in the areas of relevant art, based upon the description, teaching and guidance presented herein.

The analogs of the present invention can have anti-retroviral activity, thus providing suitable compounds and compositions for treating retroviral infections, optionally with additional pharmaceutically active ingredients, such as anti-retroviral, anti-HIV, and/or immunostimulating compounds or antiviral antibodies or fragments thereof.

By the term “anti-retroviral activity” or “anti-HIV activity” is intended the ability to inhibit at least one of:

(1) viral pro-DNA integration into host cell genome;

(2) retroviral attachment to cells;

(3) viral entry into cells;

(4) cellular metabolism which permits viral replication;

(5) inhibition of intercellular spread of the virus;

(6) synthesis and/or cellular expression of viral antigens;

(7) viral budding or maturation;

(8) activity of virus-coded enzymes (such as reverse transcriptase, integrase and proteases); and/or

(9) any known retroviral or HIV pathogenic actions, such as, for example, immunosuppression. Thus, any activity which tends to inhibit any of these mechanisms is “anti-retroviral activity” or “anti-HIV activity.”

A compound of the present invention can be used for treatment of retroviral (e.g., HIV) infection either alone, or in combination with other modes of therapy known in the art. Such modes of therapy can include chemotherapy with drugs, such as, but not limited to, at least one of AZT, 3TC, ddC, d4T, ddI, tenofovir, abacavir, nevirapine, delavirdine, emtricitabine, efavirenz, saquinavir, ritonavir, indinavir, nelfinavir, lopinavir, amprenavir, fosamprenavir, tipranavir, and atazanavir or any other antiretroviral drugs or antibodies in combination with each other, or associated with a biologically based therapeutic, such as, for example, gp41-derived peptides enfuvirtide (Fuzeon; Trimeris-Roche) and T-1249 (Trimeris), or soluble CD4, antibodies to CD4, and conjugates of CD4 or anti-CD4, or as additionally presented herein.

A compound according to the present invention can be used in treating blood products, such as those maintained in blood banks. The nation's blood supply is currently tested for antibodies to HIV. However, the test is still imperfect and samples which yield negative tests can still contain HIV virus. Treating the blood and blood products with the compounds of the present invention can add an extra margin of safety by reducing or eliminating activity of any retrovirus that may have gone undetected.

A compound according to the present invention can be used in the treatment of HIV in patients who are not adequately treated by other HIV-1 therapies. Accordingly, the invention is also drawn to a method of treating a patient in need of therapy, wherein the HIV-1 infecting said cells does not respond to other HIV-1 therapies. In another embodiment, methods of the invention are practiced on a subject infected with an HIV that is resistant to a drug used to treat HIV infection. In various applications, the HIV is resistant to one or more protease inhibitors, reverse transcriptase inhibitors, entry inhibitors, nucleoside analogs, vaccines, binding inhibitors, immunomodulators, and/or any other inhibitors. In some embodiments, the compositions and methods of the invention are practiced on a subject infected with an HIV that is resistant to one or more drugs used to treat HIV infections, for example, but not limited to, zidovudine, lamivudine, didanosine, zalcitabine, stavudine, abacavir, nevirapine, delavirdine, emtricitabine, efavirenz, saquinavir, ritonavir, lopinavir, indinavir, nelfinavir, tenofovir, amprenavir, adefovir, atazanavir, fosamprenavir, tipranavir, enfuvirtide, hydroxyurea, AL-721, ampligen, butylated hydroxytoluene; polymannoacetate, castanospermine; contracan; creme pharmatex, CS-87, penciclovir, famciclovir, acyclovir, cytofovir, ganciclovir, dextran sulfate, D-penicillamine trisodium phosphonoformate, fusidic acid, HPA-23, eflornithine, nonoxynol, pentamidine isethionate, peptide T, phenytoin, isoniazid, ribavirin, rifabutin, ansamycin, trimetrexate, SK-818, suramin, UA001, and combinations thereof.

In addition, compounds of the present invention can be used as prophylactics to prevent transmission of HIV infection between individuals. For example, the compounds can be administered orally or by injection to an HIV infected pregnant woman and/or fetus during pregnancy or immediately prior to, at, or subsequent to birth, to reduce the probability that the newborn infant becomes infected. Also, the compounds can be administered vaginally immediately prior to childbirth to prevent infection of the infant during passage through the birth canal. Further, the compounds of the present invention can be used during sexual intercourse to prevent transmission of HIV by applying a retroviral inhibiting effective amount of a topical composition including one or more compounds of Formula I to vaginal or other mucosa prior to sexual intercourse. For example, the compounds of the present invention can be used to prevent transmission of HIV from an infected male to an uninfected female or vice versa.

Pharmaceutical Compositions

Pharmaceutical compositions can comprise at least one compound of the present invention. Pharmaceutical compositions according to the present invention can also further comprise one or more additional antiviral agents such as, but not limited to, AZT (zidovudine, RETROVIR, GlaxoSmithKline), 3TC (lamivudine, EPIVIR®, GlaxoSmithKline), AZT+3TC, (COMBIVIR®, GlaxoSmithKline) AZT+3TC+abacvir (TRIZIVIR®, GlaxoSmithKline), ddI (didanosine, VIDEX®, Bristol-Myers Squibb), ddC (zalcitabine, HIVID®, Hoffmann-LaRoche), D4T (stavudine, ZERIT®, Bristol-Myers Squibb), abacavir (ZIAGEN®, GlaxoSmithKline), nevirapine (VIRAMLNE®, Boehringher Ingelheim), delavirdine (Pfizer), efavirenz (SUSTIVA®, DuPont Pharmaceuticals), tenofovir (VIREAD®, Gilead Sciences), FTC (emtricitabine, EMTRIVA®, Gilead Sciences), tenofivir+FTC (TRUVADA®, Gilead Sciences), saquinavir (INVIRASE®, FORTOVASE®, Hoffmann-La Roche), ritonavir (NORVIR®, Abbott Laboratories), indinavir (CRIXIVAN®, Merck and Company), nelfinavir (VIRACEPT®, Pfizer), amprenavir (AGENERASE®, GlaxoSmithKline), adefovir (PREVEON®, HEPSERA®, Gilead Sciences), atazanavir (REYATAZ®, Bristol-Myers Squibb), fosamprenavir (LEXIVA®, GlaxoSmithKline), hydroxyurea (HYDREA®, Bristol-Meyers Squibb), and tipranavir (APTIVUS®, Boehringer Ingelheim), or any other antiretroviral drugs or antibodies in combination with each other, or associated with a biologically based therapeutic, such as, for example, gp41-derived peptides enfuvirtide (FUZEON®, Roche and Trimeris) and T-1249, or soluble CD4, antibodies to CD4, and conjugates of CD4 or anti-CD4, or as additionally presented herein.

Additional suitable antiviral agents for optimal use with a compound of the present invention can include, but is not limited to, amphotericin B (FUNGIZONE®); Ampligen (mismatched RNA; Hemispherx Biopharma); interferon beta (BETASERON®, Chiron, Berlex); interferon alfa (INTRON A®, Schering-Plough; ROFERON A®, Hoffman-LaRoche; INFERGEN®, Amgen; WELLFERON®, GlaxoSmithKline); pegylated interferon alfa (PEGASYS®, Hoffman-LaRoche; PEG-Intron®, Schering-Plough); butylated hydroxytoluene; Carrosyn (polymannoacetate); Castanospermine; Contracan (stearic acid derivative); Creme Pharmatex (containing benzalkonium chloride); 5-unsubstituted derivative of zidovudine; penciclovir (DENAVIR®, Novartis); famciclovir (FAMVIR®, Novartis); acyclovir (ZOVIRAX®, GlaxoSmithKline); cytofovir (VISTIDE®, Gilead); ganciclovir (CYTOVENE®, Hoffman LaRoche); valacyclovir, VALTREX®, GlaxoSmithKline); dextran sulfate; D-penicillamine (3-mercapto-D-valine); FOSCARNET® (trisodium phosphonoformate; AstraZeneca); fusidic acid; glycyrrhizin (a constituent of licorice root); HPA-23 (ammonium-21-tungsto-9-antimonate); ORNIDYL® (eflornithine, Aventis); nonoxynol; pentamidine isethionate (PENTAM-300); Peptide T (octapeptide sequence, Peninsula Laboratories); Phenyloin (Pfizer); INH or isoniazid; ribavirin (REBETOL®, Schering-Plough; VIRAZOLE®, Valeant Pharmaceuticals); rifabutin, ansamycin (MYCOBUTIN®, Pfizer); CD4-IgG2 (Progenics Pharmaceuticals) or other CD4-containing or CD4-based molecules; Trimetrexate (Medimmune); suramin and analogues thereof (Bayer).

Pharmaceutical compositions of the present invention can also further comprise immunomodulators. Suitable immunomodulators for optional use with a compound of the present invention in accordance with the present invention can include, but are not limited to: ABPP (Bropririmine); anti-human interferon-α-antibody; ascorbic acid and derivatives thereof; interferon-β; Ciamexon; cyclosporin; cimetidine; CL-246,738; colony stimulating factors, including GM-CSF; dinitrochlorobenzene; HE2000 (Hollis-Eden Pharmaceuticals); inteferon-γ; glucan; hyperimmune gamma-globulin (Bayer); immuthiol (sodium diethylthiocarbamate); interleukin-1 (Hoffmann-LaRoche, Amgen), interleukin-2 (IL-2) (Chiron); isoprinosine (inosine pranobex); Krestin; LC-9018 (Yakult); lentinan (Yamanouchi); LF-1695; methionine-enkephalin; Minophagen C; muramyl tripeptide, MTP-PE; naltrexone (Barr Laboratories); RNA immunomodulator; REMUNE® (Immune Response Corporation); RETICULOSE® (Advanced Viral Research Corporation); shosaikoto; ginseng; thymic humoral factor; Thymopentin; thymosin factor 5; thymosin 1 (ZADAXIN®, SciClone); thymostimulin; TNF (tumor necrosis factor, Genentech); and vitamin preparations.

In some embodiments, the animal subject of the present invention is a mammal. By the term “mammal” is meant an individual belonging to the class Mammalia. The invention is particularly useful in the treatment of human patients.

The term “treating” means the administering to subjects a compound of the present invention for purposes which can include prevention, amelioration, or cure of a retroviral-related pathology.

Medicaments are considered to be provided “in combination” with one another if they are provided to the patient concurrently or if the time between the administration of each medicament is such as to permit an overlap of biological activity.

In some embodiments, at least one compound of the present invention comprises a single pharmaceutical composition.

Pharmaceutical compositions for administration according to the present invention can comprise at least one compound according to the present invention in a pharmaceutically acceptable form optionally combined with a pharmaceutically acceptable carrier. These compositions can be administered by any means that achieve their intended purposes. Amounts and regimens for the administration of a compound according to the present invention can be determined readily by those with ordinary skill in the clinical art of treating a retroviral pathology.

For example, administration can be by parenteral, such as subcutaneous, intravenous, intramuscular, intraperitoneal, transdermal, or buccal routes. Alternatively, or concurrently, administration can be by the oral route. The dosage administered depends upon the age, health and weight of the recipient, type of previous or concurrent treatment, if any, frequency of treatment, and the nature of the effect desired.

Compositions within the scope of this invention include all compositions comprising at least one compound according to the present invention in an amount effective to achieve its intended purpose. While individual needs vary, determination of optimal ranges of effective amounts of each component is within the skill of the art. Typical dosages comprise about 0.1 mg/kg to about 100 mg/kg body weight. In some embodiments, the dosages comprise about 1 mg/kg to about 100 mg/kg body weight of the active ingredient. In some embodiments, the dosages comprise about 1 mg/kg to about 50 mg/kg body weight. In some embodiments, the dosages comprise about 5 mg/kg to about 25 mg/kg body weight.

Therapeutic administration can also include prior, concurrent, subsequent or adjunctive administration of at least one additional compound according to the present invention or other therapeutic agent, such as an antiviral or immune stimulating agent. In such an approach, the dosage of the second drug can be the same as or different from the dosage of the first therapeutic agent. In some embodiments, the drugs are administered on alternate days in the recommended amounts of each drug.

Administration of a compound of the present invention can also optionally include previous, concurrent, subsequent or adjunctive therapy using immune system boosters or immunomodulators. In addition to the pharmacologically active compounds, a pharmaceutical composition of the present invention can also contain suitable pharmaceutically acceptable carriers comprising excipients and auxiliaries which facilitate processing of the active compounds into preparations which can be used pharmaceutically. In some embodiments, the preparations, particularly those preparations which can be administered orally and which can be used in the above-described type of administration, such as tablets, dragees, and capsules, and also preparations which can be administered rectally, such as suppositories, as well as suitable solutions for administration by injection or orally, contain from about 1 percent to about 99 percent, preferably from about 20 percent to about 75 percent of active compound(s), together with the excipient.

Pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture, and processing the mixture of granules, after adding suitable auxiliaries, if desired or necessary, to obtain tablets or dragee cores.

Suitable excipients are, e.g., fillers such as saccharides, e.g., lactose, sucrose, mannitol or sorbitol; cellulose preparations and/or calcium phosphates, such as tricalcium phosphate or calcium hydrogen phosphate; as well as binders such as starch paste, using, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone. If desired, disintegrating agents can be added such as the above-mentioned starches and also carboxymethyl starch, cross-linked polyvinylpyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions can be used, which can optionally contain gum arabic, talc, polyvinylpyrrolidone, poly(ethylene glycol) and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate are used. Dyestuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In some embodiments using soft capsules, the active compounds are dissolved or suspended in suitable liquids, such as fatty oils or liquid paraffin. In addition, stabilizers can be added.

Possible pharmaceutical preparations which can be used rectally include, for example, suppositories which consist of a combination of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, poly(ethylene glycols), or paraffin hydrocarbons.

Suitable formulations for parenteral administration include aqueous solutions of the active compounds in water-soluble form, for example, water-soluble salts. In addition, suspensions of the active compounds as appropriate oily injection suspensions can be administered. Suitable lipophilic solvents or vehicles include fatty oils, such as sesame oil, or synthetic fatty acid esters, such as ethyl oleate or triglycerides. Aqueous injection suspensions that can contain substances which increase the viscosity of the suspension include, for example, sodium carboxymethylcellulose, sorbitol, and/or dextran. Optionally, the suspension can also contain stabilizers.

A pharmaceutical formulation for systemic administration according to the invention can be formulated for enteral, parenteral or topical administration. Indeed, all three types of formulation can be used simultaneously to achieve systemic administration of the active ingredient.

Suitable formulations for oral administration include hard or soft gelatin capsules, dragees, pills, tablets, including coated tablets, elixirs, suspensions, syrups or inhalations and controlled release forms thereof.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as, for example, water or other solvents, solubilizing agents and emulsifiers such as ethyl alcohol, isopropyl alcohol, cyclodextrins such as hydroxypropyl-β-cyclodextrin, ethyl carbonate, ethyl acetate, benzyl alcohol, benzyl benzoate, propylene glycol, 1,3-butylene glycol, N,N-dimethylformamide, oils such as cottonseed, groundnut, corn, germ, olive, castor, and sesame oils, glycerol, tetrahydrofurfuryl alcohol, poly(ethylene glycols) and fatty acid esters of sorbitan, and mixtures thereof.

Suspensions, in addition to the active compounds, may contain suspending agents as, for example, ethoxylated isostearyl alcohols, poly(oxyethylene) sorbitol and sorbitan esters, cellulose, microcrystalline cellulose, aluminum metahydroxide, bentonite, agar-agar, and gum tragacanth, and combinations thereof.

Solid dosage forms in addition to those formulated for oral administration include rectal suppositories.

Prophylactic topical compositions for preventing HIV infection between individuals during childbirth or sexual intercourse include one or more compounds of Formula I and at least one pharmaceutically acceptable topical carrier or diluent. The topical composition can be, for example, in the form of an ointment, a cream, a gel, a lotion, a paste, a jelly, a spray, a foam, or a sponge. The dosage amount of a compound of Formula I in a prophylactic topical formulation is, in general, less than about 1,000 milligrams, and in some embodiments from about 0.01 milligrams to about 100 milligrams. The topical formulations can include other prophylactic ingredients. The carrier and diluents should be acceptable in the sense of being compatible with other ingredients of the formulation and not deleterious to the recipient.

Topical prophylactic formulations include those suitable for vaginal, rectal or topical administration. The formulations can, where appropriate, be conveniently presented in discrete dosage units, and can be prepared by any of the methods known in the art of pharmacy. All such methods include the step of bringing the active agent into association with liquid carriers, gels or finely divided solid carriers or both and then, if necessary, shaping the product into the desired formulation.

Prophylactic formulations suitable for vaginal administration can be presented as pessaries, tampons, creams, gels, pastes, jelly, foams, or sprays, or aqueous or oily suspensions, solutions or emulsions (liquid formulations) containing suitable carriers known in the art in addition to the active agent. Liquid formulations can contain conventional additives, such as, suspending agents, emulsifying agents, non-aqueous vehicles including edible oils, or preservatives. These formulations are useful to prevent both sexual transmission of HIV and infection of an infant during passage through the birth canal. In one example, the vaginal administration can take place prior to sexual intercourse, or immediately prior to childbirth.

In some embodiments, prophylactic formulations suitable for rectal or vaginal administration having a solid carrier are represented as unit dose suppositories. Suitable carriers include cocoa butter and other materials commonly used in the art. Suppositories can be formed, for example, mixing one or more compounds of Formula I with one or more softened or melted carriers followed by chilling and shaping in molds.

Prophylactic formulations according to the invention can also be in the form of drops formulated with an aqueous or non-aqueous base comprising one or more dispersing agents, solubilizing agents, or suspending agents. Liquid sprays can be delivered from pressurized packs.

Prophylactic formulations according to the invention can be adapted to give sustained delivery. Also, the prophylactic formulations can include other active agents, such as spermicidal agents, antimicrobial agents, and antiviral agents.

The compounds of the present invention can also be administered in the form of an implant when compounded with a biodegradable slow-release carrier. Alternatively, the compounds of the present invention can be formulated as a transdermal patch for continuous release of the active ingredient.

Suitable formulations for topical administration include creams, gels, jellies, mucilages, pastes and ointments. Suitable injectable solutions include intravenous subcutaneous and intramuscular injectable solutions. Alternatively, the compounds can be administered in the form of an infusion solution or as a nasal inhalation or spray.

The compounds of the present invention can be prepared using methods known to those skilled in the art. Betulin and betulinic acid can be obtained from commercial sources. In general, methods used in make compounds of the present invention employ protection and deprotection steps, for example, protection of hydroxy, amino and carboxy groups. Protecting groups and their chemistry are described generally in Protective Groups in Organic Synthesis, 3^{rd} ed. (eds. T. W. Greene and P. G. M. Wuts, John Wiley and Sons, Inc. (1999)). The compounds of Formula I of the present invention wherein R₂ is (ii) can be prepared in a manner similar to that exemplified by the modification of betulin as shown in Scheme 1. Betulin or dihydrobetulin can be heated overnight at 95° C. with 6-fold of the appropriate anhydride in anhydrous pyridine in the presence of 4-(N,N-dimethylamino)pyridine (DMAP). R_{z} corresponds to —COR₅, —R₆ or —CO(CH₂)_{d}NR₁₂R₁₃, wherein R₅, R₆ R₁₂, R₁₃ and d are defined above. When thin layer chromatography (TLC) indicates complete consumption of starting material, the reaction can be diluted with EtOAc and washed with 10% HCl solution. The EtOAc layer can then be dried over MgSO₄ and subjected to column chromatography.

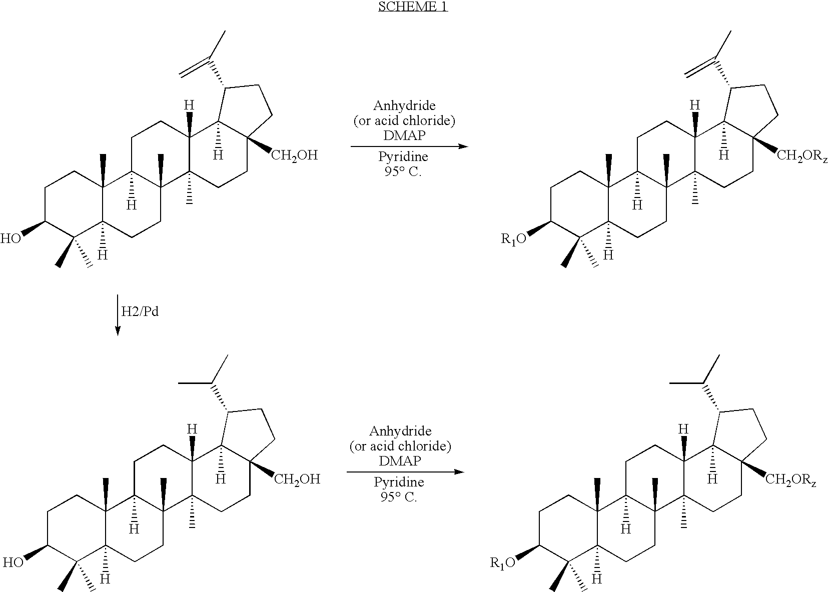

The compounds of Formula I of the present invention can be prepared in a manner similar to that exemplified by the modification of betulin as shown in Scheme 2. Scheme 2 depicts the synthesis route for compounds where R₁ is substituted or unsubstituted carboxyacyl. R_{z} corresponds to —COR₅, —R₆ or —CO(CH₂)_{d}NR₁₂R₁₃, wherein R₅, R₆ R₁₂, R₁₃ and d are defined above.

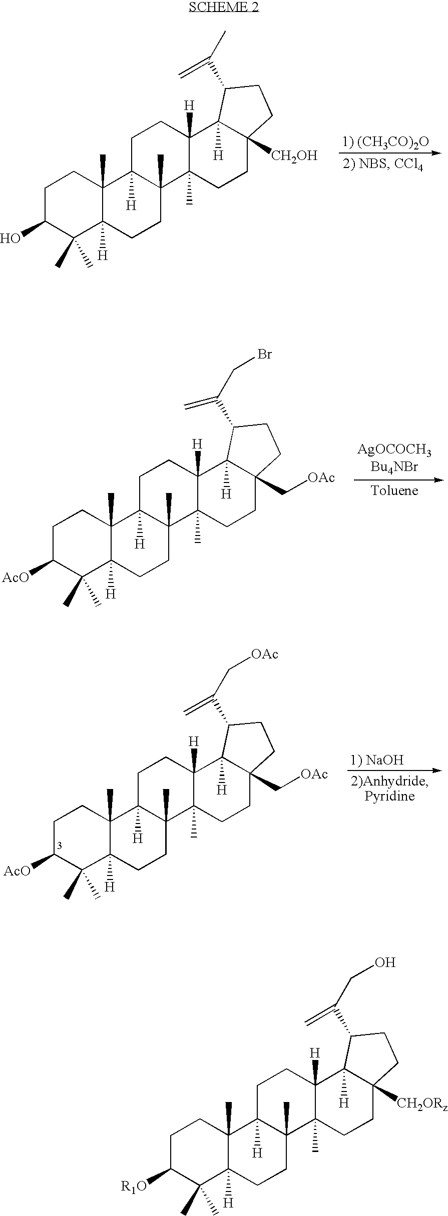

Scheme 3 depicts an alternative method of synthesizing the compounds of the present invention by the use of solid phase organic synthesis (Pathak, A., et al. Combinatorial Chem. and High Throughput Screening 5, 241-248 (2002)). Briefly, a betulin backbone can be linked to a resin via ester or amide bond formation at R₅, R₆, R₇, R₈, R₉, R₁₀, R₁₁, R₁₂ or R₁₃ (denoted by Rₐ). Any resin which allows cleavage of compounds under mild conditions can be used, e.g., 2-chlorotrityl chloride resin or Sieber amide resin. An amino acid can be introduced as a spacer between the betulin and the resin if desired. Once the betulin is immobilized onto the resin scaffold, diversity can be introduced as desired at the C-3 position by adding the acid form of the desired R₁ substituents (denoted by R_{b}).

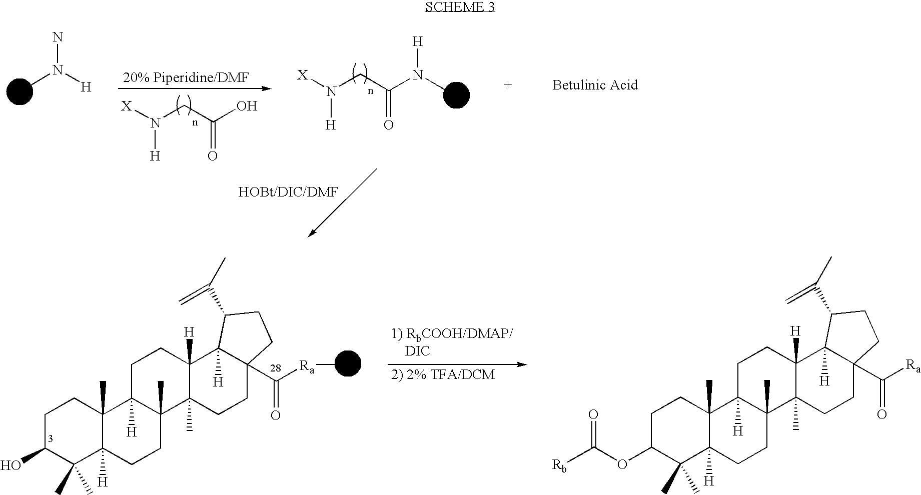

The compounds of the present invention containing modifications at the C-3 position can be prepared as shown in Scheme 4. Protection of the 28-hydroxyl group of betulin (1) with triphenylmethyl ether group yields betulin 28-O-triphenylmethyl ether (2), whose solution in pyridine is further treated with an appropriate dicarboxylic acid in the presence of DMAP at reflux. Finally, the 28-protective group is removed by refluxing with pyridinium p-toluenesulfonate (PPTS) in CH₂Cl₂-EtOH to give desired 3-O-(acyl)betulin derivatives.

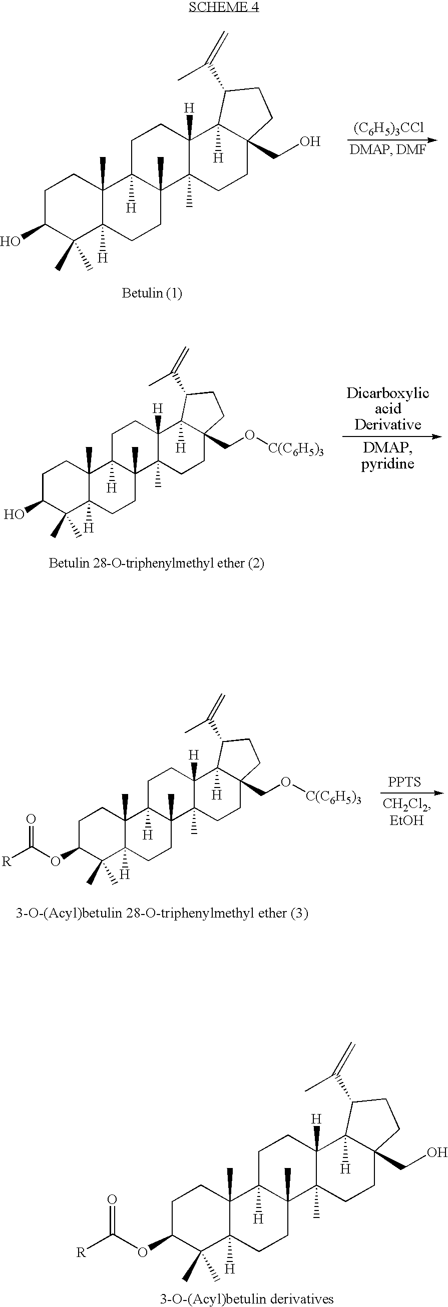

The C-28 amides of the present invention can be synthesized by the following methods. A first method of synthesis of betulinic acid amides is performed by forming C-3 protected betulinic acid C-28 acid halides as described in Scheme 5. A number of additional alcohols can be used in the first step in addition to the allylalcohol or methanol, e.g., alkyl, alkenyl or aralkyl alcohols can be used. A C-28 amide is introduced by treatment of the C-3 protected betulinic acid C-28 acid halides with the desired amine under appropriate conditions, such as in dry dichloromethane and N,N-diisopropylethylamine (Method D). The carboxy-protecting group from the first step is then removed. Deprotection steps are well-known in the art for particular protecting groups. See for example Method E and Method F as described herein.

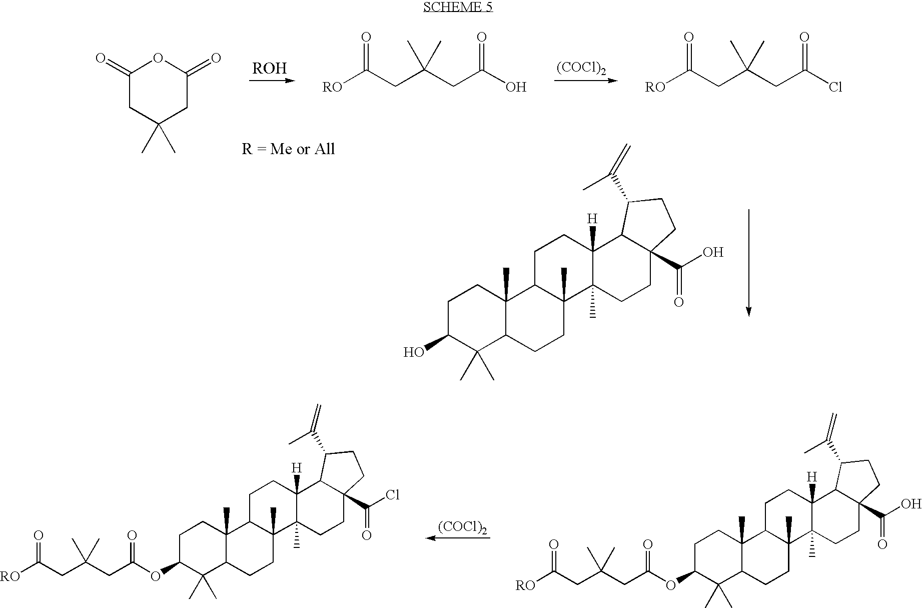

Thus, another aspect of the invention is directed to a method of synthesizing a compound of Formula I wherein R₂ is formula (v) comprising: (a) forming a monoprotected di-carboxylic acid derivative, (b) activating the non-protected carboxyl group of the di-carboxylic acid to form an acid halide, (c) reacting the acid halide of step (b) with betulinic acid to form the R₁ group at the C-3 position, (d) activating the C-28 position of the compound of (c) to form an acid halide, (e) attaching the desired amine at C-28, and (f) deprotecting the protected R₁ carboxyl group of (a).

A second method of synthesis of betulinic acid amides is shown in Scheme 6.

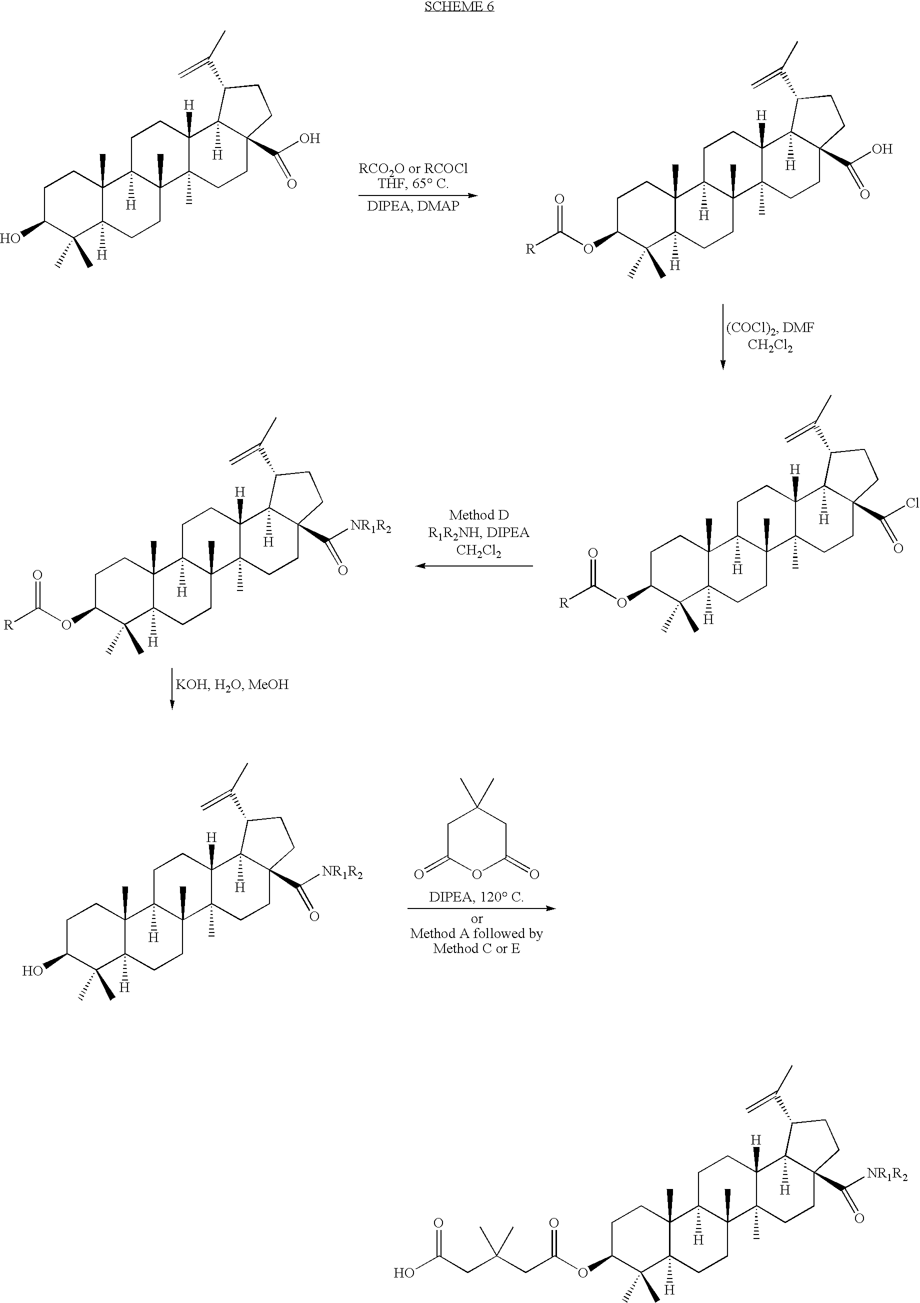

The C-3 alcohol of betulinic acid is first protected with a suitable hydroxy protecting group, such as the acetate or benzoate using either the acid anhydride or acid chloride and N,N-diisopropylethylamine (DIPEA) in tetrahydrofuran (THF) with DMAP as catalyst. The C-28 carboxylic acid is activated as an acid halide or other suitable activating group. Reagents useful for this conversion include but are not limited to oxalyl chloride, oxalyl bromide, thionyl chloride, thionyl bromide, phosphorous oxychloride, phosphorous oxybromide, phosphorous pentachloride, phosphorous pentabromide, phosphorous trichloride, phosphorous tribromide and the like. The appropriate amide is formed by treatment of the acid halide with the desired amine in dry dichloromethane and DIPEA (Method D). The C-3 acetyl group is removed by basic hydrolysis using potassium or sodium hydroxide in aqueous alcohol (Method G). The C-3 group is introduced using the appropriate anhydride to provide directly the desired compound (Method H). In some instances, the C-3 group can be introduced with methyl or allyl 3,3-dimethylglutaryl chloride in dichloromethane and DIPEA using Method A followed by removal of the C-5′ ester using either Method C for the allyl ester or Method E for the methyl ester.

Thus, another aspect of the invention is directed to a method of synthesizing a compound of Formula I wherein R₂ is formula (v), comprising: (a) protecting a C-3 alcohol of betulinic acid; (b) activating the C-3 protected betulinic acid at the C-28 carbon to form a C-3 protected, C-28 activated betulinic acid; (c) the resulting compound of (b) reacting the C-3 protected, C-28 activated betulinic acid with an appropriated amine; (d) deprotecting the the resulting compound of step (c) at its C-3 position and (e) adding an R₁ ester group at C-3.

### EXAMPLE 1

### Synthesis of Betulinic Acid C-3 Modifications

Methods to synthesize 3-O-(acyl)betulinic acid compounds are depicted in Scheme 7.

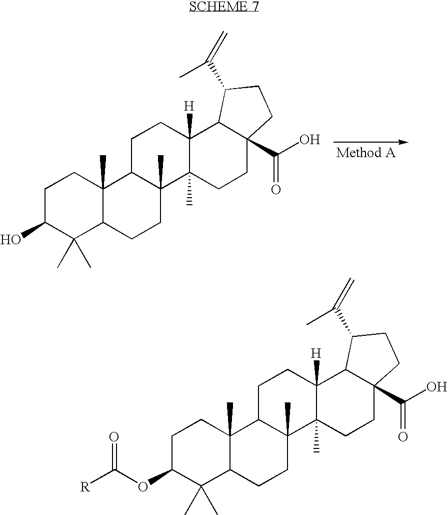

Method A: 3-O-(Acyl)betulinic acid compounds are prepared by adding betulinic acid (1 equivalent) to a stirred solution of the desired acid chloride or sulfonyl chloride (4 equivalents) in dry dichloromethane, followed by DMAP (1 equivalent) and DIPEA (4 equivalents). The reaction was heated at 40° C. overnight, diluted in EtOAc, washed successively with 1M HCl (aq), water and dried over Na₂SO₄. The combined organic layers were concentrated to dryness in vacuo. Final compounds were purified by flash column chromatography on silica gel.

### 3-O-(5′-Morpholinyl-5′-oxo-3′,3′-dimethylpentanoyl)betulinic acid

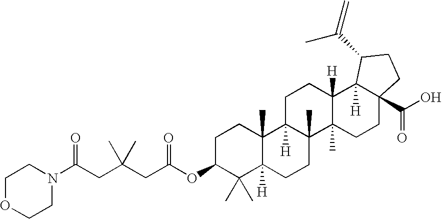

The compound was synthesized by coupling betulinic acid with 5-morpholino-5-oxo-3,3-dimethylpentanoyl chloride applying method A (47 mg, 3%); ¹H NMR (400 MHz, CDCl₃) δ ppm 0.72-1.76 (42H, m), 1.89-2.06 (3H, m), 2.12-2.23 (1H, m), 2.27 (1H, d, J=12.7 Hz), 2.39-2.49 (3H, m), 2.52 (2H, d), 2.93-3.08 (1H, m), 3.46-3.63 (4H, m), 3.64-3.78 (4H, m), 4.46 (1H, dd, J=10.8, 5.4 Hz), 4.61 (1H, s), 4.74 (1H, s).

### Synthesis of substituted 3-O-[5′-(sulfonylamino)-3′,3′-dimethyl glutaryl]betulinic acids

Substituted 3-O-[5′-(sulfonylamino)-3′,3′-dimethylglutaryl]betulinic acids were synthesized in 4 steps from betulinic acid as shown in Scheme 8.

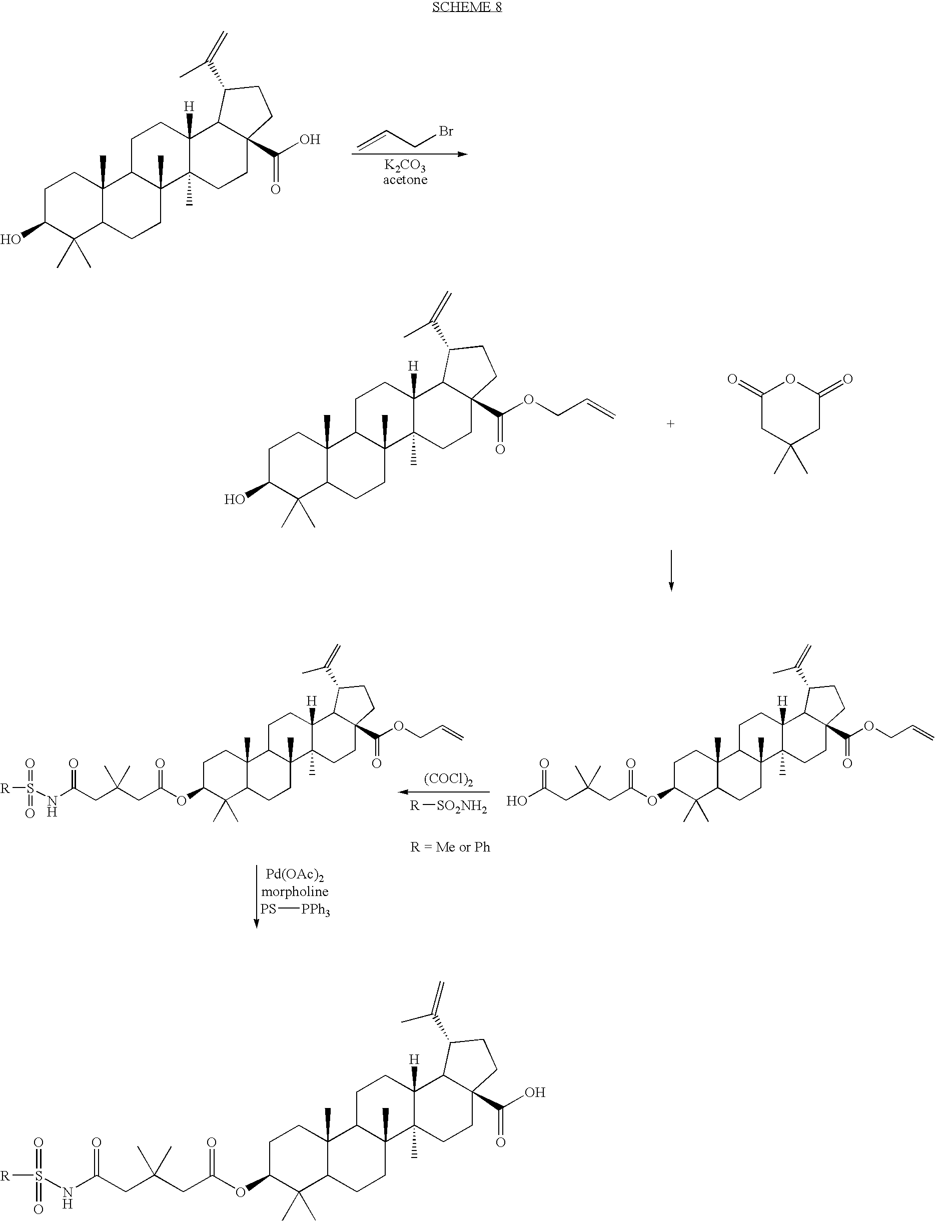

### Betulinic acid allyl ester

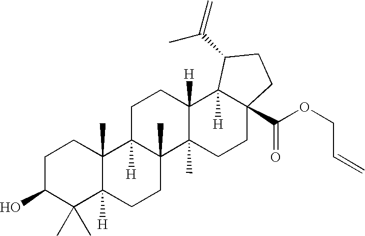

Betulinic acid (0.8 g, 1.6 mmol) and 0.28 mL (2 eq., 3.2 mmol) allyl bromide were dissolved in 10 mL of acetone. Potassium carbonate (0.69 g, 5 mmol) was then added. The resulting suspension was stirred at reflux for 3 hours. The insoluble inorganic salts were removed by filtration and the reaction mixture was concentrated under reduced pressure to yield crude product (1.04 g, quantitative) used without further purification.

### 3-O-(3′,3′-Dimethylglutaryl)betulinic acid allyl ester

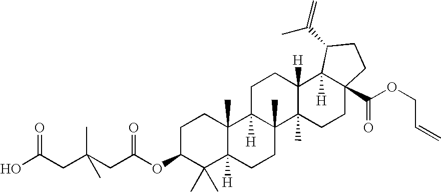

Betulinic acid allyl ester (1.04 g, 1.6 mmol), 0.45 g (2 eq., 3.2 mmol) 3,3′-dimethylglutaric anhydride and DMAP (0.19 g, 1.6 mmol) were suspended in 5 mL of pyridine under nitrogen and stirred at reflux for 25 hours. After removal of all solvent under reduced pressure an orange-brown solid was obtained. Purification by flash column chromatography (2 to 20% EtOAc in heptane) yielded 0.803 g of product, used without further purification.

### 3-O-[5′-(Phenylsulfonylamino)-3′,3′-dimethylglutaryl]betulinic acid allyl ester

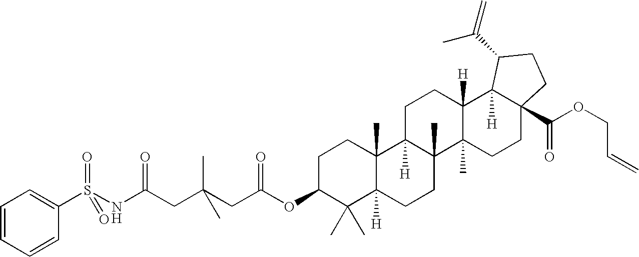

3-O-(3′,3′-Dimethylglutaryl)betulinic acid allyl ester (0.4 g, 0.62 mmol) was dissolved in 4 mL of dichloromethane under nitrogen. Oxalyl chloride (0.31 g, 1.2 mmol) was added and the reaction was left to stir at rt for 1 hour. After removal of all solvents under reduced pressure, a pale yellow solid was obtained. This solid was re-dissolved in 5 mL of dichloromethane and benzenesulfonamide (0.3 g, 1.9 mmol) was added. The reaction was stirred at rt overnight. Solvents were removed under reduced pressure and the crude product was purified by flash column chromatography (2 to 10% EtOAc in heptane) yielding 0.622 g of desired product which was used without further purification.

### 3-O-[5′-(Phenylsulfonylamino)-5′-oxo-3′,3′-dimethylpentanoyl]betulinic acid

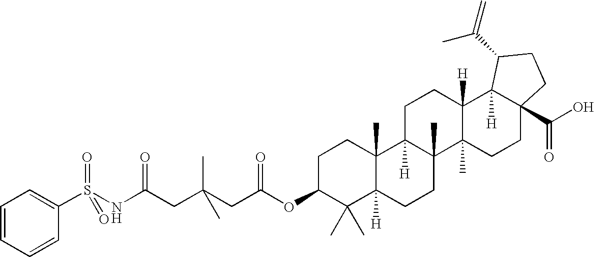

3-O-[5′-(Phenylsulfonylamino)-3′,3′-dimethylglutaryl]betulinic acid allyl ester (0.112 g, 0.14 mmol), 0.033 g (1 eq., 0.14 mmol) palladium(II) acetate, polymer bound triphenylphosphine (0.145 g, 0.432 mmol) and morpholine (0.125 mL, 0.14 mmol) were suspended in 3 mL of THF under nitrogen and stirred at 50° C. for 20 hours. After removal of all solvent under reduced pressure a brown solid was obtained. Purification with preparative HPLC yielded 27 mg of product. ¹H NMR (400 MHz, CDCl₃) δ ppm 10.46 (1H, s), 8.09 (2H, d, J=7.34 Hz), 7.42-7.69 (3H, m), 4.43-4.84 (3H, m), 3.01 (1H, d, J=4.9 Hz), 2.12-2.40 (7H, m), 1.87-2.07 (2H, m), 0.64-1.81 (43H, m); LCMS, R_{f}=4.86 min, 100% (M+Na)⁺ 760 (100%).

### 3-O-[4′-(Methylsulfonylamino)-4′-oxo-3′,3′-dimethylbutanoyl]betulinic acid

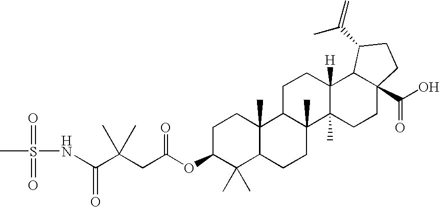

3-O-[4′-(Methylsulfonylamino)-4′-oxo-3′,3′-dimethylbutanoyl]betulinic acid can be prepared by coupling the acid chloride of allyl (3′,3′-dimethylbutanoyl)betulinic acid with methanesulfonamide followed by removal of the allyl ester.

### EXAMPLE 2

### Synthesis of 3-O-Acyl Betulinic Acid C-28 Derivatives: Preparation of Intermediates

Synthesis of C-28 derivatives of 3-O-(acyl)betulinic acid is accomplished by coupling a suitably protected O-acyl side chain on the C-3 hydroxyl of betulinic acid and reacting the resulting compound with oxalyl chloride to form the corresponding betulinic acid chloride derivative. This C-28 acid chloride is then coupled to the desired group, and subsequently is deprotected to form the targeted C-28 derivative.

Alternatively 3-O-acetylbetulinic acid is activated and coupled to the desired group. The 3-O-acetyl group is then removed by hydrolysis and the desired 3-O-acyl side chain is introduced at the C-3 position resulting in formation of the betulinic acid C-28 derivative.

### 3-O-(5′-Alkoxy-3′,3′-dimethylglutaryl)betulinic acid chloride preparations

3-O-(5′-Alkoxy-O-3′,3′-dimethylglutaryl)betulinic acid chlorides (where alkoxy=allyl or methyl) were prepared in four steps from 3,3-dimethylglutaric anhydride as shown in Scheme 9.

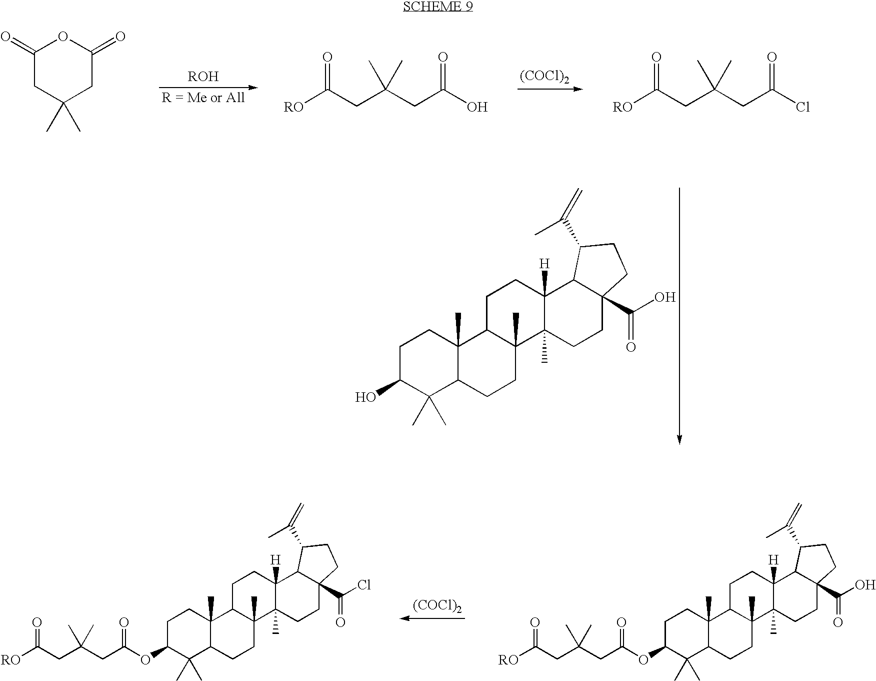

Ring opening of 3,3-dimethylglutaric anhydride with allyl alcohol or methanol followed by treatment of the resulting acids with oxalyl chloride afforded methyl or allyl 3,3-dimethylglutaryl chloride. The acid chlorides were coupled to betulinic acid and the resulting products were converted to their corresponding acid chlorides by treatment with oxalyl chloride.

### 3-O-(5′-Allyloxy-3′,3′-dimethylglutaryl)betulinic acid chloride preparation

### Mono-Allyl 3,3-dimethylglutarate

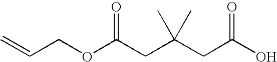

A suspension of 3,3-dimethylglutaric anhydride (5.3 g, 38 mmol) in allylic alcohol (10 mL, 145 mmol) was heated at reflux for 5 hours (solution became clear). The allylic alcohol was removed in vacuo, the residue was then diluted in EtOAc (100 mL), washed successively twice with water, dried over Na₂SO₄, and concentrated in vacuo to afford the desired compound (6.7 g, 99%) as a colorless oil which was used in the next step without further purification. ¹H NMR (400 MHz, CDCl₃): δ 1.13-1.18 (s, 6H), 2.48 (s, 2H), 2.49 (s, 2H), 4.59 (d, 2H, J=5.8 Hz), 5.25 (dd, 1H, J=10.4, 1.3 Hz), 5.32 (dd, 1H, J=17.3, 1.3 Hz), 5.9 (m, 1H).

### Allyl 3,3-dimethylglutaryl chloride

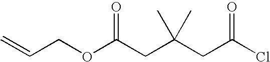

N,N-Dimethylformamide (DMF) (30 μL, 0.38 mmol) was added to a stirred solution of oxalyl chloride (16.6 mL, 175 mmol) and allyl 3,3-dimethylglutarate (3.5 g, 17.5 mmol) in dichloromethane (60 mL) at 0° C. The reaction was allowed to reach rt and was stirred for 1 hour. The volatiles were removed in vacuo. The resulting solid residue was dissolved in dichloromethane (10 mL) and concentrated to dryness in vacuo. This operation was repeated twice more, to afford the desired acid chloride (3.8 g, quantitative yield) as yellow oil, which was used without further purification.

### 3-O-(5′-Allyloxy-3′,3′-dimethylglutaryl)betulinic acid

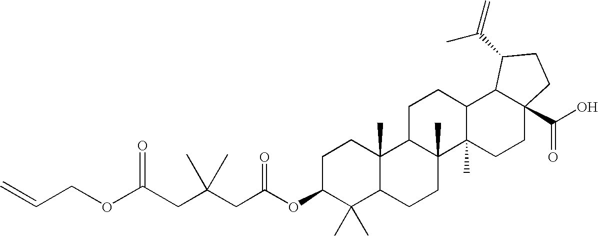

Betulinic acid (2.0 g, 4.38 mmol) was added to a stirred solution of allyl 3,3-dimethylglutaryl chloride (3.8 g, 17.5 mmol) in dry dichloromethane (60 mL) followed by DIPEA (1.53 mL, 8.76 mmol) at 0° C. The ice bath was removed and the reaction was heated at 40° C. overnight. The reaction mixture was concentrated in vacuo and the residue was diluted in EtOAc (100 mL), washed twice with 1M HCl, and dried over Na₂SO₄. The combined organic layers were concentrated to dryness in vacuo. Flash column chromatography on silica gel (EtOAc 0 to 10% in heptane) provided the desired compound (2.38 g, 85%) as a white solid. TLC (EtOAc:heptane 2:8) R_{f}=0.37; ¹H NMR (400 MHz, CDCl₃) δ ppm 10.7 (1H, s), 5.85-5.97 (1H, m), 5.27-5.36 (1H, m), 5.19-5.26 (1H, m), 4.74 (1H, d, J=1.8 Hz), 4.61 (1H, s), 4.54-4.59 (2H, m), 4.47 (1H, dd, J=11.2, 4.9 Hz), 3.01 (1H, ddd), 2.34-2.52 (4H, m), 2.12-2.23 (1H, m), 1.91-2.06 (2H, m), 0.73-1.79 (45H, m) of which 1.70 (s), 1.12 (s), 0.97 (s), 0.93 (s), 0.85 (s), 0.82 (s).

### 3-O-(5′-Allyloxy-3′,3′-dimethylglutaryl)betulinic acid chloride

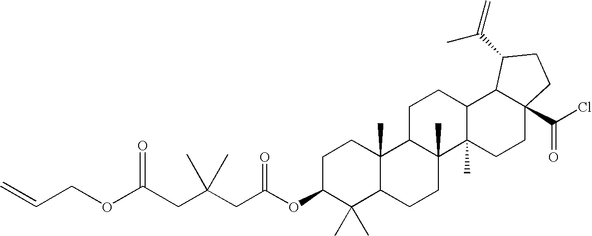

DMF (20 μL, 0.25 mmol) was added to a stirred solution of oxalyl chloride (0.62 mL, 6.51 mmol) and 3-O-(5′-allyloxy-3′,3′-dimethylglutaryl)betulinic acid (0.692 g, 1.08 mmol) in dichloromethane (15 mL) at 0° C. The reaction was allowed to reach rt and was stirred for 12 hours. The volatiles were removed in vacuo. The resulting solid residue was dissolved in dichloromethane (10 mL) and concentrated to dryness in vacuo. This operation was repeated to afford the desired acid chloride (0.75 g, quantitative yield) as an oil, which was used without further purification. A sample of acid chloride was quenched in methanol to give the methyl ester: TLC (EtOAc:heptane 2:8) R_{f}=0.50; SM R_{f}=0.37.

### 3-O-(5′-Methoxy-3′,3′-dimethylglutaryl)betulinic acid chloride preparation

### Mono-methyl 3,3-dimethylglutarate

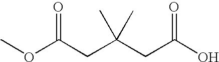

A suspension of 3,3-dimethylglutaric anhydride (9.0 g, 63.4 mmol) and DMAP (0.77 g, 6.3 mmol) in triethylamine (TEA) (8.8 mL, 63.4 mmol) and methanol (75 mL) was heated at reflux overnight. The methanol was removed in vacuo, and the residue was then dissolved in EtOAc (150 mL), washed successively with citric acid (1 M, 3×100 mL), water and dried over MgSO₄, and concentrated in vacuo to afford the desired compound (11.06 g, 100%) as a colorless oil which was used in the next step without further purification. ¹H NMR (400 MHz, CDCl₃): δ ppm 10.9 (1H, br s), 3.7 (3H, s), 2.45 (4H, d), 1.15 (6H, s).

### Methyl 3,3-dimethylglutaryl chloride

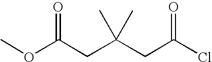

DMF (30 μL, 0.38 mmol) was added to a stirred solution of oxalyl chloride (7.7 mL, 90 mmol) and mono-methyl 3,3-dimethylglutarate (10.4 g, 60 mmol) in dichloromethane (100 mL) at 0° C. The reaction was allowed to reach rt and was stirred for 1 hour. The volatiles were removed in vacuo. The resulting solid residue was dissolved in dichloromethane (10 mL) and concentrated to dryness in vacuo. This operation was repeated twice more, to afford the desired acid chloride (11.5 g, quantitative yield) which was used without further purification.

### 3-O-(5′-Methoxy-3′,3′-dimethylglutaryl)betulinic acid

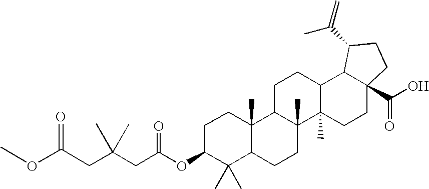

Betulinic acid (3.6 g, 7.9 mmol) was added to a stirred solution of methyl 3,3-dimethylglutaryl chloride (6.1 g, 31.7 mmol) in dry dichloromethane (30 mL) followed by DIPEA (5.5 mL, 31.7 mmol) at 0° C. The ice bath was removed and the reaction was stirred at rt overnight. The reaction mixture was concentrated in vacuo and the residue was diluted in EtOAc (100 mL), washed twice with 1M HCl, and dried over Na₂SO₄. The combined organic layers were concentrated to dryness in vacuo. Flash column chromatography on silica gel (EtOAc 2 to 5% in heptane) provided the desired compound (4.97 g, quantitative yield) as a white solid. ¹H NMR (400 MHz, CDCl₃) δ ppm 4.74 (1H, d, J=1.3 Hz), 4.61 (1H, s), 4.41-4.53 (1H, m), 3.7 (3H, s), 2.92-3.09 (1H, td, J=11.1, 4.1 Hz), 2.5-2.32 (4H, m), 2.3-1.9 (4H, m), 1.77-0.72 (44H, m).

### 3-O-(5′-Methoxy-3′,3′-dimethylglutaryl)betulinic acid chloride

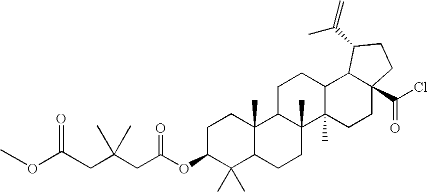

DMF (20 μL, 0.25 mmol) was added to a stirred solution of oxalyl chloride (1.03 mL, 12.0 mmol) and 3-O-(5′-methoxy-3′,3′-dimethylglutaryl)betulinic acid (1.46 g, 2.4 mmol) in dichloromethane (20 mL) at 0° C. The reaction was allowed to reach rt and was stirred for 14 hours. The volatiles were removed in vacuo. The resulting solid residue was dissolved in dichloromethane (10 mL) and concentrated to dryness in vacuo. This operation was repeated to afford the desired acid chloride (1.51 g, quantitative yield) as a pale yellow solid which was used without further purification. A sample of acid chloride was quenched in methanol to give the methyl ester: TLC (EtOAc:heptane 4:6) R_{f}=0.6.

### 3-O-Acetylbetulinic acid preparation

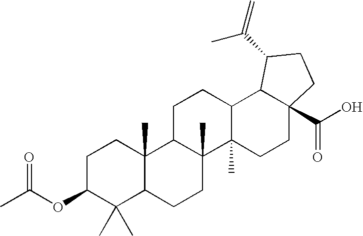

Betulinic acid (1.0 g, 2.2 mmol) was dissolved in 10 mL of dry THF and 1 mL of DIPEA. To this solution are added 0.034 g (0.27 mmol) of DMAP and 0.3 mL (3.1 mmol) of acetic anhydride. The mixture was heated at 65° C. for two hours until TLC showed complete consumption of the starting material. Minor traces of mixed anhydride were also present in the crude mixture. The reaction mixture was concentrated to dryness to yield a white solid. This solid was then suspended in 20 mL of a 0.6 M hydrochloric acid solution and heated at 100° C. for 30 minutes in order to hydrolyze any traces of undesired mixed anhydride. The white suspension was left to cool down to rt and the solid was collected by filtration. The cake was washed with 20 mL of water and dried at 50° C. under reduced pressure overnight yielding 1.06 g (2.1 mmol, 97%) of a white free flowing powder. TLC: R_{f}=0.65 (EtOAc: CH₂Cl₂ 5: 95); ¹H NMR: (250 MHz, CDCl₃); δ ppm 4.74 (1H, d, J=1.3 Hz), 4.61 (1H, s), 4.41-4.53 (1H, m), 2.92-3.09 (1H, m), 2.10-2.34 (2H, m), 1.92-2.09 (5H, m), 0.69-1.83 (38H, m).

### 3-O-Acetylbetulinic acid chloride preparation

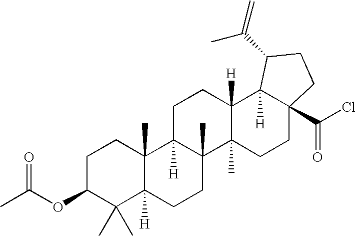

3-O-Acetylbetulinic acid (0.5 g, 1.0 mmol) was dissolved in 3 mL of dry THF under nitrogen. A few drops of DMF were added followed by slow addition of 0.3 mL (3 mmol) oxalyl chloride. The reaction was stirred at rt for two hours. All solvents were removed under reduced pressure and the resulting acid chloride was used without further purification.

### EXAMPLE 3

### Synthesis of Betulinic Acid Esters

C-28 esters of betulinic acid were prepared in two steps from 3-O-(5′-allyloxy-3′,3′-dimethylglutaryl)betulinic acid chloride as shown in Scheme 10. 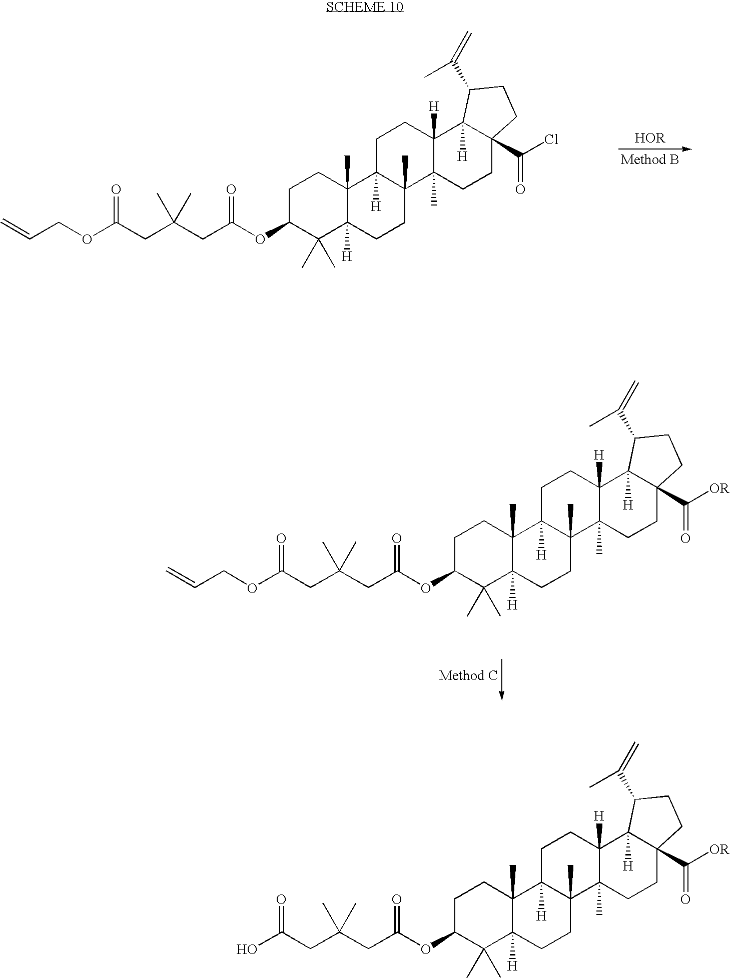
Method B: Esterification Method.

Betulinic esters were prepared by adding a solution of 3-O-(5′-allyloxy-3′,3′-dimethylglutaryl)betulinic acid chloride or 3-O-(5′-methoxy-3′,3′-dimethylglutaryl)betulinic acid chloride (1 equivalent) in dry dichloromethane to a stirred solution of the desired alcohol (2 to 5 equivalents) and DIPEA (3 to 6 equivalents) in dry dichloromethane at rt. The reaction was stirred at rt overnight, diluted in EtOAc, washed with 1M HCl, water and dried over Na₂SO₄. The combined organic layers were concentrated to dryness in vacuo and the resulting oil was purified by flash column chromatography on silica gel (hexane:EtOAc) to provide the desired betulinic ester.

Method C: Deallylation Method.

Palladium(II) acetate (1.05 equivalent) and polymer bound triphenylphosphine (3.1 equivalent) or Fibrecat palladium(II)® (0.5-1 equivalent) were added to a degassed solution of the desired allylic ester (1 equivalent) and morpholine (20 equivalents) in THF under a nitrogen atmosphere. The reaction was stirred overnight at 60° C. and allowed to cool down to rt. The resin was removed by filtration, and the organic solution was diluted with EtOAc, washed successively with 1M KHSO₄ (aq), water and dried over Na₂SO₄. The combined organic layers were concentrated to dryness in vacuo and the resulting solid purified by flash column chromatography on silica gel (hexane:EtOAc) to provide the desired deprotected acid.

### 3-O-(3′,3′-Dimethylglutaryl)betulinic acid 2-N,N-dimethylaminoethyl ester

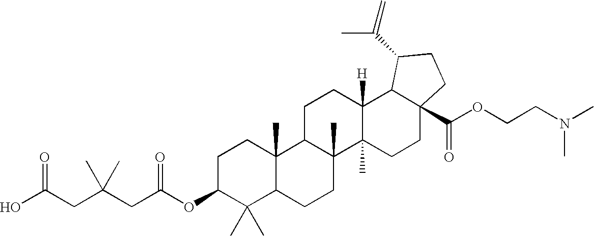

The compound was synthesized from 3-O-(5′-allyloxy-3′,3′-dimethylglutaryl)betulinic acid chloride applying method B with 2-N,N-(dimethylamino)ethanol (32%), followed by method C deprotection: (29 mg, 66%); ¹H NMR (400 MHz, CDCl₃) δ ppm 4.72 (1H, d, J=1.8 Hz), 4.59 (1H, s), 4.47 (1H, dd, J=11.0, 4.8 Hz), 4.16-4.28 (2H, m), 3.73-3.82 (2H, m), 2.93-3.04 (3H, m), 2.62-2.73 (1H, m), 2.15-2.54 (10H, m), 0.65-2.10 (45H, m); LCMS, 92% pure; R_{f}=3.20; m/z (relative intensity) 670 ([M+Na]⁺, 30%).

### 3-O-(3′,3′-Dimethylglutaryl)betulinic acid 2-cyanoethyl ester

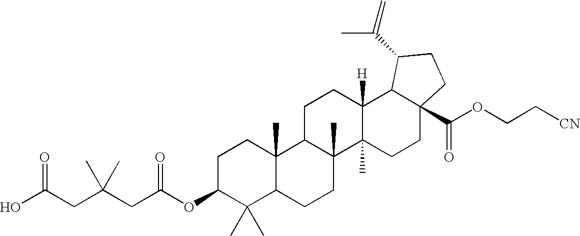

The compound was synthesized from 3-O-(5′-allyloxy-3′,3′-dimethylglutaryl)betulinic acid chloride applying method B with 2-cyanoethanol (29%), followed by method C deprotection: (16 mg, 40%); ¹H NMR (400 MHz, CDCl₃) δ ppm 4.70-4.78 (1H, m), 4.61 (1H, d, J=1.5 Hz), 4.50 (1H, dd, J=10.6, 5.1 Hz), 4.25-4.35 (2H, m), 2.91-3.06 (1H, m), 2.72 (2H, t, J=6.2 Hz), 2.37-2.53 (4H, m), 0.71-2.34 (48H, m); LCMS, 80% pure; R_{f}=3.90; m/z (relative intensity) 674 ([M+Na]⁺, 100%).

### 3-O-(3′,3′-Dimethylglutaryl)betulinic acid 2-methoxyethyl ester

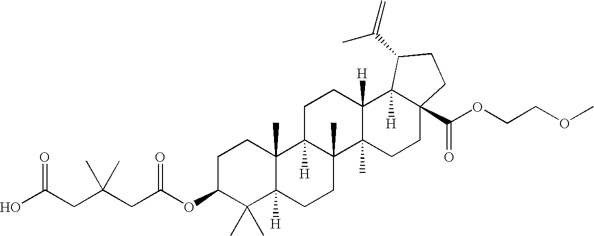

The compound was synthesized from 3-O-(5′-allyloxy-3′,3′-dimethylglutaryl)betulinic acid chloride applying method B with 2-methoxyethanol, followed by method C deprotection. ¹H NMR (400 MHz, CDCl₃); δ ppm 4.70 (1H, s), 4.62 (1H, s), 4.52-4.47 (1H, m), 4.28-4.24 (1H, m), 4.20-4.16 (1H, m), 3.58 (2H, t, J=4.8 Hz), 3.38 (3H, s), 3.04-3.02 (1H, m), 2.48-2.40 (4H, m), 2.30-2.18 (2H, m), 1.93-1.88 (2H, m), 1.87-0.61 (46H, m); LCMS, 100% R_{f}=5.10; m/z (relative intensity) 679 ([M+Na⁺] 100%).

### 3-O-(3′,3′-Dimethylglutaryl)betulinic acid (R)-3-[1-(tert-butoxycarbonyl)-pyrrolidinyl] ester

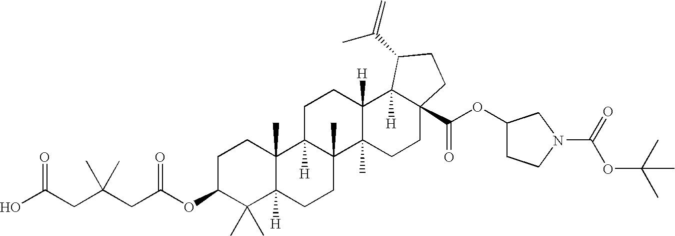

The compound was synthesized from 3-O-(5′-allyloxy-3′,3′-dimethylglutaryl)betulinic acid chloride applying method B with (R)-3-hydroxy-1-(tert-butoxycarbonyl)pyrrolidine, followed by method C deprotection. ¹H NMR (250 MHz, CDCl₃) δ ppm 0.65-2.76 (64H, m), 2.83-3.13 (1H, m), 3.54 (3H, br s), 4.50 (1H, dd, J=10.5, 5.8 Hz), 4.61 (1H, s), 4.73 (1H, d, J=1.6 Hz), 5.27 (1H, s).

### 3-O-(31,3′-Dimethylglutaryl)betulinic acid 3-(R/S)-3-(tetrahydrofuranyl) ester

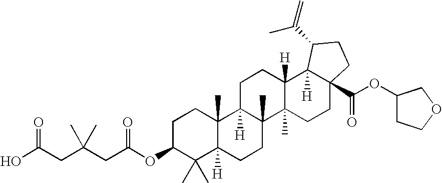

The compound was synthesized from 3-O-(5′-allyloxy-3′,3′-dimethylglutaryl)betulinic acid chloride applying method B with 3-hydroxytetrahydrofuran, followed by method C deprotection; ¹H NMR (400 MHz, CDCl₃) δ ppm 0.63-2.24 (50H, m), 2.34 (1H, d), 2.37-2.52 (3H, m), 2.86-2.99 (1H, m), 3.72 (1H, m), 3.83 (2H, dd, J=8.4, 5.1 Hz), 3.86-3.95 (1H, m), 4.42 (1H, dd, J=10.6, 5.1 Hz), 4.54 (1H, s), 4.66 (1H, s), 5.16-5.27 (1H, m).

### 3-O-(3′,3′-Dimethylglutaryl)betulinic acid ethyl ester

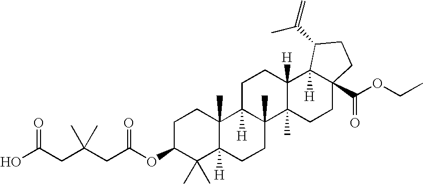

The compound was synthesized from 3-O-(5′-allyloxy-3′,3′-dimethylglutaryl)betulinic acid chloride applying method B with ethanol, followed by method C deprotection. ¹H NMR (400 MHz, CDCl₃); δ ppm 4.73 (1H, s), 4.60 (1H, s), 4.49-4.47 (1H, m), 4.19-4.10 (2H, m), 3.01-3.02 (1H, m), 2.50-2.30 (8H, m), 2.09-1.99 (1H, m), 1.87-0.61 (46H, m); LCMS, 97% R₁=4.34; m/z (relative intensity) 649 ([M+Na⁺] 100%).

### 3-O-(3′,3′-Dimethylglutaryl)betulinic acid isopropyl ester

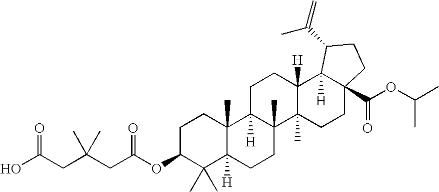

The compound was synthesized from 3-O-(5′-allyloxy-3′,3′-dimethylglutaryl)betulinic acid chloride applying method B with isopropanol, followed by method C deprotection. ¹H NMR (400 MHz, CDCl₃); δ ppm 5.04-5.00 (1H, m), 4.73 (1H, s), 4.60 (1H, s), 4.52-4.48 (1H, m), 3.04-3.02 (1H, m), 2.50-2.30 (8H, m), 2.09-1.99 (1H, m), 1.87-0.61 (49H, m); LCMS, 96% R_{f}=4.44; m/z (relative intensity) 664 ([M+Na⁺] 100%).

### 3-O-(3′,3′-Dimethylglutaryl)betulinic acid tert-butyl ester

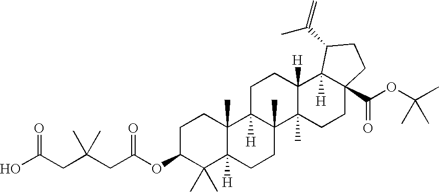

The compound was synthesized from 3-O-(5′-allyloxy-3′,3′-dimethylglutaryl)betulinic acid chloride applying method B with t-butanol, followed by method C deprotection. ¹H NMR (400 MHz, CDCl₃); δ ppm 4.73 (1H, s), 4.60 (1H, s), 4.52-4.48 (1H, m), 3.04-3.02 (1H, m), 2.50-2.30 (8H, m), 2.09-1.99 (1H, m), 1.87-0.61 (52H, m); LCMS, 95% R_{f}=4.56; m/z (relative intensity) 678 ([M+Na⁺] 100%).

### EXAMPLE 4

### Synthesis of Betulinic Acid Amides

Amides of betulinic acid were prepared either in two steps from 3-O-(5′-allyloxy-3′,3′-dimethylglutaryl)betulinic acid chloride and 3-O-(5′-methoxy-3′,3′-dimethylglutaryl)betulinic acid chloride or in 3 steps from 3-O-acetylbetulinic acid chloride as shown in Scheme 11. 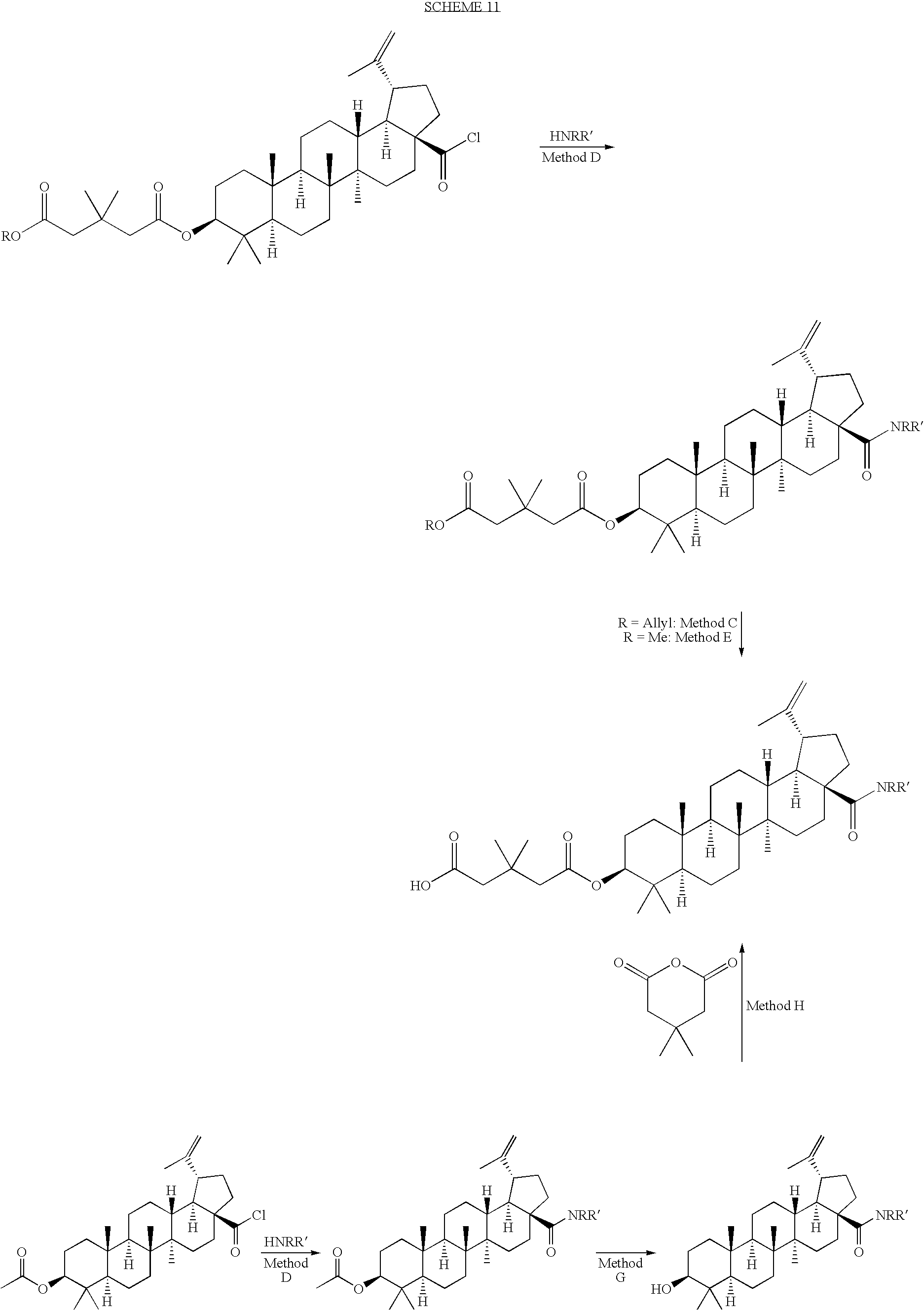
Method D: Amidation Method.

Betulinic acid amides were prepared by adding a solution of 3-O-(5′-allyloxy-3′,3′-dimethylglutaryl)betulinic acid chloride, 3-O-(5′-methoxy-3′,3′-dimethylglutaryl)betulinic acid or 3-O-acetylbetulinic acid (1 equivalent) in dry dichloromethane to a stirred solution of the desired amine (2-5 equivalents) in dry dichloromethane and DIPEA (3-6 equivalents) at rt. The reaction was stirred at rt overnight. The reaction mixture was then diluted in EtOAc, washed successively with 1 M HCl (aq.) and water, dried over Na₂SO₄. The combined organic layers were concentrated to dryness in vacuo and the resulting oil was purified by flash column chromatography on silica gel (hexane:EtOAc) to provide the desired betulinic acid derived amide.

Method E: Methyl Ester Hydrolysis Method.

2M Aqueous potassium hydroxide (2 equivalents) was added to a solution of the desired methyl ester (1 equivalent) in THF/Methanol (1:1). The reaction was stirred overnight at rt and for further 4 hours at 50° C. if not completed. Solvent was removed in vacuo, the crude product taken up in EtOAc, washed successively with 1M KHSO₄ (aq) and dried over Na₂SO₄. The combined organic layers were concentrated to dryness in vacuo and the resulting solid purified by flash column chromatography on silica gel (hexane:EtOAc) to provided the desired acid.

Method F: N-tert-Butoxycarbonyl Deprotection Method.

4N HCl in dioxane (ca. 40 equivalents) was added to a solution of the appropriate tert-butoxycarbonyl (Boc) protected amine (1 equivalent) in dioxane at 0° C. Cooling was removed and the reaction mixture was allowed to warm to rt over 20 h. The reaction mixture was concentrated to dryness in vacuo and the resulting off white solid (typical yield>90%) was used without further purification.

Method G: 3-O-Acetyl Group Removal Method.

Potassium hydroxide pellets (5 equivalents) were added to a suspension of the desired 3-O-acetylbetulinic acid amide derivative in methanol and water (7/1). The mixture was stirred at 50° C. overnight. The mixture was left cool to rt and diluted with water. The solid was collected by filtration, washed with water and dried at 60° C. under reduced pressure over night to yield the desired betulinic acid amide derivative.

Method H: Glutaric Side Chain Introduction Method.

The desired betulinic acid amide derivative and 4 equivalents of 3,3′-dimethylglutaric anhydride were suspended in neat DIPEA under nitrogen and stirred at 125° C. for 24 hours. All solvents were removed under pressure. The resulting solid was suspended in EtOAc and concentrated to dryness under reduced pressure in order to remove remaining traces of DIPEA. This solid was added to a 0.2 M solution of K₂CO₃ and stirred at 100° C. for 20 minutes. The solid was collected by filtration, washed with water and left to dry overnight at 60° C. to yield the desired material.

### 3-O-(5′-Allyloxy-3′,3′-dimethylglutaryl)betulinic acidN-2-(tert-butoxycarbonylamino)ethyl amide

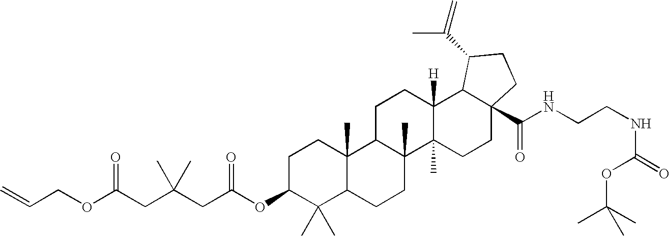

The compound was synthesized from 3-O-(5′-allyloxy-3′,3′-dimethylglutaryl)betulinic acid chloride applying method D with N-tert-butyl 2-aminoethylcarbamate; (0.36 g, 51%); ¹H NMR (400 MHz, CDCl₃) δ ppm 6.18-6.36 (1H, br m), 5.81-6.02 (1H, m), 5.16-5.42 (2H, m), 4.91-5.05 (1H, br m), 4.67-4.79 (1H, m), 4.51-4.65 (3H, m), 4.41-4.51 (1H, m), 3.04-3.42 (5H, m), 2.33-2.57 (5H, m), 1.87-2.03 (2H, m), 0.69-1.80 (53H, m).

### 3-O-(3′,3′-Dimethylglutaryl)betulinic acid N-(tert-butoxycarbonylamino) ethyl amide

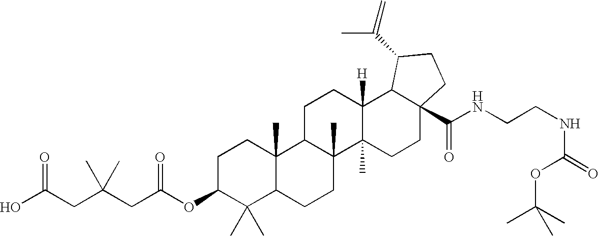

3-O-(5′-Allyloxy-3′,3′-dimethylglutaryl)betulinic acid N-(tert-butoxycarbonylamino)ethyl amide was deprotected using method C, (89 mg, 79%); ¹H NMR (400 MHz, CDCl₃) δ ppm 6.32 (1H, s), 5.02 (1H, s), 4.74 (1H, s), 4.59 (1H, s), 4.44-4.55 (1H, m), 3.23 (5H, s), 2.44 (5H, s), 0.69-2.11 (56H, m); LCMS, 97% pure; R_{f}=3.99; m/z (relative intensity) 741 (MH⁺, 40%).

### 3-O-(3′,3′-Dimethylglutaryl)betulinic acid N-2-aminoethyl amide HCl salt

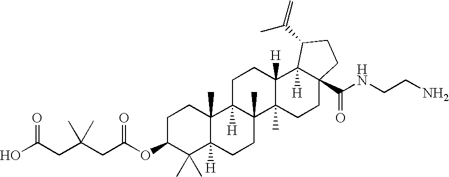

3-O-(3′,3′-Dimethylglutaryl)betulinic acid N-(tert-butoxycarbonylamino)ethyl amide was deprotected using method F. ¹H NMR (400 MHz, CD₃OD); δ ppm 4.61 (1H, s), 4.49 (1H, s), 4.48-4.40 (1H, m), 3.66-3.64 (1H, m), 3.58-3.55 (2H, m), 3.50-3.48 (1H, m), 3.34-3.32 (2H, m), 2.97-2.89 (3H, m), 2.44-2.29 (4H, m), 2.04-2.00 (1H, m), 1.79-0.61 (47H, m); LCMS, 96% R_{f}=3.20; m/z (relative intensity) 641 ([M+H⁺] 35%).

### 3-O-(3′,3′-Dimethylglutaryl)betulinic acid 1-[4-(tert-butoxycarbonyl) piperazinyl] amide

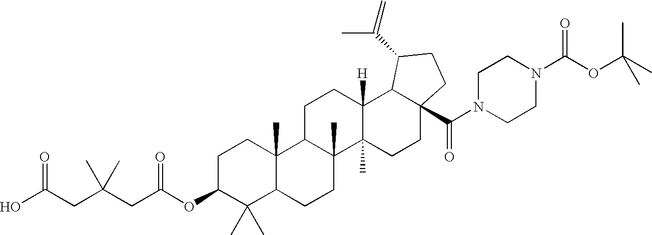

The compound was synthesized from 3-O-(5′-allyloxy-3′,3′-dimethylglutaryl)betulinic acid chloride applying method D with 1-(tert-butoxycarbonyl)piperazine (0.35 g, 60%), followed by method C deprotection: (17.2 mg, 46%); ¹H NMR (400 MHz, CDCl₃) δ ppm 4.73 (1H, d, J=1.9 Hz), 4.58 (1H, s), 4.50 (1H, m), 3.57 (4H, s), 3.39 (4H, s), 2.92-3.05 (1H, m), 2.79-2.92 (1H, m), 2.34-2.54 (4H, m), 0.70-2.13 (57H, m); LCMS, 96% pure; R_{f}=4.24; m/z (relative intensity) 789 ([M+Na]⁺, 30%).

### 3-O-(3′,3′-Dimethylglutaryl)betulinic acid 1-piperazine amide HCl salt

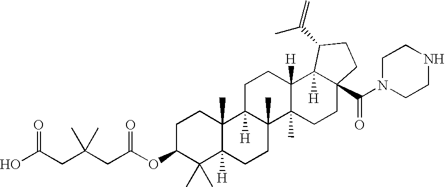

3-O-(3′,3′-Dimethylglutaryl)betulinic acid 1-[4-(tert-butoxycarbonyl) piperazinyl] amide was deprotected using method F: ¹H NMR (400 MHz, CD₃OD); δ ppm 4.72 (1H, s), 4.61 (1H, s), 4.50-4.46 (1H, m), 3.92-3.87 (4H, m), 3.22-3.20 (4H, m), 2.96-2.94 (1H, m), 2.92-2.85 (1H, m), 2.52-2.41 (3H, m), 2.14-2.02 (1H, m), 2.01-1.99 (1H, m), 1.82-0.77 (48H, m); LCMS, 100% pure, R_{f}=3.22; m/z (relative intensity) 667 ([M+H⁺] 26%).

### 3-O-(3′,3′-Dimethylglutaryl)betulinic acid N-4-[1-(tert-butoxycarbonyl)piperidinyl] amide

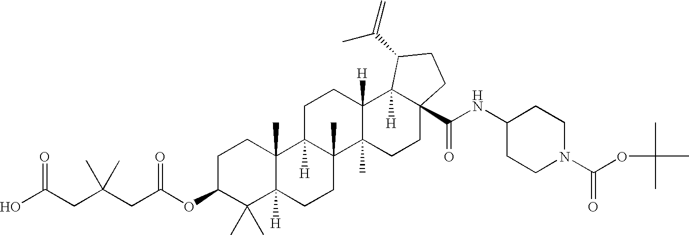

The compound was synthesized from 3-O-(5′-allyloxy-3′,3′-dimethylglutaryl)betulinic acid chloride and 4-amino-1-(tert-butoxycarbonyl) piperidine, applying method D, followed by method C deprotection. ¹H NMR (400 MHz, CD₃OD) δ ppm 0.63-1.99 (58H, m), 2.27-2.36 (2H, m), 2.36-2.44 (3H, m), 2.68-2.90 (2H, m), 3.05 (1H, m), 3.77-3.92 (1H, m), 3.97 (2H, br s), 4.35-4.46 (1H, m), 4.53 (1H, s), 4.66 (1H, s), 5.36 (1H, d, J=7.82 Hz).

### 3-O-(3′,3′-Dimethylglutaryl)betulinic acid N-4-piperidinyl amide HCl salt

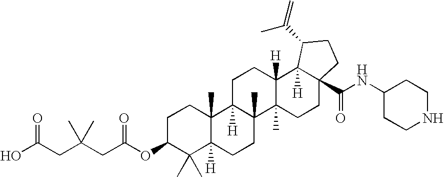

3-O-(3′,3′-Dimethylglutaryl)betulinic acid N-4-[1-(tert-butoxycarbonyl)piperidinyl] amide was deprotected using method F: ¹H NMR (400 MHz, CD₃OD) δ ppm 0.47-1.82 (48H, m), 1.86-2.17 (3H, m), 2.46-2.60 (1H, m), 2.86-3.10 (3H, m), 3.34 (2H, d, J=13.2 Hz), 3.43-3.52 (1H, m), 3.53-3.70 (5H, m), 3.78-3.89 (1H, m), 4.37 (1H, dd, J=10.0, 6.1 Hz), 4.50 (1H, s), 4.61 (1H, s).

### 3-O-(3′,3′-Dimethylglutaryl)betulinic acid 1-[(R)-3-(tert-butoxycarbonyl amino)pyrrolidinyl] amide

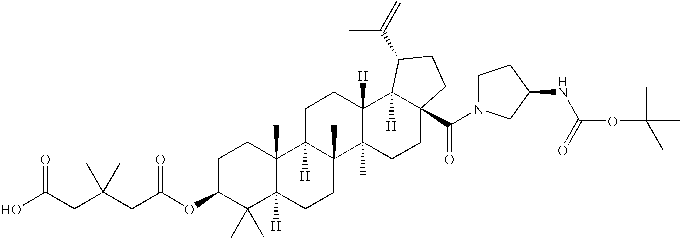

The compound was synthesized from 3-O-(5′-allyloxy-3′,3′-dimethylglutaryl)betulinic acid chloride and (R)-3-(tert-butoxycarbonyl amino)pyrrolidine, applying method D, followed by method C deprotection. ¹H NMR (400 MHz, CD₃OD) δ ppm 0.57-1.96 (56H, m), 2.12 (2H, d, J=11.7 Hz), 2.33 (1H, m), 2.36-2.46 (3H, m), 2.73 (1H, dt, J=10.8 Hz), 2.96 (1H, m), 3.24 (1H, s), 3.38-3.63 (2H, m), 3.78 (1H, br s), 4.04 (1H, br s), 4.42 (1H, m), 4.50 (1H, s), 4.65 (2H, d, J=1.8 Hz); LCMS, 100% pure; R_{f}=4.58; m/z (relative intensity) 668 ([M+H]⁺, 50%).

### 3-O-(3′,3′-Dimethylglutaryl)betulinic acid 1-[(R)-3-aminopyrrolidinyl] amide HCl salt

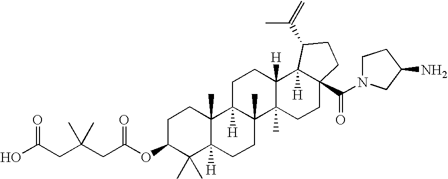

3-O-(3′,3′-Dimethylglutaryl)betulinic acid 1-[(R)-3-(tert-butoxycarbonylamino)pyrrolidinyl] amide was deprotected using method F. ¹HNMR MHz, CD₃OD) δ ppm 0.67-2.00 (48H, m), 2.15-2.33 (5H, m), 2.38 (1H, m), 2.70 (1H, m), 2.89 (1H, m), 3.16-3.23 (2H, m), 3.34-3.62 (2H, m), 3.68-3.87 (2H, m), 4.36 (1H, dd, J=10.1, 6.0 Hz), 4.49 (1H, s), 4.60 (1H, d, J=1.8 Hz); LCMS, 100% pure; R_{f}=3.20; m/z (relative intensity) 667 ([M+H]⁺, 20%).

### 3-O-(3′,3′-Dimethylglutaryl)betulinic acid 1-[(S)-3-(tert-butoxycarbonyl amino)pyrrolidinyl] amide

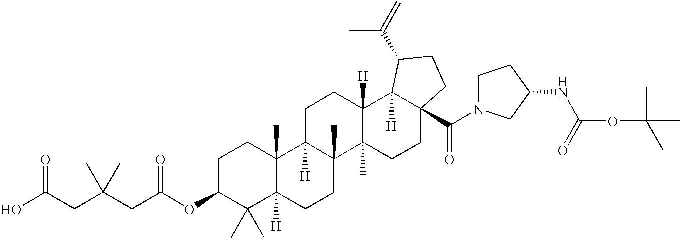

The compound was synthesized from 3-O-(5′-allyloxy-3′,3′-dimethylglutaryl)betulinic acid chloride and (S)-3-(tert-butoxycarbonyl amino)pyrrolidine, applying method D, followed by method C deprotection. ¹H NMR (400 MHz, CDCl₃) δ ppm 0.70-1.95 (54H, m), 1.97-2.12 (1H, m), 2.18 (1H, d, J=5.1 Hz), 2.34-2.43 (1H, m), 2.43-2.53 (3H, m), 2.68-2.88 (1H, m), 3.26-3.96 (4H, m), 4.14 (1H, br s), 4.50 (1H, m), 4.58 (2H, br s), 4.72 (1H, d, J=2.2 Hz); LCMS, 100% pure; R_{f}=5.00; m/z (relative intensity) 789 ([M+Na]⁺, 70%).

### 3-O-(3′,3′-Dimethylglutaryl)betulinic acid N-[(S)-3-aminopyrrolidinyl] amide HCl salt

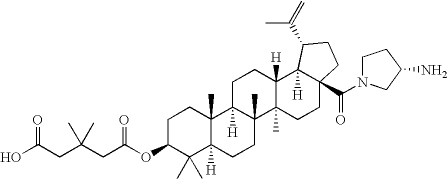

3-O-(3′,3′-Dimethylglutaryl)betulinic acid N-[(S)-3-(tert-butoxycarbonyl amino)pyrrolidinyl] amide was deprotected using method F. ¹H NMR (400 MHz, CD₃OD); δ ppm 4.52 (1H, s), 4.41 (1H, s), 4.30-4.26 (1H, m), 3.75 (2H, br s), 3.54-3.52 (2H, m), 2.84-2.80 (1H, m), 2.65-2.59 (1H, m), 2.32-2.13 (6H, m), 1.90-1.82 (2H, m), 1.56-0.61 (48H, m); LCMS, 100% pure, R_{f}=3.21; m/z (relative intensity) 667 ([M+H⁺] 15%).

### 3-O-(3′,3′-Dimethylglutaryl)betulinic acid N-[(S)-3-(tert-butoxycarbonyl) pyrrolidinyl] amide

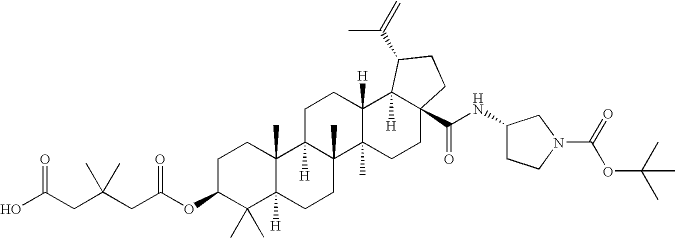

The compound was synthesized from 3-O-(5′-allyloxy-3′,3′-dimethylglutaryl)betulinic acid chloride and (S)-3-amino-1-(tert-butoxycarbonyl)pyrrolidine, applying method D, followed by method C deprotection. ¹H NMR (400 MHz, CDCl₃) δ ppm 0.68-2.02 (57H, m), 2.07-2.23 (1H, m), 2.36-2.44 (2H, m), 2.45-2.51 (2H, m), 3.02-3.52 (4H, m), 3.60 (1H, dd, J=11.7, 6.4 Hz), 4.34-4.46 (1H, m), 4.52 (1H, dd, J=10.3, 5.9 Hz), 4.60 (1H, s), 4.74 (1H, s), 5.60 (1H, d, J=6.8 Hz); LCMS, 97% pure, R_{f}=5.01; m/z (relative intensity) 789 ([M+Na]⁺, 80%).

### 3-O-(3′,3′-Dimethylglutaryl)betulinic acid N-[(S)-3-pyrrolidinyl] amide HCl salt

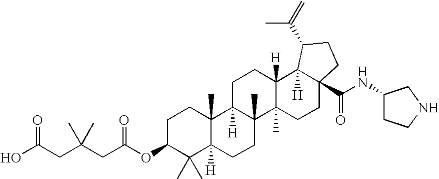

3-O-(3′,3′-Dimethylglutaryl)betulinic acid N-[(S)-3-(tert-butoxycarbonyl)pyrrolidinyl] amide was deprotected using method F. ¹H NMR (400 MHz, CD₃OD) δ ppm 4.48 (1H, d, J=2.0 Hz), 4.38 (1H, dd, J=2.4, 1.5 Hz), 4.24 (1H, dd, J=10.0, 6.1 Hz), 4.11-4.20 (1H, m), 3.24-3.34 (2H, m), 2.95 (1H, dd, J=12.2, 4.9 Hz), 2.85 (1H, td, J=10.9, 4.6 Hz), 2.32-2.42 (1H, m), 2.08-2.30 (6H, m), 1.91-1.98 (1H, m), 1.74-1.85 (1H, m), 0.56-1.70 (50H, m); LCMS, 96% pure; R_{f}=3.22; m/z (relative intensity) 668 ([M+H]⁺, 40%).

### 3-O-(3′,3′-Dimethylglutaryl)betulinic N-[(R)-3-(tert-butoxycarbonyl) pyrrolidinyl] amide

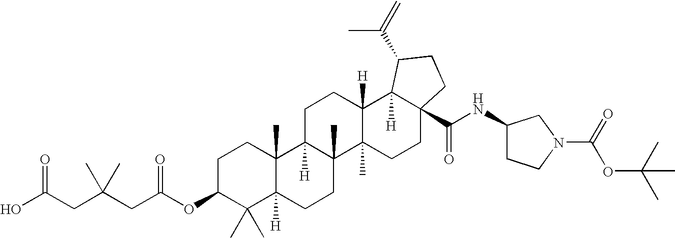

The compound was synthesized from 3-O-(5′-allyloxy-3′,3′-dimethylglutaryl)betulinic acid chloride and (R)-3-amino-1-(tert-butoxycarbonyl)pyrrolidine, applying method D, followed by method C deprotection. ¹H NMR (400 MHz, CDCl₃) δ ppm 5.59 (1H, d, J=4.4 Hz), 4.74 (1H, s), 4.60 (1H, s), 4.46-4.55 (1H, m), 4.37-4.46 (1H, m), 3.60 (1H, dd, J=11.7, 6.4 Hz), 3.45 (2H, br s), 3.12 (2H, br s), 2.37-2.51 (4H, m), 0.70-2.24 (59H, m); LCMS, 98% pure; R_{f}=4.59; m/z (relative intensity) 768 ([M+H]⁺, 20%).

### 3-O-(3′,3′-Dimethylglutaryl)betulinic acid N-[(R)-3-aminopyrrolidinyl] amide HCl salt

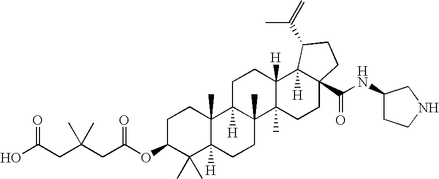

3-O-(3′,3′-Dimethylglutaryl)betulinic acid N-[(R)-3-(1-tert-butoxycarbonyl)pyrrolidinyl] amide was deprotected using method F. ¹H NMR (400 MHz, CD₃OD) δ ppm 0.67-1.81 (44H, m), 1.95 (1H, s), 2.06 (1H, d, J=13.5 Hz), 2.18-2.33 (5H, m), 2.38 (1H, m), 2.48 (1H, m), 2.97 (1H, dt, J=4.0 Hz), 3.06 (1H, d, J=8.0 Hz), 3.21 (4H, s), 3.33-3.50 (2H, m), 4.24 (1H, m), 4.36 (1H, dd, J=9.9, 6.2 Hz), 4.49 (1H, s), 4.60 (1H, s): LCMS, 100% pure; R_{f}=3.24; m/z (relative intensity) 667 ([M+H]⁺, 20%).

### 3-O-(3′,3′-Dimethylglutaryl)betulinic acid N-2-(acetamido)ethyl amide

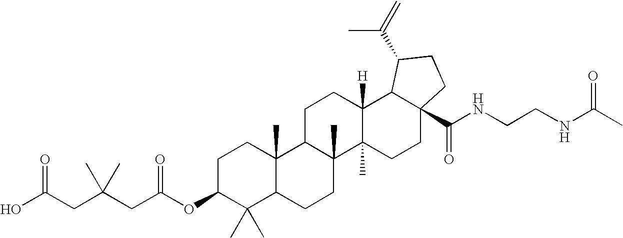

The compound was synthesized from 3-O-(5′-allyloxy-3′,3′-dimethylglutaryl)betulinic acid chloride applying method D with N-(2-aminoethyl)acetamide (0.19 g, 78%); followed by method C deprotection; (0.13 g, 84%); ¹H NMR (400 MHz, CDCl₃) δ ppm 6.66-6.84 (1H, m), 6.33-6.45 (1H, m), 4.73 (1H, d, J=2.0 Hz), 4.60 (1H, d), 4.49 (1H, m), 3.27-3.54 (4H, m), 2.99-3.18 (1H, m), 2.29-2.56 (5H, m), 0.60-2.09 (50H, m); LCMS, 94% pure; R_{f}=1.80 (2.5 min); m/z (relative intensity) 683 (MH⁺, 100%).

### 3-O-(3′,3′-Dimethylglutaryl)betulinic acid 2-methoxyethyl amide

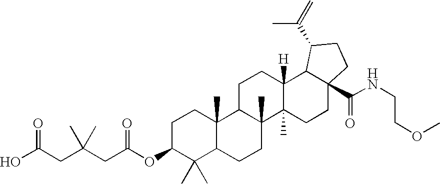

The compound was synthesized from 3-O-(5′-allyloxy-3′,3′-dimethylglutaryl)betulinic acid chloride applying method D with 2-methoxyethylamine (55 mg, 34%), followed by method C deprotection: (9.1 mg, 88%); ¹H NMR (400 MHz, CDCl₃) δ ppm 5.90-6.08 (1H, m), 4.69-4.79 (1H, m), 4.55-4.65 (1H, m), 4.45-4.55 (1H, m), 3.29-3.56 (7H, m), 3.05-3.18 (1H, m), 2.34-2.51 (5H, m), 1.89-2.02 (2H, m), 0.72-1.79 (45H, m); LCMS, 96% pure; R_{f}=3.86; m/z (relative intensity) 678 ([M+Na]⁺, 50%).

### 3-O-(3′,3′-Dimethylglutaryl)betulinic acid 4-morpholinyl amide

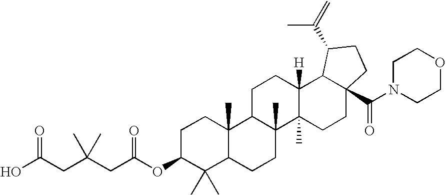

The compound was synthesized from 3-O-acetylbetulinic acid chloride applying method D with morpholine, followed by method G deprotection and method H side chain introduction. ¹H NMR (400 MHz, CDCl₃) δ ppm 4.69-4.76 (1H, m), 4.56-4.61 (1H, m), 4.44-4.55 (1H, m), 3.55-3.72 (8H, m), 2.93-3.04 (1H, m), 2.81-2.92 (1H, m), 2.35-2.52 (4H, m), 0.70-2.13 (47H, m); LCMS, 96% pure; R_{f}=3.97; m/z (relative intensity) 668 (MH⁺, 90%).

### 3-O-(3′,3′-Dimethylglutaryl)betulinic acid 1-piperidinyl amide

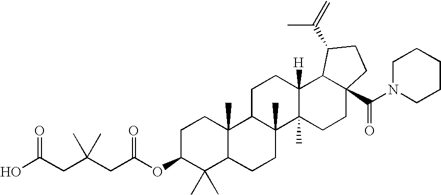

The compound was synthesized from 3-O-(5′-allyloxy-3′,3′-dimethylglutaryl)betulinic acid chloride applying method D with piperidine (49%), followed by method C deprotection: (80 mg, 90%); ¹H NMR (400 MHz, CDCl₃) δ ppm 4.68-4.74 (1H, m), 4.54-4.59 (1H, m), 4.45-4.55 (1H, m), 3.36-3.65 (4H, m), 2.96-3.07 (1H, m), 2.84-2.95 (1H, m), 2.35-2.50 (4H, m), 2.08-2.17 (1H, m), 1.93-2.03 (1H, m), 1.79-1.90 (1H, m), 0.73-1.75 (50H, m); LCMS, 97% pure; R_{f}=4.36; m/z (relative intensity) 666 (MH⁺, 100%).

### 3-O-(3′,3′-Dimethylglutaryl)betulinic amide

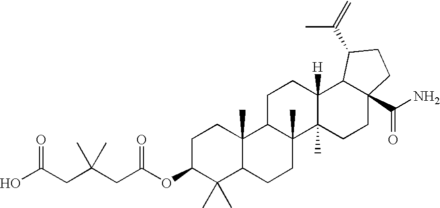

The compound was synthesized from 3-O-(5′-allyloxy-3′,3′-dimethylglutaryl)betulinic acid chloride applying method D with ammonia (62%), followed by method C deprotection; (6.6 mg, 22%); ¹H NMR (400 MHz, CDCl₃) δ ppm 6.69 (1H, s), 5.56 (1H, s), 4.71 (H, d, J=2.2 Hz), 4.58 (1H, s), 4.44-4.53 (1H, m), 3.00-3.10 (1H, m), 2.75 (1H, d, J=12.8 Hz), 2.31-2.52 (3H, m), 2.21 (1H, d, J=13.2 Hz), 1.75-2.04 (4H, m), 0.72-1.75 (43H, m); LCMS, 100% pure; R_{f}=3.77; m/z (relative intensity) 620 ([M+Na]⁺, 100%).

### 3-O-(3′,3′-Dimethylglutaryl)betulinic acid N-ethyl amide

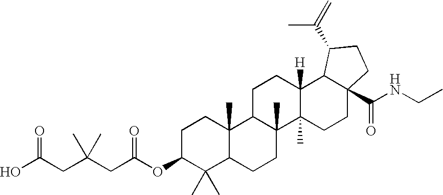

The compound was synthesized applying from 3-O-(5′-allyloxy-3′,3′-dimethylglutaryl)betulinic acid chloride method D with ethylamine (77%), followed by method C deprotection: (80 mg, 90%); ¹H NMR (400 MHz, CDCl₃) δ ppm 5.54 (1H, t, J=5.5 Hz), 4.72 (1H, d, J=2.2 Hz), 4.58 (1H, s), 4.49 (1H, dd, J=10.2, 5.9 Hz), 2.97-3.42 (3H, m), 2.32-2.54 (5H, m), 1.83-2.04 (2H, m), 0.67-1.76 (48H, m); LCMS, 96% pure; R_{f}=3.97; m/z (relative intensity) 626 ([M+H]⁺, 100%).

### 3-O-(3′,3′-Dimethylglutaryl)betulinic acid N-propyl amide

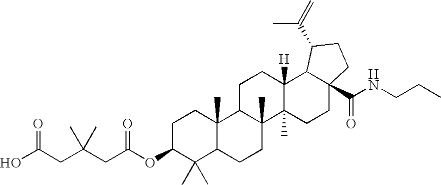

The compound was synthesized from 3-O-(5′-allyloxy-3′,3′-dimethylglutaryl)betulinic acid chloride applying method D with propylamine (52%), followed by method C deprotection: (24 mg, 25%); ¹H NMR (400 MHz, CDCl₃) δ ppm 5.63 (1H, t, J=5.9 Hz), 4.73 (1H, d, J=2.2 Hz), 4.59 (1H, s), 4.42-4.54 (1H, m), 3.20-3.34 (1H, m), 3.05-3.20 (2H, dd, J=12.3, 6.4 Hz), 2.37-2.54 (5H, m), 0.67-2.24 (52H, m); LCMS, 96% pure; R_{f}=4.06; m/z (relative intensity) 640 ([M+H]⁺, 100%).

### 3-O-(3′,3′-Dimethylglutaryl)betulinic acid N-methyl-N-propyl amide

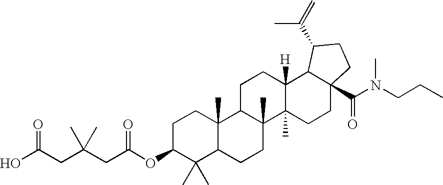

The compound was synthesized from 3-O-(5′-allyloxy-3′,3′-dimethylglutaryl)betulinic acid chloride applying method D with N-methylpropylamine (19%), followed by method C deprotection: (17 mg, 49%); ¹H NMR (400 MHz, CDCl₃) δ ppm 4.72 (1H, d, J=1.8 Hz), 4.57 (1H, s), 4.50 (1H, dd, J=10.2, 5.9 Hz), 2.78-3.13 (5H, m), 2.32-2.54 (4H, m), 0.64-2.29 (54H, m); LCMS, 97% pure; R_{f}=4.33; m/z (relative intensity) 653 ([M+H]⁺, 100%).

### 3-O-(3′,3′-Dimethylglutaryl)betulinic acid N-isopropyl amide

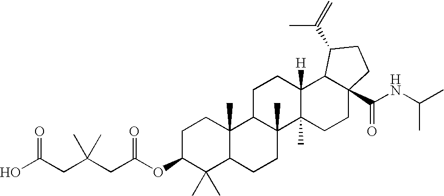

The compound was synthesized from 3-O-(5′-allyloxy-3′,3′-dimethylglutaryl)betulinic acid chloride applying method D with isopropylamine (38%), followed by method C deprotection: (45 mg, 38%); ¹H NMR (400 MHz, CD₃OD) δ ppm 4.59 (1H, d, J=2.4 Hz), 4.47 (1H, s), 4.35 (1H, dd, J=10.3, 5.9 Hz), 3.82-3.95 (1H, m), 2.94-3.05 (1H, m), 2.45-2.56 (1H, m), 2.38 (1H, d, J=14.2 Hz), 2.25-2.33 (3H, m), 1.99-2.09 (1H, m), 0.67-1.85 (53H, m); LCMS, 96% pure; R_{f}=4.08; m/z (relative intensity) 640 ([M+H]⁺, 100%).

### 3-O-(3′,3′-Dimethylglutaryl)betulinic acid N-cyclopropyl amide

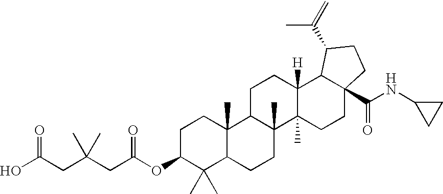

The compound was synthesized from 3-O-acetylbetulinic acid chloride applying method D with cyclopropylamine, followed by method G deprotection and method H side chain introduction. ¹H NMR (400 MHz, CD₃OD) δ ppm 4.69 (1H, d, J=2.4 Hz), 4.57 (1H, s), 4.45 (1H, dd, J=10.3, 5.9 Hz), 3.01-3.19 (1H, m), 2.32-2.68 (6H, m), 1.98-2.12 (1H, m), 0.61-1.94 (49H, m), 0.33-0.50 (2H, m); LCMS, 99% pure; R_{f}=3.97; m/z (relative intensity) 660 ([M+Na]⁺, 60%).

### 3-O-(3′,3′-Dimethylglutaryl)betulinic acid N-(2-(4-morpholinyl)ethyl) amide

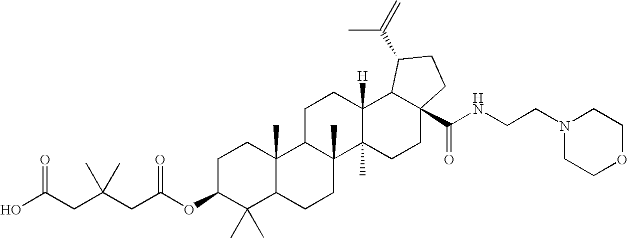

The compound was synthesized from 3-O-(5′-allyloxy-3′,3′-dimethylglutaryl)betulinic acid chloride applying method D with 2-(4-morpholinyl)ethylamine (40%) followed by method C deprotection: (62 mg, 48%); ¹H NMR (400 MHz, CD₃OD) δ ppm 4.60 (1H, s), 4.49 (1H, s), 4.36 (1H, dd, J=10.5, 5.6 Hz), 3.50-3.67 (7H, m), 3.32-3.51 (2H, m), 3.21-3.32 (1H, m), 2.90-3.07 (1H, m), 2.30-2.50 (8H, m), 1.99-2.09 (1H, m), 0.61-1.87 (47H, m); LCMS, 100% pure; R_{f}=3.23; m/z (relative intensity) 711 ([M+H]⁺, 40%).

### 3-O-(3′,3′-Dimethylglutaryl)betulinic acid N-(4-fluorophenyl) amide

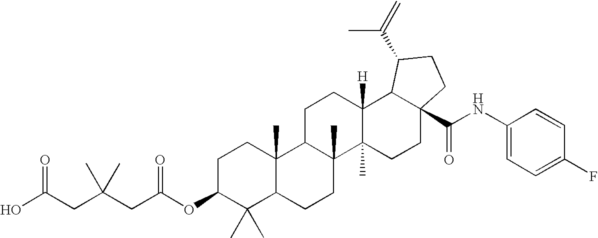

The compound was synthesized from 3-O-(5′-allyloxy-3′,3′-dimethylglutaryl)betulinic acid chloride applying method D with 4-fluoroaniline (39%), followed by method C deprotection: (40 mg, 32%); ¹H NMR (400 MHz, Acetone-d₆) δ ppm 8.74 (1H, s), 7.49-7.61 (2H, m), 6.93 (2H, t, J=8.8 Hz), 4.26-4.66 (4H, m), 2.98-3.10 (1H, m), 2.62-2.75 (2H, m), 2.17-2.41 (6H, m), 0.73-1.99 (43H, m); LCMS, 100% pure; R_{f}=4.13; m/z (relative intensity) 692 ([M+H]⁺, 100%).

### 3-O-(3′,3′-Dimethylglutaryl)betulinic acid N-(4-fluorobenzyl) amide

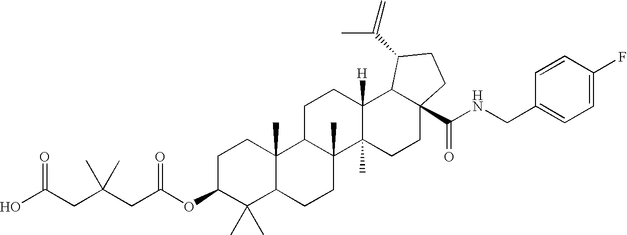

The compound was synthesized from 3-O-acetylbetulinic acid chloride applying method D with 4-fluorobenzylamine, followed by method G deprotection and method H side chain introduction. ¹H NMR (400 MHz, CD₃OD) δ ppm 8.05 (1H, t, J=6.1 Hz), 7.21 (2H, dd, J=8.3, 5.4 Hz), 6.86-6.98 (2H, m), 4.60 (1H, d, J=2.0 Hz), 4.48 (1H, s), 4.23-4.41 (2H, m), 4.12 (1H, dd, J=14.7, 5.9 Hz), 2.93-3.06 (1H, m), 2.41-2.52 (1H, m), 2.30-2.41 (2H, m), 1.99-2.09 (1H, m), 0.61-1.84 (53H, m); ¹⁹F NMR (376 MHz, Acetone-d₆) δ ppm −118.2 (s); LCMS, 100% pure; R_{f}=4.10; m/z (relative intensity) 706 ([M+H]⁺, 100%).

### 3-O-(3′,3′-Dimethylglutaryl)betulinic acid N-[(S)-(1-carboxy-3-methyl)butyl] amide

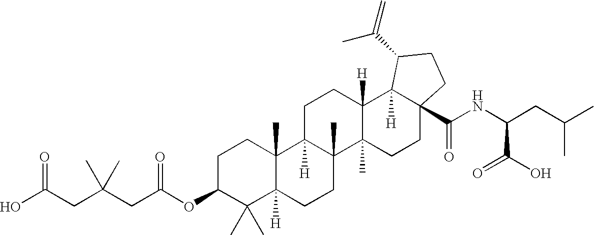

The compound was synthesized from 3-O-(5′-allyloxy-3′,3′-dimethylglutaryl)betulinic acid chloride applying method D with (S)-N-(tert-butoxycarbonyl)leucine, followed by method F Boc group deprotection and method C deallylation. ¹H NMR (400 MHz, CDCl₃) δ ppm 0.72-2.03 (57H, m), 2.36-2.51 (5H, m), 3.10 (1H, td, J=10.9, 4.6 Hz), 4.50 (1H, dd, J=10.4, 5.7 Hz), 4.59 (2H, s), 4.73 (1H, d, J=1.8 Hz), 5.89 (1H, d, J=7.7 Hz).

### 3-O-(3′,3′-Dimethylglutaryl)betulinic acid N-[(S)-(1-hydroxymethyl-3-methyl)butyl amide

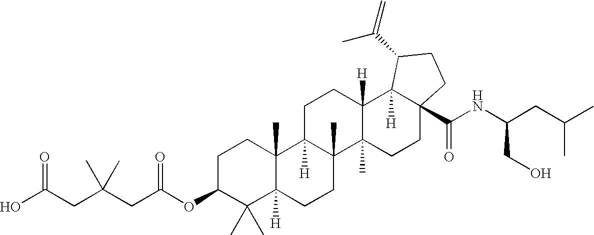

The compound was synthesized from 3-O-(5′-allyloxy-3′,3′-dimethylglutaryl)betulinic acid chloride applying method D with (S)-leucinol, followed by method C deprotection. ¹H NMR (400 MHz, CDCl₃), δ ppm 0.67-1.71 (55H, m), 1.75 (1H, dd, J=12.1, 7.7 Hz), 1.85-2.01 (2H, m), 2.31-2.41 (1H, m), 2.41-2.51 (4H, m), 3.08 (1H, dt, J=11.0, 4.0 Hz), 3.49 (1H, dd, J=11.0, 6.2 Hz), 3.64 (1H, dd, J=10.8, 3.5 Hz), 4.07 (1H, br s), 4.57 (1H, s), 4.71 (1H, d, J=1.8 Hz), 5.68 (1H, d, J=8.0 Hz).

### 3-O-(3′,3′-Dimethylglutaryl)betulinic acid N-2-hydroxyethyl amide

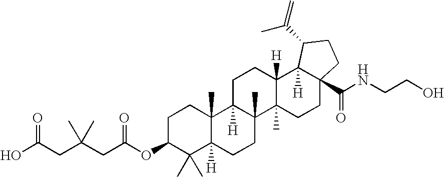

The compound was synthesized from 3-O-(5′-allyloxy-3′,3′-dimethylglutaryl)betulinic acid chloride applying method D with 2-hydroxyethylamine, followed by method C deprotection. ¹H NMR (400 MHz, CDCl₃) δ ppm 6.00 (1H, m), 4.67 (1H, s), 4.53 (1H, s), 4.46-4.42 (1H, m), 3.70-3.65 (2H, m), 3.50-3.25 (5H, m), 3.09 (1H, m), 2.48-2.25 (6H, m), 2.1-0.70 (45H, m).

### 3-O-(3′,3′-Dimethylglutaryl)betulinic acid N-(R/S)-2,3-dihydroxypropyl amide

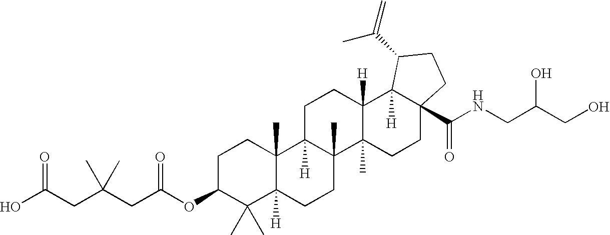

The compound was synthesized from 3-O-(5′-allyloxy-3′,3′-dimethylglutaryl)betulinic acid chloride applying method D with (R/S)-2,3-dihydroxypropylamine, followed by method C deprotection. ¹H NMR (400 MHz, CDCl₃) δ ppm 0.71-2.22 (44H, m), 2.34-2.42 (1H, m), 2.44-2.52 (3H, m), 2.97 (1H, d, J=11.2 Hz), 3.08 (1H, dt), 3.27-4.01 (10H, m), 4.50 (1H, dd, J=10.5, 5.6 Hz), 4.61 (1H, s), 4.74 (1H, s), 6.10 (1H, d, J=2.4 Hz).

### 3-O-(3′,3′-Dimethylglutaryl)betulinic acid N-methoxy-N-methyl amide

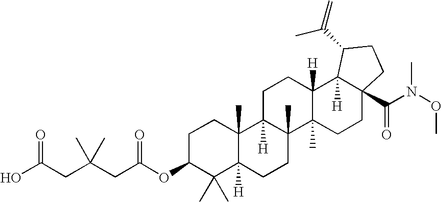

The compound was synthesized from 3-O-(5′-allyloxy-3′,3′-dimethylglutaryl)betulinic acid chloride applying method D with N,O-dimethylhydroxylamine, followed by method C deprotection. ¹H NMR (400 MHz, CDCl₃) δ ppm 0.72-1.04 (18H, m), 1.07-1.89 (29H, m), 2.02-2.17 (1H, m), 2.24-2.54 (5H, m), 2.98 (1H, dt, J=11.2, 3.7 Hz), 3.16 (3H, s), 3.66 (3H, s), 4.50 (1H, dd), 4.58 (1H, s), 4.72 (1H, d, J=2.2 Hz).

### 3-O-(3′,3′-Dimethylglutaryl)betulinic acid 4-(1,4-oxazepinyl) amide

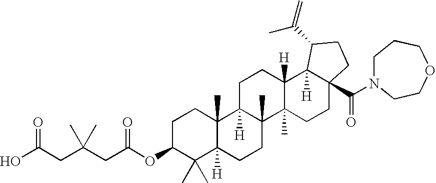

The compound was synthesized from 3-O-(5′-allyloxy-3′,3′-dimethylglutaryl)betulinic acid chloride applying method D with 1,4-oxazepine, followed by method C deprotection. ¹H NMR (400 MHz, CDCl₃) δ ppm 0.63-1.70 (45H, m), 1.69-1.96 (4H, m), 1.99-2.13 (1H, m), 2.32 (1H, d), 2.36-2.45 (3H, m), 2.78-2.99 (2H, m), 3.39-3.87 (8H, m), 4.37-4.47 (1H, m), 4.51 (1H, s), 4.66 (1H, d, J=2.2 Hz).

### 3-O-(3′,3′-Dimethylglutaryl)betulinic acid N-(2-methoxyethyl)-N-methyl amide

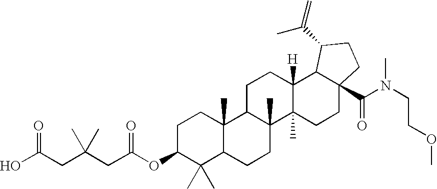

The compound was synthesized from 3-O-(5′-allyloxy-3′,3′-dimethylglutaryl)betulinic acid chloride applying method D with N-methyl-2-methoxyethylamine, followed by method C deprotection. ¹H NMR (400 MHz, CDCl₃) δ ppm 0.67-1.76 (47H, m), 1.77-1.91 (1H, m), 2.05 (1H, dd, J=10.8, 7.5 Hz), 2.26 (1H, d, J=13.5 Hz), 2.40 (1H, d), 2.45-2.51 (3H, m), 2.89 (1H, dt), 3.00 (1H, dt, J=11.2, 3.7 Hz), 3.12 (2H, br s), 3.32-3.37 (3H, m), 3.54 (2H, t, J=5.3 Hz), 3.60-3.73 (1H, m), 4.50 (1H, dd, J=10.4, 5.7 Hz), 4.57 (1H, s), 4.72 (1H, d, J=2.2 Hz).

### 3-O-(3′,3′-Dimethylglutaryl)betulinic acid N,N-bis(2-methoxyethyl) amide

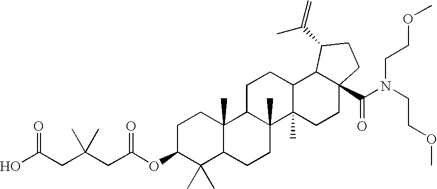

The compound was synthesized from 3-O-(5′-allyloxy-3′,3′-dimethylglutaryl)betulinic acid chloride applying method D with bis(2-methoxyethyl)amine, followed by method C deprotection. ¹H NMR (400 MHz, CDCl₃) δ ppm 0.71-1.75 (45H, m), 1.76-1.89 (1H, m), 2.04 (1H, dd, J=10.8, 7.5 Hz), 2.17 (1H, d, J=13.5 Hz), 2.40 (1H, d), 2.43-2.51 (3H, m), 2.86 (1H, dt), 2.99 (1H, dt, J=11.0, 3.3 Hz), 3.25-3.43 (7H, m, J=4.4 Hz), 3.43-3.67 (6H, m), 3.76 (1H, br s), 4.50 (1H, dd), 4.57 (1H, s), 4.72 (1H, d, J=2.2 Hz).

### 3-O-(3′,3′-Dimethylglutaryl)betulinic acid N-methyl amide

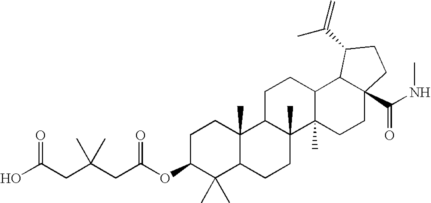

The compound was synthesized from 3-O-(5′-allyloxy-3′,3′-dimethylglutaryl)betulinic acid chloride applying method D with methylamine, followed by method C deprotection. ¹H NMR (400 MHz, CDCl₃) δ ppm 0.70-2.16 (47H, m), 2.33-2.42 (1H, m), 2.42-2.56 (4H, m), 2.80 (3H, d, J=4.8 Hz), 2.96 (1H, d), 3.14 (1H, dt, J=11.4, 3.8 Hz), 4.49 (1H, dd), 4.59 (1H, s), 4.74 (1H, d, J=1.8 Hz), 5.57 (1H, q, J=4.5 Hz).

### 3-O-(3′,3′-Dimethylglutaryl)betulinic acid N,N-dimethyl amide

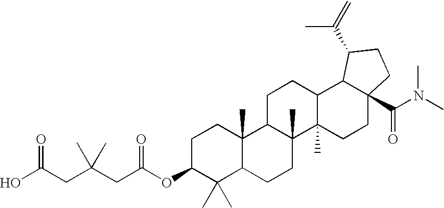

The compound was synthesized from 3-O-(5′-allyloxy-3′,3′-dimethylglutaryl)betulinic acid chloride applying method D with dimethylamine, followed by method C deprotection. ¹H NMR (400 MHz, CDCl₃) δ ppm 0.72-1.76 (45H, m), 1.78-1.93 (1H, m), 1.98-2.08 (1H, m), 2.24 (1H, d), 2.42-2.53 (3H, m), 2.88 (1H, dt), 2.88 (1H, dt), 2.94-3.10 (6H, m), 4.50 (1H, dd, J=10.4, 5.7 Hz), 4.57 (1H, s), 4.72 (1H, d, J=2.2 Hz).

### 3-O-(3′,3′-Dimethylglutaryl)betulinic acid N-2-(tert-butoxycarbonyl)hydrazide

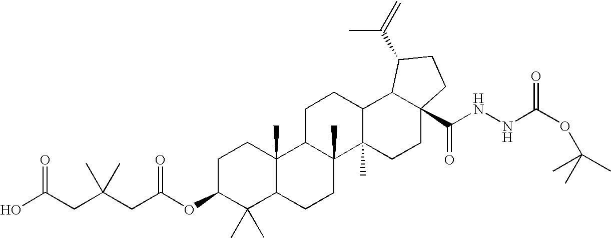

The compound was synthesized from 3-O-(5′-allyloxy-3′,3′-dimethylglutaryl)betulinic acid chloride applying method D with tert-butylcarbazate, followed by method C deprotection. ¹H NMR (400 MHz, CDCl₃) δ ppm 0.71-1.76 (53H, m), 1.83-2.07 (3H, m), 2.39 (1H, d), 2.43-2.52 (4H, m), 3.08 (1H, dt), 4.50 (1H, dd), 4.59 (1H, s), 4.73 (1H, d, J=2.2 Hz), 6.52 (1H, s), 7.40 (1H, s).

### 3-O-(3′,3′-Dimethylglutaryl)betulinic acid N-(tert-butoxycarbonylmethyl) amide

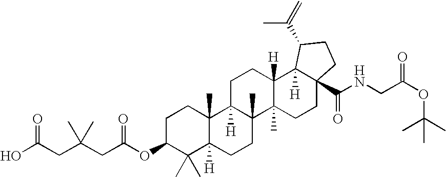

The compound was synthesized from −3-O-(5′-allyloxy-3′,3′-dimethylglutaryl)betulinic acid chloride applying method D with N-(tert-butoxycarbonyl)glycine, followed by method C deprotection. ¹H NMR (400 MHz, CDCl₃) δ ppm 6.12 (1H, t, J=5.1 Hz), 4.72 (1H, d, J=2.2 Hz), 4.58 (1H, s), 4.43-4.51 (1H, m), 3.89 (2H, dd, J=5.1, 2.9 Hz), 3.09 (1H, td, J=11.0, 4.4 Hz), 2.35-2.50 (5H, m), 1.87-2.05 (2H, m), 1.82 (1H, dd, J=11.7, 7.7 Hz), 0.70-1.75 (54H, m)

### 3-O-(3′,3′-Dimethylglutaryl)betulinic acid N-(1-hydroxy-2-methyl-2-propyl) amide

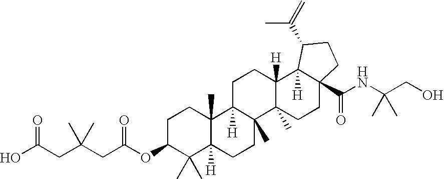

The compound was synthesized from 3-O-(5′-allyloxy-3′,3′-dimethylglutaryl)betulinic acid chloride applying method D with 1-hydroxy-2-methyl-2-propylamine, followed by method C deprotection. ¹H NMR (400 MHz, CDCl₃) δ ppm 0.64-1.76 (51H, m), 1.79-1.89 (1H, m), 1.90-2.01 (1H, m), 2.39 (1H, d), 2.42-2.50 (4H, m), 3.05 (1H, dt, J=11.1, 3.8 Hz), 3.57 (2H, s), 4.49 (1H, dd), 4.59 (1H, s), 4.72 (1H, d, J=2.2 Hz), 5.59 (1H, s).

### 3-O-(3′,3′-Dimethylglutaryl)betulinic acid N-4-hydroxycyclohexyl amide

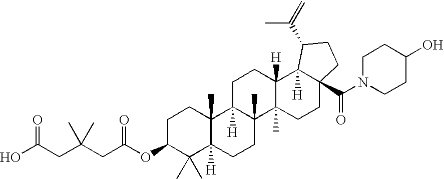

The compound was synthesized from 3-O-(5′-allyloxy-3′,3′-dimethylglutaryl)betulinic acid chloride applying method D with 4-hydroxycyclohexylamine, followed by method C deprotection. ¹H NMR (400 MHz, CDCl₃) δ ppm 0.65-1.95 (51H, m), 2.05 (1H, d), 2.33 (1H, d), 2.36-2.48 (3H, m), 2.81 (1H, dt), 2.92 (1H, dt, J=11.2, 3.4 Hz), 2.98-3.16 (2H, m), 3.80-3.90 (1H, m), 3.94 (1H, d, J=13.2 Hz), 3.99-4.16 (1H, m), 4.44 (1H, dd, J=10.3, 5.9 Hz), 4.51 (1H, s), 4.65 (1H, d, J=2.0 Hz).

### 3-O-(3′,3′-Dimethylglutaryl)betulinic acid 1-[(R)-2-(hydroxymethyl)pyrrolidinyl] amide

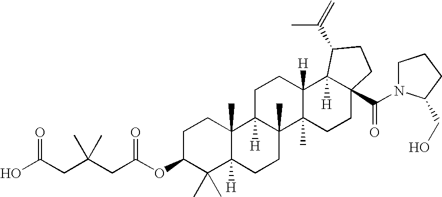

The compound was synthesized from 3-O-(5′-allyloxy-3′,3′-dimethylglutaryl)betulinic acid chloride applying method D with (R)-prolinol, followed by method C deprotection. ¹H NMR (400 MHz, CDCl₃) δ ppm 0.70-1.95 (50H, m), 2.05-2.16 (1H, m), 2.24-2.34 (1H, m), 2.40 (1H, d), 2.43-2.53 (3H, m), 2.76 (1H, dt), 2.96-3.07 (1H, m), 3.26 (1H, dt, J=11.0, 5.49 Hz), 3.56 (1H, dd, J=11.5, 7.9 Hz), 3.71 (1H, dd), 3.89 (1H, dd), 4.36 (1H, dd), 4.44-4.54 (1H, m), 4.59 (1H, s), 4.74 (1H, d, J=2.2 Hz).

### 3-O-(3′,3′-Dimethylglutaryl)betulinic acid 1-[(S)-2-(hydroxymethyl)pyrrolidinyl] amide

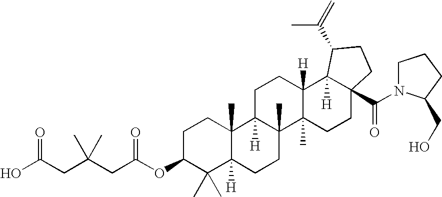

The compound was synthesized from 3-O-(5′-allyloxy-3′,3′-dimethylglutaryl)betulinic acid chloride applying method D with (S)-prolinol, followed by method C deprotection. ¹H NMR (400 MHz, CDCl₃) δ ppm 0.64-1.70 (46H, m), 1.73-1.86 (2H, m), 1.91-2.15 (4H, m), 2.26-2.47 (4H, m), 2.75-2.96 (2H, m), 3.16-3.32 (1H, m), 3.39-3.61 (2H, m), 3.69-3.83 (1H, m), 4.19-4.32 (1H, m), 4.44 (1H, dd, J=10.3, 5.9 Hz), 4.52 (1H, s), 4.65 (1H, d, J=2.4 Hz).

### 3-O-(3′,3′-Dimethylglutaryl)betulinic acid 1-[(R)-3-hydroxypyrrolidinyl] amide

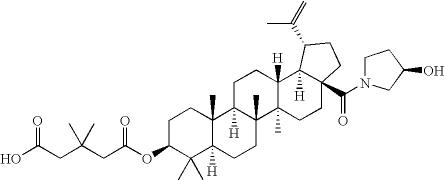

The compound was synthesized from 3-O-(5′-allyloxy-3′,3′-dimethylglutaryl)betulinic acid chloride applying method D with (R)-(+)-3-pyrrolidinol, followed by method C deprotection. ¹H NMR (400 MHz, CDCl₃) δ ppm 0.67-2.10 (49H, m), 2.20 (1H, d, J=12.1 Hz), 2.36 (1H, d), 2.41-2.51 (3H, m), 2.81-2.93 (1H, m), 2.97 (1H, dt), 3.50 (1H, dd, J=12.1, 3.7 Hz), 3.55-3.73 (3H, m), 4.37-4.44 (1H, m), 4.47 (1H, dd), 4.56 (1H, s), 4.70 (1H, d, J=2.2 Hz); LCMS, 100% pure; R_{f}=4.20; m/z (relative intensity) 668 ([M+H]⁺, 100%).

### 3-O-(31,3′-Dimethylglutaryl)betulinic acid 1-[(S)-3-hydroxypyrrolidinyl] amide

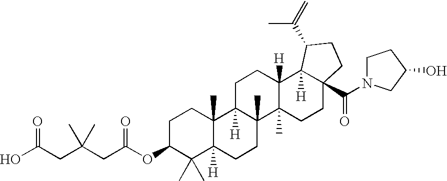

The compound was synthesized from 3-O-(5′-allyloxy-3′,3′-dimethylglutaryl)betulinic acid chloride applying method D with (S)-(−)-3-pyrrolidinol, followed by method C deprotection. ¹H NMR (250 MHz, CDCl₃) δ ppm 4.72 (1H, d, J=1.5 Hz), 4.58 (1H, s), 4.40-4.55 (2H, m), 3.38-3.81 (5H, m), 2.84-3.17 (2H, m), 2.67-2.83 (1H, m), 2.32-2.54 (4H, m), 0.64-2.29 (48H, m).

### 3-O-(3′,3′-Dimethylglutaryl)betulinic acid 1-(4-ethylpiperazinyl) amide

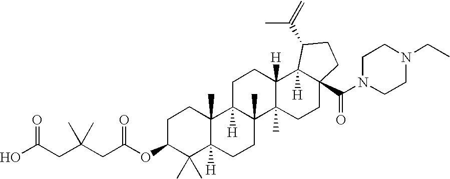

The compound was synthesized from 3-O-(5′-allyloxy-3′,3′-dimethylglutaryl)betulinic acid chloride applying method D with N-ethylpiperazine, followed by method C deprotection. ¹H NMR (400 MHz, CDCl₃); δ ppm 4.65 (1H, s), 4.52 (1H, s), 4.42-4.38 (1H, m), 2.89-2.87 (1H, m), 2.78-2.74 (2H, m), 2.62-2.60 (2H, m), 2.49-2.45 (2H, m), 2.24-2.20 (2H, m), 1.98-1.96 (1H, m), 1.84-1.79 (2H, m), 1.93-1.88 (2H, m), 1.87-0.61 (53H, m); LCMS, 100% R_{f}=3.27; m/z (relative intensity) 695 ([M+H⁺] 10%).

### 3-O-(3′,3′-Dimethylglutaryl)betulinic acid 1-(4-methylpiperazine) amide

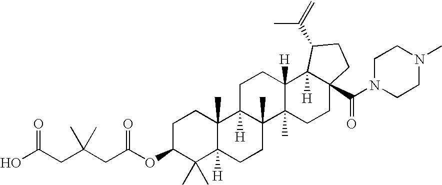

The compound was synthesized from 3-O-acetylbetulinic acid chloride applying method D with N-methylpiperazine, followed by method G deprotection and method H side chain introduction. ¹H NMR (400 MHz, DMSO-d₆) δ ppm 4.46 (1H, s), 4.35 (1H, s), 4.2 (1H, m), 2.75-2.63 (2H, m), 2.23-1.88 (13H, m), 1.81-1.72 (1H, m), 1.59-0.51 (50H, m).

### 3-O-(3′,3′-Dimethylglutaryl)betulinic acid 1-(4-benzylpiperazinyl) amide

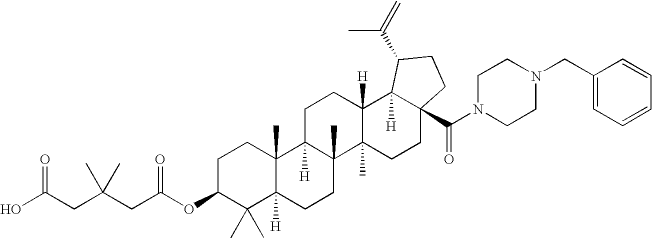

The compound was synthesized from 3-O-(5′-allyloxy-3′,3′-dimethylglutaryl)betulinic acid chloride applying method D with 1-benzylpiperazine, followed by method C deprotection. ¹H NMR (400 MHz, CDCl₃) δ ppm 0.67-1.98 (48H, m), 2.00-2.13 (1H, m), 2.17-2.70 (7H, m), 2.79-2.91 (1H, m), 2.92-3.04 (1H, m), 3.30-3.82 (5H, m), 4.49 (1H, dd, J=11.0, 4.4 Hz), 4.58 (1H, s), 4.72 (1H, d, J=1.8 Hz), 7.28-7.37 (5H, m); LCMS, 100% pure; R_{f}=4.33; m/z (relative intensity) 757 ([M+H]⁺, 70%).

### 3-O-(3′,3′-Dimethylglutaryl)betulinic acid 1-[4-(cyclopropylmethyl)piperazinyl]amide

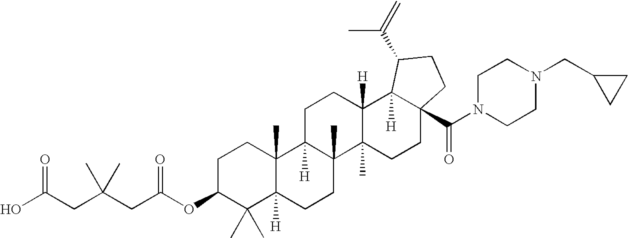

The compound was synthesized from 3-O-(5′-allyloxy-3′,3′-dimethylglutaryl)betulinic acid chloride applying method D with 1-(cyclopropylmethyl)piperazine, followed by method C deprotection. ¹H NMR (400 MHz, CDCl₃) δ ppm 4.65 (1H, s), 4.53 (1H, s), 4.40-4.47 (1H, m), 2.80-2.91 (3H, m), 2.65-2.75 (1H, m), 2.30-2.43 (4H, m), 1.87-2.00 (1H, m), 1.75 (1H, br s), 0.65-1.68 (57H, m), 0.38 (2H, d, J=4.0 Hz).

### 3-O-(3′,3′-Dimethylglutaryl)betulinic acid 1-[4-(isopropylaminocarbonyl)piperazinyl amide

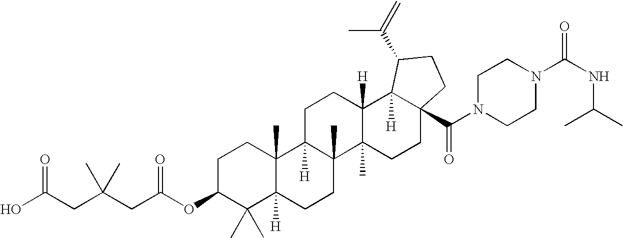

The compound was synthesized from 3-O-(5′-allyloxy-3′,3′-dimethylglutaryl)betulinic acid chloride applying method D with 1-(isopropylaminocarbonyl)piperazine, followed by method C deprotection. ¹H NMR (400 MHz, CDCl₃) δ ppm 0.63-2.06 (53H, m), 2.33 (1H, d), 2.36-2.43 (3H, m), 2.78 (1H, dt), 2.84-2.99 (1H, m), 3.25 (4H, s), 3.46-3.65 (4H, m), 3.83-3.99 (1H, m), 4.19 (1H, d, J=7.3 Hz), 4.37-4.48 (1H, m), 4.52 (1H, s), 4.66 (1H, d, J=2.0 Hz).

### 3-O-(31,3′-Dimethylglutaryl)betulinic acid 1-[4-(methylsulfonyl)piperazinyl] amide

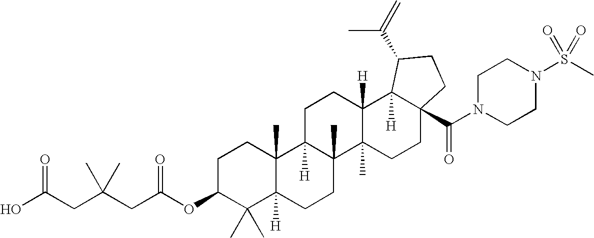

The compound was synthesized from 3-O-(5′-allyloxy-3′,3′-dimethylglutaryl)betulinic acid chloride applying method D with 1-methylsulfonylpiperazine, followed by method C deprotection. ¹H NMR (400 MHz, CDCl₃) δ ppm 0.74-2.25 (47H, m), 2.36-2.57 (4H, m), 2.74-3.09 (5H, m), 3.21 (4H, s), 3.74 (4H, s), 4.45-4.58 (1H, m), 4.62 (1H, s), 4.75 (1H, s).

### 3-O-(3′,3′-Dimethylglutaryl)betulinic acid 1-(4-acetylpiperazinyl) amide

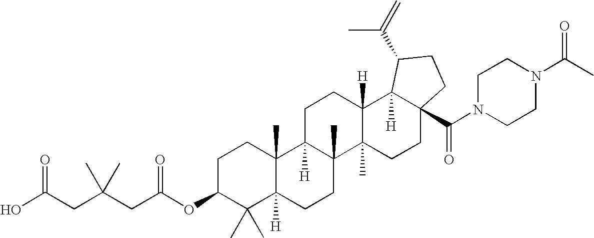

The compound was synthesized from 3-O-(5′-allyloxy-3′,3′-dimethylglutaryl)betulinic acid chloride applying method D with 1-acetylpiperazine, followed by method C deprotection. ¹H NMR (400 MHz, CDCl₃) δ ppm 0.61-1.93 (48H, m), 1.93-2.03 (2H, m), 2.30-2.36 (1H, m), 2.36-2.43 (3H, m), 2.77 (1H, d, J=2.0 Hz), 2.90 (1H, d, J=3.9 Hz), 3.25-3.70 (8H, m), 4.39-4.48 (1H, m), 4.49-4.58 (1H, m), 4.66 (1H, d, J=2.2 Hz).

### 3-O-(3′,3′-Dimethylglutaryl)betulinic acid 2-[(1S,4S)-5-(tert-butoxycarbonyl)-2,5-diazabicyclo[2.2.1]heptyl] amide

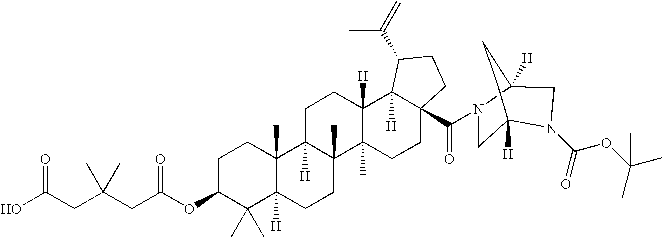

The compound was synthesized from 3-O-(5′-allyloxy-3′,3′-dimethylglutaryl)betulinic acid chloride applying method D with (1S,4S)-5-(tert-butoxycarbonyl)-2,5-diazabicyclo[2.2.1]heptane, followed by method C deprotection. ¹H NMR (400 MHz, CDCl₃) δ ppm 0.49-2.08 (60H, m), 2.23-2.40 (4H, m), 2.46-3.80 (6H, m), 4.32 (1H, s), 4.45 (1H, s), 4.58 (1H, s).

### 3-O-(3′,3′-Dimethylglutaryl)betulinic acid N-2-(2-hydroxyethoxy)ethyl amide

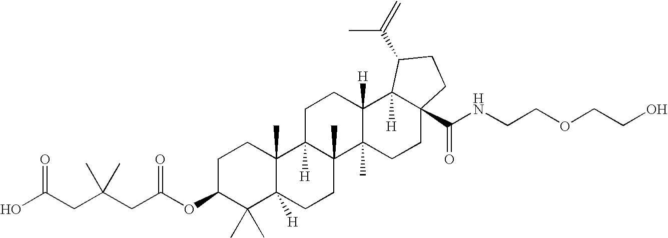

The compound was synthesized from 3-O-(5′-allyloxy-3′,3′-dimethylglutaryl)betulinic acid chloride applying method D with 2-(hydroxyethoxy)ethylamine, followed by method C deprotection. ¹H NMR (400 MHz, CDCl₃); δ ppm 5.99 (1H, s), 4.74 (1H, s), 4.59 (1H, s), 4.53-4.49 (1H, m), 3.75 (2H, s), 3.59-3.56 (5H, m), 3.52-3.50 (1H, m), 3.45-3.42 (1H, m), 2.46-2.41 (5H, m), 1.97-1.94 (2H, m), 1.75-0.76 (46H, m); LCMS, 87% R_{f}=4.49; m/z (relative intensity) 709 ([M+Na⁺] 100%).

### 3-O-(3′,3′-Dimethylglutaryl)betulinic acid N-2-cyanoethyl amide

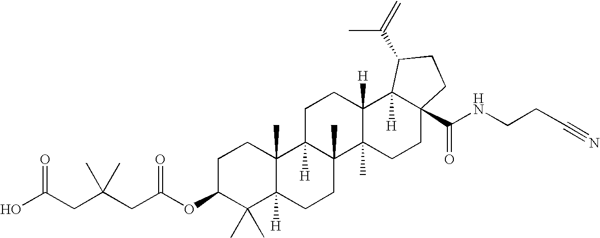

The compound was synthesized from 3-O-(5′-allyloxy-3′,3′-dimethylglutaryl)betulinic acid chloride applying method D with 2-cyanoethylamine, followed by method C deprotection. ¹H NMR (400 MHz, CDCl₃); δ ppm 6.15-6.12 (1H, m), 4.74 (1H, s), 4.60 (1H, s), 4.51-4.47 (1H, m), 3.57-3.52 (1H, m), 3.47-3.43 (1H, m), 3.12-3.06 (1H, m), 2.67-2.63 (2H, m), 2.48-2.38 (4H, m), 1.97-1.93 (2H, m), 1.78-0.77 (46H, m); LCMS, 100% pure, R_{f}=4.67; m/z (relative intensity) 674 ([M+Na⁺] 100%).

### 3-O-(3′,3′-Dimethylglutaryl)betulinic acid 1-[4-(3-(5-methylisoxazolyl)methyl)piperazinyl] amide

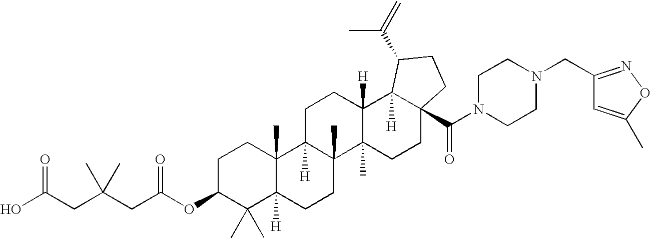

The compound was synthesized from 3-O-(5′-allyloxy-3′,3′-dimethylglutaryl)betulinic acid chloride applying method D with 4-[3-(5-methylisoxazolyl)methyl]piperazine followed by method C deprotection. ¹H NMR (400 MHz, CDCl₃); δ ppm 5.95 (1H, s), 4.65 (1H, s), 4.51 (1H, s), 4.43-4.40 (1H, m), 3.62-3.49 (7H, m), 2.92-2.88 (1H, m), 2.81-2.77 (1H, m), 2.50-2.28 (8H, m), 2.02-1.98 (1H, m), 1.89-1.84 (2H, m), 1.65-0.70 (46H, m); LCMS, 99% pure, R_{f}=3.67; m/z (relative intensity) 762 ([M+H⁺] 10%)

### 3-O-(3′,3′-Dimethylglutaryl)betulinic acid N-(2-thienylmethyl) amide

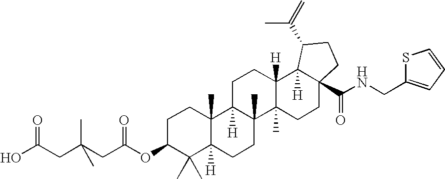

The compound was synthesized from 3-O-(5′-allyloxy-3′,3′-dimethylglutaryl)betulinic acid chloride applying method D with 2-thiophenemethylamine, followed by method C deprotection. ¹H NMR (400 MHz, CDCl₃); δ ppm 7.34 (1H, s), 6.32 (1H, m), 6.22 (1H, m), 5.94-5.91 (1H, m), 4.73 (1H, s), 4.59 (1H, s), 4.52-4.47 (2H, m), 4.37-4.32 (1H, m), 3.18-3.11 (1H, m), 2.50-2.38 (4H, m), 1.97-1.90 (2H, m), 1.76-0.75 (46H, m); LCMS, 100% pure, R_{f}=4.72; m/z (relative intensity) 695 ([M+H⁺] 90%).

### 3-O-(3′,3′-Dimethylglutaryl)betulinic acid N-(2-furanylmethyl) amide

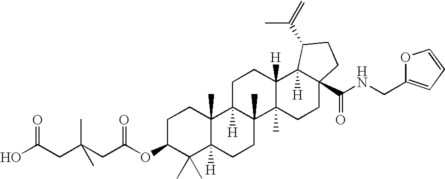

The compound was synthesized from 3-O-(5′-allyloxy-3′,3′-dimethylglutaryl)betulinic acid chloride applying method D with 2-furanemethylamine, followed by method C deprotection. ¹H NMR (400 MHz, CDCl₃); δ ppm 7.15-4.14 (1H, dd, J=1.2, 4.9 Hz), 6.88-6.85 (2H, m), 5.91-5.86 (1H, t, J=5.7 Hz), 4.67 (1H, s), 4.61-4.40 (3H, m), 3.11-3.06 (1H, m), 2.44-2.40 (4H, m), 1.91-1.81 (1H, m), 1.70-0.68 (49H, m); LCMS, 100% pure, R_{f}=4.65; m/z (relative intensity) 679 ([M+H⁺] 65%).

### 3-O-(3′,3′-Dimethylglutaryl)betulinic acid 1-(4-isopropylpiperazinyl) amide

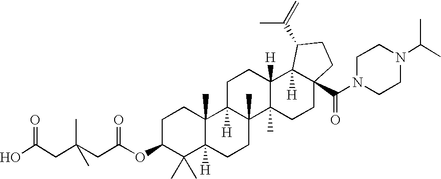

The compound was synthesized from 3-O-(5′-allyloxy-3′,3′-dimethylglutaryl)betulinic acid chloride applying method D with 4-isopropylpiperazine, followed by method C deprotection. ¹H NMR (400 MHz, CD₃OD); δ ppm 4.57 (1H, s), 4.45 (1H, s), 4.33-4.29 (1H, dd, J=6.4, 10.5 Hz), 3.34-3.27 (1H, m), 3.05 (4H, br s), 2.81-2.75 (1H, m), 2.72-2.62 (1H, m), 2.56 (2H, s), 2.36-2.24 (2H, m), 2.02-1.99 (1H, m), 1.89-1.84 (1H, m), 1.74-0.69 (56H, m); LCMS, 97% pure, R_{f}=3.57; m/z (relative intensity) 710 ([M+H⁺] 20%).

### 3-O-(3′,3′-Dimethylglutaryl)betulinic acid 4-(2,6-dimethylmorpholine) amide

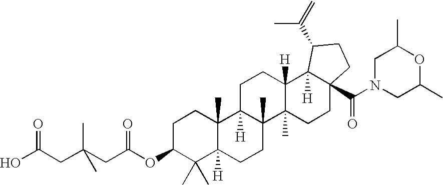

The compound was synthesized from 3-O-(5′-allyloxy-3′,3′-dimethylglutaryl)betulinic acid chloride applying method D with 2,6-dimethylmorpholine, followed by method C deprotection. ¹H NMR (400 MHz, CDCl₃); δ ppm 4.74 (1H, s), 4.59 (1H, s), 4.53-4.49 (1H, dd, J=6.4, 10.5 Hz), 3.53-3.47 (2H, m), 3.00-2.95 (1H, m), 2.89-2.84 (1H, m), 2.48-2.45 (1H, d, J=13.9 Hz), 2.47 (2H, s), 2.42-2.38 (1H, d, J=13.9 Hz), 2.20-1.91 (1H, m), 1.83-1.71 (2H, m), 1.74-0.69 (52H, m); LCMS, 100% pure, R_{f}=4.86; m/z (relative intensity) 697 ([M+H⁺] 100%).

### 3-O-(3′,3′-Dimethylglutaryl)betulinic acid N-4-(3-pyridylmethyl) piperazine amide

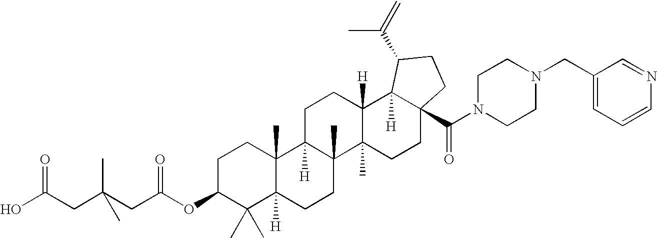

The compound was synthesized from 3-O-(5′-allyloxy-3′,3′-dimethylglutaryl)betulinic acid chloride applying method D with 4-(3-pyridinylmethyl)piperazine, followed by method C deprotection. ¹H NMR (400 MHz, CD₃OD); δ ppm 8.75 (1H, d, J=1.4 Hz), 8.69-8.67 (1H, dd, J=1.4, 5.4 Hz), 8.23-8.21 (1H, d, J=8.0 Hz), 7.72-7.69 (1H, dd, J=5.4, 8.0 Hz), 4.60 (1H, s), 4.49 (1H, s), 4.37-4.33 (1H, m), 3.14 (4H, br s), 2.86-2.79 (1H, m), 2.76-2.64 (1H, m), 2.44-2.39 (1H, d, J=19.3 Hz), 2.31-2.28 (3H, m), 2.05-2.01 (1H, m), 1.93-1.88 (1H, m), 1.74-0.69 (51H, m); LCMS, 94% pure, R_{f}=3.39; m/z (relative intensity) 759 ([M+H⁺] 20%).

### 3-O-(3′,3′-Dimethylglutaryl)betulinic acid N-4-chlorobenzyl amide

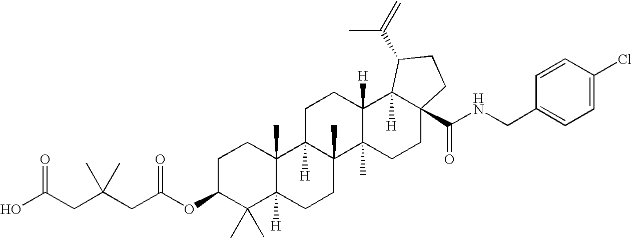

The compound was synthesized from 3-O-(5′-methoxy-3′,3′-dimethylglutaryl)betulinic acid chloride applying method D with 4-chlorobenzylamine, followed by method E deprotection. ¹H NMR (400 MHz, CDCl₃); δ ppm 7.24-7.23 (2H, d, J=7.4 Hz), 7.16-7.14 (2H, d, J=7.4 Hz), 5.88-5.85 (1H, t, J=6.0 Hz), 4.67 (1H, s), 4.53 (1H, s), 4.45-4.40 (1H, m), 4.38-4.37 (1H, d, J=6.0 Hz), 4.26-4.21 (1H, dd, J=5.6, 14.7 Hz), 3.11-3.05 (1H, dt, J=5.6, 11.1 Hz), 2.42-2.37 (3H, m), 2.34-2.31 (1H, d, J=13.9 Hz), 1.74-0.69 (48H, m); LCMS, 100% pure R_{f}=4.70; m/z (relative intensity) 722 ([M+H⁺] 100%).

### 3-O-(3′,3′-Dimethylglutaryl)betulinic acid N-3-methoxybenzylamine amide

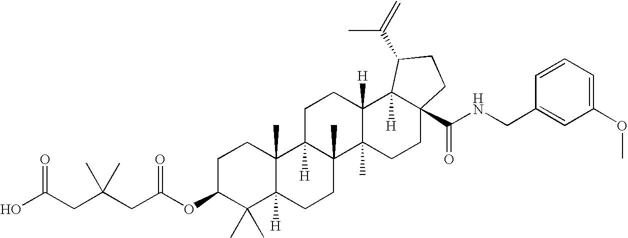

The compound was synthesized from 3-O-(5′-methoxy-3′,3′-dimethylglutaryl)betulinic acid chloride applying method D with 3-methoxybenzylamine, followed by method E deprotection. ¹H NMR (400 MHz, CDCl₃); δ ppm 7.08-7.06 (1H, m), 6.71-6.69 (1H, d, J=7.8 Hz), 6.66-6.63 (2H, m), 5.73-5.70 (1H, t, J=5.8 Hz), 4.58 (1H, s), 4.44 (1H, s), 4.36-4.28 (3H, m), 3.64 (3H, s), 3.02-2.99 (1H, dt, J=5.6, 11.1 Hz), 2.34-2.33 (1H, m), 2.31-2.28 (1H, d, J=13.9 Hz), 2.30 (2H, s), 2.25-2.22 (1H, d, J=13.9 Hz), 1.63-0.75 (47H, m); LCMS, 100% pure, R_{f}=4.56; m/z (relative intensity) 719 ([M+H⁺] 100%).

### 3-O-(3′,3′-Dimethylglutaryl)betulinic acid N-3-methylbenzylamine amide

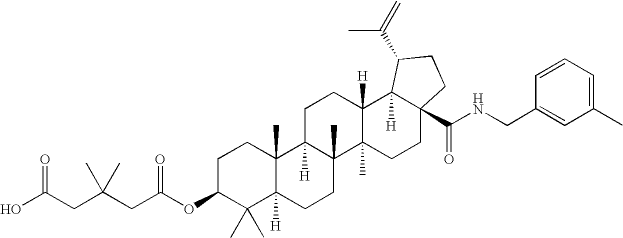

The compound was synthesized from 3-O-(5′-methoxy-3′,3′-dimethylglutaryl)betulinic acid chloride applying method D with 3-methylbenzylamine, followed by method E deprotection. ¹H NMR (400 MHz, CDCl₃); δ ppm 7.16-7.12 (1H, d, J=7.5 Hz), 7.02-6.99 (3H, m), 5.81-5.78 (1H, t, J=5.6 Hz), 4.67 (1H, m), 4.52 (1H, m), 4.44-4.41 (1H, dd, J=5.4, 10.6 Hz), 4.38-4.36 (1H, d, J=5.8 Hz), 4.27-4.22 (1H, dd, J=5.5, 14.6 Hz), 3.13-3.07 (1H, dt, J=4.3, 11.2 Hz), 2.45-2.40 (1H, m), 2.40-2.37 (1H, d, J=14.0 Hz), 2.39 (2H, s), 2.34-2.31 (1H, d, J=14.0 Hz), 2.27 (3H, s), 1.96-1.82 (2H, m), 1.72-0.68 (45H, m); LCMS, 100% pure, R_{f}=4.68; m/z (relative intensity) 703 ([M+H⁺] 100%).

### 3-O-(3′,3′-Dimethylglutaryl)betulinic acid N-3-chlorobenzylamine amide

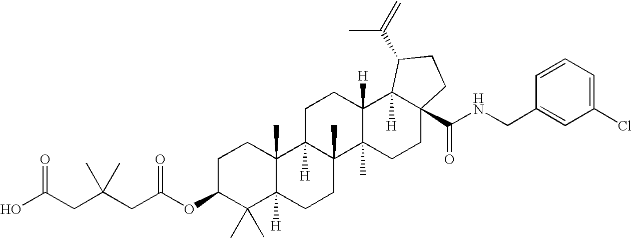

The compound was synthesized from 3-O-(5′-methoxy-3′,3′-dimethylglutaryl)betulinic acid chloride applying method D with 3-chlorobenzylamine, followed by method E deprotection. ¹H NMR (400 MHz, CDCl₃); δ ppm 7.19-7.16 (3H, m), 7.12-7.09 (1H, m), 5.92-5.88 (1H, t, J=5.9 Hz), 4.67 (1H, m), 4.52 (1H, m), 4.44-4.38 (2H, m), 4.26-4.21 (1H, dd, J=5.8, 15.0 Hz), 3.11-3.05 (1H, dt, J=4.4, 11.2 Hz), 2.42-2.35 (1H, m), 2.40-2.38 (1H, d, J=14.1 Hz), 2.39 (2H, s), 2.34-2.31 (1H, d, J=14.1 Hz), 1.91-1.83 (2H, m), 1.72-0.61 (45H, m); LCMS, 98% pure, R_{f}=4.70; m/z (relative intensity) 723 ([M+H⁺] 100%).

### 3-O-(3′,3′-Dimethylglutaryl)betulinic acid N-4-(trifluoromethyl) benzylamine amide

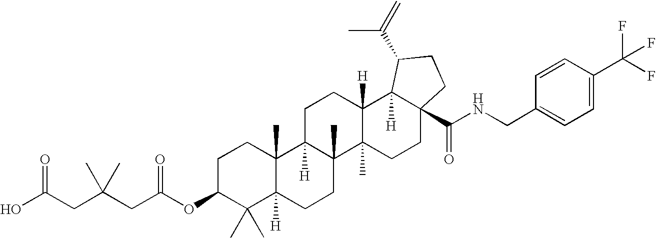

The compound was synthesized from 3-O-(5′-methoxy-3′,3′-dimethylglutaryl)betulinic acid chloride applying method D with 4-trifluoromethylbenzylamine, followed by method E deprotection. ¹H NMR (400 MHz, CDCl₃); δ ppm 7.51-7.48 (2H, d, J=8.2 Hz), 7.33-7.32 (2H, d, J=8.2 Hz), 5.97-5.95 (1H, t, J=5.9 Hz), 4.75 (1H, m), 4.60 (1H, m), 4.50-4.40 (2H, m), 4.35-4.30 (1H, dd, J=5.8, 15.1 Hz), 3.10-3.04 (1H, dt, J=4.4, 11.2 Hz), 2.42-2.35 (1H, m), 2.42-2.37 (1H, d, J=13.9 Hz), 2.39 (2H, s), 2.35-2.31 (1H, d, J=13.9 Hz), 1.90-1.84 (2H, m), 1.72-0.68 (45H, m); LCMS, 100% pure, R_{f}=4.70; m/z (relative intensity) 757 ([M+H⁺] 100%).

### 3-O-(3′,3′-Dimethylglutaryl)betulinic acid N-2-methoxybenzylamine amide

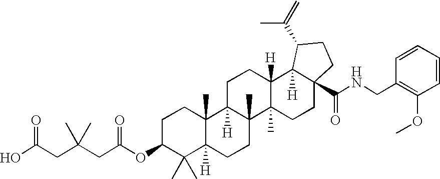

The compound was synthesized from 3-O-(5′-methoxy-3′,3′-dimethylglutaryl)betulinic acid chloride applying method D with 2-methoxybenzylamine, followed by method E deprotection. ¹H NMR (400 MHz, CDCl₃); δ ppm 7.30-7.24 (2H, m), 6.92-6.88 (1H, dt, J=0.9, 7.4 Hz), 6.88-6.86 (1H, d, J=8.1 Hz), 6.21-6.18 (1H, t, J=5.8 Hz), 4.72 (1H, m), 4.58 (1H, m), 4.53-4.45 (2H, m), 4.43-4.38 (1H, dd, J=6.0, 14.5 Hz), 3.85 (3H, s), 3.12-3.05 (1H, dt, J=4.5, 11.3 Hz), 2.49-2.41 (2H, s), 2.47-2.44 (1H, d, J=13.8 Hz), 2.40-2.37 (1H, d, J=13.8 Hz), 2.35-2.32 (1H, m), 1.95-1.91 (2H, m), 1.76-0.66 (45H, m); LCMS, 100% pure, R_{f}=4.65; m/z (relative intensity) 719 ([M+H⁺]95%).

### 3-O-(3′,3′-Dimethylglutaryl)betulinic acid N-2-methylbenzylamine amide

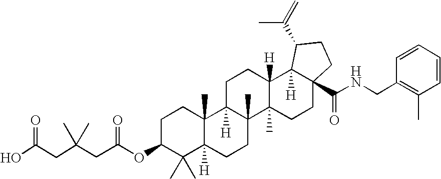

The compound was synthesized from 3-O-(5′-methoxy-3′,3′-dimethylglutaryl)betulinic acid chloride applying method D with 2-methylbenzylamine, followed by method E deprotection. ¹H NMR (400 MHz, CDCl₃); δ ppm 7.26-7.14 (4H, m), 5.67 (1H, t, J=5.3 Hz), 4.75 (1H, m), 4.60 (1H, m), 4.54-4.37 (3H, m), 3.23-3.12 (1H, dt, J=4.5, 11.3 Hz), 2.57-2.37 (5H, m), 2.33 (3H, s), 2.06-1.84 (2H, m), 1.76-0.66 (45H, m); LCMS, 100% pure, R_{f}=4.68; m/z (relative intensity) 703 ([M+H⁺]100%).

### 3-O-(3′,3′-Dimethylglutaryl)betulinic acid N-2-chlorobenzylamine amide

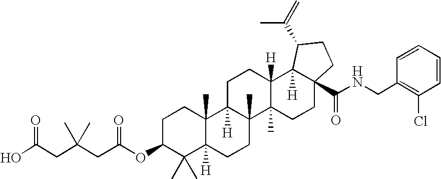

The compound was synthesized from 3-O-(5′-methoxy-3′,3′-dimethylglutaryl)betulinic acid chloride applying method D with 2-chlorobenzylamine, followed by method E deprotection. ¹H NMR (400 MHz, CDCl₃); δ ppm 7.47-7.41 (1H, m), 7.39-7.33 (1H, m), 7.26-7.19 (2H, m), 6.16 (1H, t, J=6.0 Hz), 4.73 (1H, m), 4.59 (1H, m), 4.58-4.39 (3H, m), 3.18-3.08 (1H, dt, J=4.5, 11.3 Hz), 2.52-2.30 (5H, m), 2.00-1.86 (2H, m), 1.83-0.62 (45H, m); LCMS, 100% pure, R_{f}=4.72; m/z (relative intensity) 723 ([M+H⁺] 100%).

### 3-O-(3′,3′-Dimethylglutaryl)betulinic acid N-3,4-dichlorobenzylamine amide

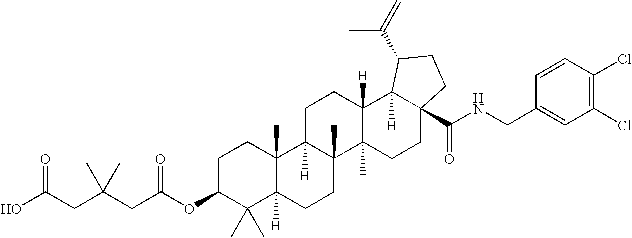

The compound was synthesized from 3-O-(5′-methoxy-3′,3′-dimethylglutaryl)betulinic acid chloride applying method D with 3,4-dichlorobenzylamine, followed by method E deprotection. ¹H NMR (400 MHz, CDCl₃); δ ppm 7.43-7.35 (2H, m), 7.14 (1H, dd, J=2.0, 8.2 Hz), 6.00 (1H, t, J=6.0 Hz), 4.75 (1H, m), 4.61 (1H, m), 4.55-4.41 (2H, m), 4.28 (1H, dd, J=5.9, 15.4 Hz), 3.20-3.08 (1H, dt, J=4.5, 11.2 Hz), 2.53-2.36 (5H, m), 2.01-1.87 (2H, m), 1.76-0.66 (45H, m); LCMS, 94% pure, R_{f}=4.79; m/z (relative intensity) 757 ([M+H⁺] 100%).

### 3-O-(3′,3′-Dimethylglutaryl)betulinic acid N-4-carboxybenzylamine amide

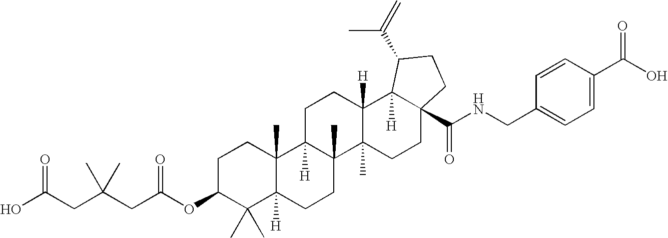

The compound was synthesized from 3-O-(5′-methoxy-3′,3′-dimethylglutaryl)betulinic acid chloride applying method D with methyl 4-aminomethylbenzoate, followed by method E deprotection. ¹H NMR (400 MHz, CDCl₃); δ ppm 8.05 (2H, d, J=8.4 Hz), 7.39 (2H, d, J=8.4 Hz), 6.09 (1H, t, J=6.0 Hz), 4.75 (1H, m), 4.67 (1H, dd, J=6.4, 15.2 Hz), 4.61 (1H, m), 4.54-4.46 (1H, m), 4.34 (1H, dd, J=5.3, 15.2 Hz), 3.21-3.12 (1H, dt, J=4.5, 11.3 Hz), 2.52-2.37 (5H, m), 2.04-1.90 (2H, m), 1.84-0.71 (46H, m); LCMS, 92% pure, R_{f}=4.28; m/z (relative intensity) 733 ([M+H⁺] 100%).

### 3-O-(3′,3′-Dimethylglutaryl)betulinic acid N-4-methylbenzylamine amide

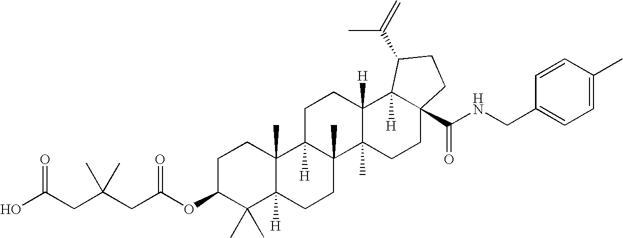

The compound was synthesized from 3-O-(5′-methoxy-3′,3′-dimethylglutaryl)betulinic acid chloride applying method D with 4-methylbenzylamine, followed by method E deprotection. ¹H NMR (400 MHz, CDCl₃); 8 ppm 7.17-7.15 (2H, d, J=8.1 Hz), 7.14-7.12 (2H, d, J=8.1 Hz), 5.84 (1H, t, J=5.6 Hz), 4.74 (1H, m), 4.59 (1H, m), 4.51-4.41 (2H, m), 4.35-4.30 (1H, dd, J=5.5, 14.5 Hz), 3.20-3.14 (1H, dt, J=4.5, 11.3 Hz), 2.51-2.37 (5H, s), 2.33 (3H, s), 2.00-1.88 (2H, m), 1.77-0.76 (45H, m); LCMS, 100% pure, R_{f}=4.67; m/z (relative intensity) 703 ([M+H⁺]100%).

### 3-O-(3′,3′-Dimethylglutaryl)betulinic acid N-4-(dimethylamino) benzylamine amide

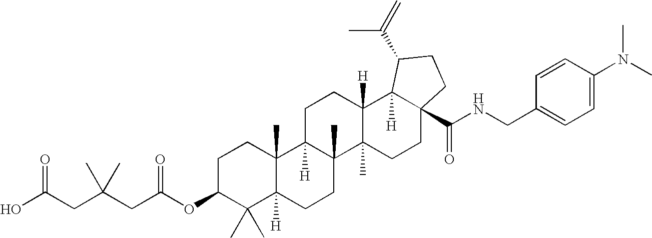

The compound was synthesized from 3-O-(5′-methoxy-3′,3′-dimethylglutaryl)betulinic acid chloride applying method D with 4-(N,N-dimethylamino)benzyl amine, followed by method E deprotection. ¹H NMR (400 MHz, CDCl₃); δ ppm 7.17-7.15 (2H, d, J=8.6 Hz), 6.77-6.65 (2H, d, J=8.6 Hz), 5.79 (1H, t, J=5.1 Hz), 4.74 (1H, m), 4.59 (1H, m), 4.50-4.46 (1H, dd, J=5.5, 10.2 Hz), 4.39-4.34 (1H, dd, J=5.4, 14.4 Hz), 4.31-4.26 (1H, dd, J=5.4, 14.4 Hz), 3.20-3.14 (1H, dt, J=4.6, 11.4 Hz), 2.95 (6H, s), 2.52-2.37 (5H, m), 2.04-1.87 (2H, m), 1.77-0.75 (45H, m); LCMS, 99% pure, R_{f}=3.92; m/z (relative intensity) 732 ([M+H⁺]100%).

### 3-O-(3′,3′-Dimethylglutaryl)betulinic acid N-3-fluorobenzylamine amide

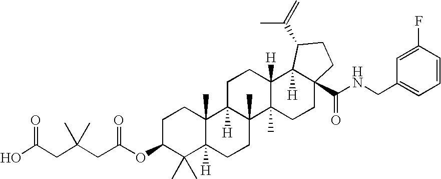

The compound was synthesized from 3-O-(5′-methoxy-3′,3′-dimethylglutaryl)betulinic acid chloride applying method D with 3-fluorobenzylamine, followed by method E deprotection. ¹H NMR (400 MHz, CDCl₃); δ ppm 7.30-7.25 (1H, m), 7.07-7.05 (1H, d, J=7.6 Hz), 6.99-6.92 (2H, m), 6.02 (1H, t, J=5.8 Hz), 4.74 (1H, m), 4.59 (1H, m), 4.50-4.45 (2H, m), 4.36-4.31 (1H, dd, J=5.8, 15.0 Hz), 3.18-3.11 (1H, dt, J=4.5, 11.4 Hz), 2.49-2.37 (5H, m), 1.99-1.91 (2H, m), 1.79-0.75 (45H, m); LCMS, 100% pure, R_{f}=4.59; m/z (relative intensity) 707 ([M+H⁺]100%).

### 3-O-(3′,3′-Dimethylglutaryl)betulinic acid N-2,4-dichlorobenzylamine amide

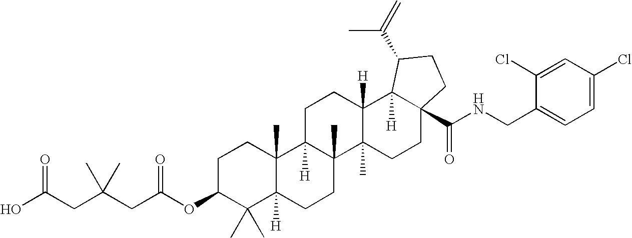

The compound was synthesized from 3-O-(5′-methoxy-3′,3′-dimethylglutaryl)betulinic acid chloride applying method D with 2,4-dichlorobenzylamine, followed by method E deprotection. ¹H NMR (400 MHz, CDCl₃); δ ppm 7.39-7.37 (2H, m), 7.21-7.19 (1H, dd, J=2.1, 8.2 Hz), 6.15 (1H, t, J=6.1 Hz), 4.72 (1H, m), 4.58 (1H, m), 4.52-4.46 (2H, m), 4.40-4.34 (1H, dd, J=5.9, 14.5 Hz), 3.10 (1H, dt, J=4.6, 11.4 Hz), 2.47-2.30 (5H, m), 1.94-1.90 (2H, m), 1.77-0.75 (45H, m); LCMS, 100% pure, R_{f}=4.81; m/z (relative intensity) 758 ([M+H⁺] 100%).

### 3-O-(3′,3′-Dimethylglutaryl)betulinic acid N-(2-pyridylmethyl) amide potassium salt

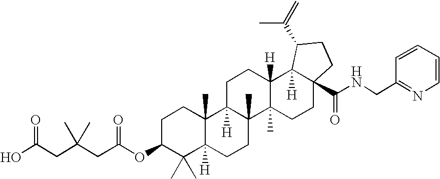

The compound was synthesized from 3-O-acetylbetulinic acid chloride applying method D with (2-pyridinylmethyl)amine, followed by method G deprotection and method H side chain introduction. ¹H NMR (400 MHz, CD₃OD) δ ppm 8.47 (1H, d, J=4.9 Hz), 7.79 (1H, td, J=7.7, 1.7 Hz), 7.37 (1H, d, J=7.8 Hz), 7.26-7.33 (1H, m), 4.70 (1H, d, J=2.0 Hz), 4.58 (1H, s), 4.34-4.53 (3H, m), 3.08 (1H, td, J=10.8, 4.4 Hz), 2.36-2.58 (3H, m), 2.16-2.26 (3H, m), 1.82-1.96 (2H, m), 0.77-1.77 (45H, m); LCMS, 100% pure, R_{f}=3.95 min.

### 3-O-(3′,3′-Dimethylglutaryl)betulinic acid N-(4-pyridylmethyl) amide potassium salt

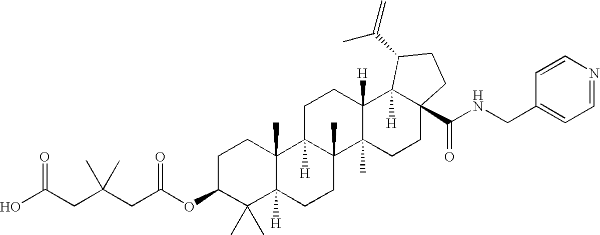

The compound was synthesized from 3-O-acetylbetulinic acid chloride applying method D with (4-pyridinylmethyl)amine, followed by method G deprotection and method H side chain introduction. ¹H NMR (400 MHz, CD₃OD) δ ppm 8.36 (2H, d, J=5.9 Hz), 7.27 (2H, d, J=5.9 Hz), 4.61 (1H, s), 4.49 (1H, s), 4.19-4.38 (3H, m), 2.98 (1H, td, J=10.8, 4.4 Hz), 2.28-2.49 (3H, m), 2.06-2.17 (3H, m), 1.70-1.85 (3H, m), 0.63-1.68 (44H, m).

### 3-O-(3′,3′-Dimethyl-5′-(4-morpholinyl)-5′-oxopentanoyl)betulinic acid 1-[4-(4-morpholinylcarbonyl)piperazinyl] amide

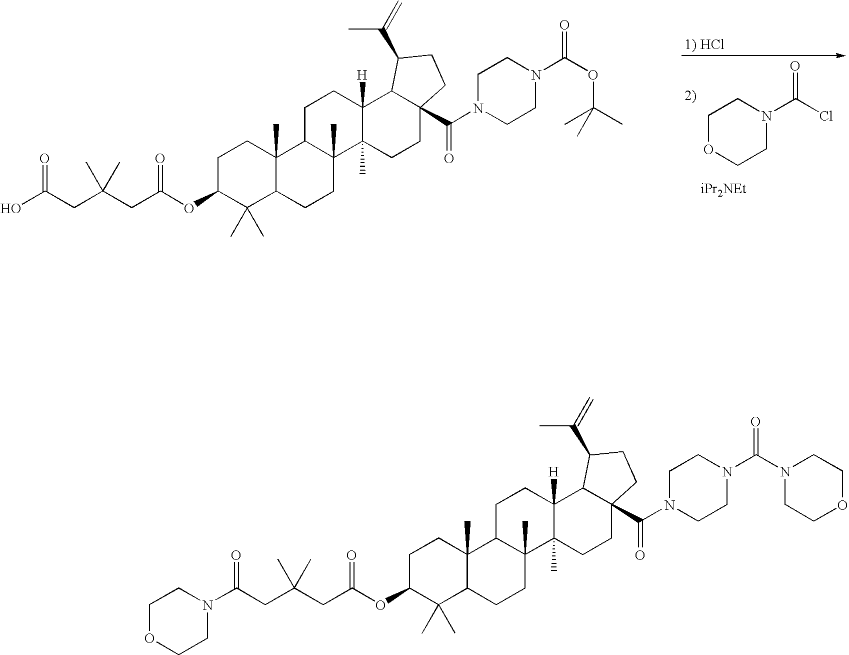

4M HCl in dioxane (0.23 mL, 0.47 mmol) was added to a solution of 3-O-(3′,3′-dimethylglutaryl)betulinic acid 1-[4-(tert-butoxycarbonyl)piperazinyl amide (36 mg, 47 μmol) in dichloromethane (3 mL) and the reaction mixture was stirred at rt for 3 days. The solvents were removed in vacuo to give the HCl salt (34 mg, quantitative) as a white solid which was used as such in the next step.

4-Morpholinecarbonyl chloride (22 mg, 17 μl, 0.14 mmol) was added to a solution of HCl salt (34 mg, 47 μmol) and DIPEA (31 mg, 42 μl, 0.24 mmol) in dichloromethane (1 mL) at rt. The reaction mixture was stirred at rt overnight then, diluted in EtOAc and washed with 2M HCl (aq). The organic phase was dried (Na₂SO₄) and concentrated in vacuo to give the desired title compound (10 mg, 25%). ¹H NMR (400 MHz, CDCl₃) δ ppm 0.70-2.15 (46H, m), 2.36-2.58 (4H, m), 2.91 (2H, d, J=45.30 Hz), 3.14-3.37 (9H, m), 3.41-3.81 (16H, m), 4.37-4.51 (1H, m), 4.58 (1H, s), 4.72 (1H, d, J=1.9 Hz); LCMS, 97% pure; R_{f}=4.05; m/z (relative intensity) 871 ([M+Na]⁺, 100%).

### 3-O-(3′,3′-Dimethylglutaryl)betulinic acid N-hydroxy amide

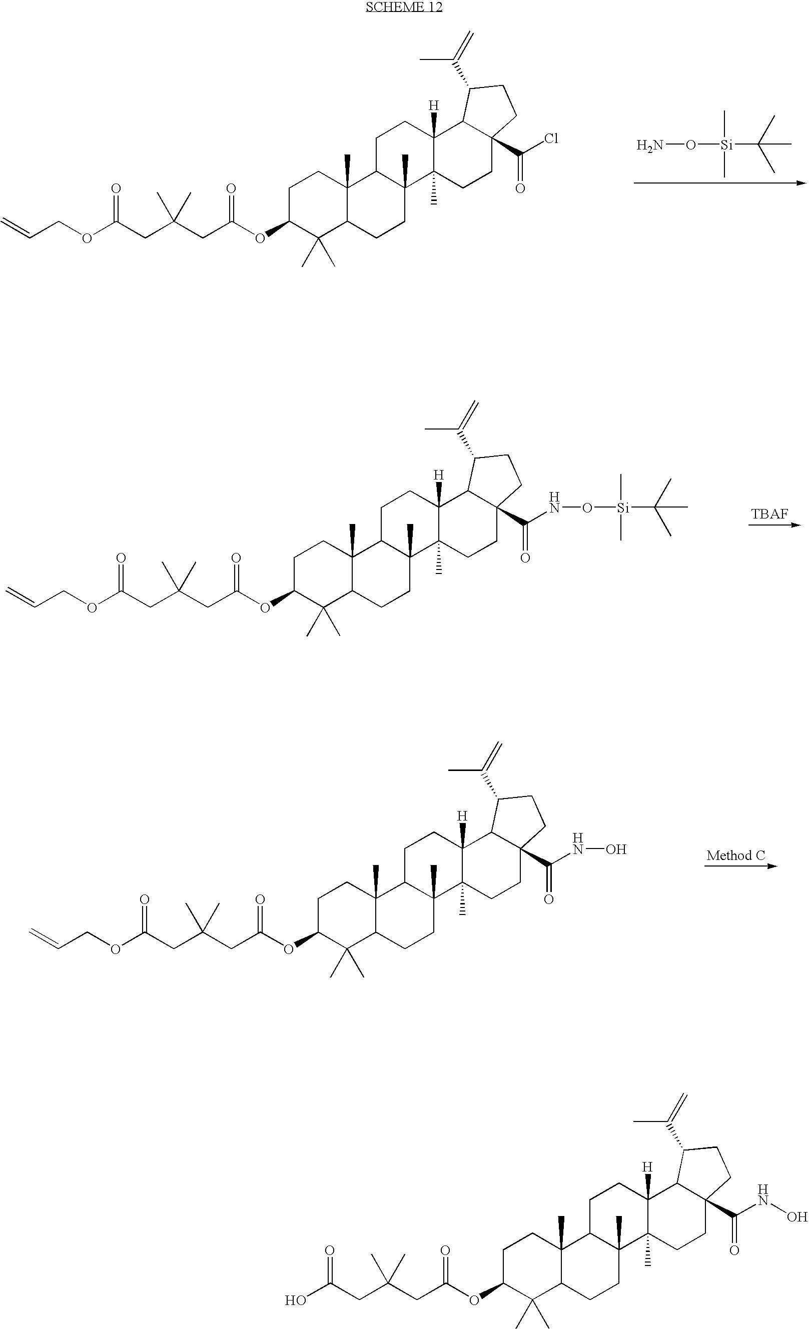

3-O-(3′,3′-Dimethylglutaryl)betulinic acid N-hydroxy amide can be prepared in three steps from 3-O-(5′-allyloxy-3′,3′-dimethylglutaryl)betulinic acid chloride as shown in scheme 12. Coupling of the acid chloride with the silyl ether of hydroxylamine followed by desilylation with tetrabutylammonium fluoride and deallylation using method C yields the N-hydroxy amide analogue.

### 3-O-(3′,3′-Dimethylglutaryl)betulinic acid N-methylsulfonyl amide

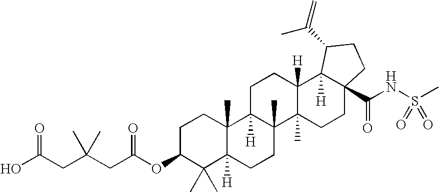

The compound was synthesized from 3-O-(5′-allyloxy-3′,3′-dimethylglutaryl)betulinic acid chloride applying method D with methanesulfonamide, followed by method C deprotection. ¹H NMR (400 MHz, CDCl₃) δ ppm 0.66-1.68 (45H, m), 1.75-1.89 (2H, m), 2.07-2.19 (2H, m), 2.33 (1H, d), 2.36-2.44 (3H, m), 2.92 (1H, dt), 3.60 (3H, s), 4.42 (1H, dd), 4.53 (1H, s), 4.66 (1H, d, J=2.2 Hz).

### 3-O-(3′,3′-Dimethylglutaryl)betulinic acid N-phenylsulfonyl amide

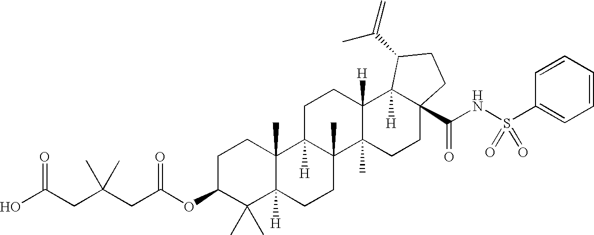

The compound was synthesized 3-O-(5′-allyloxy-3′,3′-dimethylglutaryl)betulinic acid chloride applying method D with benzenesulfonamide followed by method C deprotection. ¹H NMR (400 MHz, CDCl₃) δ ppm 0.51-2.29 (47H, m), 2.37-2.53 (5H, m), 2.90 (1H, td J=10.7, 4.4 Hz), 4.48 (1H, dd, J=5.9, 11.5 Hz), 4.55 (1H, s), 4.66 (1H, d, J=1.7 Hz), 7.52-7.58 (2H, m), 7.65 (1H, td, J=6.6, 1.2 Hz), 8.08-8.04 (2H, m), 8.47 (1H, s).

### 3-O-(4′-(Methylsulfonylamino)-4-oxo-3′,3′-dimethylbutanoyl)betulinic acid N-methylsulfonyl amide

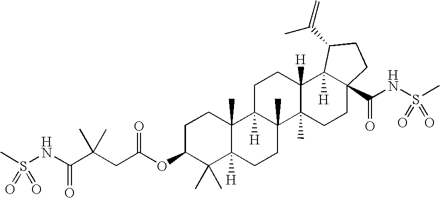

3-O-(3′,3′dimethylsuccinyl)betulinic acid was activated as the bis-acid chloride with oxalyl chloride and reacted with an excess of methanesulfonamide. ¹H NMR (400 MHz, CDCl₃) δ ppm 0.53-2.02 (47H, m), 2.34 (1H, d, J=2.9 Hz), 2.55 (1H, d), 2.64 (1H, d), 2.97 (1H, dt, J=4.4 Hz), 3.12-3.34 (6H, m), 4.46 (1H, dd, J=11.2, 5.4 Hz), 4.55 (1H, s), 4.66 (1H, s), 8.29 (1H, s), 9.31 (1H, s).

### EXAMPLE 5

### C-28 Heterocyclic Derivatives

Tetrazole compounds can be prepared in three steps from 3-O-(3′,3′-dimethylglutaryl)betulinic acid 2-cyanoethylamide as shown in Scheme 13.

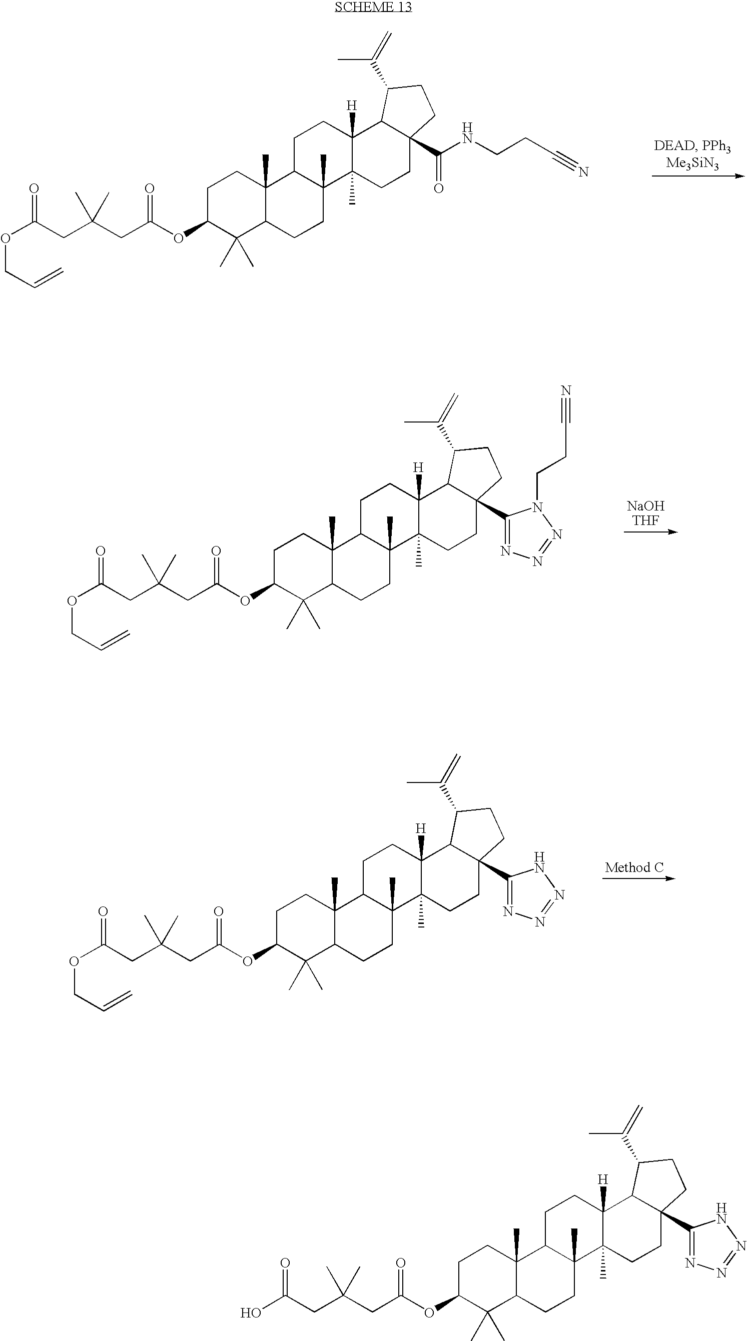

The tetrazole ring can be obtained by reaction of the activated amide with azidotrimethylsilane. Subsequent removal of the 2-cyanoethyl protecting group under basic conditions, followed by deallylation using method C affords the desired compound.

Both oxazoline and oxazole compounds can be prepared in three steps from 3-O-(5′-allyloxy-3′,3′-dimethylglutaryl)betulinic acid 2-aminoethyl amide TFA salt as shown in Scheme 14.

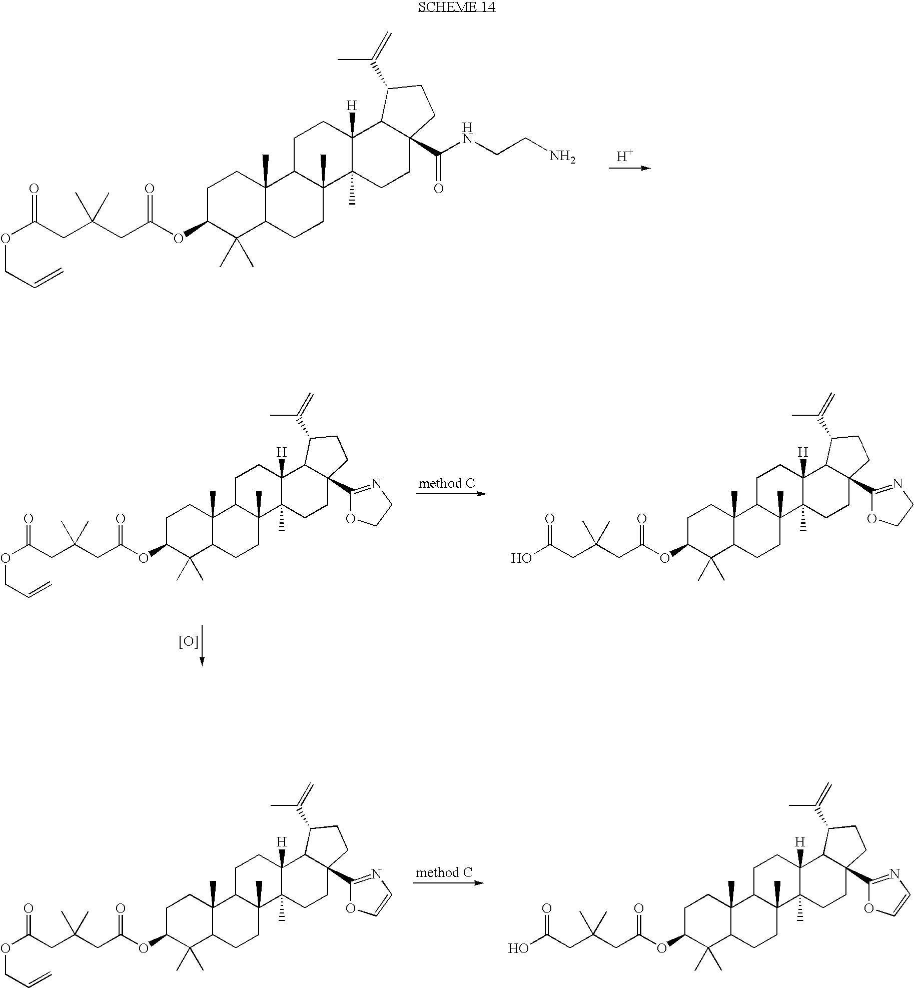

Acid mediated cyclization of the amine salt affords the oxazoline. Further aromatization with manganese(IV) oxide yields the corresponding oxazole derivative. Both compounds can be deallylated using method C.

### EXAMPLE 6

### Synthesis of Betulin C-28 O-Acyl Derivatives

Betulin C-28 O-acyls were prepared in two steps from betulin as shown in Scheme 15. 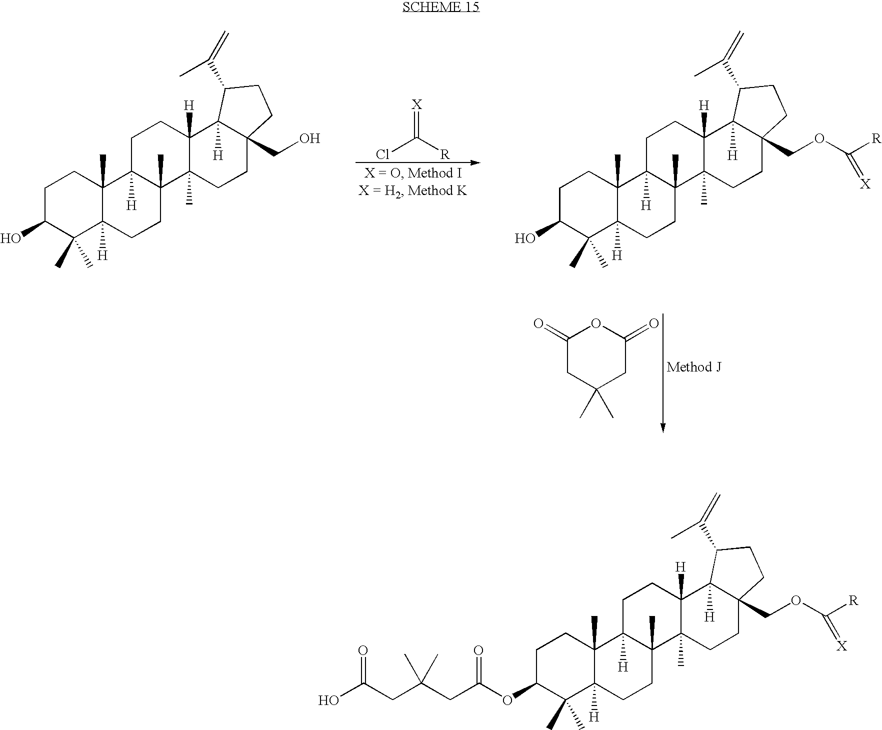
Method I: Ester Formation Method.

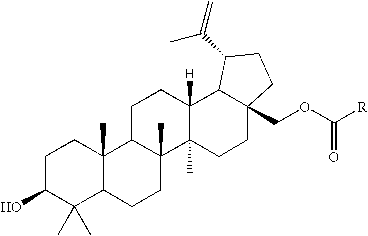

Betulin 28-O-acyl compounds were prepared by adding the desired acid chloride or anhydride (2 equivalents) and DMAP (0.5 equivalents) at 0° C. to a solution of betulin (1 equivalent) in dry pyridine. The reaction was stirred at 115° C. overnight. The reaction mixture was diluted in EtOAc, washed successively with 1M HCl aqueous solution (3×), water and dried over MgSO₄. The combined organic layers were concentrated to dryness in vacuo. Flash column chromatography on silica gel (heptane:EtOAc) provided the desired compound.
Method J: 3′,3′-Dimethylglutaric Anhydride Addition Method.

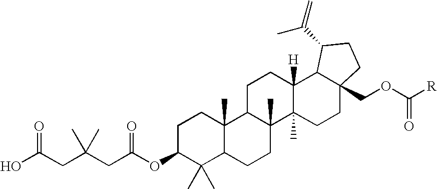

3-O-(3′,3′-Dimethylglutaryl)betulin 28-O-acyl compounds were prepared by adding 3,3-dimethylglutaric anhydride (10 equivalents) and DMAP (1 equivalent) to a solution of the desired betulin ester (1 equivalent) in dry pyridine, in presence of activated 4 Å molecular sieves. The reaction was stirred at 115° C. overnight, diluted in EtOAc, washed successively with 1M HCl aqueous solution (2×), water and dried over MgSO₄. The combined organic layers were concentrated to dryness in vacuo. Flash column chromatography on silica gel (heptane:EtOAc) provided the desired compound.

### 3-O-(3′,3′-Dimethylglutaryl)-28-O-(pivaloyl)betulin

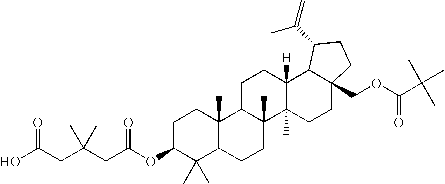

The compound was synthesized applying method I with pivaloyl chloride followed by method J glutaric side chain introduction: ¹H NMR (400 MHz, Acetone-d₆) δ ppm 4.62 (1H, d, J=2.6 Hz), 4.45-4.49 (1H, m), 4.29-4.37 (1H, m), 4.23 (1H, dd, J=11.2, 1.6 Hz), 3.71 (1H, d, J=11.3 Hz), 2.23-2.49 (6H, m), 0.66-1.97 (56H, m); LCMS, 94% pure; R_{f}=4.66; m/z (relative intensity) 692 ([M+Na]⁺, 100%).

### 3-O-(3′,3′-Dimethylglutaryl)-28-O-(isobutyryl)betulin

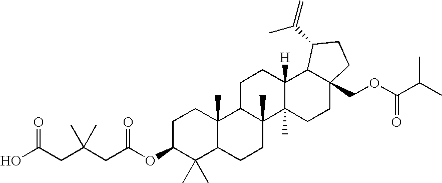

The compound was synthesized applying method I with isobutyryl chloride followed by method J glutaric side chain introduction: ¹H NMR (400 MHz, Acetone-d₆) δ ppm 4.62 (1H, d, J=2.2 Hz), 4.47 (1H, s), 4.14-4.39 (2H, m), 3.72 (1H, d, J=11.0 Hz), 2.20-2.54 (6H, m), 1.79-2.01 (5H, m), 0.61-1.80 (49H, m); LCMS 93% pure; R_{f}=4.56; m/z (relative intensity) 677 ([M+Na]⁺, 100%).

### 3-O-(3′,3′-Dimethylglutaryl)-28-O-(benzoyl)betulin

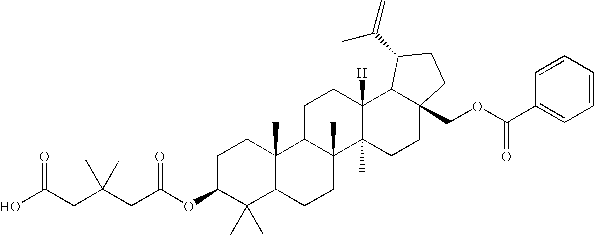

The compound was synthesized applying method I with benzoyl chloride followed by method J glutaric side chain introduction: ¹H NMR (400 MHz, Acetone-d₆) δ ppm 7.92 (2H, d, J=7.3 Hz), 7.26-7.64 (3H, m), 4.18-4.79 (4H, m), 3.75-4.13 (1H, m), 2.13-2.88 (16H, m), 0.36-2.13 (37H, m); LCMS, 100% pure; R_{f}=5.42; m/z (relative intensity) 752 ([M+Na⁺+Acetonitrile]⁺, 100%).

### 3-O-(3′,3′-Dimethylglutaryl)-28-O-((2-tert-butoxycarbonylamino)-isobutyryl)betulin

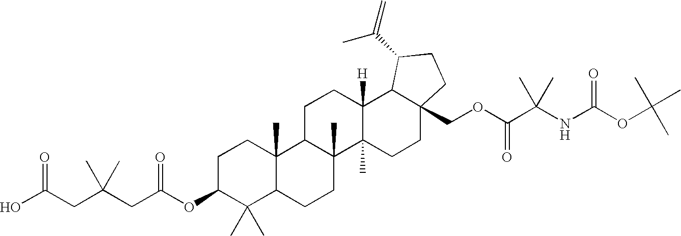

The compound can be synthesized applying method I with 2-(tert-butoxycarbonylamino)isobutyryl chloride followed by method J glutaric side chain introduction.

### 3-O-(3′,3′-Dimethylglutaryl)-28-O-(2-aminoisobutyryl)betulin

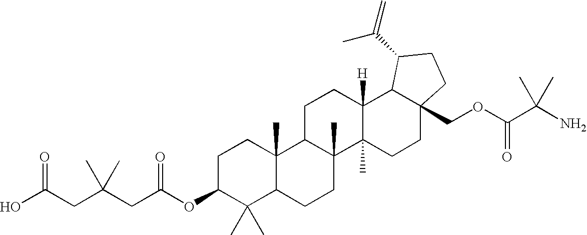

3-O-(3′,3′-Dimethylglutaryl)-28-O-((2-tert-butoxycarbonylamino)-isobutyryl)betulin can be deprotected using method F.

### EXAMPLE 7

### Synthesis of Betulin C-28 O-Ether Compounds

Method K: Synthetic Route to C-28 Ethers.

Betulin C-28 ether compounds can be prepared by adding the desired electrophile (2 equivalents) (e.g. alkyl halide or Michael acceptor) to a solution of betulin (1 equivalent) and DMAP (1.1 equivalents) in DMF. The reaction mixture is heated to reflux. The combined organic layers are concentrated to dryness in vacuo and the resulting solid is purified by flash column chromatography on silica gel (hexane:EtOAc) to provide the desired ether.

### 3-O-(3′,3′-Dimethylglutaryl)-28-O-(2-tert-butoxycarbonylmethyl)betulin

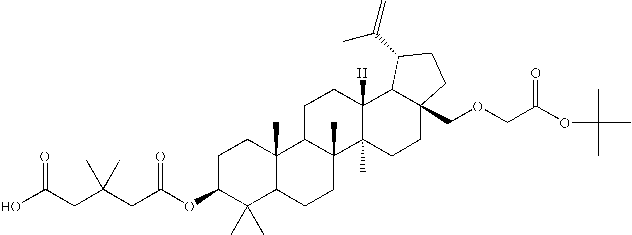

The compound is synthesized by applying method K with tert-butyl chloroacetate followed by method J glutaric side chain introduction.

### 3-O-(3′,3′-Dimethylglutaryl)-28-O-(2-cyanoethyl)betulin

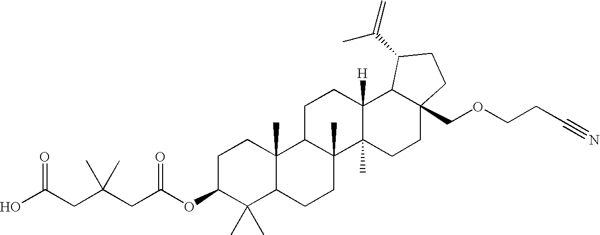

The compound can be synthesized applying method K with acrylonitrile followed by method J glutaric side chain introduction.

### EXAMPLE 8

### Synthesis of C-28 Amines (28-Aminolup-20(29)-enes) from Betulin

The C-28 amines can be synthesized starting from either betulin or betulinic acid. A method for synthesis of C-28 amines from betulin is shown in Scheme 16.

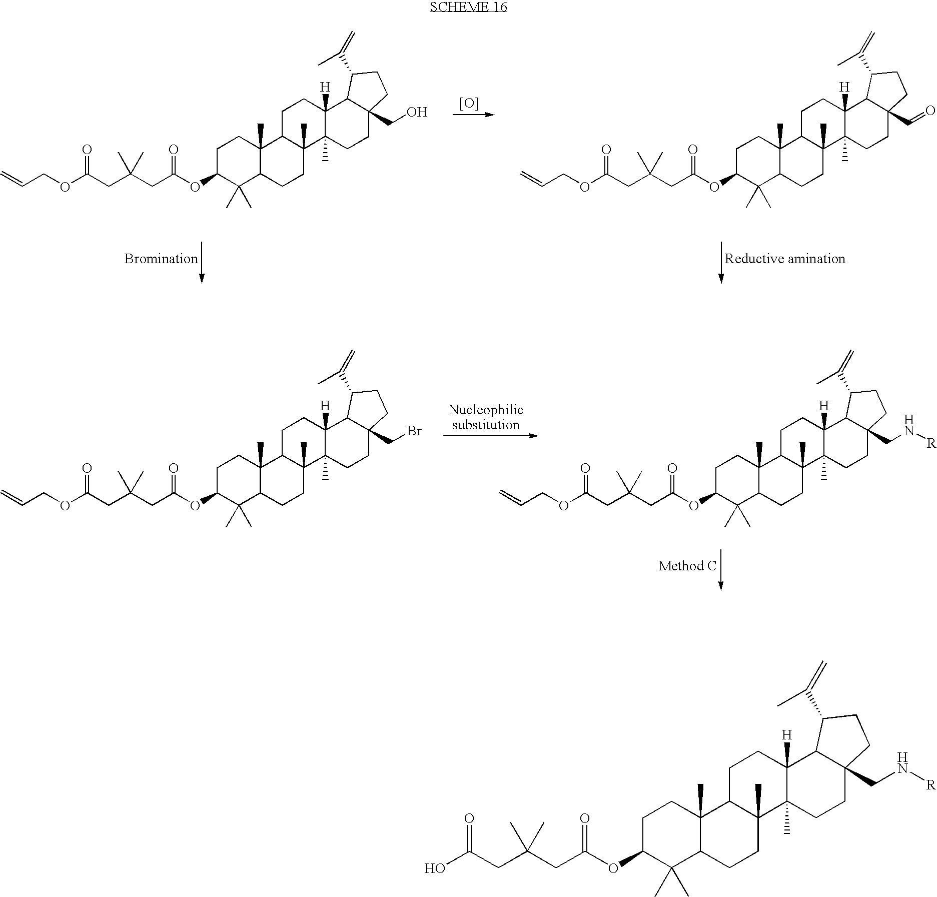

C-28-Aminolup-20(29)-enes can be prepared from 3-O-(5′-allyloxy-3′,3′-dimethylglutaryl)betulin, either via oxidation of the hydroxy group in the C-28 position to the corresponding aldehyde followed by reductive amination, or via conversion of the same hydroxyl group to an alkyl bromide, followed by displacement with a selection of amines.

3-O-(5′-Allyloxy-3′,3′-dimethylglutaryl)betulin starting material was prepared either via protection of the C-28 hydroxy of betulin with trityl ether, followed by coupling to 5-allyloxy-3,3-dimethylglutaryl chloride and removal of the trityl group (Scheme 17) or by silyl protection of the C-28 hydroxy followed by coupling with allyl 3,3-dimethylglutaryl chloride and desilylation (Scheme 18).

### A. Preparation of allyl protected 3-O-(3′,3′-dimethylglutaryl)betulin: via trityl ether

3-O-(5′-Allyloxy-3′,3′-dimethylglutaryl)betulin was synthesized in three steps from betulin as shown in Scheme 17.

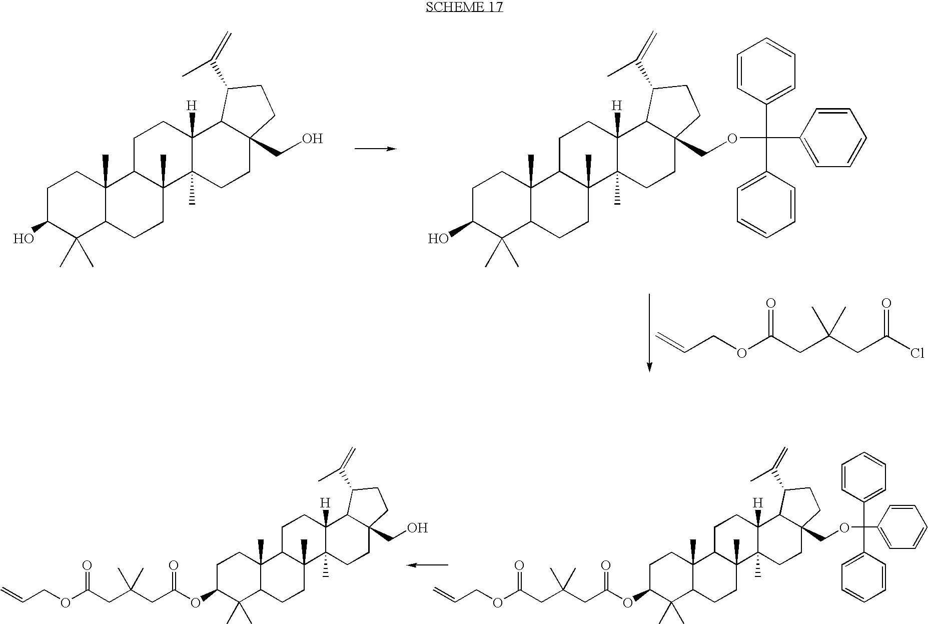

Betulin was selectively trityl protected at the C-28 hydroxy position, then coupled to allyl 3,3-dimethylglutaryl chloride. Treatment with PPTS afforded 3-O-(5′-allyloxy-3′,3′-dimethylglutaryl)betulin.

### 28-O-(Trityl)betulin

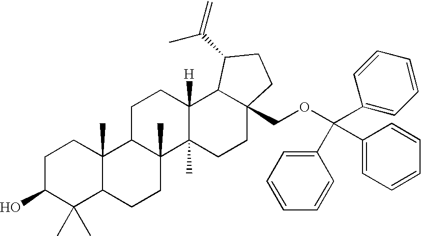

Trityl chloride (2.85 g, 10.0 mmol) and DMAP (0.97 g, 7.7 mmol) were added to a suspension of betulin (3.1 g, 7.0 mmol) in DMF (20 mL). The reaction mixture was heated to reflux for 5.5 hours. The reaction mixture was diluted in EtOAc (200 mL), washed six times with water and dried over Na₂SO₄. The combined organic layers were concentrated to dryness in vacuo and the resulting solid was purified by flash column chromatography on silica gel (EtOAc 0 to 20% in Heptane) to provide the desired trityl ether as a white solid (2.0 g, 42%): ¹H NMR (400 MHz, Acetone-d₆) δ ppm 7.81 (3H, s), 7.29-7.47 (6H, m), 7.04-7.28 (6H, m), 4.34-4.48 (2H, m), 3.10 (1H, d, J=8.8 Hz), 2.96 (1H, dd, J=10.2, 5.5 Hz), 2.82 (1H, d, J=8.8 Hz), 2.01-2.16 (3H, m), 1.87-1.94 (2H, m), 0.41-1.68 (38H, m).

### 3-O-(5′-Allyloxy-3′,3′-dimethylglutaryl)-28-O-(trityl)betulin

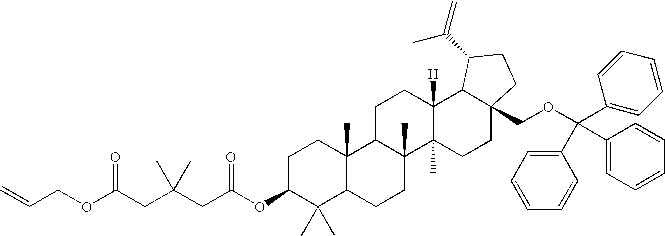

Betulin 28-O-trityl ether (2.0 g, 2.92 mmol) was added to a solution of allyl 3,3-dimethylglutaryl chloride (0.66 g, 3.06 mmol) and -DIPEA (1.04 mL, 6.0 mmol) in dry dichloromethane (20 mL) at 0° C. The reaction mixture was stirred at 40° C. overnight, diluted in dichloromethane (50 mL), washed three times with 1M Na₂CO₃, water and dried over MgSO₄. The combined organic layers were concentrated to dryness in vacuo. Flash column chromatography on silica gel (Heptane 95%:EtOAc 5%) provided the desired compound (1.0 g, 39%) as a pale oil: ¹H NMR (400 MHz, Acetone-d₆) δ ppm 7.32-7.51 (6H, m, J=7.0 Hz), 7.03-7.31 (9H, m), 5.72-5.91 (1H, m), 4.99-5.27 (2H, m), 4.22-4.51 (5H, m), 3.10 (1H, d, J=9.5 Hz), 2.82 (1H, d, J=9.1 Hz), 2.18-2.43 (5H, m), 2.00-2.16 (3H, m), 0.27-2.00 (45H, m); LCMS, 100% pure; R_{f}=5.30; m/z (relative intensity) 890 ([M+Na]⁺, 100%).

### 3-O-(5′-Allyloxy-3′,3′-dimethylglutaryl)betulin

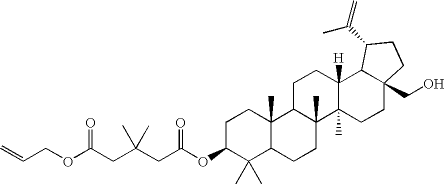

3-O-(5′-Allyloxy-3′,3′-dimethylglutaryl)betulin 28-O-trityl ether (0.98 g, 1.11 mmol) and PPTS (1.53 g, 6.62 mmol) were refluxed overnight in a 2:1 mixture EtOH/dichloromethane (18 mL). The reaction mixture was concentrated in vacuo and the residue partitioned between water and EtOAc. The organic phase was washed twice with water, dried over Na₂SO₄ and concentrated in vacuo. Flash column chromatography on silica gel (EtOAc 0 to 20% in Heptane) provided the desired compound (0.518 g, 75%) as a white solid: ¹H NMR (400 MHz, CDCl₃) δ ppm 5.82-6.00 (1H, m), 5.17-5.38 (2H, m), 4.68 (1H, d, J=2.4 Hz), 4.52-4.61 (3H, m), 4.42-4.50 (1H, m), 3.80 (1H, d, J=10.3 Hz), 3.33 (1H, d, J=10.8 Hz), 0.57-2.56 (53H, m).

### B. Preparation of allyl protected 3-O-(3′,3′-dimethylglutaryl)betulin:—via tert-butyldimethylsilyl ether

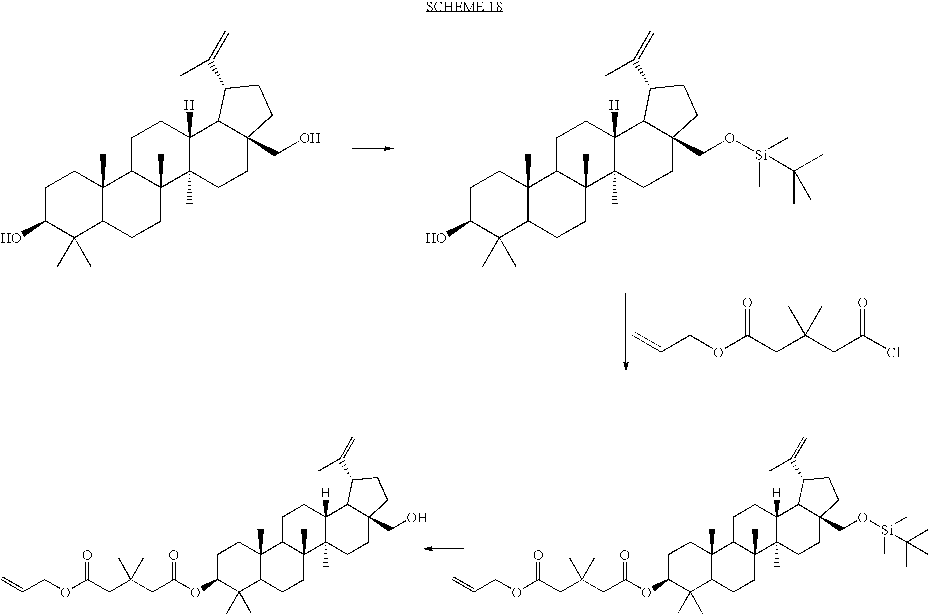

3-O-(5′-Allyloxy-3′,3′-dimethylglutaryl)betulin was synthesized in three steps from betulin as shown in Scheme 18. Betulin was selectively silyl protected at the C-28 alcohol position, then coupled to allyl 3,3-dimethylglutaryl chloride. Desilylation using tetrabutylammonium fluoride afforded 3-O-(5′-allyloxy-3′,3′-dimethylglutaryl)betulin.

### 28-O-(tert-Butyldimethylsilyl)betulin

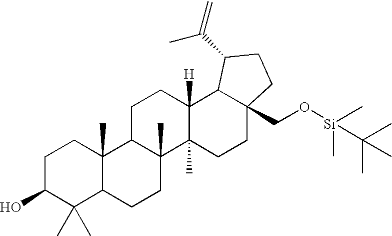

A solution of tert-butyldimethylsilyl chloride (0.79 g, 4.8 mmol) in dry DMF (10 mL) was added to a suspension of betulin (2.0 g, 4.4 mmol) and imidazole (0.4 g, 5.8 mmol) in DMF (20 mL) at 0° C. The reaction mixture was heated at 60° C. overnight (became clear solution above 45° C.). The reaction mixture was diluted in EtOAc (300 mL), washed three times with saturated NaHCO₃, four times with water, and dried over Na₂SO₄. The combined organic layers were concentrated to dryness in vacuo and the resulting solid was purified by flash column chromatography on silica gel (EtOAc 0 to 30% in Heptane) to give the desired TBDMS ether as a white solid (1.8 g, 71%). TLC (30% EtOAc:Heptane) R_{f}=0.58, ¹H NMR (400 MHz, CDCl₃) δ ppm 4.63 (1H, d, J=2.4 Hz), 4.53 (1H, s), 3.63 (1H, d, J=9.8 Hz), 3.10-3.25 (2H, m), 2.30-2.42 (1H, m), 1.80-1.96 (4H, m), 0.58-1.72 (56H, m).

### 3-O-(5′-Allyloxy-3′,3′-dimethylglutaryl)-28-O-(tert-butyldimethylsilyl) betulin

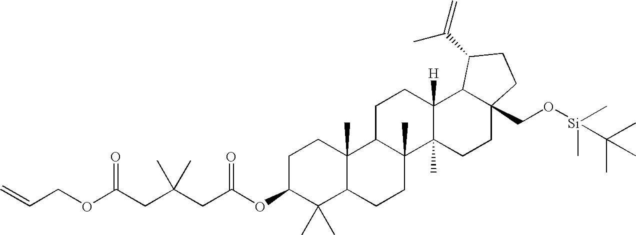

28-O-(tert-Butyldimethylsilyl)betulin ether (1.8 g, 3.2 mmol) was added at 0° C. to a solution of allyl 3,3-dimethylglutaryl chloride (0.98 g, 4.4 mmol) in dry dichloromethane (10 mL) and DIPEA (1.5 mL, 9.0 mmol). The reaction mixture was stirred at 40° C. overnight. The reaction was concentrated to dryness in vacuo and the crude solid was purified by flash column chromatography on silica gel (heptane 95%:EtOAc 5%) to give the desired compound (0.58 g, 25%) as a white solid. ¹H NMR (400 MHz, CDCl₃) δ ppm −0.06-0.06 (6H, m), 0.66-1.70 (54H, m), 1.73-1.99 (3H, m), 2.26-2.50 (5H, m), 3.21 (1H, d, J=9.8 Hz), 3.63 (1H, d, J=8.8 Hz), 4.33-4.48 (1H, m), 4.49-4.68 (4H, m), 5.19 (1H, d, J=11.7 Hz), 5.28 (1H, d, J=17.1 Hz), 5.72-6.01 (1H, m).

### 3-O-(5′-Allyloxy-3′,3′-dimethylglutaryl)betulin

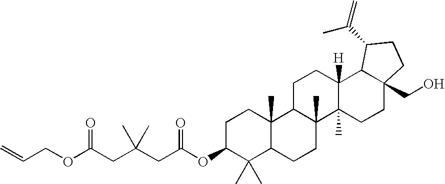

3-O-(5′-Allyloxy-3′,3′-dimethylglutaryl)betulin 28-O-TBDMS ether (0.578 g, 0.78 mmol) and tetrabutylammonium fluoride (2.1 mL, 1 M in THF, 2.17 mmol) were stirred overnight in THF (2 mL). The reaction mixture was diluted in EtOAc, and washed twice with water, dried over Na₂SO₄ and concentrated in vacuo. Flash column chromatography on silica gel (EtOAc 0 to 10% in heptane) provided the desired compound (0.402 g, 82%) as a white solid. ¹H NMR (400 MHz, CDCl₃) δ ppm 0.71-2.04 (49H, m), 2.28-2.55 (5H, m), 3.33 (1H, dd, J=10.5, 4.2 Hz), 3.80 (1H, dd, J=10.5, 3.7 Hz), 4.41-4.51 (1H, m), 4.53-4.72 (4H, m), 5.23 (1H, d, J=10.3 Hz), 5.32 (1H, d, J=17.1 Hz), 5.81-6.01 (1 H, m).

### C. Amine synthesis via nucleophilic substitution

### 28-Bromo-3-O-(5′-allyloxy-3′,3′-dimethylglutaryl)lup-20(29)-ene

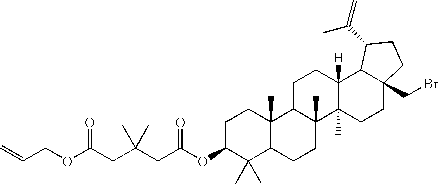

3-O-(5′-Allyloxy-3′,3′-dimethylglutaryl)betulin was reacted with triphenylphosphine and carbon tetrabromide to provide the desired halogen derivative.
Method L: Amine Introduction Via Nucleophilic Substitution.

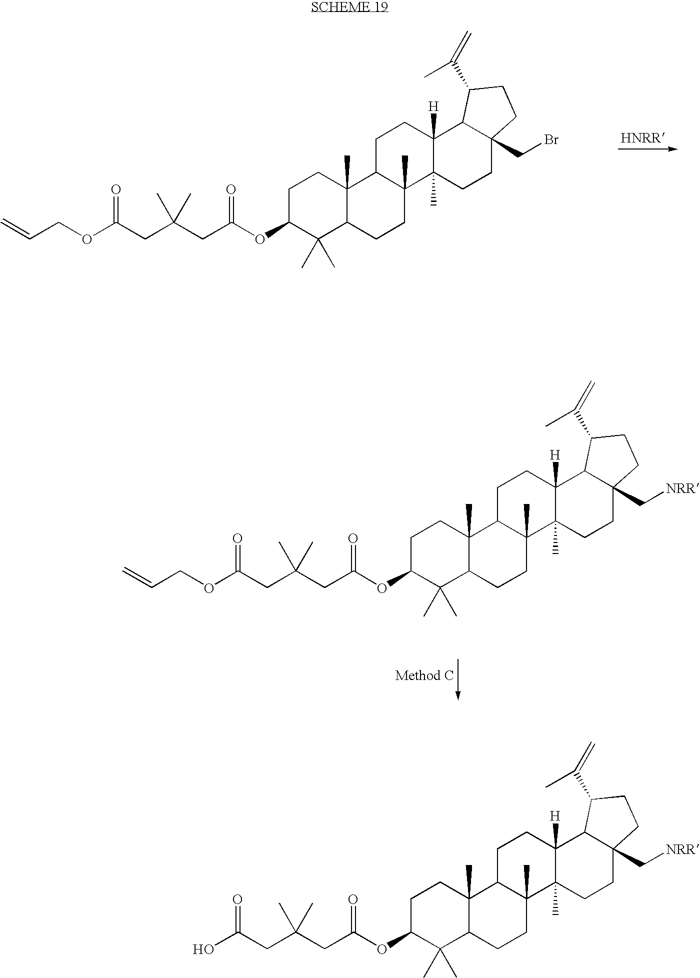

3-O-(3′,3′-Dimethylglutaryl)-28-aminolup-20(29)-enes can be prepared by reacting the desired primary or secondary amine with 28-bromo-3-O-(5′-allyloxy-3′,3′-dimethylglutaryl)lupane under standard conditions.

C. Amine Synthesis Via Reductive Amination.

Method M: Amine Introduction Via Reductive Amination.

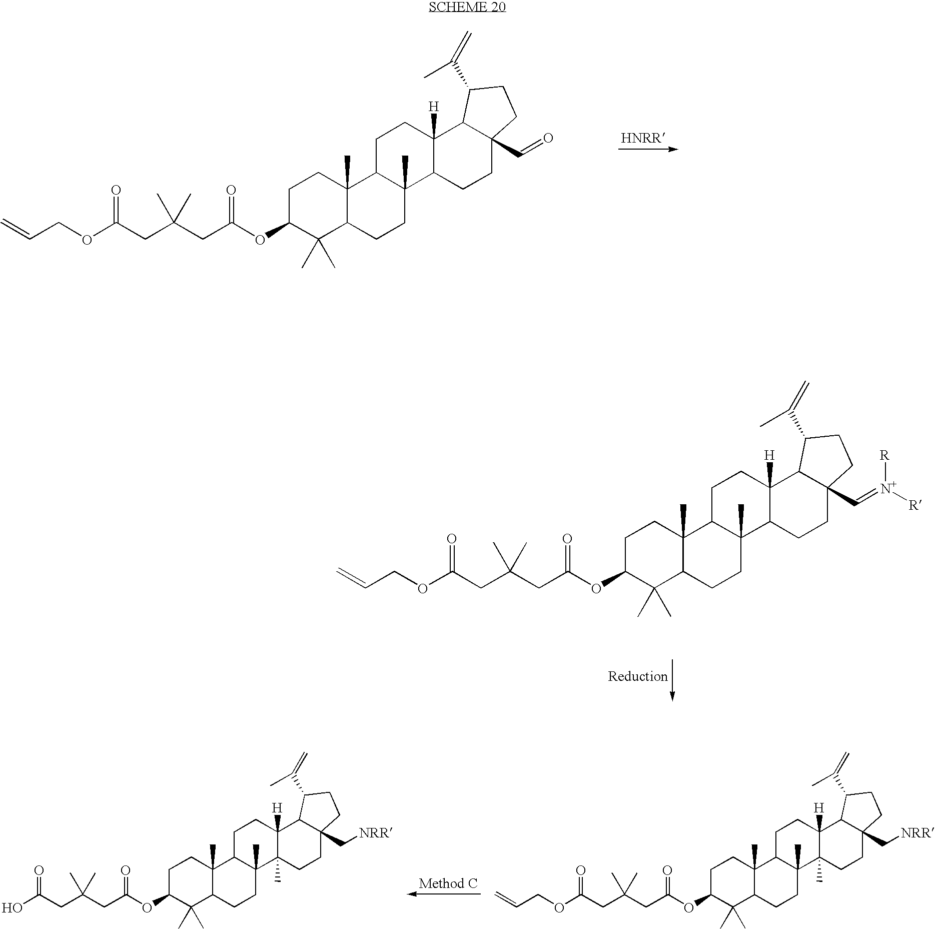

3-O-(3′,3′-Dimethylglutaryl)-28-aminolup-20(29)-enes can be obtained in two steps by reacting the desired primary or secondary amine with 3-O-(5′-allyloxy-3′,3′-dimethylglutaryl)-28-oxolup-20(29)-ene, followed by the reduction of the intermediate imine under standard conditions.

### 3-O-(5′-Allyloxy-3′,3′-dimethylglutaryl)-28-oxolup-20(29)-ene

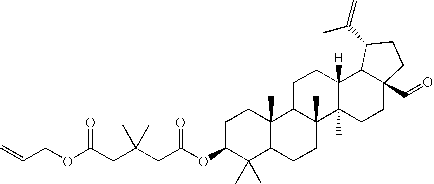

A solution of 3-O-(5′-allyloxy-3′,3′-dimethylglutaryl)betulin (370 mg) in dichloromethane (4 mL) was added to a suspension of Dess-Martin periodinate (290 mg) in dichloromethane (3 mL) and left stirring at rt for three hours. The reaction mixture was washed three times with 1M sodium hydroxide, dried over Na₂SO₄ and concentrated to yield 381 mg of crude 3-O-(5′-allyloxy-3′,3′-dimethylglutaryl)-28-oxolup-20(29)-ene. This compound was used without further purification.

### EXAMPLE 9

### Synthesis of C-28 Amines (28-Aminolup-20(29)-enes) from Betulinic Acid

3-O-(3′,3′-Dimethylglutaryl)-28-aminolup-20(29)-enes can be prepared in six steps from betulinic acid as shown in Scheme 21.

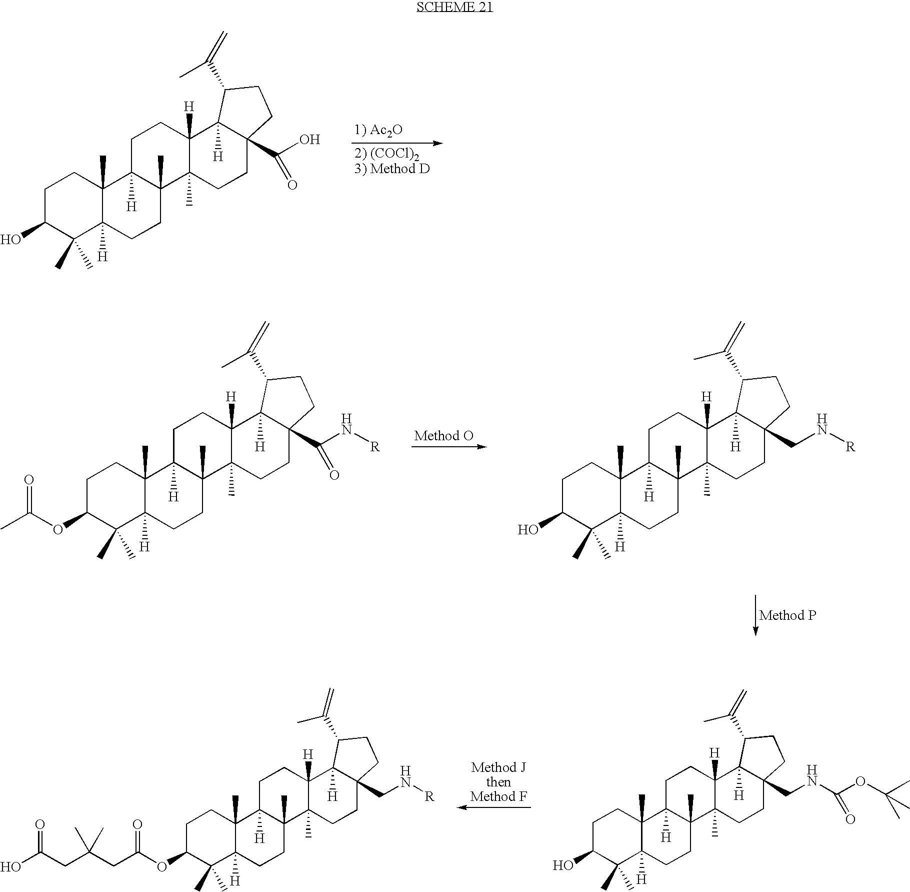

Betulinic acid was converted to the appropriate 3-O-acetylbetulinic acid C-28 amide as previously described (Scheme 11). Lithium aluminum hydride (LAH) reduction of the amides to the corresponding amines via method O was accompanied by deacetylation. The resulting amino alcohols were selectively N-Boc protected using method P. Final introduction of the glutaric side chain in the C-3 position using method J and then method F afforded the 3-O-(3′,3′-dimethylglutaryl)-28-aminolup-20(29)-enes.
Method O: Reduction of Betulinic Amides.

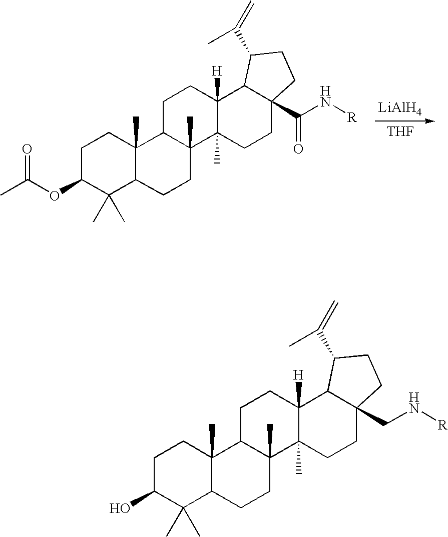

A solution of 3-O-(acetyl)betulinic acid amide (1 equivalent) in dry THF was stirred under nitrogen while adding a solution of LAH in THF (1 M in THF, 4 equivalents). The reaction mixture was heated at 45° C. for 16 hours. The reaction was carefully quenched with a solution of K₂CO₃ (1 M) and extracted several times with EtOAc. The combined organic layers were dried over Na₂SO₄ and concentrated in vacuo to give the desired compound as a white solid which was used without further purification.
Method P: Boc Protection of 28-aminolup-20(29)-ene.

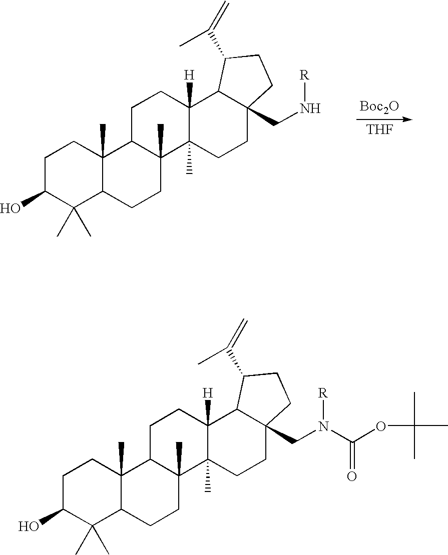

Di-tert-butyl dicarbonate (1.1 eq.) was added to a solution of 28-aminolup-20(29)-ene (1 eq.) in dry THF (5 mL) and left stirring at rt for three hours. The reaction mixture was then diluted with methanol and all organic solvents were removed in vacuo to yield a crude solid which was used without further purification.

### N-Alkylated-3-O-(3′,3′-dimethylglutaryl)-28-aminolup-20(29)-ene derivatives

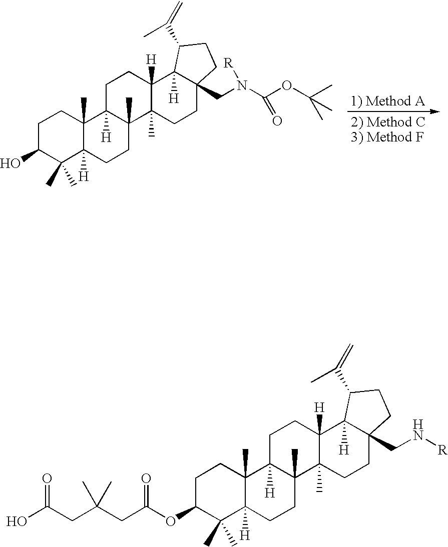

3-O-(3′,3′-Dimethylglutaryl)-28-(t-butoxycarbonylamino)lup-20(29)-enes can be prepared applying method A (acetylation with allyl 3,3′-dimethylglutaryl chloride) followed by method C (de-allylation) and method F (Boc deprotection).

### EXAMPLE 10

### Synthesis of Betulin C-28 Reverse Amides (28-Acylaminolup-20(29)-enes)

3-O-(3′,3′-Dimethylglutaryl)-28-acylaminolup-20(29)-enes can be prepared in four steps from 3-O-(acetyl)betulinic acid as shown in Scheme 22.

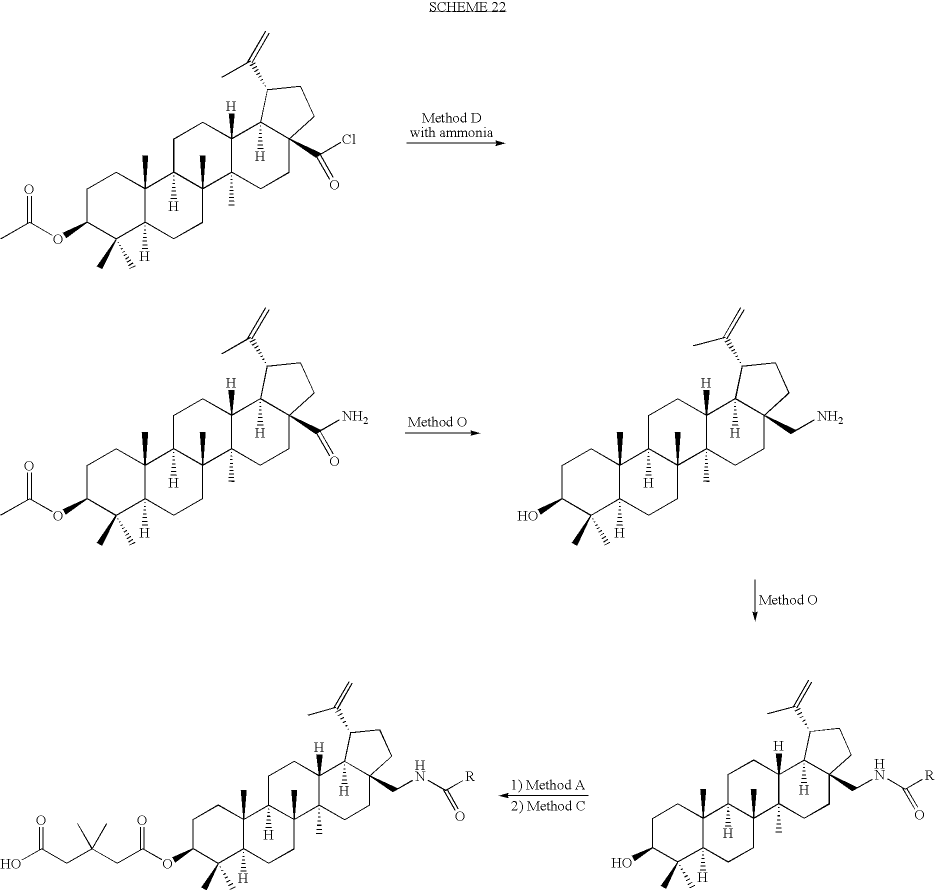

3-O-Acetylbetulinic acid was converted into the C-28 primary amide using method D. Reduction to the amino alcohol using method O was followed by selective N-acylation using method Q. Finally the glutaric side chain can be introduced using method A followed by method C to yield the desired reverse amide.
Method Q: Amide Coupling.

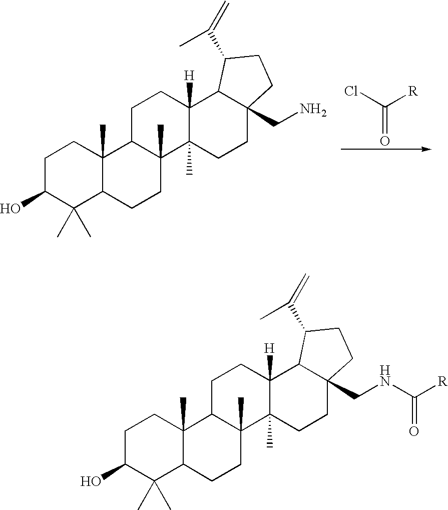

A solution of the desired acid chloride (2 equivalents) in dichloromethane was added to a solution of 28-aminolup-20(29)-ene (1 equivalent) and DIPEA in dry dichloromethane and the reaction stirred at rt for three hours. Methanol was added and the mixture diluted with dichloromethane and washed twice with 1 M HCl. The organic phase was dried over Na₂SO₄ and concentrated in vacuo to give the desired crude product, which can be used without further purification.

### 3-O-Acetylbetulinic acid amide

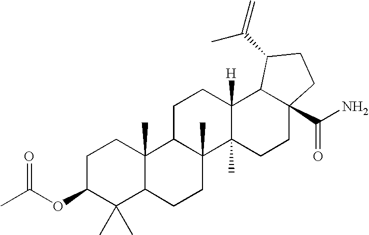

The compound was synthesized from 3-O-acetylbetulinic acid applying method D with 7 M ammonia in methanol. Purification by flash column chromatography gave the desired compound (230 mg, 43%). R_{f} 0.4 (EtOAc:Heptane 38:62).

### 28-Aminolup-20(29)-ene

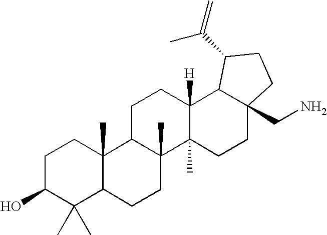

A solution of LAH in THF (1 M, 2 mL) was added to a solution of 3-O-acetylbetulinic acid amide (230 mg, 0.46 mmol) in dry THF (3 mL) and the reaction was stirred at 45° C. for 16 hours. The reaction was carefully quenched with 1 M potassium carbonate, and extracted several times with EtOAc. The organic phase was dried over Na₂SO₄ and concentrated in vacuo to give the desired crude 28-aminolup-20(29)-ene (170 mg) which was used without further purification.

### tert-Butoxycarboxamide N-[3-O-(3′,3′-dimethylglutaryl)lup-20(29)-en-28-yl]

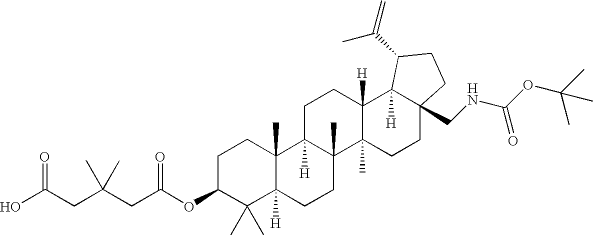

28-Aminolup-20(29)-ene was sequentially N-Boc protected using method P, acylated with allyl 3,3′-dimethylglutaryl chloride using method A and deallylated using method C. ¹H NMR (400 MHz, CDCl₃); δ ppm 4.68 (1H, m), 4.58 (1H, m), 4.52-4.47 (1H, dd, J=4.6, 10.8 Hz), 4.41-4.34 (1H, m), 3.32-3.27 (1H, dd, J=5.4, 13.4 Hz), 2.97-2.92 (1H, dd, J=6.8, 13.7 Hz), 2.49-2.38 (4H, m), 2.07-1.97 (1H, m), 1.75-0.77 (48H, m); LCMS, 87% pure, R_{f}=5.21; m/z (relative intensity) 707 ([M+Na⁺] 55%).

### 3-O-(3′,3′-dimethylglutaryl)lup-20(29)-en-28-ylamine

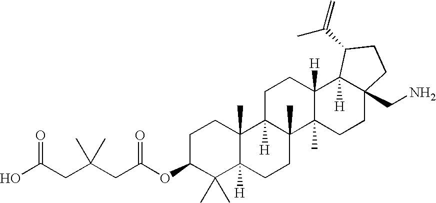

Trifluoroacetic acid (ca. 10 equivalents) was added to a solution of tert-butoxycarboxamide N-[3-O-(3′,3′-dimethylglutaryl)lup-20(29)-en-28-yl] in dichloromethane at 0° C. Cooling was removed and the reaction mixture allowed to warm to rt over 2 hrs. The reaction mixture was concentrated to dryness in vacuo, re-diluted in dichloromethane and re-evaporated. Dilution and evaporation was twice repeated. The crude contained two compounds that were separated by flash column chromatography to yield two products:

### 3-O-(3′,3′-dimethylglutaryl)lup-20(29)-en-28-ylamine

¹H NMR (250 MHz, CD₃OD); δ ppm 4.66 (1H, m), 4.59 (1H, m), 4.40-4.34 (2H, m), 3.08-3.02 (1H, m), 2.68-2.62 (1H, m), 2.42-2.28 (4H, m), 2.04-1.85 (1H, m), 1.74-0.75 (50H, m); LCMS, 95% pure, R_{f}=4.03; m/z (relative intensity) 585 ([M+H⁺] 100%).

### Trifluoromethylcarboxamide N-[3-O-(3′,3′-dimethylglutaryl)lup-20(29)-en-28-yl]

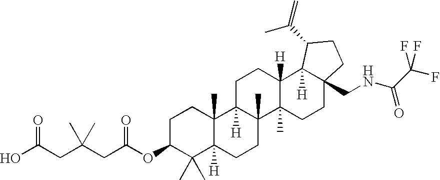

¹H NMR (250 MHz, CD₃OD); δ ppm 6.15 (1H, br s), 4.71 (1H, m), 4.62 (1H, m), 4.52-4.46 (2H, m), 3.67-3.59 (1H, dd, J=6.8, 14.7 Hz), 3.16-3.08 (1H, dd, J=5.9, 13.6 Hz), 2.50-2.36 (4H, m), 2.10-1.93 (1H, m), 1.77-0.75 (48H, m); LCMS, 95% pure, R_{f}=4.97; m/z (relative intensity) 703 ([M+Na⁺] 100%).

### EXAMPLE 11

### Pharmacological Activity

The biological evaluation of HIV-1 inhibition can be carried out as follows according to established protocols (Montefiori, D. C., et al., Clin. Microbiol. 26:231-235 (1988); Roehm, N., et al. J. Immunol. Methods 142:257-265 (1991)).

The human T-cell line, MT-2, was maintained in continuous culture with complete medium (RPMI 1640 with 10% fetal calf serum supplemented with L-glutamine at 5% CO₂ and 37° C.). Test samples were first dissolved in dimethyl sulfoxide at a concentration of 10 mg/mL to generate master stocks with dilutions made into tissue culture media to generate working stocks. The final drug concentrations used for screening were 25. 2.5, 0.25, and 0.025 μg/mL. For agents found to be active, additional dilutions were prepared for subsequent testing so that an accurate EC₅₀ value (defined below) could be determined. Test samples were prepared in duplicate (45 μL/well) and to each sample well was added 9011 of media containing MT-2 cells at 3×10⁵ cells/mL and 45 μL of virus inoculum (HIV-1 IIIIB isolate) at a concentration necessary to result in 50% killing of the cell targets at 5 days post-infection (PI). Control wells containing virus and cells only (no drug) and cells only (no virus or drug) were also prepared. A second set of samples were prepared identical to the first and were added to cells under identical conditions without virus (mock infection) for toxicity determinations (IC₅₀ defined below). In addition, AZT was also assayed during each experiment as a positive drug control. On day 5 PI, virus-induced cell killing was determined by measuring cell viability using the XTT method (Roehm, N., et al., supra). Compound toxicity was determined by XTT using the mock-infected samples. If a test sample had suppressive capability and was not toxic, its effects were reported in the following terms: IC₅₀, the concentration of test sample which is toxic to 50% of the mock-infected MT-2 cells; EC₅₀, the concentration of the test sample that is able to suppress HIV replication by 50%; and the Therapeutic index (TI) the ratio of the IC₅₀ to EC₅₀. The effective (EC₅₀) and inhibitory (IC₅₀) concentrations for anti-HIV activity and cytotoxicity, respectively, were determined (Roehm, N., et al., supra).

The biological evaluation of HIV-1 inhibition for compounds 49, 206, 218, 223, 227, 231, 235, 246, 248, 250, 252, 254, 256, 258, 260, 262, 264, 266, 291, 293, 297, 301, 309, 311, 315, 319, 321, 325, 329, 333, 337, 341, 345, 349, 353, 357, 361, 365, 369, 373, 377, 381, 409, 413, 429, 437, 441, 445, 449, 453, 457, 461, 465, 469, 473, 477, 481, 485, 493, 501, 505, 509, 672, 674, 676, 687, 689, 693, 697, 701, 705, 709, 717, 721, 725, 805, 821, 825, 829, 833, 837, 841, 845, 849, 853, 913, 1013, 1017, 1065, and 1137 was determined as described above. The anti-HIV activity (EC₅₀) for these compounds ranged from about 0.001 μM to about 0.30 μM. The cytotoxicity (IC₅₀) ranged from about 5 μM to about 100 μM. All data represented as an average of at least two experiments.

The following examples are illustrative, but not limiting, of the methods and compositions of the present invention. Other suitable modifications and adaptations of the variety of conditions and parameters normally encountered and obvious to those skilled in the art are within the spirit and scope of the invention.

Those skilled in the art will recognize that while specific embodiments have been illustrated and described, various modifications and changes can be made without departing from the spirit and scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All publications, patent applications and patents cited herein are fully incorporated by reference.

1. A compound of Formula I: 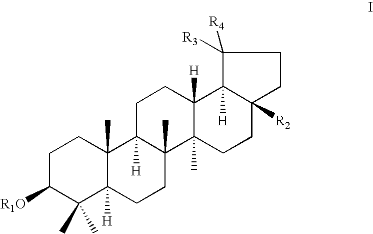
or a pharmaceutically acceptable salt or prodrug thereof, wherein:
R₁ is C₃-C₂₀ alkanoyl, carboxyalkanoyl, carboxyalkenoyl, alkoxycarbonylalkanoyl, alkenyloxycarbonylalkanoyl, cyanoalkanoyl, hydroxyalkanoyl, aminocarbonylalkanoyl, hydroxyaminocarbonylalkanoyl, monoalkylaminocarbonylalkanoyl, dialkylaminocarbonylalkanoyl, heteroarylalkanoyl, heterocyclylalkanoyl, heterocycylcarbonylalkanoyl, heteroarylaminocarbonylalkanoyl, heterocyclylaminocarbonylalkanoyl, cyanoaminocarbonylalkanoyl, alkylsulfonylaminocarbonylalkanoyl, arylsulfonylaminocarbonylalkanoyl, sulfoaminocarbonylalkanoyl, phosphonoaminocarbonylalkanoyl, phosphono, sulfo, phosphonoalkanoyl, sulfoalkanoyl, alkylsulfonylalkanoyl, or alkylphosphonoalkanoyl;
R₂ is formyl, carboxyalkenyl, heterocyclyl, heteroaryl, —CH₂SR₁₄, —CH₂SOR₁₄, —CH₂SO₂R₁₄, 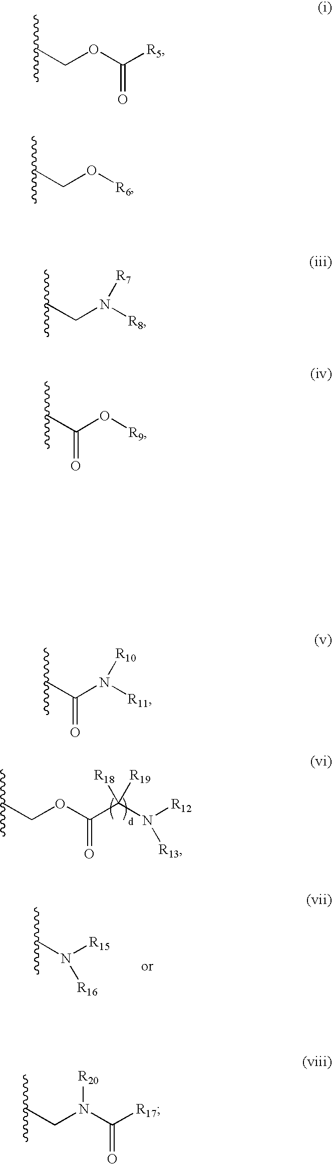
R₃ is hydrogen, hydroxyl, isopropenyl, isopropyl, 1′-hydroxyisopropyl, 1′-haloisopropyl, 1′-thioisopropyl, 1′-trifluoromethylisopropyl, 2′-hydroxyisopropyl, 2′-haloisopropyl, 2′-thioisopropyl, 2′-trifluoromethylisopropyl, 1′-hydroxyethyl, 1′-(alkoxy)ethyl, 1′-(alkoxyalkoxy)ethyl, 1′-(arylalkoxy)ethyl; 1′-(arylcarbonyloxy)ethyl, acetyl, 1′-(hydroxyl)-1′-(hydroxyalkyl)ethyl, (2′-oxo)tetrahydrooxazolyl, 1′,2′-epoxyisopropyl, 2′-haloisopropenyl, 2′-hydroxyisopropenyl, 2′-aminoisopropenyl, 2′-thioisopropenyl, 3′-haloisopropenyl, 3′-hydroxyisopropenyl, 3′-aminoisopropenyl, 3′-thioisopropenyl, 1′-alkoxyethyl, 1′-hydroxyiminoethyl, 1′-alkoxyimino, or 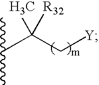
wherein Y is —SR₃₃ or —NR₃₃R₃₄;
R₃₂ is hydrogen or hydroxy;
R₃₃ and R₃₄ are independently hydrogen, alkyl, alkanoyl, arylalkyl, heteroarylalkyl, arylsulfonyl or arylaminocarbonyl; or
R₃₃ and R₃₄ can be taken together with the nitrogen to which they are attached to form a heterocycle, wherein the heterocycle can optionally include one or more additional nitrogen, sulfur or oxygen atoms;
m is zero to three;
R₄ is hydrogen; or
R₃ and R₄ can be taken together to form oxo, alkylimino, alkoxyimino or benzyloxyimino;
R₅ is C₂-C₂₀ alkyl, alkenyl, alkynyl, carboxy(C₂-C₂₀)alkyl, amino, aminoalkyl, dialkylamino, monoalkylaminoalkyl, dialkylaminoalkyl, alkoxyalkyl, cyanoalkyl, alkylthioalkyl, cycloalkyl, cycloalkylalkyl, aryl, arylalkyl, heterocyclyl, heteroaryl, heterocyclylalkyl, heteroarylalkyl, sulfo, phosphono, sulfoalkyl, phosphonoalkyl, alkylsulfonyl, alkylphosphono, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, arylphosphonoaminocarbonylalkyl, alkylphosphonoaminocarbonylalkyl, or hydroxyimino(amino)alkyl;
R₆ is hydrogen, phosphono, sulfo, alkyl, sulfoalkyl, phosphonoalkyl, alkylsulfonyl, alkylphosphono, cycloalkyl, cycloalkenyl, heterocyclyl, aryl, heteroaryl, carboxyalkyl, alkoxycarbonylalkyl, cyanoalkyl; CH₂CONR₇R₈, trialkylsilyl, ethoxyethyl, or tetrahydropyranyl ether;
R₇ and R₈ are independently hydrogen, alkyl, aminoalkyl, monoalkylaminoalkyl, dialkylaminoalkyl, carboxyalkyl, alkoxyalkyl, alkoxyalkoxyalkyl, alkoxycarbonylaminoalkoxyalkyl, alkoxycarbonylaminoalkyl, aminoalkoxyalkyl, alkylcarbonylaminoalkyl, heterocyclyl, heterocyclylalkyl, aryl, arylalkyl, arylcarbonylaminoalkyl, alkylsulfonyl, arylsulfonyl, heteroarylsulfonyl, heterocyclylsulfonyl, or cycloalkyl, or R₇ and R₈ can together with the nitrogen atom to which they are attached form a heterocyclyl or heteroaryl group, wherein the heterocyclyl or heteroaryl can optionally include one or more additional nitrogen, sulfur or oxygen atoms;
R₉ is hydrogen, phosphono, sulfo, alkyl, alkenyl, trialkylsilyl, cycloalkyl, carboxyalkyl, alkoxycarbonyloxyalkyl, aminoalkyl, monoalkylaminoalkyl, dialkylaminoalkyl, alkoxyalkyl, cyanoalkyl, phosphonoalkyl, sulfoalkyl, alkylsulfonyl, alkylphosphono, aryl, heteroaryl, heterocyclyl, arylalkyl, heteroarylalkyl, heterocyclylalkyl, or dialkoxyalkyl;
R₁₀ and R₁₁ are independently hydrogen, alkyl, amino, aminoalkyl, monoalkylaminoalkyl, dialkylaminoalkyl, carboxyalkyl, alkanoyloxyalkyl, alkoxyalkyl, hydroxyalkyl, cyanoalkyl, alkoxyalkoxyalkyl, alkoxycarbonylaminoalkoxyalkyl, alkoxycarbonylaminoalkyl, alkoxycarbonylalkyl, hydroxyalkoxyalkyl, aminoalkoxyalkyl, alkylcarbonylaminoalkyl, heterocyclyl, heterocyclylalkyl, aryl, heteroarylalkyl, arylalkyl, arylcarbonylaminoalkyl, alkylsufonyl, arylsulfonyl, alkylsulfonylaminoalkyl, arylsulfonylaminoalkyl, or cycloalkyl, or alkyl interrupted by one or more oxygen atoms, or R₁₀ and R₁ can together with the nitrogen atom to which they are attached form a heterocyclyl group, wherein the heterocyclyl can optionally include one or more additional nitrogen, sulfur or oxygen atoms;
R₁₂ and R₁₃ are independently hydrogen, alkyl, alkenyl, alkylamino, alkynyl, alkoxy, alkoxycarbonyl, alkoxyaminoalkyl, cycloalkyloxo, heterocyclylaminoalkyl, cycloalkyl, cyanoalkyl, cyano, sulfo, phosphono, sulfoalkyl, phosphonoalkyl, alkylsulfonyl, alkylphosphono, alkoxyalkyl, heteroaryl, heteroarylalkyl, dialkylaminoalkyl, heterocyclylalkyl, or R₁₂ and R₁₃ can together with the nitrogen atom to which they are attached form a heterocyclyl group or a heteroaryl group, wherein the heterocyclyl or heteroaryl can optionally include one or more additional nitrogen, sulfur or oxygen atoms, or R₁₂ and R₁₃ can together with the nitrogen atom to which they are attached form an alkylazo group, and d is one to six;
R₁₄ is hydrogen, alkyl, alkenyl, arylalkyl, carboxyalkyl, carboxyalkenyl, alkoxycarbonylalkyl, alkenyloxycarbonylalkyl, cyanoalkyl, hydroxyalkyl, carboxybenzyl, aminocarbonylalkyl;
R₁₅ and R₁₆ are independently hydrogen, alkyl, alkoxycarbonyl, alkoxyaminoalkyl, cyclo(oxo)alkyl, cycloalkylcarbonyl, heterocyclylaminoalkyl, cycloalkyl, cyanoalkyl, cyano, sulfo, phosphono, sulfoalkyl, phosphonoalkyl, alkylsulfonyl, alkylphosphono, alkoxyalkyl, heterocyclylalkyl, or R₁₅ and R₁₆ can together with the nitrogen atom to which they are attached form a heterocyclyl group, wherein the heterocyclyl can optionally include one or more additional nitrogen, sulfur or oxygen atoms, or R₁₅ and R₁₆ can together with the nitrogen atom to which they are attached form an alkylazo group;
R₁₇ is hydrogen, alkyl, perhaloalkyl, alkoxy, alkenyl, carboxyalkyl, amino, aminoalkyl, monoalkylaminoalkyl, dialkylaminoalkyl, alkoxyalkyl, alkoxycarbonyl, cyanoalkyl, alkylthioalkyl, alkylsulfinylalkyl, alkylsulfonylalkyl, cycloalkyl, cycloalkylalkyl, aryl, arylalkyl, heterocyclyl, heteroaryl, heterocyclylalkyl, heteroarylalkyl, alkanoylaminoalkyl, aminocarbonylalkyl, alkylaminocarbonylalkyl, dialkylaminocarbonylalkyl, heterocyclylcarbonylalkyl, cycloalkylcarbonylalkyl, heteroarylalkylaminocarbonylalkyl, arylalkylaminocarbonylalkyl, heterocyclylalkylaminocarbonylalkyl, carboxyalkylaminocarbonylalkyl, arylsulfonylaminocarbonylalkyl, alkylsulfonylaminocarbonylalkyl, or hydroxyimino(amino)alkyl;
R₁₈ and R₁₉ are independently hydrogen, methyl or ethyl; d is one to six; and
R₂₀ is hydrogen, C₁-C₆ alkyl, or aryl;
wherein any alkyl, aryl, cycloalkyl, heterocyclyl, or heteroaryl group, or any substitutent which includes any of these groups, is optionally substituted;
when R₁ is C₃-C₂₀ alkanoyl, carboxyalkanoyl or alkoxycarbonyl, and R₃ is isopropenyl, isopropyl, 2′-hydroxyisopropyl, 2′-haloisopropyl, or 2′-thioisopropyl, and R₂ is formula is formula (i), formula (ii) or formula (Iv), then R₅ cannot be C₂-C₂₀ alkyl or carboxy(C₂-C₂₀)alkyl, or R₆ cannot be hydrogen or carboxyalkyl, or R₉ cannot be hydrogen;
when R₁ is carboxyalkanoyl, and R₃ is isopropenyl, isopropyl, isobutyl, isobutenyl, or 2′-hydroxyisopropyl, and R₂ is formula (ii), formula (Iv) or formula (v), then R₆ cannot be alkyl, R₉ cannot be alkyl or carboxyalkyl, and R₁₀ and R₁₁ cannot be carboxyalkyl;
when R₁ is carboxyalkenoyl, R₂ is formula (ii), and R₃ is isopropenyl, then R₆ cannot be hydrogen; and
when R₁ is 3′,3′-dimethylsuccinyl, R₂ is formula (Iv), and R₉ is hydrogen, then R₃ cannot be 1′-hydroxyethyl, 1′-(oxo)ethyl or 1′-(alkoxy)ethyl.

2. A compound according to claim 1, wherein R₁ is carboxyalkanoyl.

3. A compound according to claim 2, wherein R₁ is a carboxyalkanoyl selected from the group consisting of

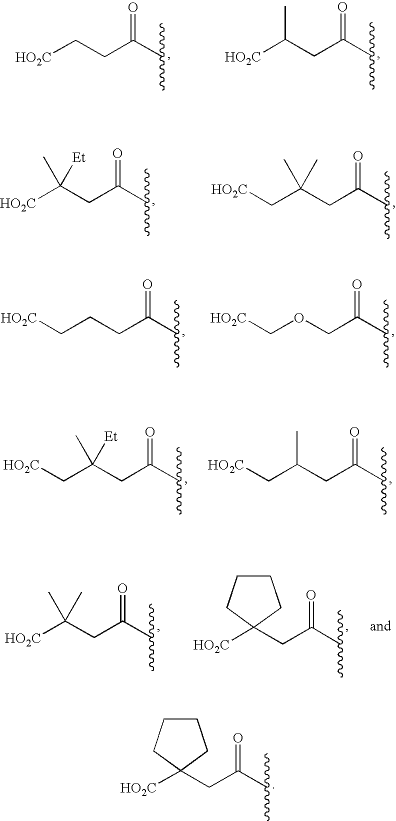

4. A compound according to claim 3, wherein R₁ is a carboxyalkanoyl, wherein said carboxyalkanoyl is succinyl, glutaryl, 3′-methylglutaryl, 3′-methylsuccinyl, 3′,3′-dimethylsuccinyl, or 3′,3′-dimethylglutaryl.

5. A compound according to claim 1, wherein R₁ is alkenyloxycarbonylalkanoyl, wherein said alkenyloxycarbonylalkanoyl is a C₁-C₄ alkene ester of succinyl, glutaryl, 3′-methylglutaryl, 3′-methylsuccinyl, 3′,3′-dimethylsuccinyl or 3′,3′-dimethylglutaryl.

6. A compound according to claim 5, wherein the C₁-C₄ alkene ester is an allyl ester of succinyl, glutaryl, 3′-methylglutaryl, 3′-methylsuccinyl, 3′,3′-dimethylsuccinyl or 3′,3′-dimethylglutaryl.

7. A compound according to claim 1, wherein R₁ is alkoxycarbonylalkanoyl, wherein said alkoxycarbonylalkanoyl is a C₁-C₄ alkyl ester of succinyl, glutaryl, 3′-methylglutaryl, 3′-methylsuccinyl, 3′,3′-dimethylsuccinyl or 3′,3′-dimethylglutaryl.

8. A compound according to claim 7, wherein the C₁-C₄ alkyl ester is an ethyl or propyl ester of succinyl, glutaryl, 3′-methylglutaryl, 3′-methylsuccinyl, 3′,3′-dimethylsuccinyl or 3′,3′-dimethylglutaryl.

9. A compound according to claim 1, wherein R₁ is alkanoyl, wherein said alkanoyl is tert-butylcarbonyl or isopropylcarbonyl.

10. A compound according to claim 1, wherein R₁ is carboxyalkanoyl, wherein said carboxyalkanoyl is 2′,2′-dimethylmalonyl, 2′,3′-dihydroxysuccinyl, 2′,2′,3′,3′-tetramethylsuccinyl, 3′-methylsuccinyl, or 2′,2′-dimethylsuccinyl.

11. A compound according to claim 1, wherein R₁ is carboxyalkenoyl, wherein said carboxyalkenoyl is alk-2-enyloyl.

12. A compound according to claim 1, wherein R₁ is cyanoalkanoyl, wherein said cyanoalkanoylalkanoyl is 4′-cyanopropanoyl or 4′-cyanobutanoyl.

13. A compound according to claim 1, wherein R₁ is hydroxyalkanoyl, wherein said hydroxyalkanoyl is 3′,3′-dimethyl-4′-hydroxybutanoyl.

14. A compound according to claim 1, wherein R₁ is aminocarbonylalkanoyl, wherein said aminocarbonylalkanoyl is 4′-amino-3′,3′-dimethylsuccinyl or 4′-aminosuccinyl.

15. A compound according to claim 1, wherein R₁ is alkylsulfonylaminocarbonylalkanoyl, wherein said alkylsulfonylaminocarbonylalkanoyl is 4′-methylsulfonylamino-3′,3′-dimethylsuccinyl.

16. A compound according to claim 1, wherein R₁ is arylsulfonylaminocarbonylalkanoyl, wherein said arylsulfonylaminocarbonylalkanoyl is 4′-phenylsulfonylamino-3′,3′-dimethylsuccinyl.

17. A compound according to claim 1, wherein R₁ is heterocycloalkanoyl, wherein said heteroarylalkanoyl is tetrazolylalkanoyl.

18. A compound according to claim 1, wherein R₁ is phosphonoalkyl, wherein said phosphonoalkyl is C₁-C₆ phosphonoalkyl.

19. A compound according to claim 1, wherein R₁ is sulfoalkyl, wherein said sulfoalkyl is C₁-C₆ sulfoalkyl.

20. A compound of claim 1, wherein R₂ is heterocyclyl, and said heterocyclyl is selected from the group consisting of oxazolyl, morpholinyl, piperidinyl, piperazinyl, dihydropyrrolyl, piperidinyl, and dihydrofuranyl.

21. A compound of claim 1 wherein R₂ is (i) and R₅ is alkyl, wherein said alkyl is selected from the group consisting of C₁-C₆ alkyl.

22. A compound of any one of claim 1 wherein R₂ is (i) and R₅ is alkenyl, wherein said alkenyl is selected from the group consisting of propen-2-yl, buten-2-yl, and penten-2-yl.

23. A compound of any one of claim 1 wherein R₂ is (i) and R₅ is C₂-C₁₀ carboxyalkyl, wherein said C₂-C₁₀ carboxyalkyl is 2′-carboxy-2′,2′-dimethylethyl or 3′-carboxy-3′,3′-dimethylpropyl.

24. A compound of any one of claim 1 wherein R₂ is (i) and R₅ is heterocyclyl, or heterocyclylalkyl.

25. A compound of claim 24, wherein said heterocyclyl is tetrazolyl, morpholinyl, pyridinyl, imidazolyl, isoxazolyl, or furanyl.

26. A compound of claim 24, wherein said heterocycloalkyl is a heterocyclo(C₁-C₆)alkyl.

27. A compound of claim 1, wherein R₂ is (ii) and R₆ is cycloalkyl or heterocycloalkyl.

28. A compound of claim 1, wherein R₂ is (ii) and R₆ is methylpyridinyl or cycloocten-2-yl.

29. A compound of claim 1, wherein R₂ is (ii) and R₆ is carboxyalkyl.

30. A compound of claim 1, wherein R₂ is (ii) and R₆ is alkoxycarbonylalkyl.

31. A compound of claim 1, wherein R₂ is (ii) and R₆ is cyanoalkyl.

32. A compound of claim 1, wherein R₂ is (iii) and R₇ and R₈ are independently alkoxyalkylamine or hydrogen.

33. A compound of claim 1, wherein R₂ is (iii) and R₇ and R₈ together with the nitrogen atom to which they are attached form a heterocyclyl group, wherein the heterocyclyl group can optionally include one or more additional nitrogen, sulfur or oxygen groups.

34. A compound of claim 33, wherein said heterocyclyl group is pyrrolyl, morpholinyl, or piperazinyl.

35. A compound of claim 1 wherein R₂ is (v) and R₁₀ and R₁₁ are both hydrogen.

36. A compound of claim 1 wherein R₂ is (v) and R₁₀ and R₁₁ are independently alkyl, aminoalkyl, aminoalkoxyalkyl, alkoxycarbonylamino, alkoxycarbonylalkyl, cyanoalkyl, alkylsulfonyl, alkoxyalkyl, cycloalkyl, alkoxycarbonylaminoalkoxyalkyl, alkoxycarbonylaminoalkyl, alkylcarbonylaminoalkyl, alkoxyalkoxyalkyl, or dialkylaminoalkyl.

37. The compound of claim 36, wherein R₂ is (v), one of R₁₀ and R₁₁ is hydrogen, and one of R₁₀ and R₁₁ is alkyl, wherein the alkyl group is selected from methyl, 2-hydroxyethyl, 2-hydroxy-2-methylpropyl, propyl, ethyl, isopropyl, (R)-2-[2,3-dihydroxypropyl], (S)-2-[2,3-dihydroxypropyl], (S)-2-[1-hydroxy-4-methylpentyl)], (R)-2-[1-hydroxy-4-methylpentyl)], or (S)-1-carboxy-3-methylbutyl.

38. The compound of claim 36, wherein R₂ is (v), one of R₁₀ and R₁₁ is hydrogen, and one of R₁₀ and R₁₁ is aminoalkyl, wherein the aminoalkyl is 2-(1-amino-2-methylpropyl).

39. The compound of claim 36, wherein R₂ is (v), one of R₁₀ and R₁₁ is hydrogen, and one of R₁₀ and R₁₁ is alkoxyalkyl, wherein the alkoxyalkyl group is 2-methoxyethyl or 2-hydroxyethoxyethyl.

40. The compound of claim 36, wherein R₂ is (v), one of R₁₀ and R₁₁ is hydrogen, and one of R₁₀ and R₁₁ is alkoxycarbonylaminoalkyl, wherein the alkoxycarbonylaminoalkyl group is 2-(tert-butoxycarbonylamino)ethyl.

41. The compound of claim 36, wherein R₂ is (v), one of R₁₀ and R₁₁ is hydrogen, and one of R₁₀ and R₁₁ is dialkylaminoalkyl, wherein the dialkylaminoalkyl group is 2-N,N-dimethylaminoethyl, 2-N,N-dimethylaminopropyl, (1R,3R)-3-N,N-dimethylaminocyclopentyl, or (1S,3S)-3-N,N-dimethylaminocyclopentyl.

42. A compound of claim 1 wherein R₂ is (v) and one of R₁₀ and R₁₁ is hydrogen, and one of R₁₀ and R₁₁ is cycloalkyl, heterocyclyl, aryl, arylalkyl, arylcarbonylaminoalkyl, arylsulfonyl, heterocyclylheterocyclylalkyl, heterocyclylarylalkyl, arylaminoalkyl, aminocycloalkyl, or heterocycloalkyl.

43. The compound of claim 42, wherein R₂ is (v), one of R₁₀ and R₁₁ is hydrogen, and one of R₁₀ and R₁₁ is cycloalkyl, wherein the cycloalkyl group is cyclopropyl.

44. The compound of claim 42, wherein R₂ is (v), one of R₁₀ and R₁₁ is hydrogen, and one of R₁₀ and R₁₁ is heterocyclyl, wherein the heterocyclyl group is selected from (S)-1-[(tert-butoxycarbonyl)pyrrolidinyl], (R)-1-[(tert-butoxycarbonyl)pyrrolidinyl], (S)-3-pyrrolidinyl, (R)-3-pyrrolidinyl. (S)-3-(1-methylpyrrolidinyl), (R)-3-(1-methylpyrrolidinyl), (S)-3-(1-acetylpyrrolidinyl), (R)-3-(1-acetylpyrrolidinyl), (S)-3-(1-methylsulfonylpyrrolidinyl), (R)-3-(1-methylsulfonylpyrrolidinyl), 4-(1-(tert-butoxycarbonyl)piperdinyl), 4-piperidinyl, 4-(1-methylpiperidinyl), or 4-[1-(1-hydroxyethyl)piperidinyl)].

45. The compound of claim 42, wherein R₂ is (v), one of R₁₀ and R₁₁ is hydrogen, and one of R₁₀ and R₁₁ is aryl, wherein the aryl group is 4-fluorophenyl, 2-(1,3,4-thiadiazolyl)methyl, or 2,3-dichlorobenzyl, 4-azido-2,3,5,6-tetrafluorobenzyl.

46. The compound of claim 42, wherein R₂ is (v), one of R₁₀ and R₁₁ is hydrogen, and one of R₁₀ and R₁₁ is arylalkyl, wherein the arylalkyl group is selected from 4-fluorobenzyl, 3-fluorobenzyl, 2-fluorobenzyl, 4-chlorobenzyl, 3-chlorobenzyl, 2-chlorobenzyl, 4-methylbenzyl, 3-methylbenzyl, 2-methylbenzyl, 4-methyoxybenzyl, 3-methoxybenzyl, 2-methoxybenzyl, 4-N,N-dimethylaminobenzyl, 4-trifluoromethylbenzyl, 4-carboxybenzyl, 3,4-dichlorobenzyl, 2,4-dichlorobenzyl, 2-pyridinylmethyl, 3-pyridinylmethyl, 4-pyridinylmethyl, 2-benzyl, 3-trifluoromethylbenzyl, 4-tert-butylbenzyl, 4-aminobenzyl, 4-acetamidobenzyl, (R)-1-phenylethyl, (S)-1-phenylethyl, (R)-2-hydroxy-1-phenylethyl, (S)-2-hydroxy-1-phenylethyl, or 2-phenylethyl.

47. The compound of claim 42, wherein R₂ is (v), one of R₁₀ and R₁₁ is hydrogen, and one of R₁₀ and R₁₁ is heterocycloalkyl, wherein the heterocycloalkyl group is selected from 4-(1-methylimidazolyl)methyl, 3-(5-methylisoxazolyl)methyl, 3-(4-morpholinyl)propyl, 3-(1-imidazolyl)propyl, 2-(4-methylmorpholinyl)methyl, 2-morpholinylmethyl, or 2-(4-tert-butoxycarbonyl morpholinyl)methyl.

48. The compound of claim 42, wherein R₂ is (v), one of R₁₀ and R₁₁ is hydrogen, and one of R₁₀ and R₁₁ heterocyclylarylalkyl, wherein the heterocyclylarylalkyl group is selected from 4-(4-morpholinyl)benzyl or 4-(4-methylpiperazinyl)benzyl.

49. The compound of claim 42, wherein R₂ is (v), one of R₁₀ and R₁₁ is hydrogen, and one of R₁₀ and R₁₁ heterocyclylheterocyclylalkyl, wherein the heterocyclylheterocyclylalkyl group is 3-[6-(4-morpholinyl)pyridinyl]methyl.

50. The compound of claim 42, wherein R₂ is (v), one of R₁₀ and R₁₁ is hydrogen, and one of R₁₀ and R₁₁ arylaminoalkyl, wherein the arylaminoalkyl is 2-[(4-azido-2,3,5,6-tetrafluorobenzoyl)amino]ethyl.

51. The compound of claim 42, wherein R₂ is (v), one of R₁₀ and R₁₁ is hydrogen, and one of R₁₀ and R₁₁ aminocycloalkyl, wherein the aminocycloalkyl is (1R,3R)-3-aminocyclopentyl, (1S,3S)-3-aminocyclopentyl, (1r,4r)-4-aminocyclohexyl, or (1s,4s)-4-aminocyclohexyl.

52. The compound of claim 42, wherein R₂ is (v), one of R₁₀ and R₁₁ is hydrogen, and one of R₁₀ and R₁₁ dialkylaminocycloalkyl, wherein the dialkylaminocycloalkyl is (1r,4r)-4-N,N-dimethylaminocyclohexyl or (1s,4s)-4-N,N-dimethylaminocyclohexyl.

53. A compound of claim 1 wherein R₂ is (v) and R₁₀ and R₁₁ are taken together to form a heterocyclyl group, wherein said heterocyclyl group can optionally include one or more additional nitrogen, sulfur or oxygen atoms.

54. The compound of claim 1 wherein R₂ is (v) and R₁₀ and R₁₁ are taken together to form one of 4-(tert-butoxycarbonyl)piperazinyl, morpholinyl, piperidinyl, piperazinyl, 4-(4-morpholinylcarbonyl)piperazinyl, 4-methylpiperazinyl, 4-ethylpiperazinyl, 4-isopropylpiperazinyl, 4-(cyclopropylmethyl)piperazinyl, 4-benzylpiperazinyl, 4-[3-(5-methylisoxazolyl)methyl]piperazinyl, 4-(4-pyridinylmethyl)piperazinyl, 4-acetylpiperazinyl, 4-(isopropylaminocarbonyl)piperazinyl, 4-(methylsulfonyl)piperazinyl, 4-cyclopropylpiperazinyl, 4-(2-methoxyethylaminocarbonyl)piperazinyl, 4-(2-hydroxyethyl)piperazinyl, 4-(2-methoxyethyl)piperazinyl, 4-(3-dimethylaminopropyl)piperazinyl, 4-(aminocarbonyl)piperazinyl, 4-(aminosulfonyl)piperazinyl, 3-oxopiperazinyl, 4-methyl-3-oxopiperazinyl, 4-(hydroxyethyl)-3-oxopiperazinyl, 4-(2-hydroxybenzoyl)piperazinyl, 4-[3-(1,2,4-oxadiazolyl)methyl]piperazinyl, 4-[4-(dimethylaminosulfonyl)benzyl]piperazinyl, 4-[1-(1,2,3,4-tetrahydronaphthyl)]piperazinyl, 4-[4-(acetamidobenzyl)]piperazinyl, (1S,4S)-5-methyl-2,5-diazabicyclo[2.2.1]heptanyl, (1R,4R)-5-methyl-2,5-diazabicyclo[2.2.1]heptanyl, (1S,4S)-2,5-diazabicyclo[2.2.1]heptanyl, (1R,4R)-2,5-diazabicyclo[2.2.1]heptanyl, (1S,4S)-5-(tert-butoxycarbonyl)-2,5-diazabicyclo[2.2.1]heptanyl, (1R,4R)-5-(tert-butoxycarbonyl)-2,5-diazabicyclo[2.2.1]heptanyl, 4-(4-azido-2,3,5,6-tetrafluorobenzyl)piperazinyl, pyrrolidinyl, (R,S)-3-hydroxypyrrolidinyl, (R)-3-hydroxypyrrolidinyl, (S)-3-hydroxypyrrolidinyl, (R)-3-(tert-butoxycarbonylamino)pyrrolidinyl, (S)-3-(tert-butoxycarbonylamino)pyrrolidinyl, (R)-3-aminopyrrolidinyl, (S)-3-aminopyrrolidinyl, (R)-2-(hydroxymethyl)pyrrolidinyl, (S)-2-(hydroxymethyl)pyrrolidinyl, (S)-2-(hydroxymethyl)pyrrolidinyl, (S)-2-(hydroxymethyl)pyrrolidinyl, (S)-2-(hydroxymethyl)pyrrolidinyl, (R)-3-N-methylaminopyrrolidinyl, (S)-3-N-methylaminopyrrolidinyl, (R)-3-N,N-dimethylaminopyrrolidinyl, (S)-3-N,N-dimethylaminopyrrolidinyl, (R)-3-N,N-diethylaminopyrrolidinyl, (S)-3-N,N-diethylaminopyrrolidinyl, (R)-3-N-ethylaminopyrrolidinyl, (S)-3-N-ethylaminopyrrolidinyl, (R)-3-(4-morpholinyl)pyrrolidinyl, (S)-3-(4-morpholinyl)pyrrolidinyl, (R)-3-(1-pyrrolidinyl)pyrrolidinyl, (S)-3-(1-pyrrolidinyl)pyrrolidinyl, 4-aminopiperidinyl, 4-oxopiperidinyl, 4-hydroxypiperidinyl, 4-N,N-diaminopiperidinyl, 4-(4-morpholinyl)piperidinyl, 4-acetamidopiperidinyl, 4-(methylsulfonamide)piperidinyl, (R)-3-acetamidopyrrolidinyl, (S)-3-acetamidopyrrolidinyl, (R)-3-(cyclopropanecarboxamido)pyrrolidinyl, (S)-3-(cyclopropanecarboxamido)pyrrolidinyl, (R)-3-(2-hydroxyacetamido)pyrrolidinyl, (S)-3-(2-hydroxyacetamido)pyrrolidinyl, (R)-3-(methylsulfonamido)pyrrolidinyl, (S)-3-(methylsulfonamido)pyrrolidinyl, (R)-2-(aminomethyl)pyrrolidinyl, (S)-2-(aminomethyl)pyrrolidinyl, (R)-2-(N,N-dimethylaminomethyl)pyrrolidinyl, (S)-2-(N,N-dimethylaminomethyl)pyrrolidinyl, (R)-2-(acetamidomethyl)pyrrolidinyl, (S)-2-(acetamidomethyl)pyrrolidinyl, (R)-2-(methylsulfonamidomethyl)pyrrolidinyl, (S)-2-(methylsulfonamidomethyl)pyrrolidinyl, (R)-2-(N,N-diethylaminomethyl)pyrrolidinyl, (S)-2-(N,N-diethylaminomethyl)pyrrolidinyl, (R)-2-(4-morpholinylmethyl)pyrrolidinyl, (S)-2-(4-morpholinylmethyl)pyrrolidinyl, 2,6-dimethylmorpholinyl, 1,4-oxazepanyl, thiomorpholinyl, thiomorpholinyl 1-oxide, or thiomorpholinyl 1,1-dioxide.

55. A compound of claim 1, wherein R₂ is (vi) and R₁₂ and R₁₃ are hydrogen.

56. A compound of claim 1, wherein R₂ is (vi) and one of R₁₂ and R₁₃ are hydrogen and one of R₁₂ and R₁₃ is alkylamino, alkenyl, alkynyl, alkoxy, alkoxyalkyl, alkoxycarbonyl, cycloalkyl, cycloalkyloxo, heteroaryl, heteroarylalkyl, dialkylaminoalkyl, or cyanoalkyl.

57. A compound of claim 1, wherein R₂ is (vi) and R₁₂ and R₁₃ can together with the nitrogen atom to which they are attached form a heterocyclyl or heteroaryl, wherein the heterocyclyl or heteroaryl group can optionally include one or more additional nitrogen, sulfur or oxygen atoms.

58. A compound according to claim 1, wherein R₃ is R₃ is hydroxyl, isopropenyl, isopropyl, 1′-hydroxyisopropyl, 1′-haloisopropyl, 1′-thioisopropyl, 1′-trifluoromethylisopropyl, 2′-hydroxyisopropyl, 2′-haloisopropyl, 2′-thioisopropyl, 2′-trifluoromethylisopropyl, 1′-hydroxyethyl, 1′-(alkoxy)ethyl, 1′-(alkoxyalkoxy)ethyl, 1′-(arylalkoxy)ethyl; 1′-(arylcarbonyloxy)ethyl, acetyl, 1′-(hydroxyl)-1′-(hydroxyalkyl)ethyl, (2′-oxo)tetrahydrooxazolyl, or 1′,2′-epoxyisopropyl.

59. A compound according to claim 1, wherein R₄ is hydrogen, R₃ is 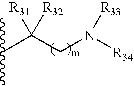
R₃₁ is hydrogen, R₃₂ is methyl, R₃₃ and R₃₄ are independently hydrogen, alkyl, alkanoyl, arylalkyl, heteroarylalkyl, arylsulfonyl or arylaminocarbonyl; or R₃₃ and R₃₄ can be taken together with the nitrogen to which they are attached to form heterocyclyl, wherein the heterocyclyl can optionally include one or more additional nitrogen, sulfur or oxygen atoms; and m is zero to three.

60. A compound according to claim 1, wherein R₂ is (i), and R₃ is isopropenyl.

61. A compound according to claim 1, wherein R₂ is (ii), and R₃ is isopropenyl.

62. A compound according to claim 1, wherein R₂ is (iii), and R₃ is isopropenyl.

63. A compound according to claim 1, wherein R₂ is (iv), and R₃ is isopropenyl.

64. A compound according to claim 1, wherein R₂ is (v), and R₃ is isopropenyl.

65. A compound according to claim 1, wherein
R₁ is succinyl, glutaryl, 3′-methylglutaryl, 3′-methylsuccinyl, 3′,3′-dimethylsuccinyl or 3′,3′-dimethylglutaryl, or an alkyl or allyl ester of succinyl, glutaryl, 3′-methylglutaryl, 3′-methylsuccinyl, 3′,3′-dimethylsuccinyl or 3′,3′-dimethylglutaryl;
R₂ is (i), (ii) or (iv); and
R₃ is isopropenyl.

66. A compound according to claim 56, wherein R₂ is (i), and R₅ is a heteroarylalkyl.

67. A compound according to claim 56, wherein R₂ is (ii), and R₆ is a heteroaryl.

68. A compound according to claim 56, wherein R₂ is (iv), and R₉ is cyanoalkyl.

69. A compound according to claim 1, wherein
R₁ is succinyl, glutaryl, 3′-methylglutaryl, 3′-methylsuccinyl, 3′,3′-dimethylsuccinyl or 3′,3′-dimethylglutaryl, or an alkyl or allyl ester of succinyl, glutaryl, 3′-methylglutaryl, 3′-methylsuccinyl, 3′,3′-dimethylsuccinyl or 3′,3′-dimethylglutaryl;
R₂ is (iii), (v) or (vi); and
R₃ is isopropenyl.

70. A compound according to claim 69, wherein R₂ is (iii), and R₇ and R₈ taken together with the nitrogen to which they are attached to form a heterocycloalkyl or heteroaryl.

71. A compound according to claim 69, wherein R₂ is (v), and R₁₀ and R₁₁ taken together with the nitrogen to which they are attached to form a heterocycloalkyl or heteroaryl.

72. A compound according to claim 69, wherein R₂ is (vi), and R₁₂ and R₁₃ taken together with the nitrogen to which they are attached to form a heterocycloalkyl or heteroaryl.

73. A pharmaceutical composition comprising a compound according to claim 1, and a pharmaceutically acceptable carrier.

74. A pharmaceutical composition according to claim 73, further comprising an antiviral agent or an immunostimulating agent.

75. A pharmaceutical composition according to claim 74, wherein said antiviral agent is selected from the group consisting of one or more of zidovudine, lamivudine, zalcitabine, stavudine, didanosine, tenofovir, abacavir, nevirapine, delavirdine, emtricitabine, efavirenz, saquinavir, ritonavir, indinavir, nelfinavir, lopinavir, amprenavir, fosamprenavir, tipranavir, atazanavir, enfuvirtide, hydroxyurea, interleukin-2, gamma globulin, amantadine, guanidine hydroxybenzimidazole, interferon-α, interferon-β, interferon-γ, a thiosemicarbazone, methisazone, rifampin, ribavirin, a pyrimidine analog, a purine analog, foscarnet, phosphonoacetic acid, acyclovir, a dideoxynucleoside, and ganciclovir.

76. A method of synthesizing a compound of Formula I wherein R₂ is formula (v), comprising
(a) forming a monoprotected di-carboxylic acid derivative;
(b) activating the non-protected carboxyl group of the di-carboxylic acid to form an acid halide;
(c) reacting the acid halide of step (b) with betulinic acid to form the R₁ group at the C-3 position;
(d) activating the C-28 position of the compound of (c) to form an acid halide;
(e) attaching the desired amine at C-28; and
(f) deprotecting the protected R₁ carboxyl group of (a).

77. A method of synthesizing a compound of Formula I wherein R₂ is formula (v), comprising:
(a) protecting a C-3 alcohol of betulinic acid;
(b) activating the C-3 protected betulinic acid at the C-28 carbon to form a C-3 protected, C-28 activated betulinic acid;
(c) the resulting compound of (b) reacting the C-3 protected, C-28 activated betulinic acid with an appropriated amine;
(d) deprotecting the the resulting compound of step (c) at its C-3 position and (e) adding an R₁ ester group at C-3.

78. A method for inhibiting a retroviral infection in cells or tissue of an animal comprising administering an effective retroviral inhibiting amount of a pharmaceutical composition according to claim 1.

79. A method according to claim 78 wherein said retroviral infection does not respond to other therapies.

80. A method for inhibiting a retroviral infection in cells or tissue of an animal comprising administering an effective retroviral inhibiting amount of a pharmaceutical composition according to claim 75.

81. A method according to claim 80, wherein said retroviral infection does not respond to other therapies.

82. The method according to claim 78 wherein said composition is administered to provide said compound in an amount ranging from about 0.1 mg/kg to about 100 mg/kg body weight.

83. The method according to claim 82 wherein said composition is administered to provide said compound in an amount ranging from about 1 mg/kg to about 50 mg/kg body weight.

84. The method according to claim 78 wherein said animal is a human.

85. A method of inhibiting a retroviral infection by contacting a cell with a compound of claim 1.

86. A method of preventing transmission of HIV infection from an HIV infected pregnant woman to a fetus, comprising administering to said woman and/or said fetus a retroviral inhibiting effective amount of a compound of claim 1 during pregnancy or immediately prior to, at, or subsequent to birth.

87. A method of preventing transmission of HIV infection during sexual intercourse, comprising applying a retroviral inhibiting effective amount of one or more compounds of claim 1 to vaginal or other mucosa prior to sexual intercourse.
